(19) United States
(12) Patent Application Publication
(10) Pub. No.: US 2025/0163075 A1
(43) Pub. Date: May 22, 2025

(54) HETEROBIFUNCTIONAL TARGETED PROTEIN DEGRADERS

(71) Applicants: St. Jude Children's Research Hospital, Memphis, TN (US); Max-Planck-Gesellschaft zur Förderung der Wissenschaften e.V., Munich (DE)

(72) Inventors: Richard E. Lee, Cordova, TN (US); Suresh Dharuman, Collierville, TN (US); Daniel C. Scott, Marion, AR (US); Jason M. Ochoada, Collierville, TN (US); Rajendra P. Tangallapally, Collierville, TN (US); Brenda A. Schulman, Munich (DE)

(21) Appl. No.: 18/955,510

(22) Filed: Nov. 21, 2024

Related U.S. Application Data
(60) Provisional application No. 63/601,891, filed on Nov. 22, 2023.

(51) Int. Cl. C07D 495/14; A61K 31/496; A61K 31/506; A61K 31/551; C07D 417/14; C07D 471/04; C07D 519/00

(52) U.S. Cl. CPC C07D 495/14; A61K 31/496; A61K 31/506; A61K 31/551; C07D 417/14; C07D 471/04; C07D 519/00

(57) ABSTRACT

The present disclosure relates to compounds that bind to the kelch domain-containing protein 2 (KLHDC2) E3 ligase active site and heterobifunctional targeted protein degraders comprising the compounds. Methods of using these degraders in the treatment of cancer is also described. This abstract is intended as a scanning tool for purposes of searching in the particular art and is not intended to be limiting of the present invention.

### CROSS-REFERENCE TO RELATED APPLICATIONS

This application claims the benefit of U.S. Application No. 63/601,891, filed on Nov. 22, 2023, the contents of which are incorporated herein by reference in their entirety.

### STATEMENT REGARDING FEDERALLY SPONSORED RESEARCH

This invention was made with government support under grant number CA021765 awarded by the National Institutes of Health. The government has certain rights in the invention.

### BACKGROUND

The ubiquitin-proteasome system (UPS) plays a cardinal role in maintaining intracellular protein homeostasis by eliminating misfolded, damaged, and worn-out proteins (Amm et al. (2014) J Med Chem 57: 10486-10498). This process consists of a cascade of distinct steps, starting with ubiquitin activation by enzyme E1. Ubiquitin is then passed to the E2 or ubiquitin-conjugating enzyme by trans-thioesterification. Subsequently, E3 ubiquitin ligase promotes the transfer of ubiquitin onto a lysine of the substrate protein. Ubiquitin's own internal lysine residues allow binding of additional ubiquitins, resulting in polyubiquitin tags, which serve as a signal for protein degradation via the 26S proteasome (Kleiger and Mayor (2014) Trends Cel Biol 24: 352-359).

Hijacking the UPS and utilizing its functions to degrade the selected protein of interest (POI) has been made possible by proteolysis-targeting chimeras (PROTACs) (Burslem and Crews (2020) Cell 181: 102-114). These hetero-bifunctional molecules are composed of a POI ligand connected to an E3 ubiquitin ligase ligand by a linker (Pettersson and Crews (2019) Drug Discov Today Tech 31: 15-27). A functional PROTAC instigates the formation of a ternary complex POI-PROTAC-E3 ligase, which results in the ubiquitination of the POI, followed by proteasomal degradation (Scheepstra et al. (2019) Comput Struct Biotechnol J 17: 160-176). This new modality began accumulating recognition and significance in medicinal chemistry since 2001 when the first proof-of-concept experiments were published (Sakamoto et al. (2001) Proc Natl Acad Sci 98: 8554-8559; Burslem and Crews (2020) Cell 181: 102-114).

The human genome includes two members of the E1 enzyme family, roughly 40 E2s, and more than 600 E3 ubiquitin ligases (Kleiger and Mayor (2014) Trends Cel Biol 24: 352-359). E3 ligases represent a crucial element in protein ubiquitination due to their role in substrate selection and modulation of the cascade's efficiency (Buetow and Huang (2016) Nat Rev Mol Cel Biol 17: 626-642; Zheng and Shabek (2017) Annu Rev Biochem 86: 129-157). They are categorized into three classes, based on their mechanism of ubiquitin transfer. The first and the most abundant class includes approximately 600 RING (Really Interesting New Gene) E3 ligases, which catalyze the direct transfer of ubiquitin from E2 to a substrate. In contrast, the less represented E3 classes HECT (Homologous to E6AP C-terminus) and RBR (RING-between-RING) form a thioester intermediate with ubiquitin via a catalytic cysteine before the transfer to the substrate protein (Buetow and Huang (2016) Nat Rev Mol Cel Biol 17: 626-642). Although the understanding of substrate recognition and regulation of ubiquitination is incomplete, the genome's selection of roughly 600 E3 ligases is capable of ubiquitinating a much larger number of protein substrates in a controlled manner with ample specificity (Fisher and Phillips (2018) Curr Opin Chem Biol 44: 47-55).

Despite the vast selection of known E3 ligases, only a handful have been successfully utilized in PROTAC compounds (Burslem and Crews (2020) Cell 181: 102-114). Thus, there remains a need for compounds that selectively bind an E3 ligase and uses thereof to generate targeted heterobifunctional compounds.

### SUMMARY

In accordance with the purpose(s) of the invention, as embodied and broadly described herein, the invention, in one aspect, relates to compounds that bind to the kelch domain-containing protein 2 (KLHDC2) E3 ligase active site, heterobifunctional targeted protein degraders comprising the compounds, and methods of using these protein degraders in the treatment of various disorders such as, for example, cancer.

Thus, in one aspect, disclosed are compounds having a structure represented by a formula:

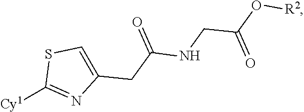

wherein R² is selected from hydrogen, C1-C4 alkyl, C4-C6 cycloalkyl, —(C1-C4 alkyl)OC(O)(C1-C4 alkyl), and —CH₂C₆H₅; wherein Cy¹ is a 9- or 10-membered heterobicycle, a 10-membered biaryl, or a 9- or 10-membered heterobiaryl, and is substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹; wherein each occurrence of R¹⁰, when present, is independently selected from hydrogen, C1-C4 alkyl, —(C1-C4 alkyl)O(C1-C4 alkyl), —(C1-C4 alkyl)O(C1-C4 alkyl)N₃, and —(C1-C4 alkyl)Cy²; wherein Cy², when present, is a C2-C5 heterocycloalkyl substituted with 0, 1, 2, or 3 groups independently selected from halogen, —N₃, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 alkoxy, C1-C4 alkylamino, (C1-C4)(C1-C4) dialkylamino, C1-C4 aminoalkyl, —C(O)H, —C(O)(C1-C4 alkyl), —CO₂H, and —CO₂(C1-C4 alkyl); wherein each occurrence of R¹¹, when present, is independently selected from C1-C4 alkyl, C1-C4 hydroxyalkyl, and Ar¹; and wherein Ar¹, when present, is a C6 aryl or a C2-C5 heteroaryl, and is substituted with 0, 1, 2, or 3 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl, or a pharmaceutically acceptable salt thereof.

Also disclosed are compounds having a structure represented by a formula:

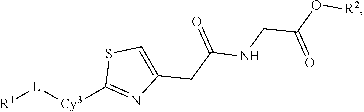

wherein L is a linker; wherein R¹ is a residue of a small molecule having a molecular weight of from about 150 g/mol to about 600 g/mol and a binding affinity (Kᵢ) to a target protein of at least about 20 μM; wherein R² is selected from hydrogen, C1-C4 alkyl, C4-C6 cycloalkyl, —(C1-C4 alkyl)OC(O)(C1-C4 alkyl), and —CH₂C₆H₅; and wherein Cy³ is a 9- or 10-membered heterobicycle, a 10-membered biaryl, or a 9- or 10-membered heterobiaryl, and is substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹; wherein each occurrence of R¹⁰, when present, is independently selected from hydrogen, C1-C4 alkyl, —(C1-C4 alkyl)O(C1-C4 alkyl), —(C1-C4 alkyl)O(C1-C4 alkyl)N₃, and —(C1-C4 alkyl)Cy²; wherein Cy², when present, is a C2-C5 heterocycloalkyl substituted with 0, 1, 2, or 3 groups independently selected from halogen, —N₃, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 alkoxy, C1-C4 alkylamino, (C1-C4)(C1-C4) dialkylamino, C1-C4 aminoalkyl, —C(O)H, —C(O)(C1-C4 alkyl), —CO₂H, and —CO₂(C1-C4 alkyl); wherein each occurrence of R¹¹, when present, is independently selected from C1-C4 alkyl, C1-C4 hydroxyalkyl, and Ar¹; wherein Ar¹, when present, is a C6 aryl or a C2-C5 heteroaryl, and is substituted with 0, 1, 2, or 3 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl, or a pharmaceutically acceptable salt thereof.

Also disclosed are pharmaceutical compositions comprising a therapeutically effective amount of a disclosed compound or a pharmaceutically acceptable salt thereof, and a pharmaceutically acceptable carrier.

Also disclosed are methods of degrading a target protein in a cell using the ubiquitin E3 ligase KLHDC2, the method comprising contacting the cell with an effective amount of a disclosed compound.

Also disclosed are methods of degrading a target protein in a subject in need thereof, the method comprising administering to the subject an effective amount of a disclosed compound.

Also disclosed are methods of treating a disorder of uncontrolled cellular proliferation in a subject in need thereof, the method comprising administering to the subject an effective amount of a disclosed compound or a pharmaceutically acceptable salt thereof.

Also disclosed are kits comprising a disclosed compound, and one or more selected from: (a) an agent known to treat a disorder of uncontrolled cellular proliferation; (b) instructions for administering the compound in connection with treating a disorder of uncontrolled cellular proliferation; and (c) instructions for treating a disorder of uncontrolled cellular proliferation.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The accompanying figures, which are incorporated in and constitute a part of this specification, illustrate several aspects and together with the description serve to explain the principles of the invention.

FIG. 1A-D shows representative data demonstrating that the exemplary compounds exhibit targeted protein degradation. Specifically, an ubiquitylation assay was conducted to monitor the ability of DCN1-5377-KLHDC2 ternary complex to inhibit ubiquitylation of a model KLHDC2 substrate (see FIG. 1A, left). Referring to FIG. 1A, right, the activity of model substrate ubiquitylation was quantified and normalized to 100% in the absence of a ternary complex. Data was fit to the Morrision equation to estimate Ki. The data in FIG. 1B was generated the same as for FIG. 1A, but with DCN1-5393-KLHDC2 or DCN2-5393-KLHDC2, respectively. Referring to FIG. 1C, U2OS cells were dosed with 2 mM of the indicated PROTACs. 24 hours later, cells were harvested and subjected to western blotting with the indicated antibodies. The data in FIG. 1D were generated the same as for FIG. 1C, but, when indicated, cells were dosed alone or in combination with 1 mM MLN4924.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage form can comprise a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO-moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. For example, the cycloalkyl group and heterocycloalkyl group can be substituted with 0, 1, 2, 3, or 4 groups independently selected from C1-C4 alkyl, C3-C7 cycloalkyl, C1-C4 alkoxy, —NH₂, (C1-C4) alkylamino, (C1-C4)(C1-C4) dialkylamino, ether, halogen, —OH, C1-C4 hydroxyalkyl, —NO₂, silyl, sulfo-oxo, —SH, and C1-C4 thioalkyl, as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. For example, the cycloalkenyl group and heterocycloalkenyl group can be substituted with 0, 1, 2, 3, or 4 groups independently selected from C1-C4 alkyl, C3-C7 cycloalkyl, C1-C4 alkoxy, C2-C4 alkenyl, C3-C6 cycloalkenyl, C2-C4 alkynyl, aryl, heteroaryl, aldeyhyde, —NH₂, (C1-C4) alkylamino, (C1-C4)(C1-C4) dialkylamino, carboxylic acid, ester, ether, halogen, —OH, C1-C4 hydroxyalkyl, ketone, azide, —NO₂, silyl, sulfo-oxo, —SH, and C1-C4 thioalkyl, as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” or “CO” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The terms “pseudohalide,” “pseudohalogen,” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl.

Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl”, “heteroaryl”, “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxy” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN or —C≡N.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A'S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position.

Combinations of substituents envisioned by this invention are those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{∘}3, —OSiR^{∘}3, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

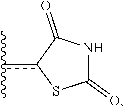

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

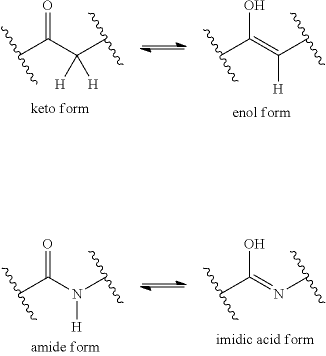 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N-unsubstituted, 5-A³ as shown below.

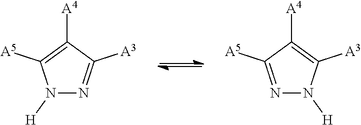

Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

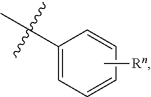

which is understood to be equivalent to a formula:

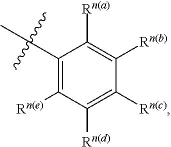

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

In one aspect, disclosed are compounds that bind to the kelch domain-containing protein 2 (KLHDC2) E3 ligase active site, which can be used to prepare heterobifunctional targeted protein degraders. In a further aspect, the disclosed heterobifunctional targeted protein degraders can be useful in treating a variety of different disorders including, but not limited to, cancer.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, disclosed are compounds having a structure represented by a formula:

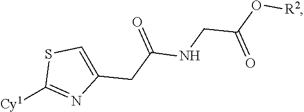

wherein R² is selected from hydrogen, C1-C4 alkyl, C4-C6 cycloalkyl, —(C1-C4 alkyl)OC(O)(C1-C4 alkyl), and —CH₂C₆H₅; wherein Cy¹ is a 9- or 10-membered heterobicycle, a 10-membered biaryl, or a 9- or 10-membered heterobiaryl, and is substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹; wherein each occurrence of R¹⁰, when present, is independently selected from hydrogen, C1-C4 alkyl, —(C1-C4 alkyl)O(C1-C4 alkyl), —(C1-C4 alkyl)O(C1-C4 alkyl)N₃, and —(C1-C4 alkyl)Cy²; wherein Cy², when present, is a C2-C5 heterocycloalkyl substituted with 0, 1, 2, or 3 groups independently selected from halogen, —N₃, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 alkoxy, C1-C4 alkylamino, (C1-C4)(C1-C4) dialkylamino, C1-C4 aminoalkyl, —C(O)H, —C(O)(C1-C4 alkyl), —CO₂H, and —CO₂(C1-C4 alkyl); wherein each occurrence of R¹¹, when present, is independently selected from C1-C4 alkyl, C1-C4 hydroxyalkyl, and Ar¹; and wherein Ar¹, when present, is a C6 aryl or a C2-C5 heteroaryl, and is substituted with 0, 1, 2, or 3 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl, or a pharmaceutically acceptable salt thereof.

In various aspects, the compound has a structure represented by a formula selected from:

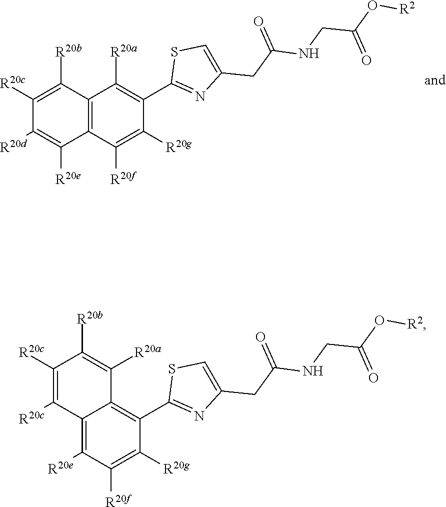

wherein each of R^{20a}, R^{20b}, R^{20c}, R^{20d}, R^{20e}, R^{20f}, and R^{20g} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹, provided that at least three of R^{20a}, R^{20b}, R^{20c}, R^{20d}, R^{20e}, R^{20f}, and R^{20g} are hydrogen, —or a pharmaceutically acceptable salt thereof. In a further aspect, each occurrence of R¹⁰, when present, is —(C1-C4 alkyl)O(C1-C4 alkyl). In a still further aspect, each of R^{20a}, R^{20b}, R^{20c}, R^{20d}, R^{20e}, R^{20f}, and R^{20g} is independently selected from hydrogen, halogen, and C1-C4 alkyl. In a still further aspect, R^{20b} is selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In yet a further aspect, each of R^{20a}, R^{20c}, R^{20d}, R^{20e}, R^{20f}, and R^{20g} is hydrogen.

In various aspects, R^{20c} is selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, each of R^{20a}, R^{20b}, R^{20d}, R^{20e}, R^{20f}, and R^{20g} is hydrogen.

In various aspects, the compound has a structure represented by a formula:

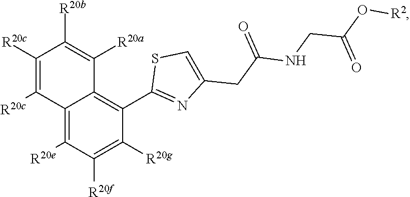

or a pharmaceutically acceptable salt thereof.

In various aspects, the compound has a structure represented by a formula:

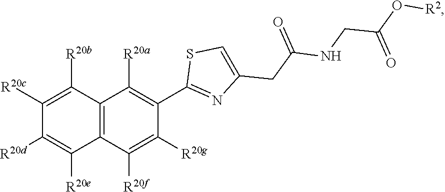

or a pharmaceutically acceptable salt thereof.

In various aspects, the compound has a structure represented by a formula selected from:

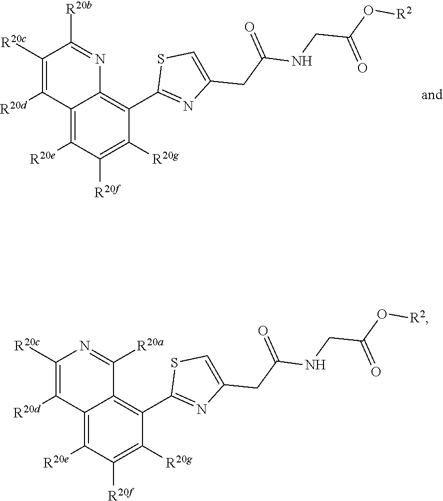

wherein each of R^{20a}, R^{20b}, R^{20c}, R^{20d}, R^{20e}, R^{20f}, and R^{20g} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹, provided that at least two of R^{20a}, R^{20b}, R^{20c}, R^{20d}, R^{20e}, R^{20f}, and R^{20g} are hydrogen, or a pharmaceutically acceptable salt thereof. In a further aspect, each occurrence of R¹⁰, when present, is —(C1-C4 alkyl)O(C1-C4 alkyl). In a still further aspect, each of R^{21a}, R^{21b}, and R^{21c} is independently selected from hydrogen, halogen, and C1-C4 alkyl.

In various aspects, the compound has a structure represented by a formula selected from:

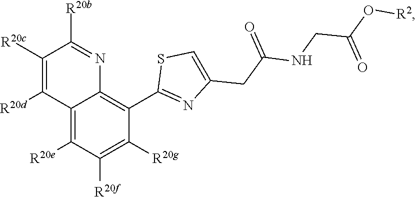

or a pharmaceutically acceptable salt thereof.

In various aspects, the compound has a structure represented by a formula selected from:

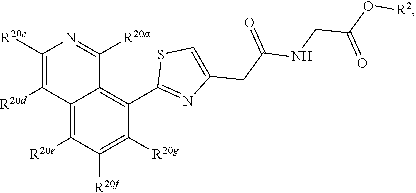

or a pharmaceutically acceptable salt thereof.

In various aspects, the compound has a structure represented by a formula:

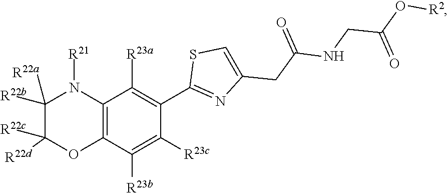

wherein R²¹ is selected from hydrogen and C1-C4 alkyl; and wherein each of R^{22a}, R^{22b}, R^{22c}R^{22a}, R^{23a}, R^{23b}, and R^{23c} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹, provided that at least three of R²¹, R^{22a}, R^{22b}, R^{22c}, R^{22a}, R^{23a}, R^{23b}, and R^{23c} are hydrogen, or a pharmaceutically acceptable salt thereof. In a further aspect, each occurrence of R¹⁰, when present, is —(C1-C4 alkyl)O(C1-C4 alkyl). In a still further aspect, each of R^{22a}, R^{22b}, R^{22c}, R^{22d}, R^{23a}, R^{23b}, and R^{23c} is independently selected from hydrogen, halogen, and C1-C4 alkyl. In yet a further aspect, each of R^{20a}, R^{20c}, R^{20d}, R^{20e}, R^{20f}, and R^{20g} is hydrogen.

In various aspects, the compound has a structure represented by a formula:

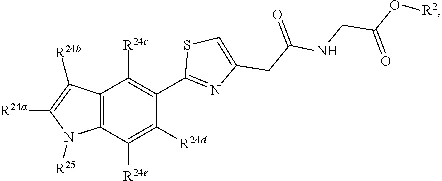

wherein each of R^{24a}, R^{24b}, R^{24c}, R^{24d}, and R^{24e} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹; and wherein R²⁵ is selected from hydrogen and C1-C4 alkyl, provided that at least two of R^{24a}, R^{24b}, R^{24c}, R^{24d}, R^{24e}, and R²⁵ are hydrogen, or a pharmaceutically acceptable salt thereof. In a further aspect, each occurrence of R¹⁰, when present, is —(C1-C4 alkyl)O(C1-C4 alkyl). In a still further aspect, each of R^{24a}, R^{24b}, R^{24c}, R^{24d}, R^{24e}, and R²⁵ is independently selected from hydrogen, halogen, and C1-C4 alkyl. In yet a further aspect, each of R^{24a}, R^{24b}, R^{24c}, R^{24d}, R^{24e}, and R²⁵ is hydrogen.

In various aspects, the compound is selected from:

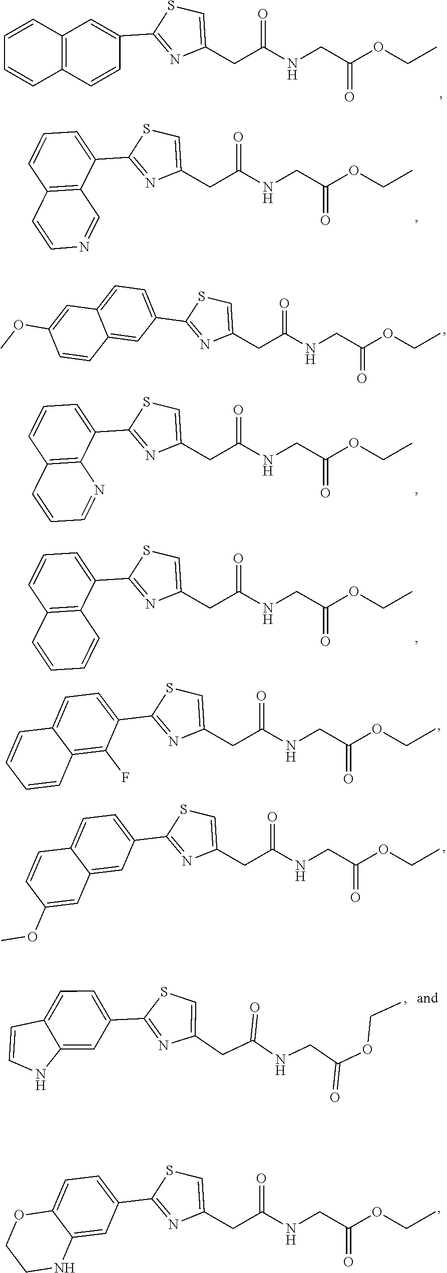

or a pharmaceutically acceptable salt thereof.

In various aspects, the compound is selected from:

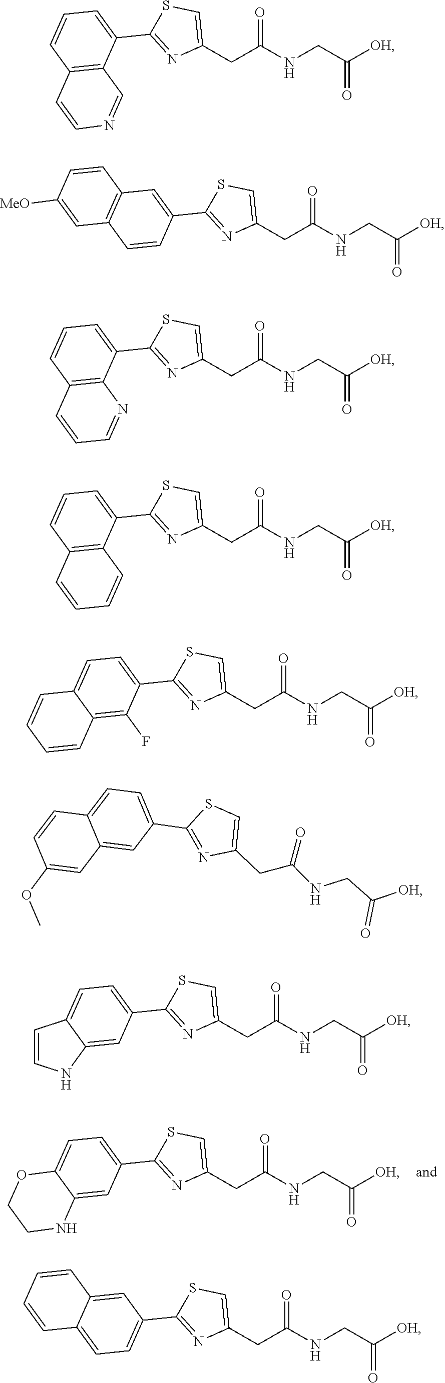

or a pharmaceutically acceptable salt thereof.

In various aspects, the compound is selected from:

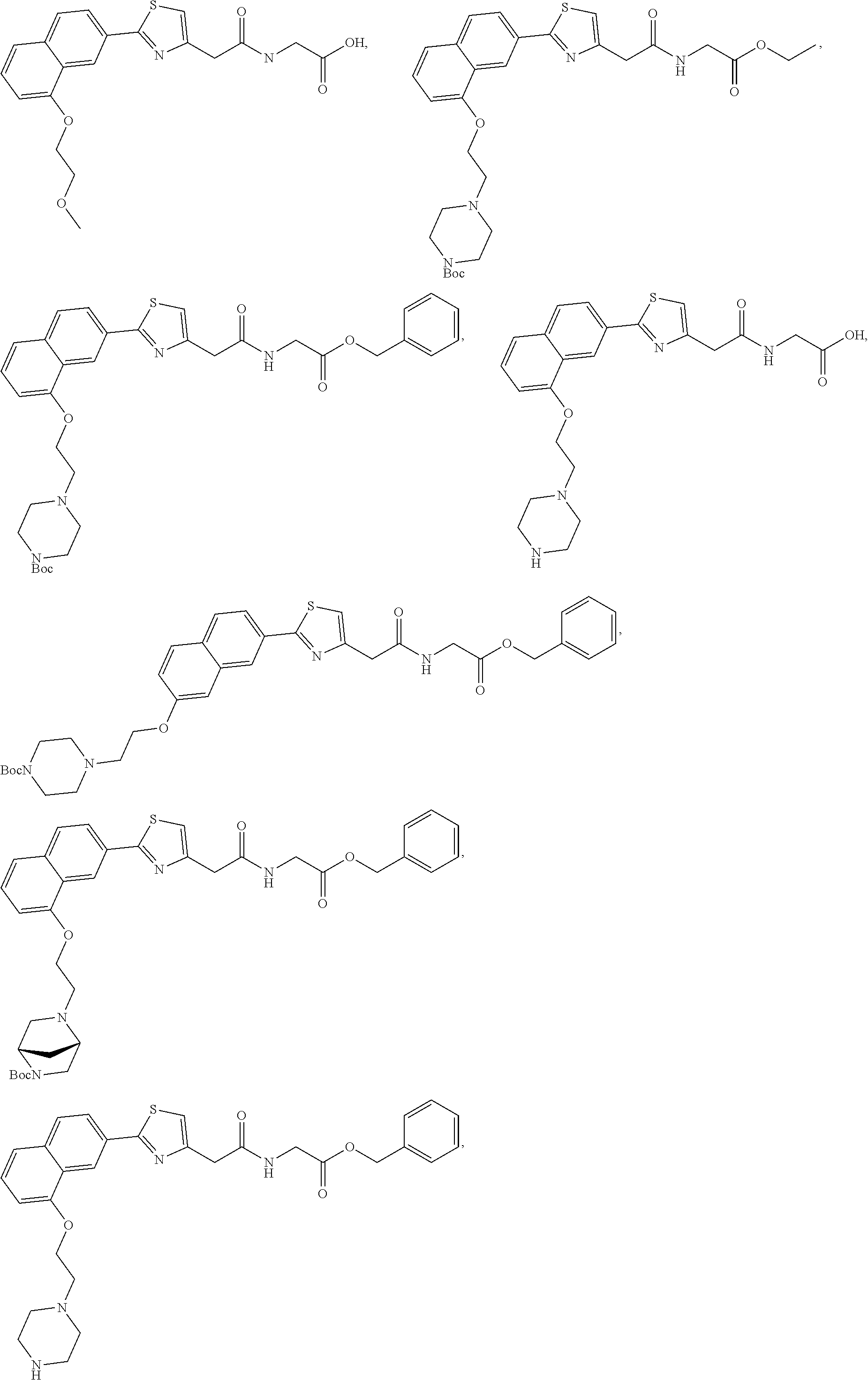

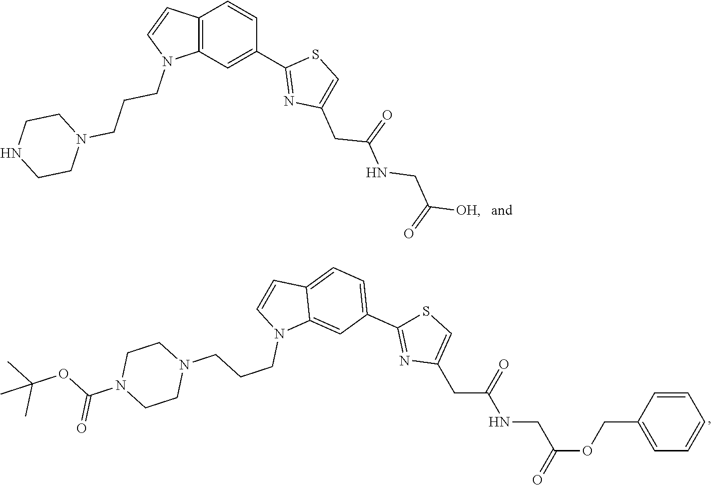 [SMILES: O=C(O)CNC(=O)Cc1csc(-c2ccc3ccn(CCCN4CCNCC4)c3c2)n1 ; "and" ; CC(C)(C)OC(=O)N1CCN(CCCn2ccc3ccc(-c4nc(CC(=O)NCC(=O)OCc5ccccc5)cs4)cc32)CC1]

or a pharmaceutically acceptable salt thereof.

### A. R² Groups

In one aspect, R² is selected from hydrogen, C1-C4 alkyl, C4-C6 cycloalkyl, —(C1-C4 alkyl)OC(O)(C1-C4 alkyl), and —CH₂C₆H₅. In a further aspect, R² is selected from hydrogen, methyl, ethyl, n-propyl, isopropyl, cyclobutyl, cyclopentyl, —CH₂OC(O)CH₃, —CH₂CH₂OC(O)CH₃, —CH₂CH₂OC(O)CH₂CH₃, —CH₂OC(O)CH(CH₃)₂, —CH₂CH₂OC(O)CH₂CH₂CH₃, —CH₂OC(O)CH₂CH₂CH₃, and —CH₂C₆H₅. In a still further aspect, R² is selected from hydrogen, methyl, ethyl, cyclopentyl, —CH₂OC(O)CH₃, —CH₂CH₂OC(O)CH₃, —CH₂CH₂OC(O)CH₂CH₃, and —CH₂C₆H₅. In yet a further aspect, R² is selected from hydrogen, methyl, cyclopentyl, —CH₂OC(O)CH₃, and —CH₂C₆H₅.

In various aspects, R² is selected from C1-C4 alkyl and C4-C6 cycloalkyl. In a further aspect, R² is selected from methyl, ethyl, n-propyl, isopropyl, cyclobutyl, and cyclopentyl. In a still further aspect, R² is selected from methyl, ethyl, cyclobutyl, and cyclopentyl. In yet a further aspect, R² is selected from methyl and cyclopentyl.

In various aspects, R² is selected from hydrogen and C4-C6 cycloalkyl. In a further aspect, R² is selected from hydrogen, cyclobutyl, and cyclopentyl. In a still further aspect, R² is selected from hydrogen and cyclobutyl. In yet a further aspect, R² is selected from hydrogen and cyclopentyl.

In various aspects, R² is C4-C6 cycloalkyl. In a further aspect, R² is cyclobutyl and cyclopentyl. In a still further aspect, R² is cyclobutyl. In yet a further aspect, R² is cyclopentyl.

In various aspects, R² is selected from hydrogen and C1-C4 alkyl. In a further aspect, R² is selected from hydrogen, methyl, ethyl, n-propyl, and isopropyl. In a still further aspect, R² is selected from hydrogen, methyl, and ethyl. In yet a further aspect, R² is selected from hydrogen and methyl.

In various aspects, R² is C1-C4 alkyl. In a further aspect, R² is selected from methyl, ethyl, n-propyl, and isopropyl. In a still further aspect, R² is selected from methyl and ethyl. In yet a further aspect, R² is methyl.

In various aspects, R² is —CH₂C₆H₅.

In various aspects, R² is selected from hydrogen, methyl, cyclopentyl, and —CH₂C₆H₅. In a further aspect, R² is selected from methyl, cyclopentyl, and —CH₂C₆H₅.

In various aspects, R² is hydrogen.

b. R¹⁰ Groups

In one aspect, each occurrence of R¹⁰, when present, is independently selected from hydrogen, C1-C4 alkyl, —(C1-C4 alkyl)O(C1-C4 alkyl), —(C1-C4 alkyl)O(C1-C4 alkyl)N₃, and —(C1-C4 alkyl)Cy². In a further aspect, each occurrence of R¹⁰, when present, is independently selected from hydrogen, methyl, ethyl, n-propyl, isopropyl, —CH₂OCH₃, —CH₂CH₂OCH₃, —CH₂CH₂OCH₂CH₃, —CH₂OCH(CH₃)₂, —CH₂CH₂OCH₂CH₂CH₃, —CH₂OCH₂CH₂CH₃, —CH₂OCH₂N₃, —CH₂CH₂OCH₂N₃, —CH₂CH₂OCH₂CH₂N₃, —CH₂OCH(CH₃)CH₂N₃, —CH₂CH₂OCH₂CH₂CH₂N₃, —CH₂OCH₂CH₂CH₂N₃, —CH₂Cy², —CH₂CH₂Cy², —CH(CH₃)CH₂Cy², and —CH₂CH₂CH₂Cy². In a still further aspect, each occurrence of R¹⁰, when present, is independently selected from hydrogen, methyl, ethyl, —CH₂OCH₃, —CH₂CH₂OCH₃, —CH₂CH₂OCH₂CH₃, —CH₂OCH₂N₃, —CH₂CH₂OCH₂N₃, —CH₂CH₂OCH₂CH₂N₃, —CH₂Cy², and —CH₂CH₂Cy². In yet a further aspect, each occurrence of R¹⁰, when present, is independently selected from hydrogen, methyl, —CH₂OCH₃, —CH₂OCH₂N₃, and —CH₂Cy².

In various aspects, each occurrence of R¹⁰, when present, is selected from —(C1-C4 alkyl)O(C1-C4 alkyl) and —(C1-C4 alkyl)O(C1-C4 alkyl)N₃. In a further aspect, each occurrence of R¹⁰, when present, is independently selected from —CH₂OCH₃, —CH₂CH₂OCH₃, —CH₂CH₂OCH₂CH₃, —CH₂OCH(CH₃)₂, —CH₂CH₂OCH₂CH₂CH₃, —CH₂OCH₂CH₂CH₃, —CH₂OCH₂N₃, —CH₂CH₂OCH₂N₃, —CH₂CH₂OCH₂CH₂N₃, —CH₂OCH(CH₃)CH₂N₃, —CH₂CH₂OCH₂CH₂CH₂N₃, and —CH₂OCH₂CH₂CH₂N₃. In a still further aspect, each occurrence of R¹⁰, when present, is independently selected from —CH₂OCH₃, —CH₂CH₂OCH₃, —CH₂CH₂OCH₂CH₃, —CH₂OCH₂N₃, —CH₂CH₂OCH₂N₃, and —CH₂CH₂OCH₂CH₂N₃. In yet a further aspect, each occurrence of R¹⁰, when present, is independently selected from —CH₂OCH₃ and —CH₂OCH₂N₃.

In various aspects, each occurrence of R¹⁰, when present, is selected from —CH₂OCH₃, —CH₂CH₂OCH₃, and —CH₂CH₂OCH₂CH₂N₃. In a further aspect, each occurrence of R¹⁰, when present, is selected from —CH₂OCH₃ and —CH₂CH₂OCH₂CH₂N₃. In a still further aspect, each occurrence of R¹⁰, when present, is selected from —CH₂CH₂OCH₃ and —CH₂CH₂OCH₂CH₂N₃. In yet a further aspect, each occurrence of R¹⁰, when present, is selected from —CH₂OCH₃ and —CH₂CH₂OCH₃.

In various aspects, each occurrence of R¹⁰, when present, is —(C1-C4 alkyl)O(C1-C4 alkyl). In a further aspect, each occurrence of R¹⁰, when present, is independently selected from —CH₂OCH₃, —CH₂CH₂OCH₃, —CH₂CH₂OCH₂CH₃, —CH₂OCH(CH₃)₂, —CH₂CH₂OCH₂CH₂CH₃, and —CH₂OCH₂CH₂CH₃. In a still further aspect, each occurrence of R¹⁰, when present, is independently selected from —CH₂OCH₃, —CH₂CH₂OCH₃, and —CH₂CH₂OCH₂CH₃. In yet a further aspect, each occurrence of R¹⁰, when present, is —CH₂OCH₃.

In various aspects, each occurrence of R¹⁰, when present, is independently selected from hydrogen and C1-C4 alkyl. In a further aspect, each occurrence of R¹⁰, when present, is independently selected from hydrogen, methyl, ethyl, n-propyl, and isopropyl. In a still further aspect, each occurrence of R¹⁰, when present, is independently selected from hydrogen, methyl, and ethyl. In yet a further aspect, each occurrence of R¹⁰, when present, is independently selected from hydrogen and ethyl. In an even further aspect, each occurrence of R¹⁰, when present, is independently selected from hydrogen and methyl.

In various aspects, each occurrence of R¹⁰, when present, is C1-C4 alkyl. In a further aspect, each occurrence of R¹⁰, when present, is independently selected from methyl, ethyl, n-propyl, and isopropyl. In a still further aspect, each occurrence of R¹⁰, when present, is independently selected from methyl and ethyl. In yet a further aspect, each occurrence of R¹⁰, when present, is ethyl. In an even further aspect, each occurrence of R¹⁰, when present, is methyl.

In various aspects, each occurrence of R¹⁰, when present, is hydrogen.

c. R¹¹ Groups

In one aspect, each occurrence of R¹¹, when present, is independently selected from C1-C4 alkyl, C1-C4 hydroxyalkyl, and Ar². In a further aspect, each occurrence of R¹¹ when present, is independently selected from methyl, ethyl, n-propyl, isopropyl, —CH₂OH, —CH₂CH₂OH, —CH(CH₃)CH₂OH, —CH₂CH₂CH₂OH, and Ar². In a still further aspect, each occurrence of R¹¹, when present, is independently selected from methyl, ethyl, —CH₂OH, —CH₂CH₂OH, and Ar². In yet a further aspect, each occurrence of R¹¹, when present, is independently selected from methyl, —CH₂OH, and Ar².

In various aspects, each occurrence of R¹¹, when present, is independently selected from C1-C4 alkyl and C1-C4 hydroxyalkyl. In a further aspect, each occurrence of R¹¹, when present, is independently selected from methyl, ethyl, n-propyl, isopropyl, —CH₂OH, —CH₂CH₂OH, —CH(CH₃)CH₂OH, and —CH₂CH₂CH₂OH. In a still further aspect, each occurrence of R¹¹, when present, is independently selected from methyl, ethyl, —CH₂OH, and —CH₂CH₂OH. In yet a further aspect, each occurrence of R¹¹, when present, is independently selected from methyl and —CH₂OH.

In various aspects, each occurrence of R¹¹, when present, is independently selected from C1-C4 alkyl and Ar². In a further aspect, each occurrence of R¹¹, when present, is independently selected from methyl, ethyl, n-propyl, isopropyl, and Ar². In a still further aspect, each occurrence of R¹¹, when present, is independently selected from methyl, ethyl, and Ar². In yet a further aspect, each occurrence of R¹¹, when present, is independently selected from methyl and Ar².

In various aspects, each occurrence of R¹¹, when present, is independently selected from C1-C4 hydroxyalkyl and Ar². In a further aspect, each occurrence of R¹¹, when present, is independently selected from —CH₂OH, —CH₂CH₂OH, —CH(CH₃)CH₂OH, —CH₂CH₂CH₂OH, and Ar². In a still further aspect, each occurrence of R¹¹, when present, is independently selected from —CH₂OH, —CH₂CH₂OH, and Ar². In yet a further aspect, each occurrence of R¹¹, when present, is independently selected from —CH₂OH and Ar².

In various aspects, each occurrence of R¹¹, when present, is independently C1-C4 hydroxyalkyl. In a further aspect, each occurrence of R¹¹, when present, is independently selected from —CH₂OH, —CH₂CH₂OH, —CH(CH₃)CH₂OH, and —CH₂CH₂CH₂OH. In a still further aspect, each occurrence of R¹¹, when present, is independently selected from —CH₂OH and —CH₂CH₂OH. In yet a further aspect, each occurrence of R¹¹, when present, is —CH₂OH.

In various aspects, each occurrence of R¹¹, when present, is independently C1-C4 alkyl. In a further aspect, each occurrence of R¹¹, when present, is independently selected from methyl, ethyl, n-propyl, and isopropyl. In a still further aspect, each occurrence of R¹¹ when present, is independently selected from methyl and ethyl. In yet a further aspect, each occurrence of R¹¹, when present, is methyl.

In various aspects, each occurrence of R¹¹, when present, is Ar².

d. R^{20A}, R^{20B}, R^{20C}, R^{20D}, R^{20E}, R^{20F}, and R^{20G} Groups

In one aspect, each of R^{20a}, R^{20b}, R^{20c}, R^{20d}, R^{20e}, R^{20f} and R^{20g} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹, provided that at least three of R^{20a}, R^{20b}, R^{20c}, R^{20d}, R^{20e}, R^{20f} and R^{20g} are hydrogen. In a further aspect, each of R^{20a}, R^{20b}, R^{20c}, R^{20d}, R^{20e}, R^{20f} and R^{20g} is independently selected from hydrogen, —F, —Cl, —CN, —NH₂, —NO₂, methyl, ethyl, propyl, isopropyl, ethenyl, 1-propenyl, 2-propenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)(CF₃), —OCH₃, —OCH₂CH₃, —OCH₂CH₂CH₃, —OCH(CH₃)₂, —CH₂NH₂, —CH₂CH₂NH₂, —CH₂CH₂CH₂NH₂, —CH(CH₃)CH₂NH₂, —N(CH₃)₂, —N(CH₃)CH₂CH₃, —N(CH₃)CH₂CH₂CH₃, —N(CH₃)CH(CH₃)₂, —N(CH₂CH₃)₂, —N(CH₂CH₃)CH₂CH₂CH₃, —N(CH₂CH₃)CH(CH₃)₂, —N(CH₂CH₂CH₃)₂, —N(CH₂CH₂CH₃)CH(CH₃)₂, —N(CH(CH₃)₂)₂, —NHCH₃, —NHCH₂CH₃, —NHCH₂CH₂CH₃, —NHCH(CH₃)₂, —OR¹⁰, and —NHC(O)R¹¹. In a further aspect, each of R^{20a}, R^{20b}, R^{20c}, R^{20d}, R^{20e}, R^{20f} and R^{20g} is independently selected from hydrogen, —F, —Cl, —CN, —NH₂, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —CH₂NH₂, —CH₂CH₂NH₂, —N(CH₃)₂, —N(CH₃)CH₂CH₃, —N(CH₂CH₃)₂, —NHCH₃, —NHCH₂CH₃, —OR¹⁰, and —NHC(O)R¹¹. In a still further aspect, each of R^{20a}, R^{20b}, R^{20c}, R^{20d}, R^{20e}, R^{20f} and R^{20g} is independently selected from hydrogen, —F, —Cl, —CN, —NH₂, —NO₂, methyl, —CH₂F, —CH₂Cl, —CH₂CN, —CH₂OH, —OCF₃, —OCH₃, —CH₂NH₂, —N(CH₃)₂, —NHCH₃, —OR¹⁰, and —NHC(O)R¹¹.

In one aspect, each of R^{20a}, R^{20b}, R^{20c}, R^{20d}, R^{20e}, R^{20f} and R^{20g} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹, provided that at least two of R^{20a}, R^{20b}, R^{20e}, R^{20a}, R^{20e}, R^{20f}, and R^{20g} are hydrogen. In various aspects, each of R²⁰, R^{20b}, R^{20e}, R^{20a}, R^{20e}, R^{20f} and R^{20g} is independently selected from hydrogen, —F, —Cl, —CN, —NH₂, —NO₂, methyl, ethyl, propyl, isopropyl, ethenyl, 1-propenyl, 2-propenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)(CF₃), —OCH₃, —OCH₂CH₃, —OCH₂CH₂CH₃, —OCH(CH₃)₂, —CH₂NH₂, —CH₂CH₂NH₂, —CH₂CH₂CH₂NH₂, —CH(CH₃)CH₂NH₂, —N(CH₃)₂, —N(CH₃)CH₂CH₃, —N(CH₃)CH₂CH₂CH₃, —N(CH₃)CH(CH₃)₂, —N(CH₂CH₃)₂, —N(CH₂CH₃)CH₂CH₂CH₃, —N(CH₂CH₃)CH(CH₃)₂, —N(CH₂CH₂CH₃)₂, —N(CH₂CH₂CH₃)CH(CH₃)₂, —N(CH(CH₃)₂)₂, —NHCH₃, —NHCH₂CH₃, —NHCH₂CH₂CH₃, —NHCH(CH₃)₂, —OR¹⁰, and —NHC(O)R¹¹. In a further aspect, each of R^{20a}, R^{20b}, R^{20e}, R^{20a}, R^{20e}, R^{20f} and R^{20g} is independently selected from hydrogen, —F, —Cl, —CN, —NH₂, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —CH₂NH₂, —CH₂CH₂NH₂, —N(CH₃)₂, —N(CH₃)CH₂CH₃, —N(CH₂CH₃)₂, —NHCH₃, —NHCH₂CH₃, —OR¹⁰, and —NHC(O)R¹¹. In a still further aspect, each of R^{20a}, R^{20b}R^{20e}, R^{20a}, R^{20e}, R^{20f} and R^{20g} is independently selected from hydrogen, —F, —Cl, —CN, —NH₂, —NO₂, methyl, —CH₂F, —CH₂Cl, —CH₂CN, —CH₂OH, —OCF₃, —OCH₃, —CH₂NH₂, —N(CH₃)₂, —NHCH₃, —OR¹⁰, and —NHC(O)R¹¹.

In various aspects, each of R^{20a}, R^{20b}, R^{20c}, R^{20d}, R^{20e}, R^{20f} and R^{20g} is independently selected from hydrogen, halogen, and C1-C4 alkyl. In a further aspect, each of R^{20a}, R^{20b}, R^{20c}, R^{20d}, R^{20e}, R^{20f} and R^{20g} is independently selected from hydrogen, —F, —Cl, methyl, ethyl, propyl, and isopropyl. In a still further aspect, each of R^{20a}, R^{20b}, R^{20c}, R^{20d}, R^{20e}, R^{20f}, and R^{20g} is independently selected from hydrogen, —F, —Cl, methyl, and ethyl. In yet a further aspect, each of R^{20a}, R^{20b}, R^{20c}, R^{20d}, R^{20e}, R^{20f} and R^{20g} is independently selected from hydrogen, —F, —Cl, and methyl.

In various aspects, each of R^{21a}, R^{21b}, and R^{21c} is independently selected from hydrogen, halogen, and C1-C4 alkyl. In a further aspect, each of R^{21a}, R^{21b}, and R^{21c} is independently selected from hydrogen, —F, —Cl, methyl, ethyl, propyl, and isopropyl. In a still further aspect, each of R^{21a}, R^{21b}, and R^{21c} is independently selected from hydrogen, —F, —Cl, methyl, and ethyl. In yet a further aspect, each of R^{21a}, R^{21b}, and R^{21c} is independently selected from hydrogen, —F, —Cl, and methyl.

In various aspects, each of R^{20a}, R^{20b}, R^{20c}, R^{20d}, R^{20e}, R^{20f}, and R^{20g} is independently selected from hydrogen and C1-C4 alkyl. In a further aspect, each of R^{20a}, R^{20b}, R^{20c}, R^{20d}, R^{20e}, R^{20f}, and R^{20g} is independently selected from hydrogen, methyl, ethyl, propyl, and isopropyl. In a still further aspect, each of R^{20a}, R^{20b}, R^{20c}, R^{20d}, R^{20e}, R^{20f}, and R^{20g} is independently selected from hydrogen, methyl, and ethyl. In yet a further aspect, each of R^{20a}, R^{20b}, R^{20c}, R^{20d}, R^{20e}, R^{20f}, and R^{20g} is independently selected from hydrogen and methyl.

In various aspects, each of R^{20a}, R^{20b}, R^{20c}, R^{20d}, R^{20e}, R^{20f}, and R^{20g} is independently selected from hydrogen and halogen. In a further aspect, each of R^{20a}, R^{20b}, R^{20c}, R^{20d}, R^{20e}, R^{20f}, and R^{20g} is independently selected from hydrogen, —F, —Cl, and —Br. In a still further aspect, each of R^{20a}, R^{20b}, R^{20c}, R^{20d}, R^{20e}, R^{20f}, and R^{20g} is independently selected from hydrogen, —F, and —Cl. In yet a further aspect, each of R^{20a}, R^{20b}, R^{20c}, R^{20d}, R^{20e}, R^{20f}, and R^{20g} is independently selected from hydrogen and —F. In an even further aspect, each of R^{20a}, R^{20b}, R^{20c}, R^{20a}, R^{20e}, R^{20f}, and R^{20g} is independently selected from hydrogen and —Cl.

In various aspects, each of R^{20a}, R^{20b}, R^{20c}, R^{20d}, R^{20e}, R^{20f}, and R^{20g} is independently selected from hydrogen, C1-C4 alkyl, and C2-C4 alkenyl. In a further aspect, each of R^{20a}, R^{20b}, R^{20c}, R^{20a}, R^{20e}, R^{20f}, and R^{20g} is independently selected from hydrogen, methyl, ethyl, propyl, isopropyl, ethenyl, 1-propenyl, and 2-propenyl. In a still further aspect, each of R^{20a}, R^{20b}, R^{20c}, R^{20d}, R^{20e}, R^{20f}, and R^{20g} is independently selected from hydrogen, methyl, ethyl, and ethenyl.

In various aspects, each of R^{20a}, R^{20b}, R^{20c}, R^{20d}, R^{20e}, R^{20f}, and R^{20g} is independently selected from hydrogen, halogen, and C1-C4 haloalkyl. In a further aspect, each of R^{20a}, R^{20b}, R^{20e}, R^{20a}, R^{20c}, R^{20d}, and R^{20g} is independently selected from hydrogen, —F, —C1, —CH₂F, —CH₂CH₂F, —CH₂CH₂CH₂F, —CH(CH₃)CH₂F, —CH₂Cl, —CH₂CH₂Cl, —CH₂CH₂CH₂Cl, and —CH(CH₃)CH₂Cl. In a still further aspect, each of R^{20a}, R^{20b}, R^{20c}, R^{20d}, R^{20e}, R^{20f}, and R^{20g} is independently selected from hydrogen, —F, —Cl, —CH₂F, —CH₂CH₂F, —CH₂Cl, and —CH₂CH₂Cl. In yet a further aspect, each of R^{20a}, R^{20b}, R^{20c}, R^{20d}, R^{20e}, R^{20f}, and R^{20g} is independently selected from hydrogen, —F, —Cl, —CH₂F, and —CH₂Cl.

In various aspects, each of R^{20a}, R^{20b}, R^{20c}, R^{20d}, R^{20e}, R^{20f} and R^{20g} is independently selected from hydrogen, —CN, and C1-C4 cyanoalkyl. In a further aspect, each of R²⁰, R^{20b}, R^{20c}, R^{20d}, R^{20e}, R^{20f} and R^{20g} is independently selected from hydrogen, —CN, —CH₂CN, —CH₂CH₂CN, —CH₂CH₂CH₂CN and —CH(CH₃)CH₂CN. In a still further aspect, each of R^{20a}, R^{20b}, R^{20c}, R^{20d}, R^{20e}, R^{20f} and R^{20g} is independently selected from hydrogen, —CN, —CH₂CN, and —CH₂CH₂CN. In yet a further aspect, each of R^{20a}, R^{20b}, R^{20c}, R^{20a}, R^{20e}, R^{20f}, and R^{20g} is independently selected from hydrogen, —CN, and —CH₂CN.

In various aspects, each of R^{20a}, R^{20b}, R^{20c}, R^{20d}, R^{20e}, R^{20f} and R^{20g} is independently selected from hydrogen, C1-C4 hydroxyalkyl, and —OR¹⁰. In a further aspect, each of R^{20a}, R^{20b}, R^{20c}, R^{20d}, R^{20e}, R^{20f} and R^{20g} is independently selected from hydrogen, —CH₂OH, —CH₂CH₂OH, —CH₂CH₂CH₂OH, —CH(CH₃)CH₂OH, and —OR¹⁰. In a still further aspect, each of R^{20a}, R^{20b}, R^{20c}, R^{20d}, R^{20e}, R^{20f} and R^{20g} is independently selected from hydrogen, —CH₂OH, —CH₂CH₂OH, and —OR¹⁰. In an even further aspect, each of R^{20a}, R^{20b}R^{20e}, R^{20a}, R^{20e}, R^{20f}, and R^{20g} is independently selected from hydrogen, —CH₂OH, and —OR¹⁰.

In various aspects, each of R^{20a}, R^{20b}, R^{20c}, R^{20d}, R^{20e}, R^{20f} and R^{20g} is independently selected from hydrogen, —NH₂, C1-C4 alkylamino, (C1-C4)(C1-C4) dialkylamino, C1-C4 aminoalkyl, and —NHC(O)R¹¹. In a further aspect, each of R^{20a}, R^{20b}R^{20c}, R^{20d}, R^{20e}, R^{20f}, and R^{20g} is independently selected from hydrogen, —NH₂, —N(CH₃)₂, —N(CH₃)CH₂CH₃, —N(CH₃)CH₂CH₂CH₃, —N(CH₃)CH(CH₃)₂, —N(CH₂CH₃)₂, —N(CH₂CH₃)CH₂CH₂CH₃, —N(CH₂CH₃)CH(CH₃)₂, —N(CH₂CH₂CH₃)₂, —N(CH₂CH₂CH₃)CH(CH₃)₂, —N(CH(CH₃)₂)₂, —NHCH₃, —NHCH₂CH₃, —NHCH₂CH₂CH₃, —NHCH(CH₃)₂, —CH₂NH₂, —CH₂CH₂NH₂, —CH₂CH₂CH₂NH₂, —CH(CH₃)CH₂NH₂, and —NHC(O)R¹¹. In a still further aspect, each of R^{20a}, R^{20b}, R^{20c}, R^{20d}, R^{20e}, R^{20f} and R^{20g} is independently selected from hydrogen, —NH₂, —N(CH₃)₂, —N(CH₃)CH₂CH₃, —N(CH₃)CH(CH₃)₂, —N(CH₂CH₃)₂, —NHCH₃, —NHCH₂CH₃, —CH₂NH₂, —CH₂CH₂NH₂, and —NHC(O)R¹¹. In an even further aspect, each of R^{20a}, R^{20b}, R^{20c}, R^{20d}, R^{20e}, R^{20f}, and R^{20g} is independently selected from hydrogen, —NH₂, —N(CH₃)₂, —NHCH₃, —CH₂NH₂, and —NHC(O)R¹¹.

In various aspects, R^{20b} is selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a further aspect, R^{20b} is selected from —F, —Cl, —CN, —NH₂, —NO₂, methyl, ethyl, propyl, isopropyl, ethenyl, 1-propenyl, 2-propenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)(CF₃), —OCH₃, —OCH₂CH₃, —OCH₂CH₂CH₃, —OCH(CH₃)₂, —CH₂NH₂, —CH₂CH₂NH₂, —CH₂CH₂CH₂NH₂, —CH(CH₃)CH₂NH₂, —N(CH₃)₂, —N(CH₃)CH₂CH₃, —N(CH₃)CH₂CH₂CH₃, —N(CH₃)CH(CH₃)₂, —N(CH₂CH₃)₂, —N(CH₂CH₃)CH₂CH₂CH₃, —N(CH₂CH₃)CH(CH₃)₂, —N(CH₂CH₂CH₃)₂, —N(CH₂CH₂CH₃)CH(CH₃)₂, —N(CH(CH₃)₂)₂, —NHCH₃, —NHCH₂CH₃, —NHCH₂CH₂CH₃, —NHCH(CH₃)₂, —OR¹⁰, and —NHC(O)R¹¹. In a further aspect, R^{20b} is selected from —F, —Cl, —CN, —NH₂, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —CH₂NH₂, —CH₂CH₂NH₂, —N(CH₃)₂, —N(CH₃)CH₂CH₃, —N(CH₂CH₃)₂, —NHCH₃, —NHCH₂CH₃, —OR¹⁰, and —NHC(O)R¹¹. In a still further aspect, R^{20b} is selected from —F, —Cl, —CN, —NH₂, —NO₂, methyl, —CH₂F, —CH₂Cl, —CH₂CN, —CH₂OH, —OCF₃, —OCH₃, —CH₂NH₂, —N(CH₃)₂, —NHCH₃, —OR¹⁰, and —NHC(O)R¹¹.

In various aspects, R^{20c} is selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a further aspect, R^{20c} is selected from —F, —Cl, —CN, —NH₂, —NO₂, methyl, ethyl, propyl, isopropyl, ethenyl, 1-propenyl, 2-propenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)(CF₃), —OCH₃, —OCH₂CH₃, —OCH₂CH₂CH₃, —OCH(CH₃)₂, —CH₂NH₂, —CH₂CH₂NH₂, —CH₂CH₂CH₂NH₂, —CH(CH₃)CH₂NH₂, —N(CH₃)₂, —N(CH₃)CH₂CH₃, —N(CH₃)CH₂CH₂CH₃, —N(CH₃)CH(CH₃)₂, —N(CH₂CH₃)₂, —N(CH₂CH₃)CH₂CH₂CH₃, —N(CH₂CH₃)CH(CH₃)₂, —N(CH₂CH₂CH₃)₂, —N(CH₂CH₂CH₃)CH(CH₃)₂, —N(CH(CH₃)₂)₂, —NHCH₃, —NHCH₂CH₃, —NHCH₂CH₂CH₃, —NHCH(CH₃)₂, —OR¹⁰, and —NHC(O)R¹¹. In a further aspect, R^{20e} is selected from —F, —Cl, —CN, —NH₂, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —CH₂NH₂, —CH₂CH₂NH₂, —N(CH₃)₂, —N(CH₃)CH₂CH₃, —N(CH₂CH₃)₂, —NHCH₃, —NHCH₂CH₃, —OR¹⁰, and —NHC(O)R¹¹. In a still further aspect, R^{20c} is selected from —F, —Cl, —CN, —NH₂, —NO₂, methyl, —CH₂F, —CH₂Cl, —CH₂CN, —CH₂OH, —OCF₃, —OCH₃, —CH₂NH₂, —N(CH₃)₂, —NHCH₃, —OR¹⁰, and —NHC(O)R¹¹.

In various aspects, each of R^{20a}, R^{20c}, R^{20a}, R^{20e}, R^{20f}, and R^{20g} is hydrogen.

In various aspects, each of R^{20a}, R^{20b}, R^{20d}, R^{20e}, R^{20f} and R^{20g} is hydrogen.

e. R²¹, R^{22a}, R^{22B}, R^{22C}, R^{22D}, R^{23A}, R^{23B}, and R^{23C} Groups

In one aspect, R²¹ is selected from hydrogen and C1-C4 alkyl; and each of R^{22a}, R^{22b}, R^{22c}, R^{22d}, R^{23a}, R^{23b}, and R^{23c} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹, provided that at least three of R²¹, R^{22a}, R^{22b}, R^{22c}, R^{22d}, R^{23a}, R^{23b}, and R^{23c} are hydrogen.

In various aspects, R²¹ is selected from hydrogen and C1-C4 alkyl. In a further aspect, R²¹ is selected from hydrogen, methyl, ethyl, n-propyl, and isopropyl. In a still further aspect, R²¹ is selected from hydrogen, methyl, and ethyl. In yet a further aspect, R²¹ is selected from hydrogen and ethyl. In an even further aspect, R²¹ is selected from hydrogen and methyl.

In various aspects, R²¹ is C1-C4 alkyl. In a further aspect, R²¹ is selected from methyl, ethyl, n-propyl, and isopropyl. In a still further aspect, R²¹ is selected from methyl and ethyl. In yet a further aspect, R²¹ is ethyl. In an even further aspect, R²¹ is methyl.

In various aspects, R²¹ is hydrogen.

In various aspects, each of R^{22a}, R^{22b}, R^{22c}, R^{22d}, R^{23a}, R^{23b}, and R^{23c} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R^{1D}, provided that at least three of R²¹, R^{22a}, R^{22b}, R^{22c}, R^{22a}, R^{23a}, R^{23b}, and R^{23c} are hydrogen. In a further aspect, each of R^{22a}, R^{22b}, R^{22c}, R^{22d}, R^{23a}, R^{23b}, and R^{23c} is independently selected from hydrogen, —F, —C1, —CN, —NH₂, —NO₂, methyl, ethyl, propyl, isopropyl, ethenyl, 1-propenyl, 2-propenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)(CF₃), —OCH₃, —OCH₂CH₃, —OCH₂CH₂CH₃, —OCH(CH₃)₂, —CH₂NH₂, —CH₂CH₂NH₂, —CH₂CH₂CH₂NH₂, —CH(CH₃)CH₂NH₂, —N(CH₃)₂, —N(CH₃)CH₂CH₃, —N(CH₃)CH₂CH₂CH₃, —N(CH₃)CH(CH₃)₂, —N(CH₂CH₃)₂, —N(CH₂CH₃)CH₂CH₂CH₃, —N(CH₂CH₃)CH(CH₃)₂, —N(CH₂CH₂CH₃)₂, —N(CH₂CH₂CH₃)CH(CH₃)₂, —N(CH(CH₃)₂)₂, —NHCH₃, —NHCH₂CH₃, —NHCH₂CH₂CH₃, —NHCH(CH₃)₂, —OR¹⁰, and —NHC(O)R¹¹. In a further aspect, each of R^{22a}, R^{22b}, R^{22e}, R^{22d}, R^{23a}, R^{23b}, and R^{23c} is independently selected from hydrogen, —F, —Cl, —CN, —NH₂, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —CH₂NH₂, —CH₂CH₂NH₂, —N(CH₃)₂, —N(CH₃)CH₂CH₃, —N(CH₂CH₃)₂, —NHCH₃, —NHCH₂CH₃, —OR¹⁰, and —NHC(O)R¹¹. In a still further aspect, each of R^{22a}, R^{22b}, R^{22c}, R^{22d}, R^{23a}, R^{23b}, and R^{23c} is independently selected from hydrogen, —F, —Cl, —CN, —NH₂, —NO₂, methyl, —CH₂F, —CH₂Cl, —CH₂CN, —CH₂OH, —OCF₃, —OCH₃, —CH₂NH₂, —N(CH₃)₂, —NHCH₃, —OR¹⁰, and —NHC(O)R¹¹.

In various aspects, each of R^{22a}, R^{22b}, R^{22c}, R^{22d}, R^{23a}, R^{23b}, and R^{23c} is independently selected from hydrogen, halogen, and C1-C4 alkyl. In a further aspect, each of R^{22a}, R^{22b}, R^{22c}, R^{22d}, R^{23a}, R^{23b}, and R^{23c} is independently selected from hydrogen, —F, —Cl, methyl, ethyl, propyl, and isopropyl. In a still further aspect, each of R^{22a}, R^{22b}, R^{22c}, R^{22d}, R^{23a}, R^{23b}, and R^{23c} is independently selected from hydrogen, —F, —Cl, methyl, and ethyl. In yet a further aspect, each of R^{22a}, R^{22b}, R^{22c}, R^{22d}, R^{23a}, R^{23b}, and R^{23c} is independently selected from hydrogen, —F, —Cl, and methyl.

In various aspects, each of R^{22a}, R^{22b}, R^{22c}, R^{22d}, R^{23a}, R^{23b}, and R^{23c} is independently selected from hydrogen and C1-C4 alkyl. In a further aspect, each of R^{22a}, R^{22b}, R^{22c}, R^{22d}, R^{23a}, R^{23b}, and R^{23c} is independently selected from hydrogen, methyl, ethyl, propyl, and isopropyl. In a still further aspect, each of R^{22a}, R^{22b}, R^{22c}, R^{22d}, R^{23a}, R^{23b}, and R^{23c} is independently selected from hydrogen, methyl, and ethyl. In yet a further aspect, each of R^{22a}, R^{22b}, R^{22c}, R^{22d}, R^{23a}, R^{23b}, and R^{23c} is independently selected from hydrogen and methyl.

In various aspects, each of R^{22a}, R^{22b}, R^{22c}, R^{22d}, R^{23a}, R^{23b}, and R^{23c} is independently selected from hydrogen and halogen. In a further aspect, each of R^{22a}, R^{22b}, R^{22c}, R^{22d}, R^{23a}, R^{23b}, and R^{23c} is independently selected from hydrogen, —F, —Cl, and —Br. In a still further aspect, each of R^{22a}, R^{22b}, R^{22c}, R^{22d}, R^{23a}, R^{23b}, and R^{23c} is independently selected from hydrogen, —F, and —Cl. In yet a further aspect, each of R^{22a}, R^{22b}, R^{22e}, R^{22a}, R^{23a}, R^{23b}, and R^{23c} is independently selected from hydrogen and —F. In an even further aspect, each of R^{22a}, R^{22b}, R^{22c}, R^{22d}, R^{23a}, R^{23b}, and R^{23c} is independently selected from hydrogen and —Cl.

In various aspects, each of R^{22a}, R^{22b}, R^{22c}, R^{22d}, R^{23a}, R^{23b}, and R^{23c} is independently selected from hydrogen, C1-C4 alkyl, and C2-C4 alkenyl. In a further aspect, each of R^{22a}, R^{22b}, R^{22c}, R^{22d}, R^{23a}, R^{23b}, and R^{23c} is independently selected from hydrogen, methyl, ethyl, propyl, isopropyl, ethenyl, 1-propenyl, and 2-propenyl. In a still further aspect, each of R^{22a}, R^{22b}, R^{22c}, R^{22d}, R^{23a}, R^{23b}, and R^{23c} is independently selected from hydrogen, methyl, ethyl, and ethenyl.

In various aspects, each of R^{22a}, R^{22b}, R^{22c}, R^{22d}, R^{23a}, R^{23b}, and R^{23c} is independently selected from hydrogen, halogen, and C1-C4 haloalkyl. In a further aspect, each of R^{22a}, R^{22b}, R^{22c}, R^{22d}, R^{23a}, R^{23b}, and R^{23c} is independently selected from hydrogen, —F, —C1, —CH₂F, —CH₂CH₂F, —CH₂CH₂CH₂F, —CH(CH₃)CH₂F, —CH₂Cl, —CH₂CH₂Cl, —CH₂CH₂CH₂Cl, and —CH(CH₃)CH₂Cl. In a still further aspect, each of R^{22a}, R^{22b}, R^{22c}, R^{22d}, R^{23a}, R^{23b}, and R^{23c} is independently selected from hydrogen, —F, —Cl, —CH₂F, —CH₂CH₂F, —CH₂Cl, and —CH₂CH₂Cl. In yet a further aspect, each of R^{22a}, R^{22b}, R^{22c}, R^{22d}, R^{23a}, R^{23b}, and R^{23c} is independently selected from hydrogen, —F, —Cl, —CH₂F, and —CH₂Cl.

In various aspects, each of R^{22a}, R^{22b}, R^{22c}, R^{22d}, R^{23a}, R^{23b}, and R^{23c} is independently selected from hydrogen, —CN, and C1-C4 cyanoalkyl. In a further aspect, each of R^{22a}, R^{22b}, R^{22c}, R^{22d}, R^{23a}, R^{23b}, and R^{23c} is independently selected from hydrogen, —CN, —CH₂CN, —CH₂CH₂CN, —CH₂CH₂CH₂CN and —CH(CH₃)CH₂CN. In a still further aspect, each of R^{22a}, R^{22b}, R^{22c}, R^{22d}, R^{23a}, R^{23b}, and R^{23c} is independently selected from hydrogen, —CN, —CH₂CN, and —CH₂CH₂CN. In yet a further aspect, each of R^{22a}, R^{22b}, R^{22c}, R^{22d}, R^{23a}, R^{23b}, and R^{23c} is independently selected from hydrogen, —CN, and —CH₂CN.

In various aspects, each of R^{22a}, R^{22b}, R^{22c}, R^{22d}, R^{23a}, R^{23b}, and R^{23c} is independently selected from hydrogen, C1-C4 hydroxyalkyl, and —OR¹⁰. In a further aspect, each of R^{22a}, R^{22b}, R^{22c}, R^{22d}, R^{23a}, R^{23b}, and R^{23c} is independently selected from hydrogen, —CH₂OH, —CH₂CH₂OH, —CH₂CH₂CH₂OH, —CH(CH₃)CH₂OH, and —OR¹⁰. In a still further aspect, each of R^{22a}, R^{22b}, R^{22c}, R^{22d}, R^{23a}, R^{23b}, and R^{23c} is independently selected from hydrogen, —CH₂OH, —CH₂CH₂OH, and —OR¹⁰. In an even further aspect, each of R^{22a}, R^{22b}, R^{22c}, R^{22d}, R^{23a}, R^{23b}, and R^{23c} is independently selected from hydrogen, —CH₂OH, and —OR¹⁰.

In various aspects, each of R^{22a}, R^{22b}, R^{22c}, R^{22d}, R^{23a}, R^{23b}, and R^{23c} is independently selected from hydrogen, —NH₂, C1-C4 alkylamino, (C1-C4)(C1-C4) dialkylamino, C1-C4 aminoalkyl, and —NHC(O)R¹¹. In a further aspect, each of R^{22a}, R^{22b}, R^{22c}, R^{22d}, R^{23a}, R^{23b}, and R^{23c} is independently selected from hydrogen, —NH₂, —N(CH₃)₂, —N(CH₃)CH₂CH₃, —N(CH₃)CH₂CH₂CH₃, —N(CH₃)CH(CH₃)₂, —N(CH₂CH₃)₂, —N(CH₂CH₃)CH₂CH₂CH₃, —N(CH₂CH₃)CH(CH₃)₂, —N(CH₂CH₂CH₃)₂, —N(CH₂CH₂CH₃)CH(CH₃)₂, —N(CH(CH₃)₂)₂, —NHCH₃, —NHCH₂CH₃, —NHCH₂CH₂CH₃, —NHCH(CH₃)₂, —CH₂NH₂, —CH₂CH₂NH₂, —CH₂CH₂CH₂NH₂, —CH(CH₃)CH₂NH₂, and —NHC(O)R¹¹. In a still further aspect, each of R^{22a}, R^{22b}, R^{22c}, R^{22a}, R^{23a}, R^{23b}, and R^{23e} is independently selected from hydrogen, —NH₂, —N(CH₃)₂, —N(CH₃)CH₂CH₃, —N(CH₃)CH(CH₃)₂, —N(CH₂CH₃)₂, —NHCH₃, —NHCH₂CH₃, —CH₂NH₂, —CH₂CH₂NH₂, and —NHC(O)R¹¹. In an even further aspect, each of R^{22a}, R^{22b}, R^{22e}, R^{22a}, R^{23a}, R^{23b} and R^{23c} is independently selected from hydrogen, —NH₂, —N(CH₃)₂, —NHCH₃, —CH₂NH₂, and —NHC(O)R¹¹.

In various aspects, each of R^{22a}, R^{22b}, R^{22c}, R^{22d}, R^{23a}, R^{23b}, and R^{23c} is hydrogen.

f. R^{24A}, R^{24B}, R^{24C}, R^{24D}, R^{24E}, and R²⁵ Groups

In one aspect, each of R^{24a}, R^{24b}, R^{24c}, R^{24d}, and R^{24e} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹; and R²⁵ is selected from hydrogen and C1-C4 alkyl, provided that at least two of R^{24a}, R^{24b}, R^{24c}, R^{24d}, R^{24e}, and R²⁵ are hydrogen.

In various aspects, each of R^{24a}, R^{24b}, R^{24c}, R^{24d}, and R^{24e} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a further aspect, each of R^{24a}, R^{24b}, R^{24c}, R^{24d}, and R^{24e} is independently selected from hydrogen, —F, —Cl, —CN, —NH₂, —NO₂, methyl, ethyl, propyl, isopropyl, ethenyl, 1-propenyl, 2-propenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)(CF₃), —OCH₃, —OCH₂CH₃, —OCH₂CH₂CH₃, —OCH(CH₃)₂, —CH₂NH₂, —CH₂CH₂NH₂, —CH₂CH₂CH₂NH₂, —CH(CH₃)CH₂NH₂, —N(CH₃)₂, —N(CH₃)CH₂CH₃, —N(CH₃)CH₂CH₂CH₃, —N(CH₃)CH(CH₃)₂, —N(CH₂CH₃)₂, —N(CH₂CH₃)CH₂CH₂CH₃, —N(CH₂CH₃)CH(CH₃)₂, —N(CH₂CH₂CH₃)₂, —N(CH₂CH₂CH₃)CH(CH₃)₂, —N(CH(CH₃)₂)₂, —NHCH₃, —NHCH₂CH₃, —NHCH₂CH₂CH₃, —NHCH(CH₃)₂, —OR¹⁰, and —NHC(O)R¹¹. In a further aspect, each of R^{24a}, R^{24b}, R^{24c}, R^{24d}, and R^{24e} is independently selected from hydrogen, —F, —Cl, —CN, —NH₂, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —CH₂NH₂, —CH₂CH₂NH₂, —N(CH₃)₂, —N(CH₃)CH₂CH₃, —N(CH₂CH₃)₂, —NHCH₃, —NHCH₂CH₃, —OR¹⁰, and —NHC(O)R¹¹. In a still further aspect, each of R^{24a}, R^{24b}, R^{24c}, R^{24a}, and R^{24e} is independently selected from hydrogen, —F, —Cl, —CN, —NH₂, —NO₂, methyl, —CH₂F, —CH₂Cl, —CH₂CN, —CH₂OH, —OCF₃, —OCH₃, —CH₂NH₂, —N(CH₃)₂, —NHCH₃, —OR¹⁰, and —NHC(O)R¹¹.

In various aspects, each of R^{24a}, R^{24b}, R^{24c}, R^{24a}, and R^{24e} is independently selected from hydrogen, halogen, and C1-C4 alkyl. In a further aspect, each of R^{24a}, R^{24b}R^{24c}, R^{24d}, and R^{24e} is independently selected from hydrogen, —F, —Cl, methyl, ethyl, propyl, and isopropyl. In a still further aspect, each of R^{24a}, R^{24b}, R^{24c}, R^{24d}, and R^{24e} is independently selected from hydrogen, —F, —Cl, methyl, and ethyl. In yet a further aspect, each of R^{24a}, R^{24b}, R^{24c}, R^{24d}, and R^{24e} is independently selected from hydrogen, —F, —Cl, and methyl.

In various aspects, each of R^{24a}, R^{24b}, R^{24c}, R^{24d}, and R^{24e} is independently selected from hydrogen and C1-C4 alkyl. In a further aspect, each of R^{24a}, R^{24b}, R^{24c}, R^{24d} and R^{24e} is independently selected from hydrogen, methyl, ethyl, propyl, and isopropyl. In a still further aspect, each of R^{24a}, R^{24b}, R^{24c}, R^{24d}, and R^{24e} is independently selected from hydrogen, methyl, and ethyl. In yet a further aspect, each of R^{24a}, R^{24b}, R^{24c}, R^{24d}, and R^{24e} is independently selected from hydrogen and methyl.

In various aspects, each of R^{24a}, R^{24b}, R^{24c}, R^{24d}, and R^{24e} is independently selected from hydrogen and halogen. In a further aspect, each of R^{24a}, R^{24b}, R^{24c}, R^{24d}, and R^{24e} is independently selected from hydrogen, —F, —Cl, and —Br. In a still further aspect, each of R^{24a}, R^{24b}, R^{24C}, R^{24d}, and R^{24e} is independently selected from hydrogen, —F, and —Cl. In yet a further aspect, each of R^{24a}, R^{24b}, R^{24c}, R^{24d}, and R^{24e} is independently selected from hydrogen and —F. In an even further aspect, each of R^{24a}, R^{24b}, R^{24c}, R^{24d}, and R^{24e} is independently selected from hydrogen and —Cl.

In various aspects, each of R^{24a}, R^{24b}, R^{24c}, R^{24d}, and R^{24e} is independently selected from hydrogen, C1-C4 alkyl, and C2-C4 alkenyl. In a further aspect, each of R^{24a}, R^{24b}, R^{24c}, R^{24d}, and R^{24e} is independently selected from hydrogen, methyl, ethyl, propyl, isopropyl, ethenyl, 1-propenyl, and 2-propenyl. In a still further aspect, each of R^{24a}, R^{24b}R^{24c}, R^{24d}, and R^{24e} is independently selected from hydrogen, methyl, ethyl, and ethenyl.

In various aspects, each of R^{24a}, R^{24b}, R^{24c}, R^{24d}, and R^{24e} is independently selected from hydrogen, halogen, and C1-C4 haloalkyl. In a further aspect, each of R^{24a}, R^{24b}, R^{24c}, R^{24d}, and R^{24e} is independently selected from hydrogen, —F, —Cl, —CH₂F, —CH₂CH₂F, —CH₂CH₂CH₂F, —CH(CH₃)CH₂F, —CH₂Cl, —CH₂CH₂Cl, —CH₂CH₂CH₂Cl, and —CH(CH₃)CH₂Cl. In a still further aspect, each of R^{24a}, R^{24b}, R^{24c}, R^{24a}, and R^{24e} is independently selected from hydrogen, —F, —Cl, —CH₂F, —CH₂CH₂F, —CH₂Cl, and —CH₂CH₂Cl. In yet a further aspect, each of R^{24a}, R^{24b}, R^{24e}, R^{24d}, and R^{24e} is independently selected from hydrogen, —F, —Cl, —CH₂F, and —CH₂Cl.

In various aspects, each of R^{24a}, R^{24b}, R^{24c}, R^{24d}, and R^{24e} is independently selected from hydrogen, —CN, and C1-C4 cyanoalkyl. In a further aspect, each of R^{24a}, R^{24b}R^{24c}, R^{24d}, and R^{24e} is independently selected from hydrogen, —CN, —CH₂CN, —CH₂CH₂CN, —CH₂CH₂CH₂CN and —CH(CH₃)CH₂CN. In a still further aspect, each of R^{24a}, R^{24b}, R^{24c}, R^{24d}, and R^{24e} is independently selected from hydrogen, —CN, —CH₂CN, and —CH₂CH₂CN.

In yet a further aspect, each of R^{24a}, R^{24b}, R^{24c}, R^{24d}, and R^{24e} is independently selected from hydrogen, —CN, and —CH₂CN.

In various aspects, each of R^{24a}, R^{24b}, R^{24c}, R^{24d}, and R^{24e} is independently selected from hydrogen, C1-C4 hydroxyalkyl, and —OR¹⁰. In a further aspect, each of R^{24a}, R^{24b}, R^{24c}, R^{24d}, and R^{24e} is independently selected from hydrogen, —CH₂OH, —CH₂CH₂OH, —CH₂CH₂CH₂OH, —CH(CH₃)CH₂OH, and —OR¹⁰. In a still further aspect, each of R^{24a}, R^{24b}R^{24c}, R^{24d}, and R^{24e} is independently selected from hydrogen, —CH₂OH, —CH₂CH₂OH, and —OR¹⁰. In an even further aspect, each of R^{24a}, R^{24b}, R^{24c}, R^{24d}, and R^{24e} is independently selected from hydrogen, —CH₂OH, and —OR¹⁰.

In various aspects, each of R^{24a}, R^{24b}, R^{24c}, R^{24d}, and R^{24e} is independently selected from hydrogen, —NH₂, C1-C4 alkylamino, (C1-C4)(C1-C4) dialkylamino, C1-C4 aminoalkyl, and —NHC(O)R¹¹. In a further aspect, each of R^{24a}, R^{24b}, R^{24c}, R^{24d}, and R^{24e} is independently selected from hydrogen, —NH₂, —N(CH₃)₂, —N(CH₃)CH₂CH₃, —N(CH₃)CH₂CH₂CH₃, —N(CH₃)CH(CH₃)₂, —N(CH₂CH₃)₂, —N(CH₂CH₃)CH₂CH₂CH₃, —N(CH₂CH₃)CH(CH₃)₂, —N(CH₂CH₂CH₃)₂, —N(CH₂CH₂CH₃)CH(CH₃)₂, —N(CH(CH₃)₂)₂, —NHCH₃, —NHCH₂CH₃, —NHCH₂CH₂CH₃, —NHCH(CH₃)₂, —CH₂NH₂, —CH₂CH₂NH₂, —CH₂CH₂CH₂NH₂, —CH(CH₃)CH₂NH₂, and —NHC(O)R¹¹. In a still further aspect, each of R^{24a}, R^{24b}, R^{24c}, R^{24d}, and R^{24e} is independently selected from hydrogen, —NH₂, —N(CH₃)₂, —N(CH₃)CH₂CH₃, —N(CH₃)CH(CH₃)₂, —N(CH₂CH₃)₂, —NHCH₃, —NHCH₂CH₃, —CH₂NH₂, —CH₂CH₂NH₂, and —NHC(O)R¹¹. In an even further aspect, each of R^{24a}, R^{24b}, R^{24c}, R^{24d}, and R^{24e} is independently selected from hydrogen, —NH₂, —N(CH₃)₂, —NHCH₃, —CH₂NH₂, and —NHC(O)R¹¹.

In various aspects, each of R^{24a}, R^{24b}, R^{24c}, R^{24d}, and R^{24e} is hydrogen.

In various aspects, R²⁵ is selected from hydrogen and C1-C4 alkyl. In a further aspect, R²⁵ is selected from hydrogen, methyl, ethyl, n-propyl, and isopropyl. In a still further aspect, R²⁵ is selected from hydrogen, methyl, and ethyl. In yet a further aspect, R²⁵ is selected from hydrogen and ethyl. In an even further aspect, R²⁵ is selected from hydrogen and methyl.

In various aspects, R²⁵ is C1-C4 alkyl. In a further aspect, R²⁵ is selected from methyl, ethyl, n-propyl, and isopropyl. In a still further aspect, R²⁵ is selected from methyl and ethyl. In yet a further aspect, R²⁵ is ethyl. In an even further aspect, R²⁵ is methyl.

In various aspects, R²⁵ is hydrogen.

In various aspects, each of R²⁴, R^{24b}, R^{24c}, R^{24d}, R^{24e}, and R²⁵ is hydrogen.

g. AR¹ Groups

In one aspect, Ar¹, when present, is a C6 aryl or a C2-C5 heteroaryl, and is substituted with 0, 1, 2, or 3 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl. In a further aspect, Ar¹, when present, is a C6 aryl or a C2-C5 heteroaryl, and is substituted with 0, 1, or 2 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl. In a still further aspect, Ar¹, when present, is a C6 aryl or a C2-C5 heteroaryl, and is substituted with 0 or 1 group selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl. In yet a further aspect, Ar¹, when present, is a C6 aryl or a C2-C5 heteroaryl, and is monosubstituted with a group selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl. In an even further aspect, Ar¹, when present, is a C6 aryl or a C2-C5 heteroaryl, and is unsubstituted.

In various aspects, aspect, Ar¹, when present, is a C6 aryl substituted with 0, 1, 2, or 3 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl. In a further aspect, Ar¹, when present, is a C6 aryl substituted with 0, 1, or 2 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl. In a still further aspect, Ar¹, when present, is a C6 aryl substituted with 0 or 1 group selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl. In yet a further aspect, Ar¹, when present, is a C6 aryl monosubstituted with a group selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl. In an even further aspect, Ar¹, when present, is an unsubstituted C6 aryl.

In various aspects, aspect, Ar¹, when present, is a C2-C5 heteroaryl substituted with 0, 1, 2, or 3 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl. Examples of C2-C5 heteroaryls include, but are not limited to, thiophene, furan, pyrrole, oxazole, isoxazole, isothiazole, pyridine, pyrimidine, pyridazine, pyrazine, triazine, and purine. In a further aspect, Ar¹, when present, is a C2-C5 heteroaryl substituted with 0, 1, or 2 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl. In a still further aspect, Ar¹, when present, is a C2-C5 heteroaryl substituted with 0 or 1 group selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl. In yet a further aspect, Ar¹, when present, is a C2-C5 heteroaryl monosubstituted with a group selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl. In an even further aspect, Ar¹, when present, is an unsubstituted C2-C5 heteroaryl.

h. CY¹ Groups

In one aspect, Cy¹ is a 9- or 10-membered heterobicycle, a 10-membered biaryl, or a 9- or 10-membered heterobiaryl, and is substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a further aspect, Cy¹ is a 9- or 10-membered heterobicycle, a 10-membered biaryl, or a 9- or 10-membered heterobiaryl, and is substituted with 0, 1, 2, or 3 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy¹ is a 9- or 10-membered heterobicycle, a 10-membered biaryl, or a 9- or 10-membered heterobiaryl, and is substituted with 0, 1, or 2 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In yet a further aspect, Cy¹ is a 9- or 10-membered heterobicycle, a 10-membered biaryl, or a 9- or 10-membered heterobiaryl, and is substituted with 0 or 1 group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In an even further aspect, Cy¹ is a 9- or 10-membered heterobicycle, a 10-membered biaryl, or a 9- or 10-membered heterobiaryl, and is monosubstituted with a group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy¹ is a 9- or 10-membered heterobicycle, a 10-membered biaryl, or a 9- or 10-membered heterobiaryl, and is unsubstituted.

In various aspects, Cy¹ is a 9- or 10-membered heterobicycle, and is substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a further aspect, Cy¹ is a 9- or 10-membered heterobicycle, and is substituted with 0, 1, 2, or 3 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy¹ is a 9- or 10-membered heterobicycle, and is substituted with 0, 1, or 2 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In yet a further aspect, Cy¹ is a 9- or 10-membered heterobicycle, and is substituted with 0 or 1 group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R^{D}. In an even further aspect, Cy¹ is a 9- or 10-membered heterobicycle, and is monosubstituted with a group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy¹ is a 9- or 10-membered heterobicycle, and is unsubstituted.

In various aspects, Cy¹ is a 9-membered heterobicycle substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a further aspect, Cy¹ is a 9-membered heterobicycle substituted with 0, 1, 2, or 3 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy¹ is a 9-membered heterobicycle substituted with 0, 1, or 2 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In yet a further aspect, Cy¹ is a 9-membered heterobicycle substituted with 0 or 1 group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R^{D}.

In an even further aspect, Cy¹ is a 9-membered heterobicycle monosubstituted with a group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy¹ is an unsubstituted 9-membered heterobicycle.

In various aspects, Cy¹ is a 10-membered heterobicycle substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R^{D}. In a further aspect, Cy¹ is a 10-membered heterobicycle substituted with 0, 1, 2, or 3 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy¹ is a 10-membered heterobicycle substituted with 0, 1, or 2 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In yet a further aspect, Cy¹ is a 10-membered heterobicycle substituted with 0 or 1 group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹.

In an even further aspect, Cy¹ is a 10-membered heterobicycle monosubstituted with a group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy¹ is an unsubstituted 10-membered heterobicycle.

In various aspects, Cy¹ is a 10-membered heterobicycle substituted with 0, 1, 2, 3, or 4 groups independently selected from halogen, C1-C4 alkyl, —OR¹⁰, and —NHC(O)R¹¹. In a further aspect, Cy¹ is a 10-membered heterobicycle substituted with 0, 1, 2, or 3 groups independently selected from halogen, C1-C4 alkyl, —OR¹⁰, and —NHC(O)R¹¹. In a still further aspect, Cy¹ is a 10-membered heterobicycle substituted with 0, 1, or 2 groups independently selected from halogen, C1-C4 alkyl, —OR¹⁰, and —NHC(O)R¹¹. In yet a further aspect, Cy¹ is a 10-membered heterobicycle substituted with 0 or 1 group selected from halogen, C1-C4 alkyl, —OR¹⁰, and —NHC(O)R¹¹. In an even further aspect, Cy¹ is a 10-membered heterobicycle monosubstituted with a group selected from halogen, C1-C4 alkyl, —OR¹⁰, and —NHC(O)R¹¹. In a still further aspect, Cy¹ is an unsubstituted 10-membered heterobicycle.

In various aspects, Cy¹ is a 10-membered heterobicycle having a structure:

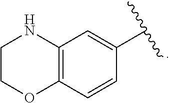 [SMILES: *c1ccc2c(c1)NCCO2]

In various aspects, Cy¹ is a 10-membered biaryl substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a further aspect, Cy¹ is a 10-membered biaryl substituted with 0, 1, 2, or 3 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy¹ is a 10-membered biaryl substituted with 0, 1, or 2 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In yet a further aspect, Cy¹ is a 10-membered biaryl substituted with 0 or 1 group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In an even further aspect, Cy¹ is a 10-membered biaryl monosubstituted with a group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy¹ is an unsubstituted 10-membered biaryl.

In various aspects, Cy¹ is a 10-membered biaryl substituted with 0, 1, 2, 3, or 4 groups independently selected from halogen, C1-C4 alkyl, —OR¹⁰, and —NHC(O)R¹¹. In a further aspect, Cy¹ is a 10-membered biaryl substituted with 0, 1, 2, or 3 groups independently selected from halogen, C1-C4 alkyl, —OR¹⁰, and —NHC(O)R¹¹. In a still further aspect, Cy¹ is a 10-membered biaryl substituted with 0, 1, or 2 groups independently selected from halogen, C1-C4 alkyl, —OR¹⁰, and —NHC(O)R¹¹. In yet a further aspect, Cy¹ is a 10-membered biaryl substituted with 0 or 1 group selected from halogen, C1-C4 alkyl, —OR¹⁰, and —NHC(O)R¹¹. In an even further aspect, Cy¹ is a 10-membered biaryl monosubstituted with a group selected from halogen, C1-C4 alkyl, —OR¹⁰, and —NHC(O)R¹¹.

In an even further aspect, Cy¹ is an unsubstituted 10-membered biaryl.

In various aspects, Cy¹ is a 10-membered biaryl having a structure selected from:

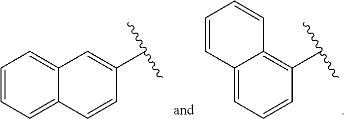 [SMILES: *c1ccc2ccccc2c1 ; *c1cccc2ccccc12]

In various aspects, Cy¹ is a 9- or 10-membered heterobiaryl, and is substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a further aspect, Cy¹ is a 9- or 10-membered heterobiaryl, and is substituted with 0, 1, 2, or 3 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy¹ is a 9- or 10-membered heterobiaryl, and is substituted with 0, 1, or 2 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In yet a further aspect, Cy¹ is a 9- or 10-membered heterobiaryl, and is substituted with 0 or 1 group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In an even further aspect, Cy¹ is a 9- or 10-membered heterobiaryl, and is monosubstituted with a group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy¹ is a 9- or 10-membered heterobiaryl, and is unsubstituted.

In various aspects, Cy¹ is a 9-membered heterobiaryl substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a further aspect, Cy¹ is a 9- or 10-membered heterobiaryl, and is substituted with 0, 1, 2, or 3 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy¹ is a 9-membered heterobiaryl substituted with 0, 1, or 2 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In yet a further aspect, Cy¹ is a 9-membered heterobiaryl substituted with 0 or 1 group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In an even further aspect, Cy¹ is a 9-membered heterobiaryl monosubstituted with a group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy¹ is an unsubstituted 9-membered heterobiaryl.

In various aspects, Cy¹ is a 10-membered heterobiaryl substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a further aspect, Cy¹ is a 10-membered heterobiaryl substituted with 0, 1, 2, or 3 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy¹ is a 10-membered heterobiaryl substituted with 0, 1, or 2 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In yet a further aspect, Cy¹ is a 10-membered heterobiaryl substituted with 0 or 1 group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹.

In an even further aspect, Cy¹ is a 10-membered heterobiaryl monosubstituted with a group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy¹ is an unsubstituted 10-membered heterobiaryl.

In various aspects, Cy¹ is a 9- or 10-membered heterobiaryl substituted with 0, 1, 2, 3, or 4 groups independently selected from halogen, C1-C4 alkyl, —OR¹⁰, and —NHC(O)R¹¹. In a further aspect, Cy¹ is a 9- or 10-membered heterobiaryl substituted with 0, 1, 2, or 3 groups independently selected from halogen, C1-C4 alkyl, —OR¹⁰, and —NHC(O)R¹¹. In a still further aspect, Cy¹ is a 9- or 10-membered heterobiaryl substituted with 0, 1, or 2 groups independently selected from halogen, C1-C4 alkyl, —OR¹⁰, and —NHC(O)R¹¹. In yet a further aspect, Cy¹ is a 9- or 10-membered heterobiaryl substituted with 0 or 1 group selected from halogen, C1-C4 alkyl, —OR¹⁰, and —NHC(O)R¹¹. In an even further aspect, Cy¹ is a 9- or 10-membered heterobiaryl monosubstituted with a group selected from halogen, C1-C4 alkyl, —OR¹⁰, and —NHC(O)R¹¹. In a still further aspect, Cy¹ is a 9- or 10-membered heterobiaryl, and is unsubstituted.

In various aspects, Cy¹ is a 9- or 10-membered heterobiaryl having a structure selected from:

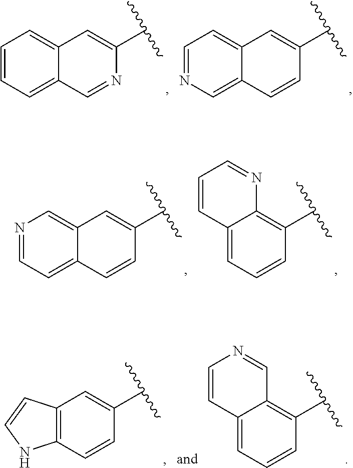

i. CY² Groups

In one aspect, Cy², when present, is a C2-C5 heterocycloalkyl substituted with 0, 1, 2, or 3 groups independently selected from halogen, —N₃, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 alkoxy, C1-C4 alkylamino, (C1-C4)(C1-C4) dialkylamino, C1-C4 aminoalkyl, —C(O)H, —C(O)(C1-C4 alkyl), —CO₂H, and —CO₂(C1-C4 alkyl). Examples of C2-C5 heteroaryls include, but are not limited to, thiophene, furan, pyrrole, oxazole, isoxazole, isothiazole, pyridine, pyrimidine, pyridazine, pyrazine, triazine, and purine. In a further aspect, Cy², when present, is a C2-C5 heterocycloalkyl substituted with 0, 1, or 2 groups independently selected from halogen, —N₃, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 alkoxy, C1-C4 alkylamino, (C1-C4)(C1-C4) dialkylamino, C1-C4 aminoalkyl, —C(O)H, —C(O)(C1-C4 alkyl), —CO₂H, and —CO₂(C1-C4 alkyl). In a still further aspect, Cy², when present, is a C2-C5 heterocycloalkyl substituted with 0 or 1 group selected from halogen, —N₃, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 alkoxy, C1-C4 alkylamino, (C1-C4)(C1-C4) dialkylamino, C1-C4 aminoalkyl, —C(O)H, —C(O)(C1-C4 alkyl), —CO₂H, and —CO₂(C1-C4 alkyl). In yet a further aspect, Cy², when present, is a C2-C5 heterocycloalkyl monosubstituted with a group selected from halogen, —N₃, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 alkoxy, C1-C4 alkylamino, (C1-C4)(C1-C4) dialkylamino, C1-C4 aminoalkyl, —C(O)H, —C(O)(C1-C4 alkyl), —CO₂H, and —CO₂(C1-C4 alkyl). In a further aspect, Cy², when present, is an unsubstituted C2-C5 heterocycloalkyl.

### 2. Example Compounds

In one aspect, a compound can be present as one or more of the following structures:

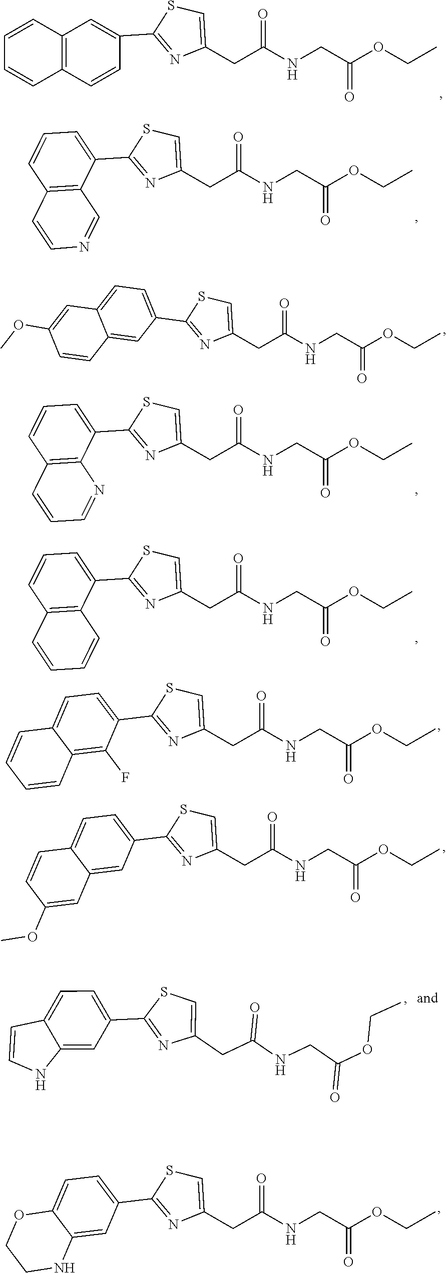

or a pharmaceutically acceptable salt thereof.

In one aspect, a compound can be present as one or more of the following structures:

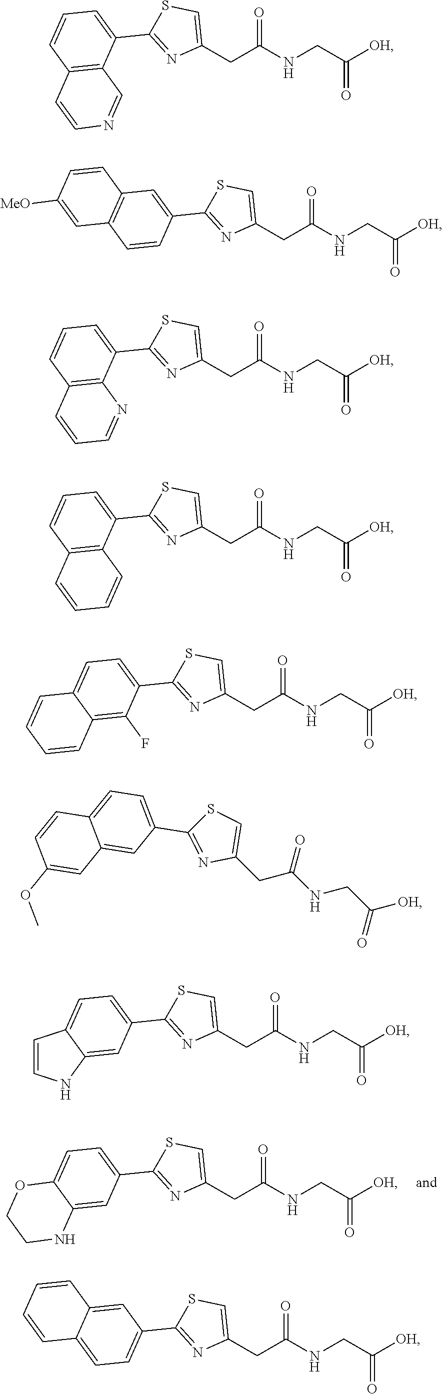

or a pharmaceutically acceptable salt thereof.

In one aspect, a compound can be present as one or more of the following structures:

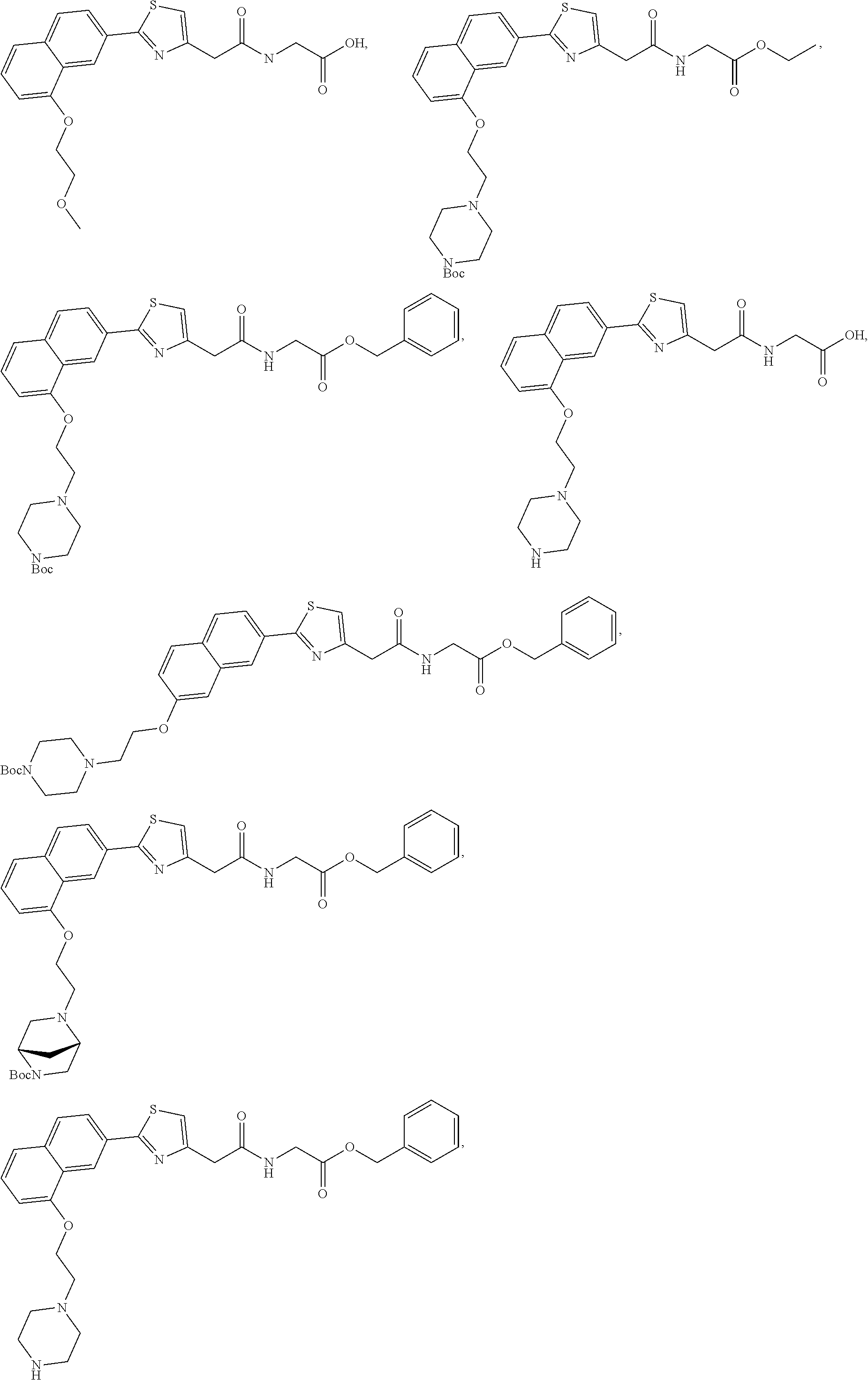

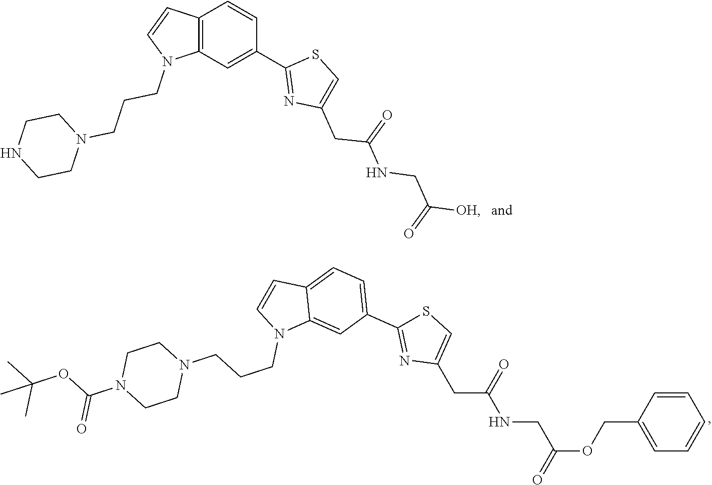 [SMILES: O=C(O)CNC(=O)Cc1csc(-c2ccc3ccn(CCCN4CCNCC4)c3c2)n1 ; "and" ; CC(C)(C)OC(=O)N1CCN(CCCn2ccc3ccc(-c4nc(CC(=O)NCC(=O)OCc5ccccc5)cs4)cc32)CC1]

or a pharmaceutically acceptable salt thereof.

### C. Heterobifunctional Compounds

In one aspect, disclosed are heterobifunctional compounds useful as protein degraders. As would be understood by one of skill in the art, a protein degrader can be useful in treating a variety of different disorders including, but not limited to, cancer.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, disclosed are compounds having a structure represented by a formula:

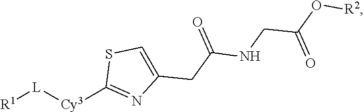

wherein L is a linker; wherein R¹ is a residue of a small molecule having a molecular weight of from about 150 g/mol to about 600 g/mol and a binding affinity (Kᵢ) to a target protein of at least about 20 μM; wherein R² is selected from hydrogen, C1-C4 alkyl, C4-C6 cycloalkyl, —(C1-C4 alkyl)OC(O)(C1-C4 alkyl), and —CH₂C₆H₅; and wherein Cy³ is a 9- or 10-membered heterobicycle, a 10-membered biaryl, or a 9- or 10-membered heterobiaryl, and is substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹; wherein each occurrence of R¹⁰, when present, is independently selected from hydrogen, C1-C4 alkyl, —(C1-C4 alkyl)O(C1-C4 alkyl), —(C1-C4 alkyl)O(C1-C4 alkyl)N₃, and —(C1-C4 alkyl)Cy²; wherein Cy², when present, is a C2-C5 heterocycloalkyl substituted with 0, 1, 2, or 3 groups independently selected from halogen, —N₃, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 alkoxy, C1-C4 alkylamino, (C1-C4)(C1-C4) dialkylamino, C1-C4 aminoalkyl, —C(O)H, —C(O)(C1-C4 alkyl), —CO₂H, and —CO₂(C1-C4 alkyl); wherein each occurrence of R¹¹, when present, is independently selected from C1-C4 alkyl, C1-C4 hydroxyalkyl, and Ar¹; wherein Ar¹, when present, is a C6 aryl or a C2-C5 heteroaryl, and is substituted with 0, 1, 2, or 3 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl, or a pharmaceutically acceptable salt thereof.

In various aspects, the compound has a structure represented by a formula selected from:

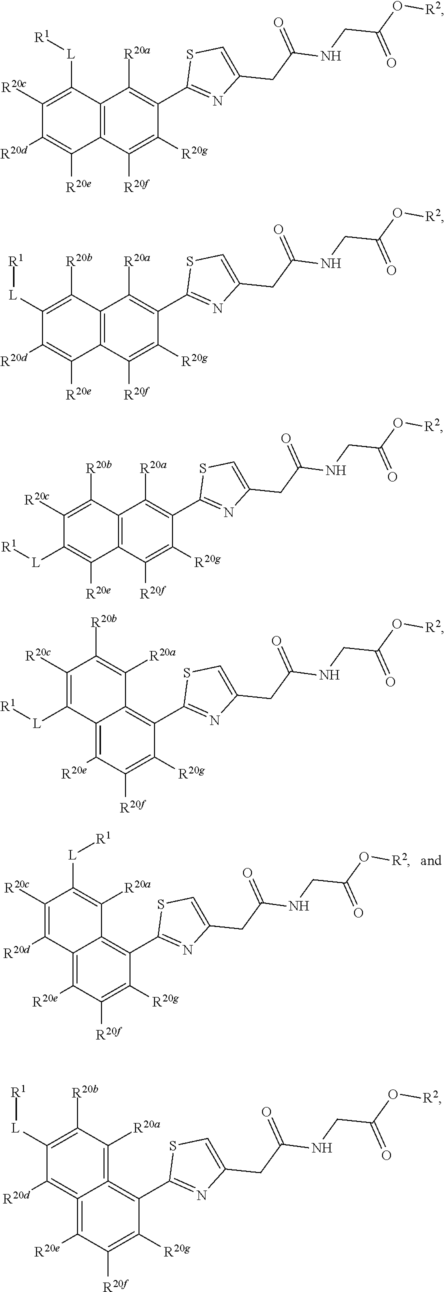

wherein each of R^{20a}, R^{20b}, R^{20c}, R^{20d}, R^{20e}, R^{20f}, and R^{20g} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹, provided that at least two of R^{20a}, R^{20b}, R^{20c}, R^{20a}, R^{20e}, R^{20f}, and R^{20g} are hydrogen, or a pharmaceutically acceptable salt thereof. In a further aspect, each of R^{20a}, R^{20b}, R^{20c}, R^{20a}, R^{20e}, R^{20f}, and R^{20g} is independently selected from hydrogen, halogen, and C1-C4 alkyl.

In various aspects, the compound has a structure represented by a formula

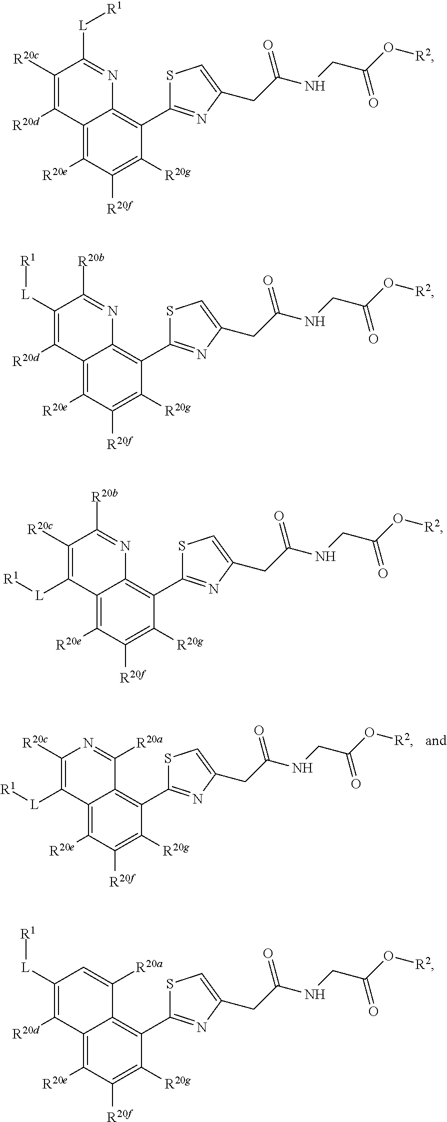

wherein each of R^{20a}, R^{20b}, R^{20c}, R^{20d}, R^{20e}, R^{20f}, and R^{20g} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹, provided that at least one of R^{20a}, R^{20b}, R^{20c}, R^{20d}, R^{20e}, R^{20f}, and R^{20g} is hydrogen, or a pharmaceutically acceptable salt thereof. In a further aspect, each of R^{20a}, R^{20b}, R^{20c}, R^{20d}, R^{20e}, R^{20f}, and R^{20g} is independently selected from hydrogen, halogen, and C1-C4 alkyl.

In various aspects, the compound has a structure represented by a formula selected from:

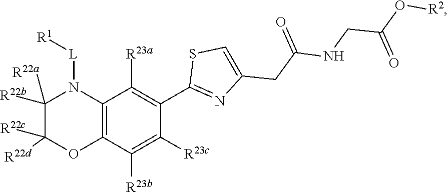

wherein each of R^{22a}, R^{22b}, R^{22c}, R^{22d}, R^{23a}, R^{23b}, and R^{23c} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹, provided that at least three of R^{22a}, R^{22b}, R^{22c}, R^{22d}, R^{23a}, R^{23b}, and R^{23c} are hydrogen, or a pharmaceutically acceptable salt thereof. In a further aspect, each of R^{22a}, R^{22b}, R^{22c}, R^{22d}, R^{23a}, R^{23b}, and R^{23c} is independently selected from hydrogen, halogen, and C1-C4 alkyl.

In various aspects, the compound has a structure represented by a formula selected from:

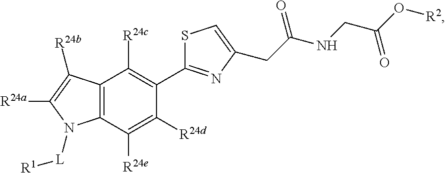

wherein each of R^{24a}, R^{24b}, R^{24c}, R^{24d}, and R^{24e} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹, provided that at least one of R^{24a}, R^{24b}, R^{24e}, R^{24d}, and R^{24c} are hydrogen, or a pharmaceutically acceptable salt thereof. In a further aspect, each of R^{24a}, R^{24b}, R^{24c}, R^{24d}, and R^{24e} is independently selected from hydrogen, halogen, and C1-C4 alkyl.

In various aspects, the compound has a structure represented by a formula

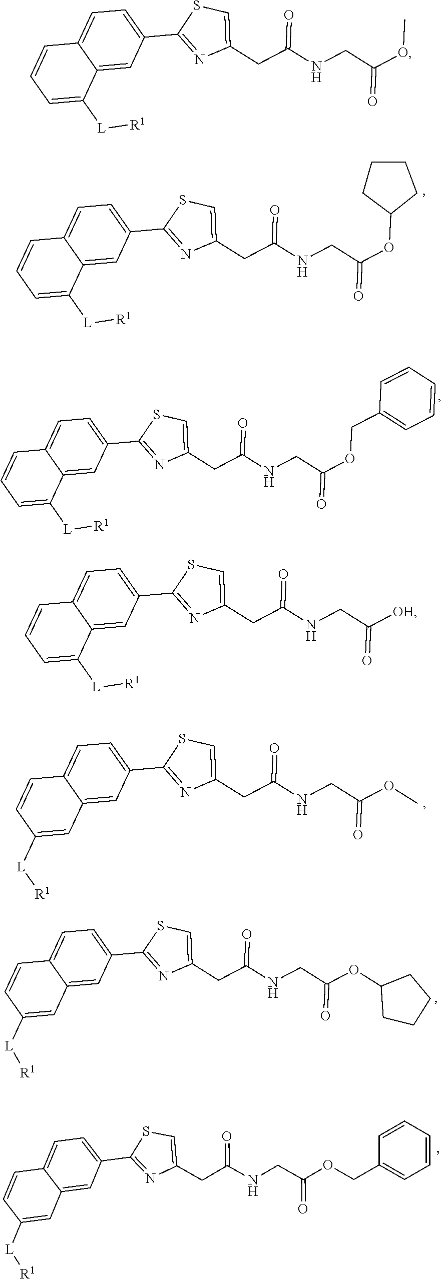

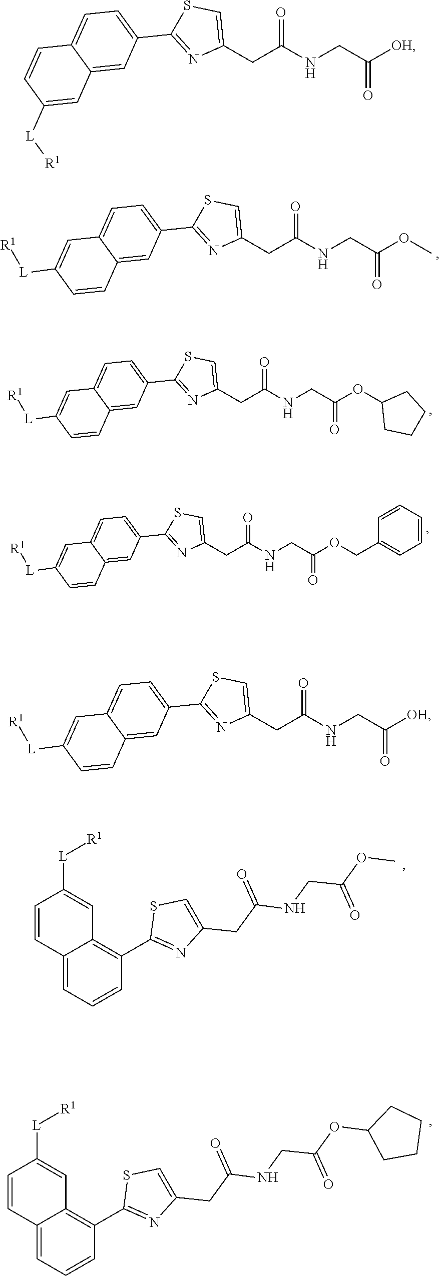

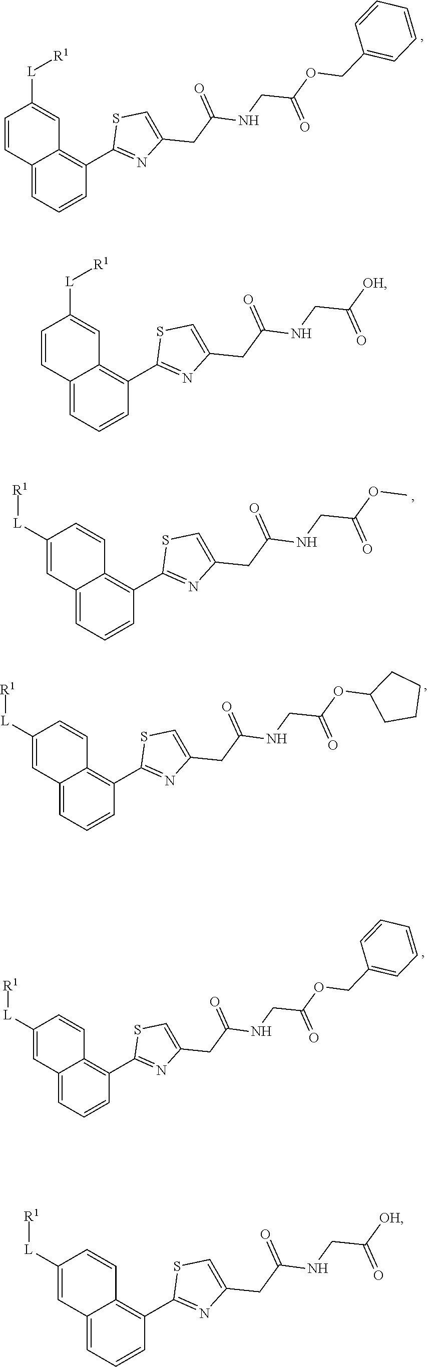

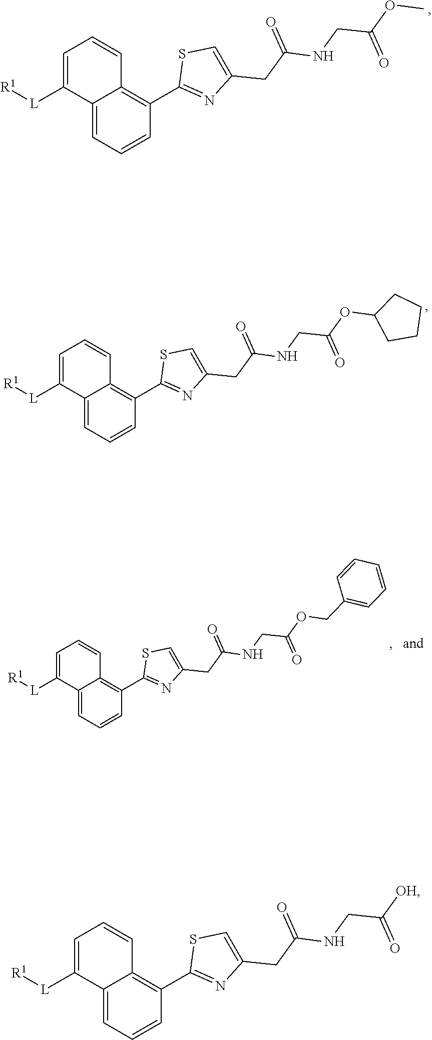

or a pharmaceutically acceptable salt thereof.

In various aspects, the compound has a structure represented by a formula

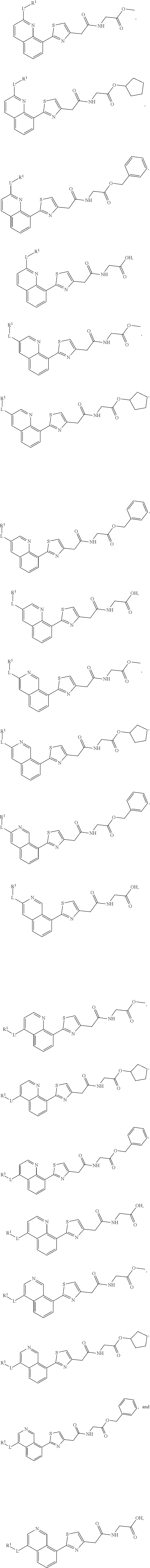

or a pharmaceutically acceptable salt thereof.

In various aspects, the compound is selected from:

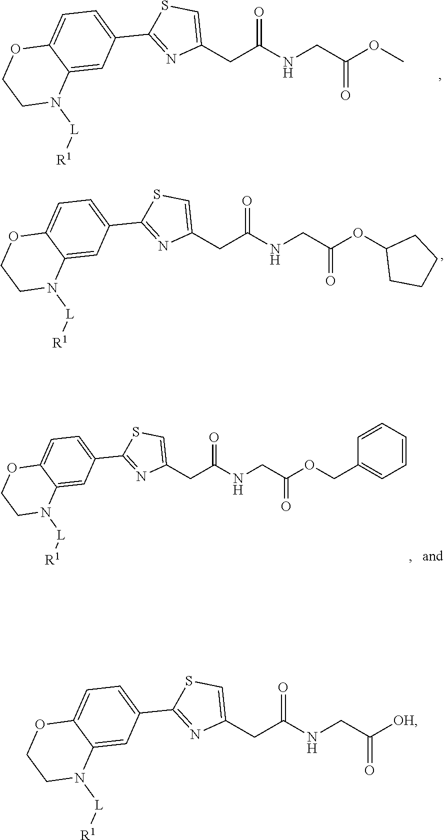

or a pharmaceutically acceptable salt thereof.

In various aspects, the compound is selected from:

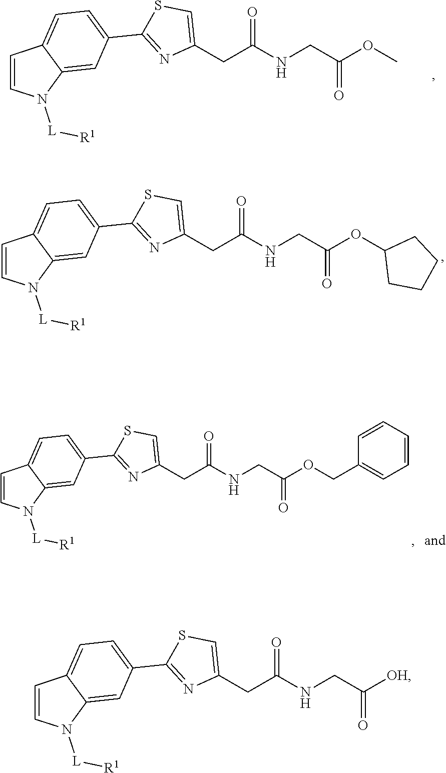

or a pharmaceutically acceptable salt thereof.

In various aspects, the compound has a structure represented by a formula:

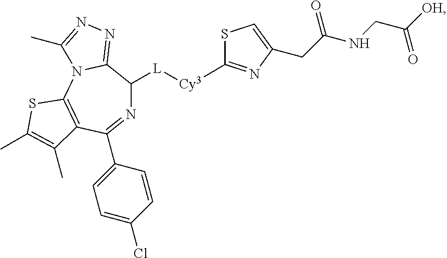

or a pharmaceutically acceptable salt thereof.

In various aspects, the compound has a structure represented by a formula selected from:

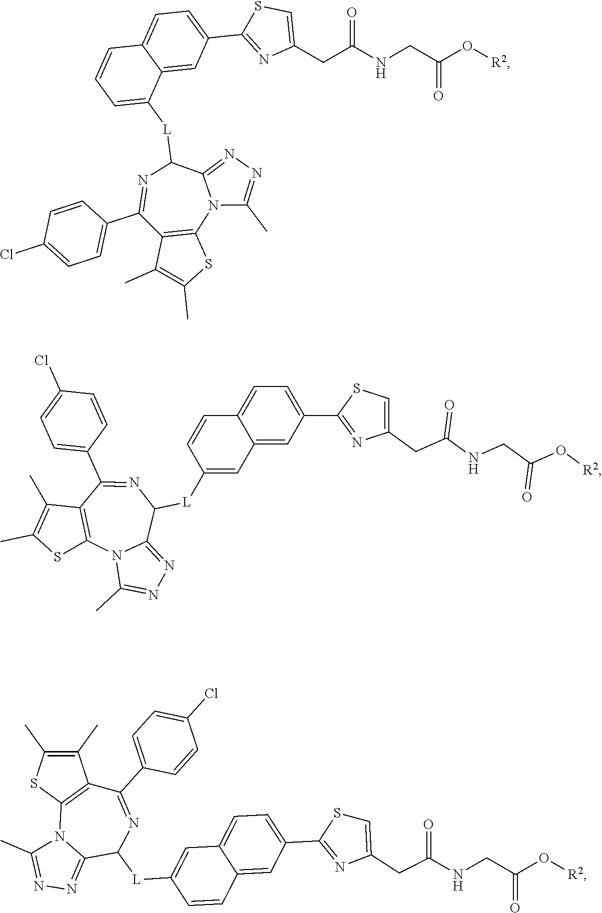

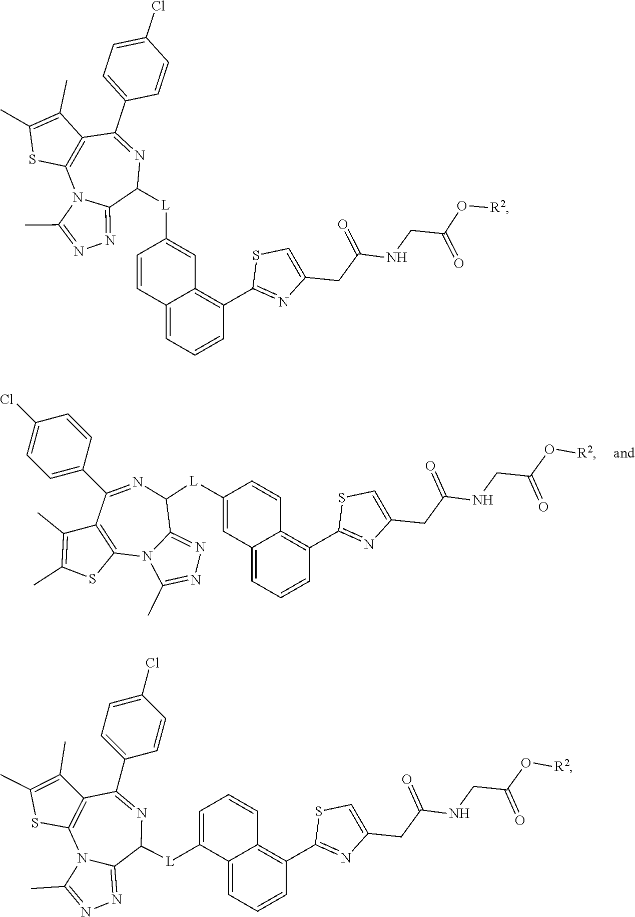

or a pharmaceutically acceptable salt thereof.

In various aspects, the compound has a structure represented by a formula

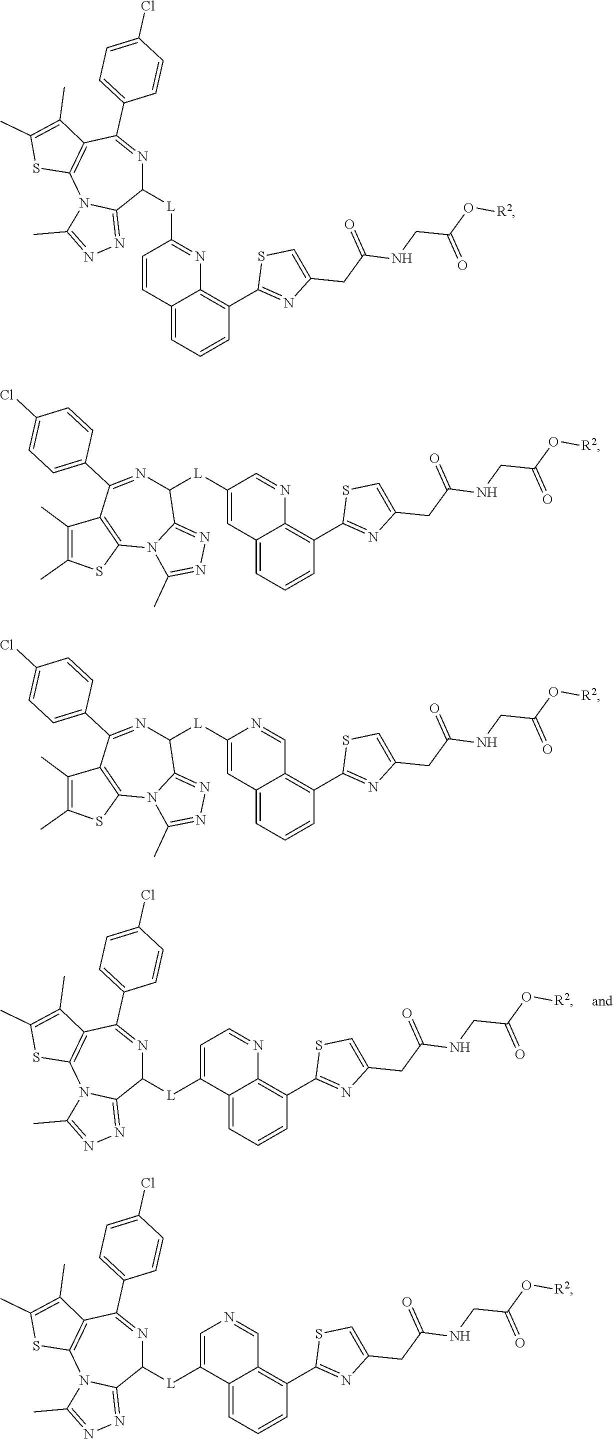

or a pharmaceutically acceptable salt thereof.

In various aspects, the compound has a structure represented by a formula:

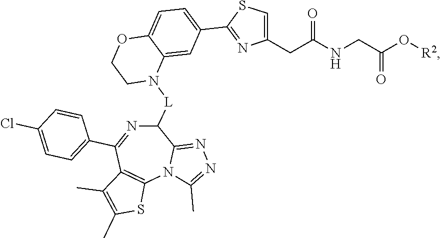

or a pharmaceutically acceptable salt thereof.

In various aspects, the compound has a structure represented by a formula:

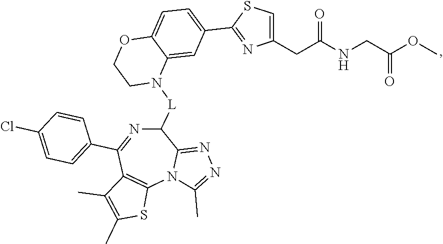

or a pharmaceutically acceptable salt thereof.

In various aspects, the compound is selected from:

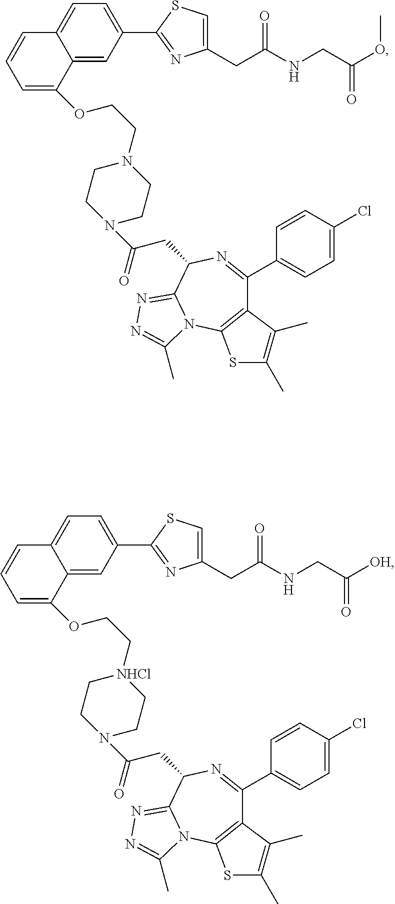

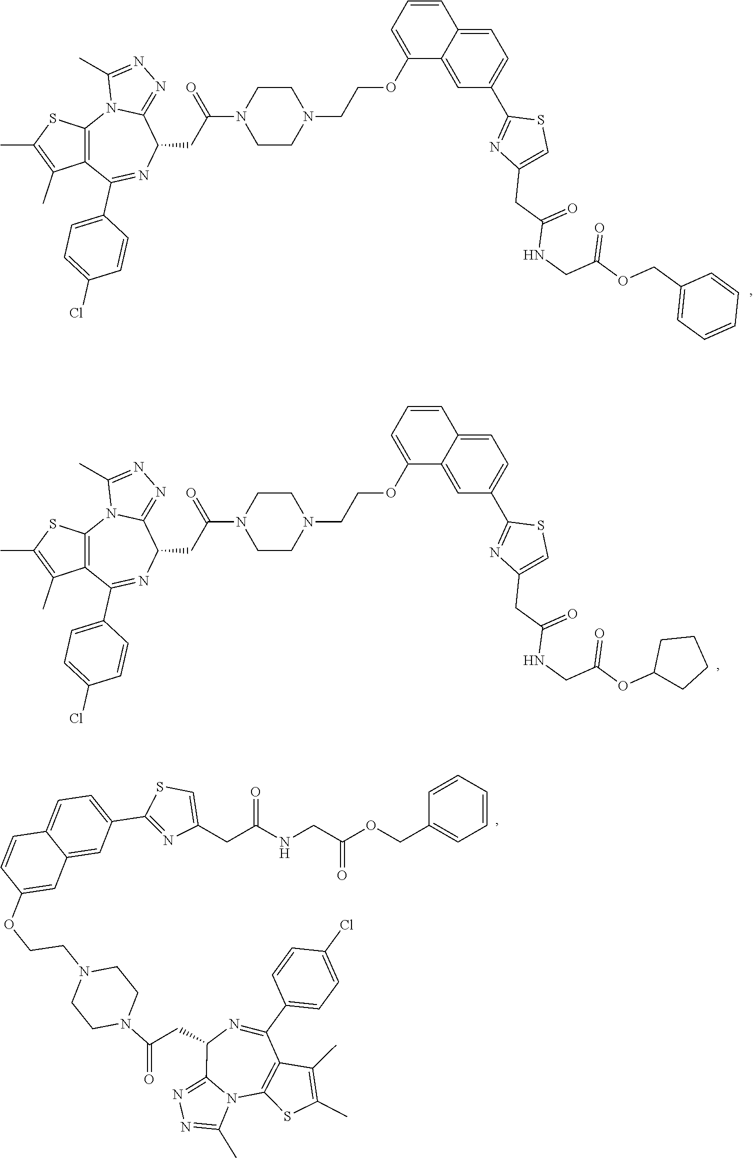 [SMILES: Cc1sc2c(c1C)C(c1ccc(Cl)cc1)=N[C@@H](CC(=O)N1CCN(CCOc3cccc4ccc(-c5nc(CC(=O)NCC(=O)OCc6ccccc6)cs5)cc34)CC1)c1nnc(C)n1-2 ; Cc1sc2c(c1C)C(c1ccc(Cl)cc1)=N[C@@H](CC(=O)N1CCN(CCOc3cccc4ccc(-c5nc(CC(=O)NCC(=O)OC6CCCC6)cs5)cc34)CC1)c1nnc(C)n1-2 ; Cc1sc2c(c1C)C(c1ccc(Cl)cc1)=N[C@@H](CC(=O)N1CCN(CCOc3ccc4ccc(-c5nc(CC(=O)NCC(=O)OCc6ccccc6)cs5)cc4c3)CC1)c1nnc(C)n1-2]

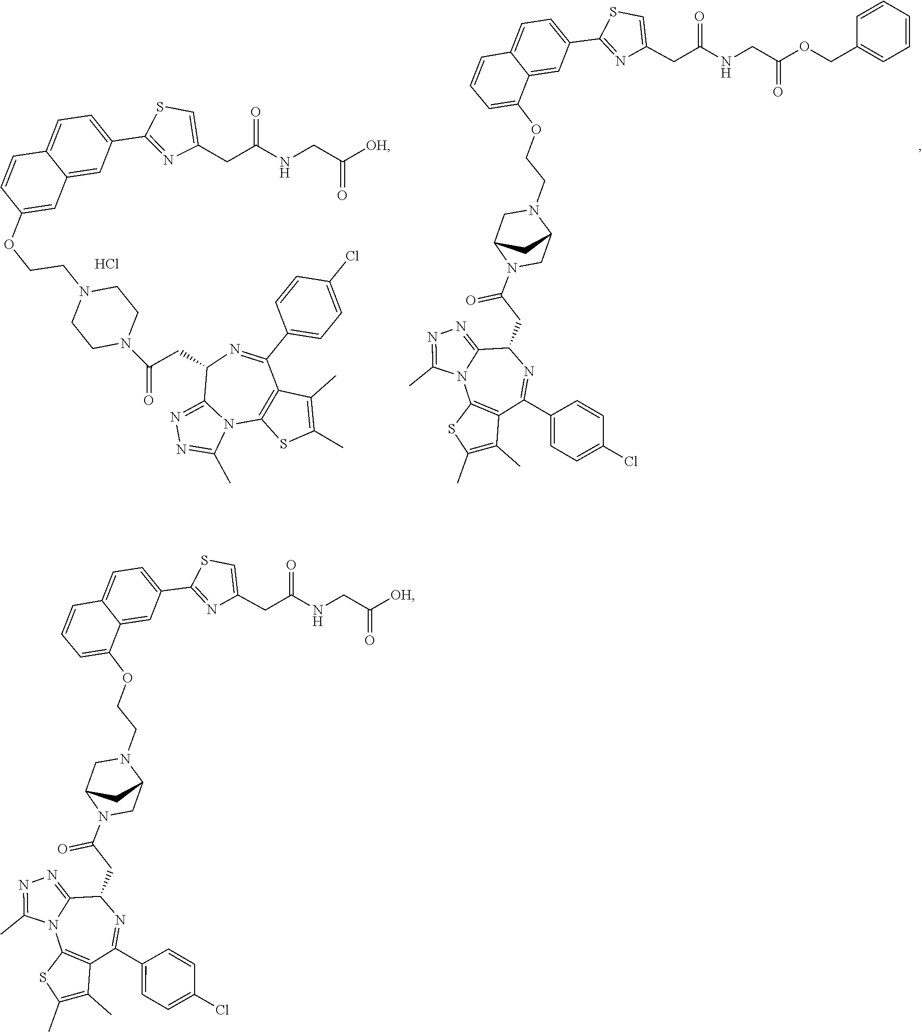 [SMILES: Cc1sc2c(c1C)C(c1ccc(Cl)cc1)=N[C@@H](CC(=O)N1CCN(CCOc3ccc4ccc(-c5nc(CC(=O)NCC(=O)O)cs5)cc4c3)CC1)c1nnc(C)n1-2 ; Cl ; Cc1sc2c(c1C)C(c1ccc(Cl)cc1)=N[C@@H](CC(=O)N1C[C@@H]3C[C@H]1CN3CCOc1cccc3ccc(-c4nc(CC(=O)NCC(=O)OCc5ccccc5)cs4)cc13)c1nnc(C)n1-2 ; Cc1sc2c(c1C)C(c1ccc(Cl)cc1)=N[C@@H](CC(=O)N1C[C@@H]3C[C@H]1CN3CCOc1cccc3ccc(-c4nc(CC(=O)NCC(=O)O)cs4)cc13)c1nnc(C)n1-2]

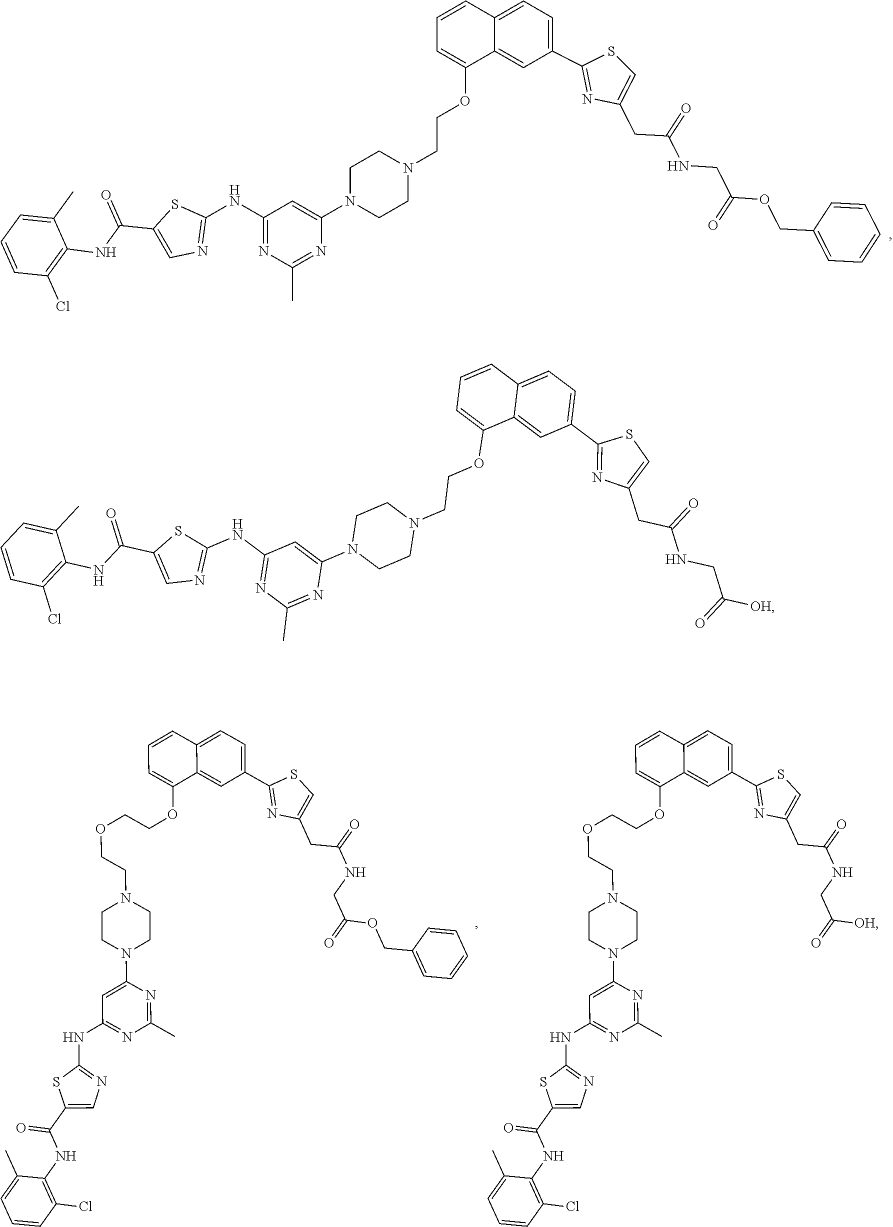 [SMILES: Cc1nc(Nc2ncc(C(=O)Nc3c(C)cccc3Cl)s2)cc(N2CCN(CCOc3cccc4ccc(-c5nc(CC(=O)NCC(=O)OCc6ccccc6)cs5)cc34)CC2)n1 ; Cc1nc(Nc2ncc(C(=O)Nc3c(C)cccc3Cl)s2)cc(N2CCN(CCOc3cccc4ccc(-c5nc(CC(=O)NCC(=O)O)cs5)cc34)CC2)n1 ; Cc1nc(Nc2ncc(C(=O)Nc3c(C)cccc3Cl)s2)cc(N2CCN(CCOCCOc3cccc4ccc(-c5nc(CC(=O)NCC(=O)OCc6ccccc6)cs5)cc34)CC2)n1 ; Cc1nc(Nc2ncc(C(=O)Nc3c(C)cccc3Cl)s2)cc(N2CCN(CCOCCOc3cccc4ccc(-c5nc(CC(=O)NCC(=O)O)cs5)cc34)CC2)n1]

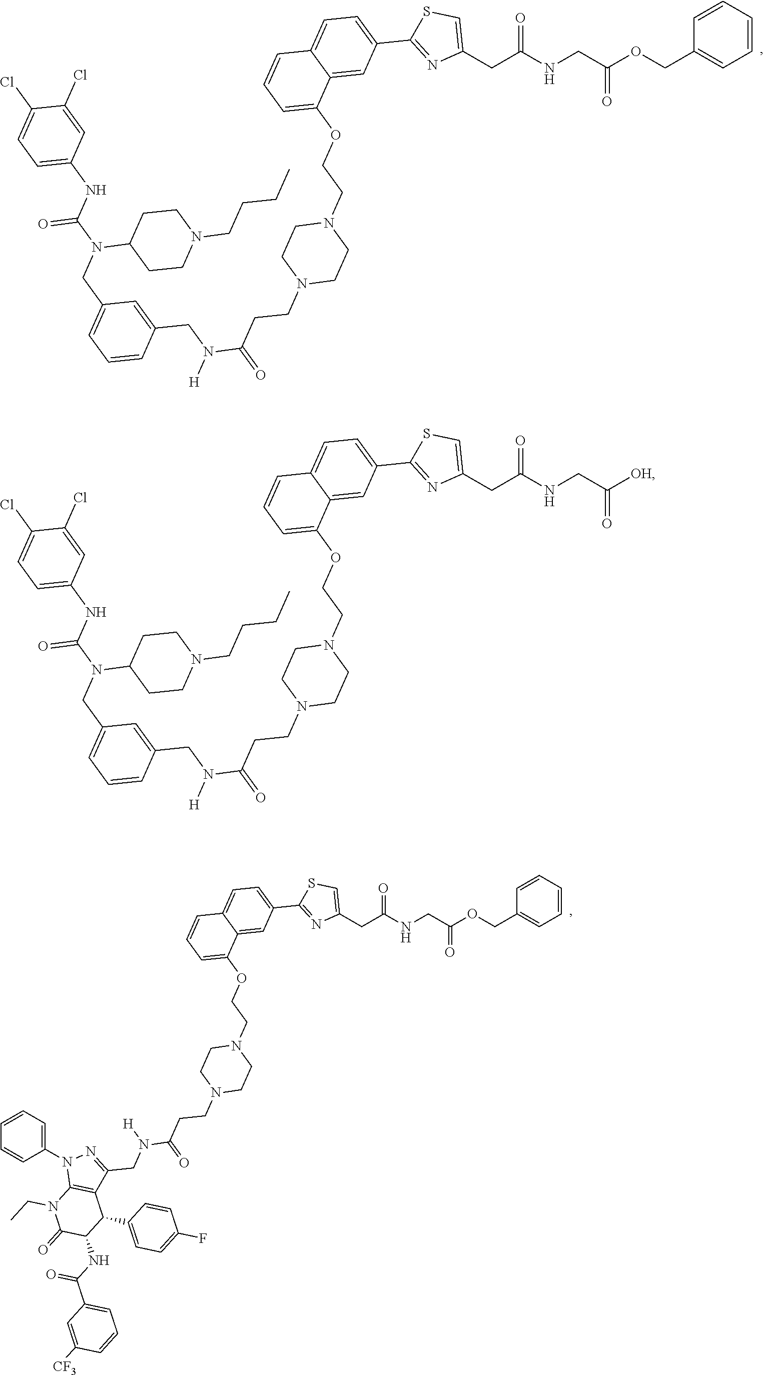 [SMILES: CCCCN1CCC(N(Cc2cccc(CNC(=O)CCN3CCN(CCOc4cccc5ccc(-c6nc(CC(=O)NCC(=O)OCc7ccccc7)cs6)cc45)CC3)c2)C(=O)Nc2ccc(Cl)c(Cl)c2)CC1 ; CCCCN1CCC(N(Cc2cccc(CNC(=O)CCN3CCN(CCOc4cccc5ccc(-c6nc(CC(=O)NCC(=O)O)cs6)cc45)CC3)c2)C(=O)Nc2ccc(Cl)c(Cl)c2)CC1 ; CCN1C(=O)[C@@H](NC(=O)c2cccc(C(F)(F)F)c2)[C@@H](c2ccc(F)cc2)c2c(CNC(=O)CCN3CCN(CCOc4cccc5ccc(-c6nc(CC(=O)NCC(=O)OCc7ccccc7)cs6)cc45)CC3)nn(-c3ccccc3)c21]

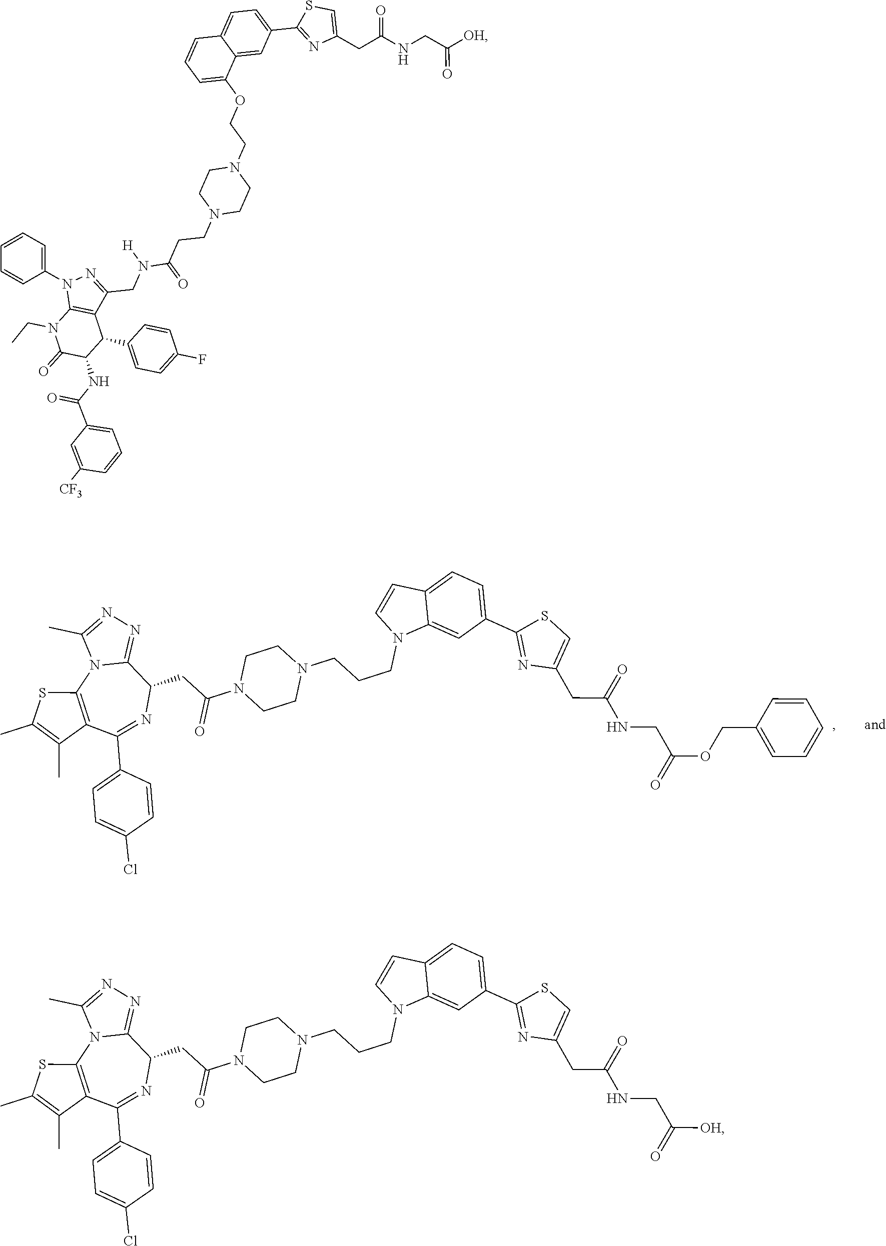 [SMILES: CCN1C(=O)[C@@H](NC(=O)c2cccc(C(F)(F)F)c2)[C@@H](c2ccc(F)cc2)c2c(CNC(=O)CCN3CCN(CCOc4cccc5ccc(-c6nc(CC(=O)NCC(=O)O)cs6)cc45)CC3)nn(-c3ccccc3)c21 ; Cc1sc2c(c1C)C(c1ccc(Cl)cc1)=N[C@@H](CC(=O)N1CCN(CCCn3ccc4ccc(-c5nc(CC(=O)NCC(=O)OCc6ccccc6)cs5)cc43)CC1)c1nnc(C)n1-2 ; Cc1sc2c(c1C)C(c1ccc(Cl)cc1)=N[C@@H](CC(=O)N1CCN(CCCn3ccc4ccc(-c5nc(CC(=O)NCC(=O)O)cs5)cc43)CC1)c1nnc(C)n1-2]

or a pharmaceutically acceptable salt thereof.
a. L Groups

In one aspect, L is a linker. As used herein, the term “linker” refers to a chemical group ranging in length from 4 to 16 atoms comprising carbon, nitrogen, and oxygen, that can be further covalently bound to the KLHDC2 ligase binding ligand as further described herein. In another embodiment, the linker can be bound to the KLHDC2 ligase binding ligand at one end and to the target protein binding moiety (i.e., a residue of a small molecule having a particular binding affinity to a target protein) at the other end. As would be understood by one of skill in the art, the linker can be used to link the KLHDC2 ligase binding ligand to the target protein binding moiety using conventional chemistry such as, for example, by reacting a nucleophilic group on the target protein binding moiety (e.g., alcohol, amine, sulfhydryl, hydroxyl group) with an electrophilic group (e.g., a carboxylic acid) on the linker to which the KLHDC2 ligase binding ligand is attached, thereby creating a covalent bond between the KLHDC2 ligase binding ligand and the target protein binding moiety through the linker. Thus, in various aspects, L is a linker having a molecular weight of from about 100 g/mol to about 200 g/mol. In various further aspects, L is a linker having a length of from four to sixteen atoms comprising carbon, nitrogen, and oxygen.

In various aspects, L is a structure represented by a formula:

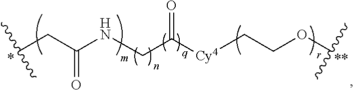

wherein * is connected to R¹ and ** is connected to Cy³; wherein m is 0 or 1; wherein n is 1, 2, or 3; wherein q is 0 or 1; wherein r is 1, 2, 3, or 4; and wherein Cy⁴ is a structure selected from:

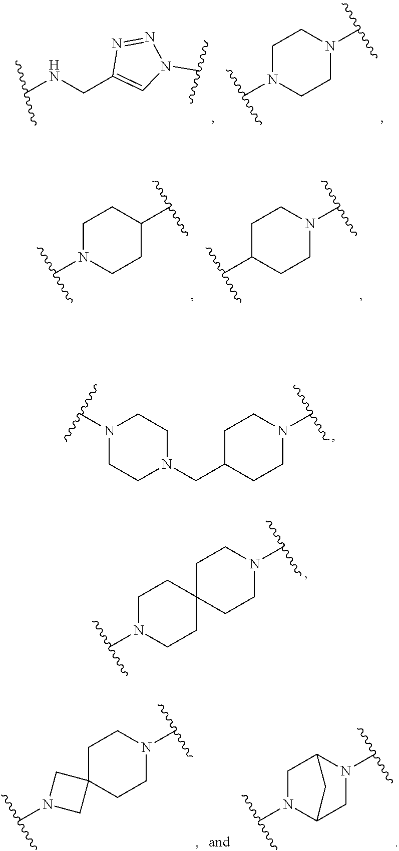

In various aspects, L is a structure represented by a formula:

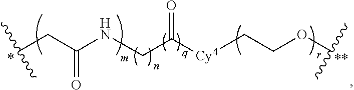

wherein * is connected to R¹ and ** is connected to Cy³; wherein m is 0 or 1; wherein n is 1, 2, or 3; wherein q is 0 or 1; wherein r is 1, 2, 3, or 4; and wherein Cy⁴ is a structure selected from:

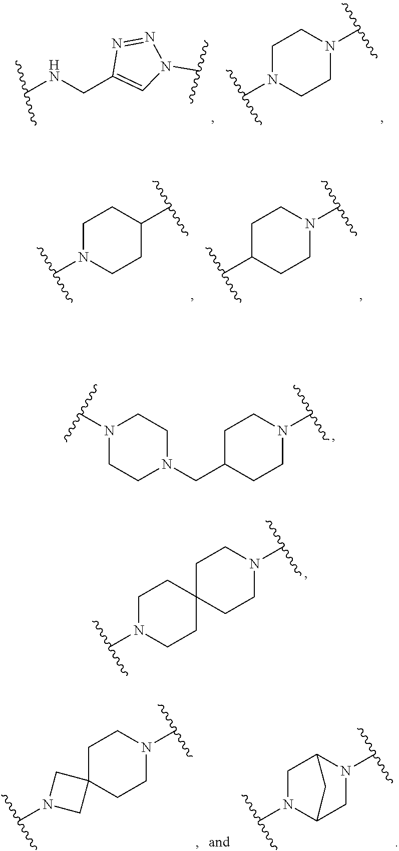

In one aspect, m is 0 or 1. In a further aspect, m is 0. In a still further aspect, m is 1.

In one aspect, n is 1, 2, or 3. In a further aspect, n is 1 or 2. In a still further aspect, n is 2 or 3. In yet a further aspect, n is 1 or 3. In an even further aspect, n is 1. In a still further aspect, n is 2. In yet a further aspect, n is 3.

In one aspect, q is 0 or 1. In a further aspect, q is 0. In a still further aspect, q is 1.

In one aspect, r is 1, 2, 3, or 4. In a further aspect, r is 1, 2, or 3. In a still further aspect, r is 2, 3, or 4. In yet a further aspect, r is 1, 3, or 4. In an even further aspect, r is 1, 2, or 4. In a still further aspect, r is 1 or 2. In yet a further aspect, r is 2 or 3. In an even further aspect, r is 1 or 3. In a still further aspect, r is 3 or 4. In yet a further aspect, r is 1 or 4. In an even further aspect, r is 2 or 4. In a still further aspect, r is 1. In yet a further aspect, r is 2. In an even further aspect, r is 3. In a still further aspect, r is 4.

In various aspects, L is a structure represented by a formula:

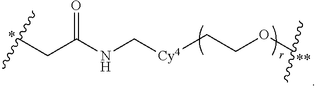

In various aspects, L is a structure represented by a formula:

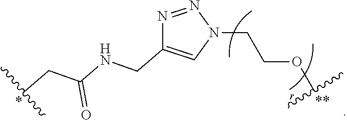

In various aspects, L is a structure selected from:

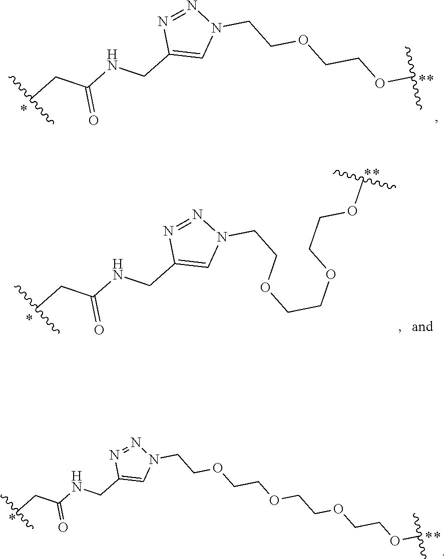 [SMILES: *CC(=O)NCc1cn(CCOCCO*)nn1 ; *CC(=O)NCc1cn(CCOCCOCCO*)nn1 ; *CC(=O)NCc1cn(CCOCCOCCOCCO*)nn1]

In various aspects, L is a structure represented by a formula:

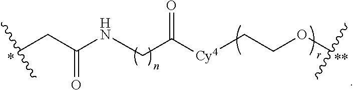

In various aspects, L is a structure represented by a formula:

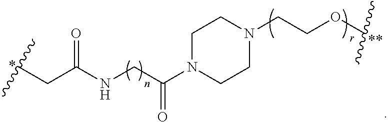

In various aspects L is a structure selected from:

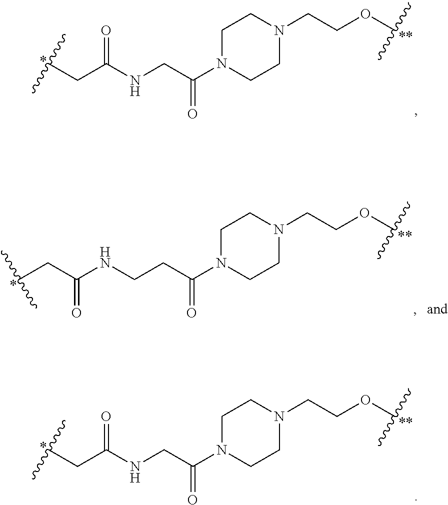 [SMILES: *CC(=O)NCC(=O)N1CCN(CCO*)CC1 ; *CC(=O)NCCC(=O)N1CCN(CCO*)CC1 ; *CC(=O)NCC(=O)N1CCN(CCO*)CC1]

In various aspects, L is a structure represented by a formula:

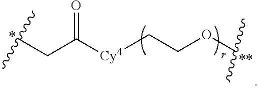

In various aspects, L is a structure represented by a formula:

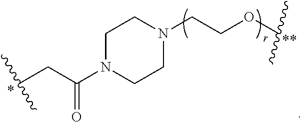

In various aspects, L is a structure represented by a formula:

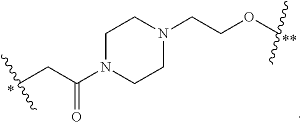 [SMILES: *CC(=O)N1CCN(CCO*)CC1]

In various aspects, L is a structure represented by a formula:

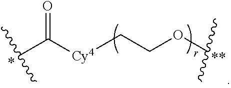

In various aspects, L is a structure represented by a formula:

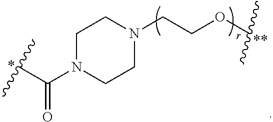

In various aspects, L is a structure:

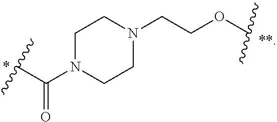 [SMILES: *OCCN1CCN(C(*)=O)CC1]

In various aspects, L is a structure represented by a formula:

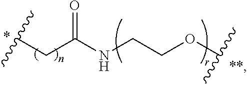

wherein * is connected to R¹ and ** is connected to Cy³; wherein n is 1, 2, or 3; and wherein r is 1, 2, 3, or 4. In a further aspect, n is 2.

In various aspects, L is a structure represented by a formula:

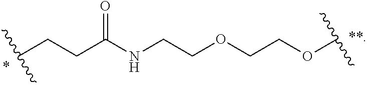 [SMILES: *CCC(=O)NCCOCCO*]

In various aspects, L is a structure represented by a formula:

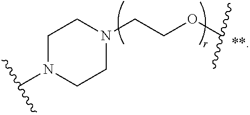

In various aspects, L is a structure represented by a formula:

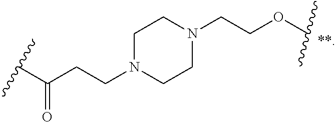 [SMILES: *OCCN1CCN(CCC(*)=O)CC1]

b. R¹ Groups

In one aspect, R¹ is a residue of a small molecule having a molecular weight of from about 150 g/mol to about 600 g/mol and a binding affinity (Kᵢ) to a target protein of at least about 20 μM. As used herein, the term “target protein” refers to a protein targeted by the disclosed heterobifunctional compounds and, in particular, by the protein binding moiety, R¹. A target protein refers to any protein involved in the metabolism or catabolism of a cell and/or organ of a subject, especially including proteins that modulate a disease state or condition to be treated with the disclosed heterobifunctional compounds. Target proteins can also include proteins from microbes, such as bacteria, viruses, fungi, and protozoa. In general, target proteins can include, for example, structural proteins; receptors; enzymes; cell surface proteins; proteins pertinent to the integrated function of a cell, including proteins involved in catalysis, aromatase activity, motor activity, helicase activity, metabolic processes (such as anabolism and catabolism), antioxidant activity, proteolysis, biosynthesis, kinase activity, oxidoreductase activity, transferase activity, hydrolase activity, lyase activity, isomerase activity, ligase activity, enzyme regulator activity, signal transducer activity, structural molecule activity, binding activity (to a protein, lipid or carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport (including protein transporter activity, nuclear transport, ion transporter activity, channel transporter activity, carrier activity, permease activity, secretion activity, electron transporter activity), pathogenesis, chaperone regulator activity, nucleic acid binding activity, transcription regulator activity, extracellular organization and biogenesis activity, and translation regulator activity. Target proteins can include proteins from eukaryotes and prokaryotes, including humans and other animals, microbes, plants, and viruses, among numerous others.

Non-limiting examples of target proteins include, but are not limited to, B7, B7-1, and B7-2 (providing second signals to T cells), TINFR1m, TNFR2, NADPH oxidase, BclIBax, and other partners in the apoptosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, carbonic anhydrase, chemokine receptors, JAW STAT, RXR and similar, HIV 1 protease, HIV 1 integrase, influenza neuramimidase, hepatitis B reverse transcriptase, sodium channel, protein P-glycoprotein (and MRP), tyrosine kinases, CD23, CD124, tyrosine kinase p56 lck, CD4, CD5, IL-2 receptor, IL-1 receptor, TNF-alphaR, ICAM1, Cat+ channels, VCAM, VLA-4 integrin, selectins, CD40/CD40L, newokinins and receptors, inosine monophosphate dehydrogenase, p38 MAP Kinase, Ras1Raf1MEWERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, adenosine receptors, adenosine kinase and AMP deaminase, purinergic receptors (P2Y1, P2Y2, P2Y4, P2Y6, P2X1-7), famesyltransferases, geranylgeranyl transferase, TrkA a receptor for NGF, beta-amyloid, tyrosine kinase Flk-IIKDR, vitronectin receptor, integrin receptor, Her-21 neu, telomerase inhibition, tumor associated protein (TMP), Bcr-Abl tyrosine kinase, cytosolic phospholipaseA2 and EGF receptor tyrosine kinase. Additional protein targets include, but are not limited to, ecdysone 20-monooxygenase, ion channel of the GABA gated chloride channel, acetylcholinesterase, voltage-sensitive sodium channel protein, calcium release channel, and chloride channels. Additional target proteins include, but are not limited to, acetyl-CoA carboxylase, adenylosuccinate synthetase, protoporphyrinogen oxidase, and enolpyruvylshikimate-phosphate synthase. Exemplary target proteins can also include drug resistant and multiple drug resistance (MDR) proteins.

As further detailed herein, exemplary residues of a small molecule having a molecular weight of from about 150 g/mol to about 600 g/mol and a binding affinity (Kᵢ) to a target protein of at least about 20 μM (e.g., R¹ as described herein) include, but are not limited to:

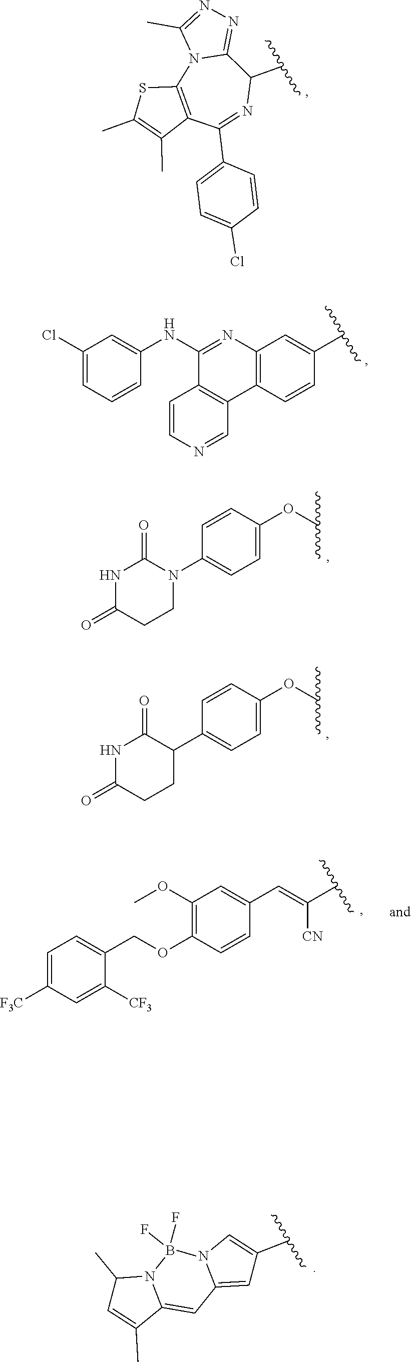

In various aspects, R¹ is a structure selected from:

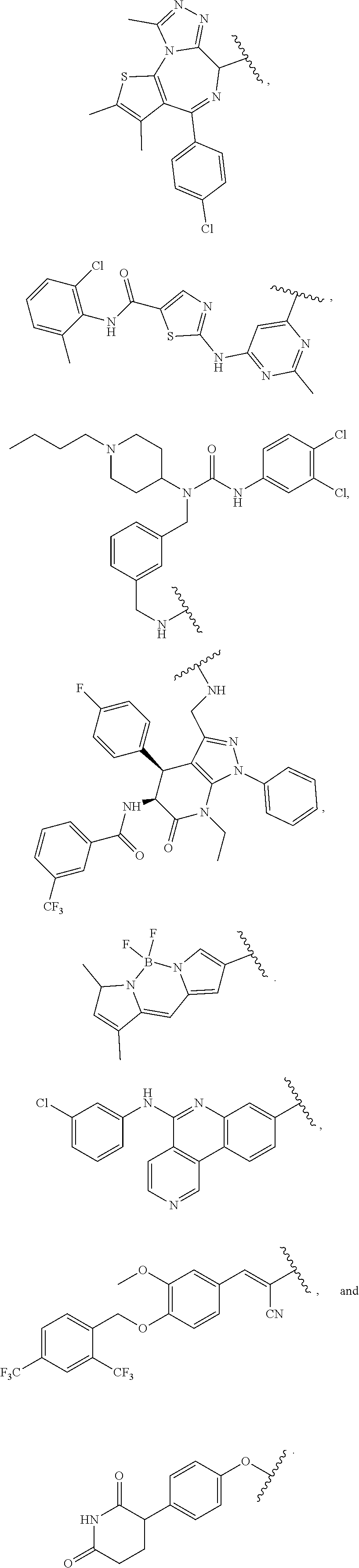

In various aspects, R¹ is a structure:

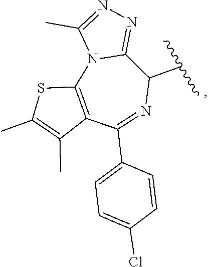 [SMILES: *C1N=C(c2ccc(Cl)cc2)c2c(sc(C)c2C)-n2c(C)nnc21]

In various aspects, R¹ is a structure:

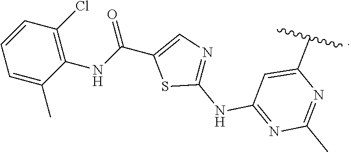 [SMILES: *c1cc(Nc2ncc(C(=O)Nc3c(C)cccc3Cl)s2)nc(C)n1]

In various aspects, R¹ is a structure:

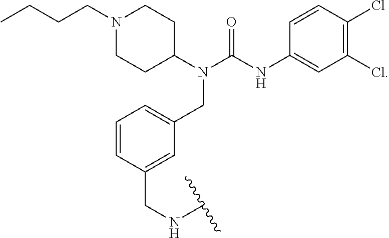 [SMILES: *NCc1cccc(CN(C(=O)Nc2ccc(Cl)c(Cl)c2)C2CCN(CCCC)CC2)c1]

In various aspects, R¹ is a structure:

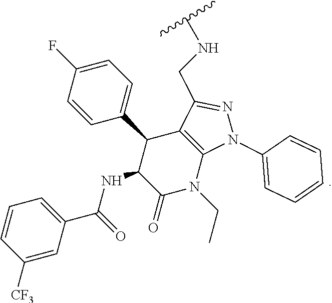 [SMILES: *NCc1nn(-c2ccccc2)c2c1[C@H](c1ccc(F)cc1)[C@H](NC(=O)c1cccc(C(F)(F)F)c1)C(=O)N2CC]

c. CY³ Groups

In one aspect, Cy³ is a 9- or 10-membered heterobicycle, a 10-membered biaryl, or a 9- or 10-membered heterobiaryl, and is substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a further aspect, Cy³ is a 9- or 10-membered heterobicycle, a 10-membered biaryl, or a 9- or 10-membered heterobiaryl, and is substituted with 0, 1, 2, or 3 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy³ is a 9- or 10-membered heterobicycle, a 10-membered biaryl, or a 9- or 10-membered heterobiaryl, and is substituted with 0, 1, or 2 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In yet a further aspect, Cy³ is a 9- or 10-membered heterobicycle, a 10-membered biaryl, or a 9- or 10-membered heterobiaryl, and is substituted with 0 or 1 group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In an even further aspect, Cy³ is a 9- or 10-membered heterobicycle, a 10-membered biaryl, or a 9- or 10-membered heterobiaryl, and is monosubstituted with a group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy³ is a 9- or 10-membered heterobicycle, a 10-membered biaryl, or a 9- or 10-membered heterobiaryl, and is unsubstituted.

In various aspects, Cy³ is a 9- or 10-membered heterobicycle, and is substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a further aspect, Cy³ is a 9- or 10-membered heterobicycle, and is substituted with 0, 1, 2, or 3 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy³ is a 9- or 10-membered heterobicycle, and is substituted with 0, 1, or 2 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In yet a further aspect, Cy³ is a 9- or 10-membered heterobicycle, and is substituted with 0 or 1 group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In an even further aspect, Cy³ is a 9- or 10-membered heterobicycle, and is monosubstituted with a group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy³ is a 9- or 10-membered heterobicycle, and is unsubstituted.

In various aspects, Cy³ is a 9-membered heterobicycle substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a further aspect, Cy³ is a 9-membered heterobicycle substituted with 0, 1, 2, or 3 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy³ is a 9-membered heterobicycle substituted with 0, 1, or 2 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In yet a further aspect, Cy³ is a 9-membered heterobicycle substituted with 0 or 1 group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹.

In an even further aspect, Cy³ is a 9-membered heterobicycle monosubstituted with a group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy³ is an unsubstituted 9-membered heterobicycle.

In various aspects, Cy³ is a 10-membered heterobicycle substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a further aspect, Cy³ is a 10-membered heterobicycle substituted with 0, 1, 2, or 3 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy³ is a 10-membered heterobicycle substituted with 0, 1, or 2 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In yet a further aspect, Cy³ is a 10-membered heterobicycle substituted with 0 or 1 group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1—C4 alkylamino, (C1-C4)(C1-C4) dialkylamino, C1-C4 aminoalkyl, —OR¹⁰, and —NHC(O)R¹¹.

In an even further aspect, Cy³ is a 10-membered heterobicycle monosubstituted with a group selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy³ is an unsubstituted 10-membered heterobicycle.

In various aspects, Cy³ is a 10-membered heterobicycle substituted with 0, 1, 2, 3, or 4 groups independently selected from halogen, C1-C4 alkyl, —OR¹⁰, and —NHC(O)R¹¹. In a further aspect, Cy³ is a 10-membered heterobicycle substituted with 0, 1, 2, or 3 groups independently selected from halogen, C1-C4 alkyl, —OR¹⁰, and —NHC(O)R¹¹. In a still further aspect, Cy³ is a 10-membered heterobicycle substituted with 0, 1, or 2 groups independently selected from halogen, C1-C4 alkyl, —OR¹⁰, and —NHC(O)R¹¹. In yet a further aspect, Cy³ is a 10-membered heterobicycle substituted with 0 or 1 group selected from halogen, C1-C4 alkyl, —OR¹⁰, and —NHC(O)R¹¹. In an even further aspect, Cy³ is a 10-membered heterobicycle monosubstituted with a group selected from halogen, C1-C4 alkyl, —OR¹⁰, and —NHC(O)R¹¹. In a still further aspect, Cy³ is an unsubstituted 10-membered heterobicycle.

In various aspects, Cy³ is a 10-membered biaryl substituted with 0, 1, 2, 3, or 4 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a further aspect, Cy³ is a 10-membered biaryl substituted with 0, 1, 2, or 3 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy³ is a 10-membered biaryl substituted with 0, 1, or 2 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹.

In yet a further aspect, Cy³ is a 10-membered biaryl substituted with 0 or 1 group selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In an even further aspect, Cy³ is a 10-membered biaryl monosubstituted with a group selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy³ is an unsubstituted 10-membered biaryl.

In various aspects, Cy³ is a 10-membered biaryl substituted with 0, 1, 2, 3, or 4 groups independently selected from halogen, C1-C4 alkyl, —OR¹⁰, and —NHC(O)R¹¹. In a further aspect, Cy³ is a 10-membered biaryl substituted with 0, 1, 2, or 3 groups independently selected from halogen, C1-C4 alkyl, —OR¹⁰, and —NHC(O)R¹¹. In a still further aspect, Cy³ is a 10-membered biaryl substituted with 0, 1, or 2 groups independently selected from halogen, C1-C4 alkyl, —OR¹⁰, and —NHC(O)R¹¹. In yet a further aspect, Cy³ is a 10-membered biaryl substituted with 0 or 1 group selected from halogen, C1-C4 alkyl, —OR¹⁰, and —NHC(O)R¹¹. In an even further aspect, Cy³ is a 10-membered biaryl monosubstituted with a group selected from halogen, C1-C4 alkyl, —OR¹⁰, and —NHC(O)R¹¹. In a still further aspect, Cy³ is an unsubstituted 10-membered biaryl.

In various aspects, Cy³ is a 9-membered heterobiaryl substituted with 0, 1, 2, 3, or 4 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a further aspect, Cy³ is a 9-membered heterobiaryl substituted with 0, 1, 2, or 3 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy³ is a 9-membered heterobiaryl substituted with 0, 1, or 2 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In yet a further aspect, Cy³ is a 9-membered heterobiaryl substituted with 0 or 1 group selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In an even further aspect, Cy³ is a 9-membered heterobiaryl monosubstituted with a group selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy³ is an unsubstituted 9-membered heterobiaryl.

In various aspects, Cy³ is a 10-membered heterobiaryl substituted with 0, 1, 2, 3, or 4 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1—C4)(C1-C4) dialkylamino, C1-C4 aminoalkyl, —OR¹⁰, and —NHC(O)R¹¹. In a further aspect, Cy³ is a 10-membered heterobiaryl substituted with 0, 1, 2, or 3 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy³ is a 10-membered heterobiaryl substituted with 0, 1, or 2 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In yet a further aspect, Cy³ is a 10-membered heterobiaryl substituted with 0 or 1 group selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In an even further aspect, Cy³ is a 10-membered heterobiaryl monosubstituted with a group selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹. In a still further aspect, Cy³ is an unsubstituted 10-membered heterobiaryl.

In various aspects, Cy³ is a 10-membered heterobiaryl substituted with 0, 1, 2, 3, or 4 groups independently selected from halogen, C1-C4 alkyl, —OR¹⁰, and —NHC(O)R¹¹. In a further aspect, Cy³ is a 10-membered heterobiaryl substituted with 0, 1, 2, or 3 groups independently selected from halogen, C1-C4 alkyl, —OR¹⁰, and —NHC(O)R¹¹. In a still further aspect, Cy³ is a 10-membered heterobiaryl substituted with 0, 1, or 2 groups independently selected from halogen, C1-C4 alkyl, —OR¹⁰, and —NHC(O)R¹¹. In yet a further aspect, Cy³ is a 10-membered heterobiaryl substituted with 0 or 1 group selected from halogen, C1-C4 alkyl, —OR¹⁰, and —NHC(O)R¹¹. In an even further aspect, Cy³ is a 10-membered heterobiaryl monosubstituted with a group selected from halogen, C1-C4 alkyl, —OR¹⁰, and —NHC(O)R¹¹. In a still further aspect, Cy³ is an unsubstituted 10-membered heterobiaryl.

d. Cy⁴ Groups

In one aspect, Cy⁴ is a structure selected from:

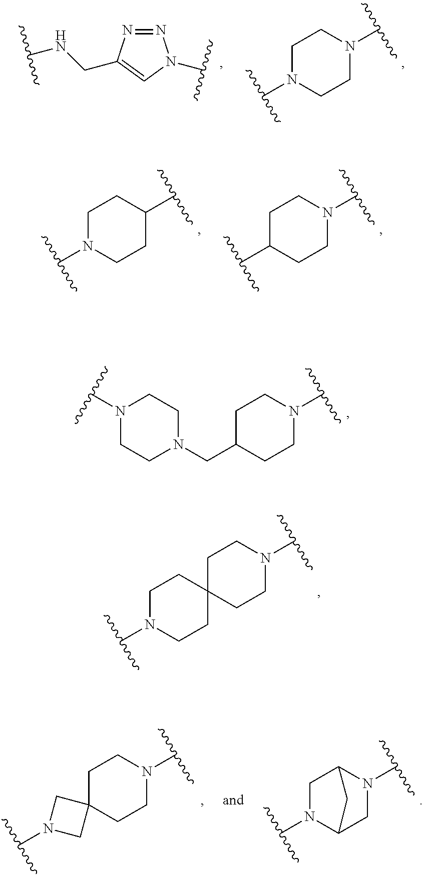

In various aspects, Cy⁴ is a structure:

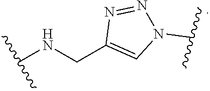 [SMILES: *NCc1cn(*)nn1]

In various aspects, Cy⁴ is a structure:

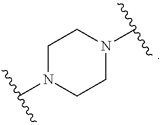 [SMILES: *N1CCN(*)CC1]

### 2. Example Compounds

In one aspect, a compound can be present as one or more of the following structures:

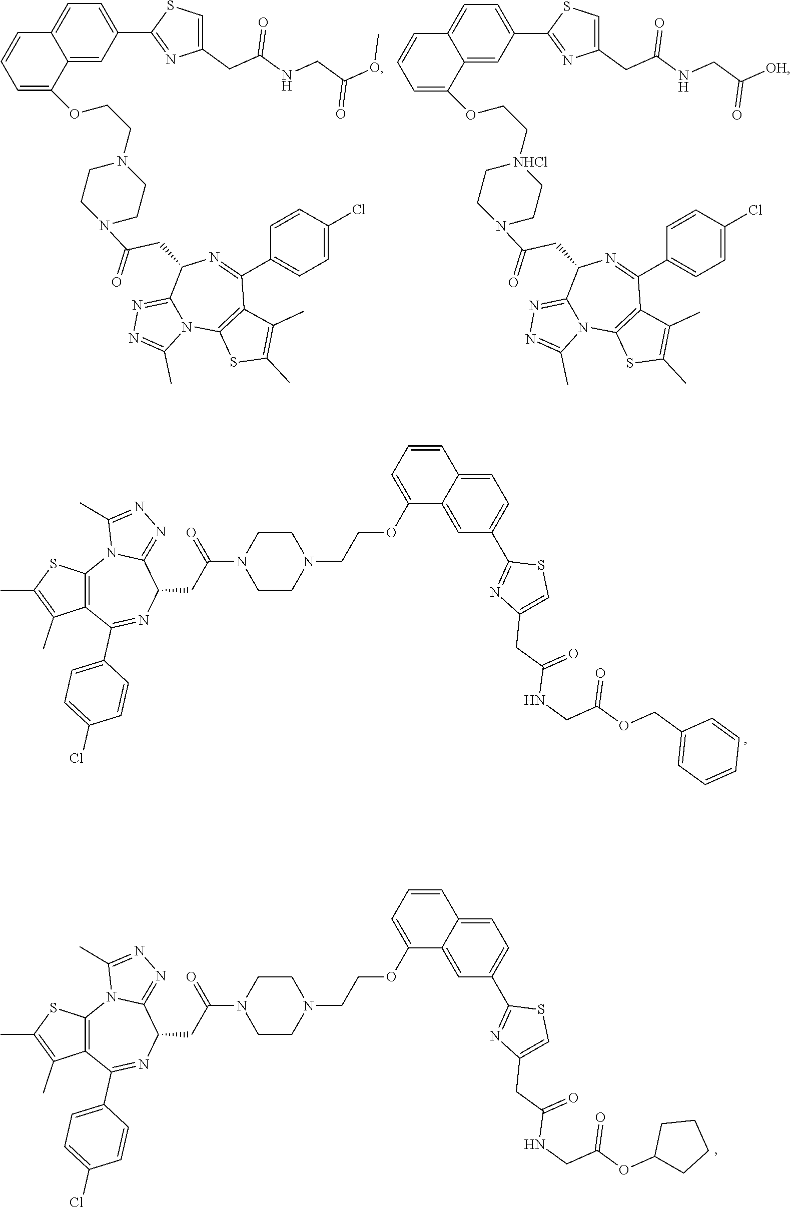

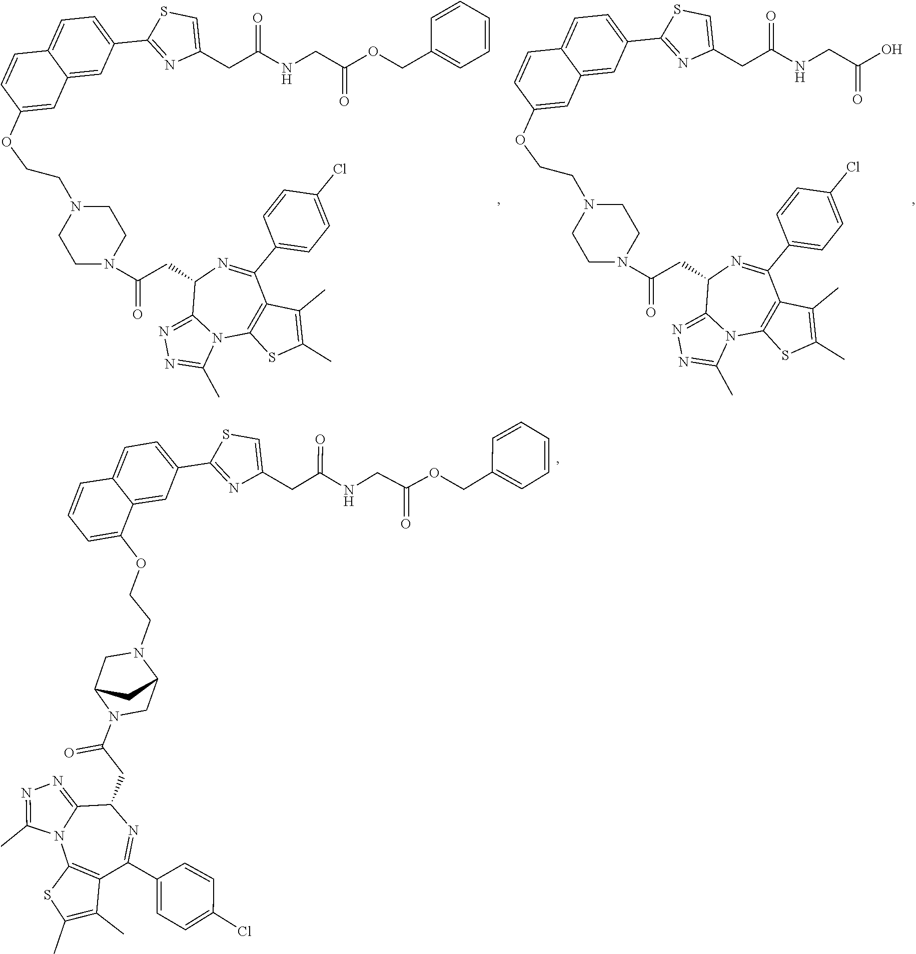

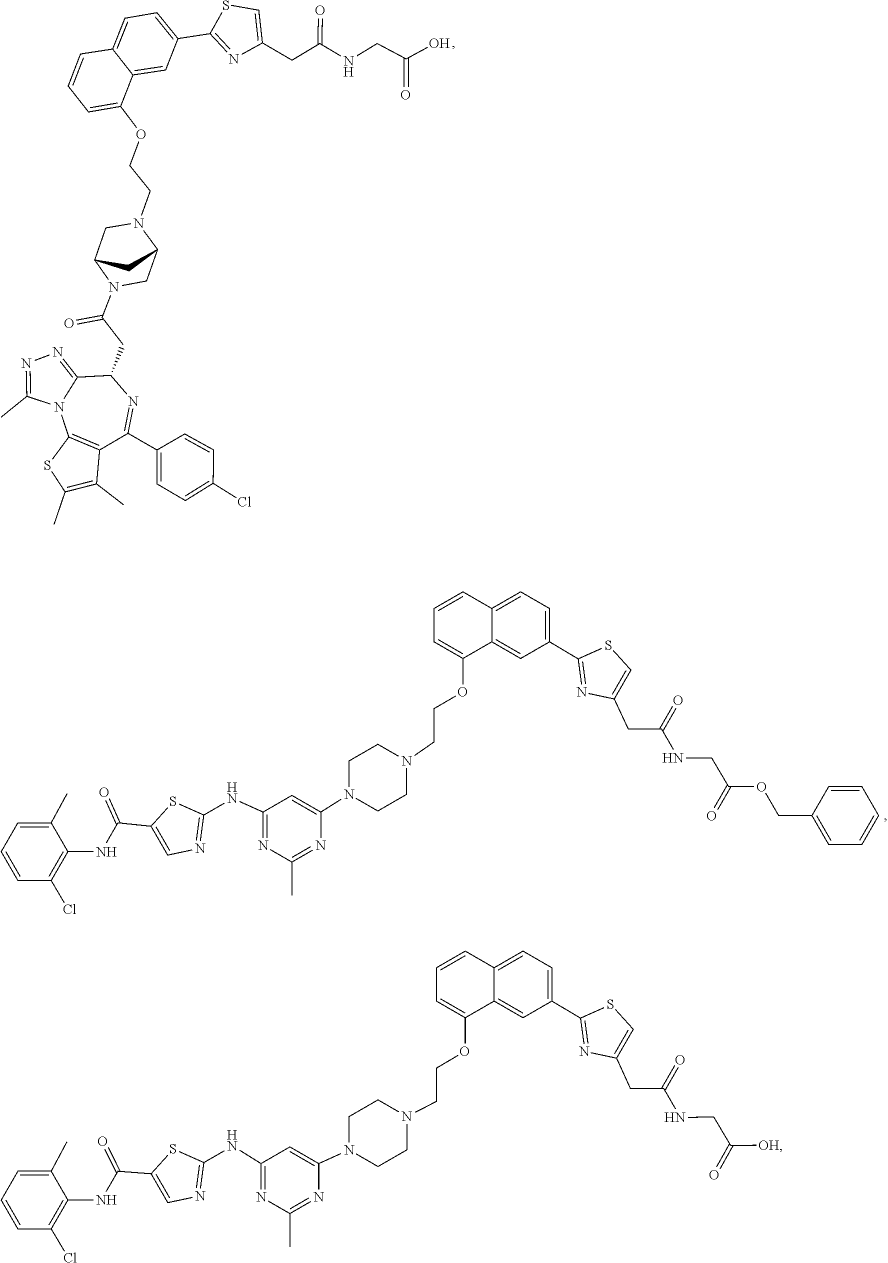 [SMILES: Cc1sc2c(c1C)C(c1ccc(Cl)cc1)=N[C@@H](CC(=O)N1C[C@@H]3C[C@H]1CN3CCOc1cccc3ccc(-c4nc(CC(=O)NCC(=O)O)cs4)cc13)c1nnc(C)n1-2 ; Cc1nc(Nc2ncc(C(=O)Nc3c(C)cccc3Cl)s2)cc(N2CCN(CCOc3cccc4ccc(-c5nc(CC(=O)NCC(=O)OCc6ccccc6)cs5)cc34)CC2)n1 ; Cc1nc(Nc2ncc(C(=O)Nc3c(C)cccc3Cl)s2)cc(N2CCN(CCOc3cccc4ccc(-c5nc(CC(=O)NCC(=O)O)cs5)cc34)CC2)n1]

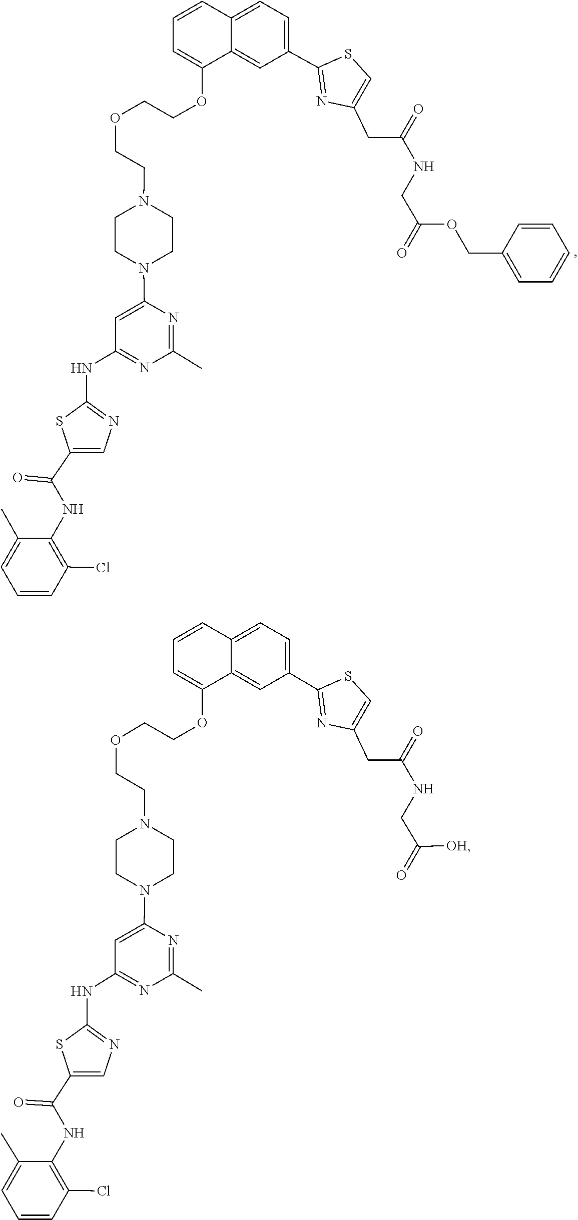 [SMILES: Cc1nc(Nc2ncc(C(=O)Nc3c(C)cccc3Cl)s2)cc(N2CCN(CCOCCOc3cccc4ccc(-c5nc(CC(=O)NCC(=O)OCc6ccccc6)cs5)cc34)CC2)n1 ; Cc1nc(Nc2ncc(C(=O)Nc3c(C)cccc3Cl)s2)cc(N2CCN(CCOCCOc3cccc4ccc(-c5nc(CC(=O)NCC(=O)O)cs5)cc34)CC2)n1]

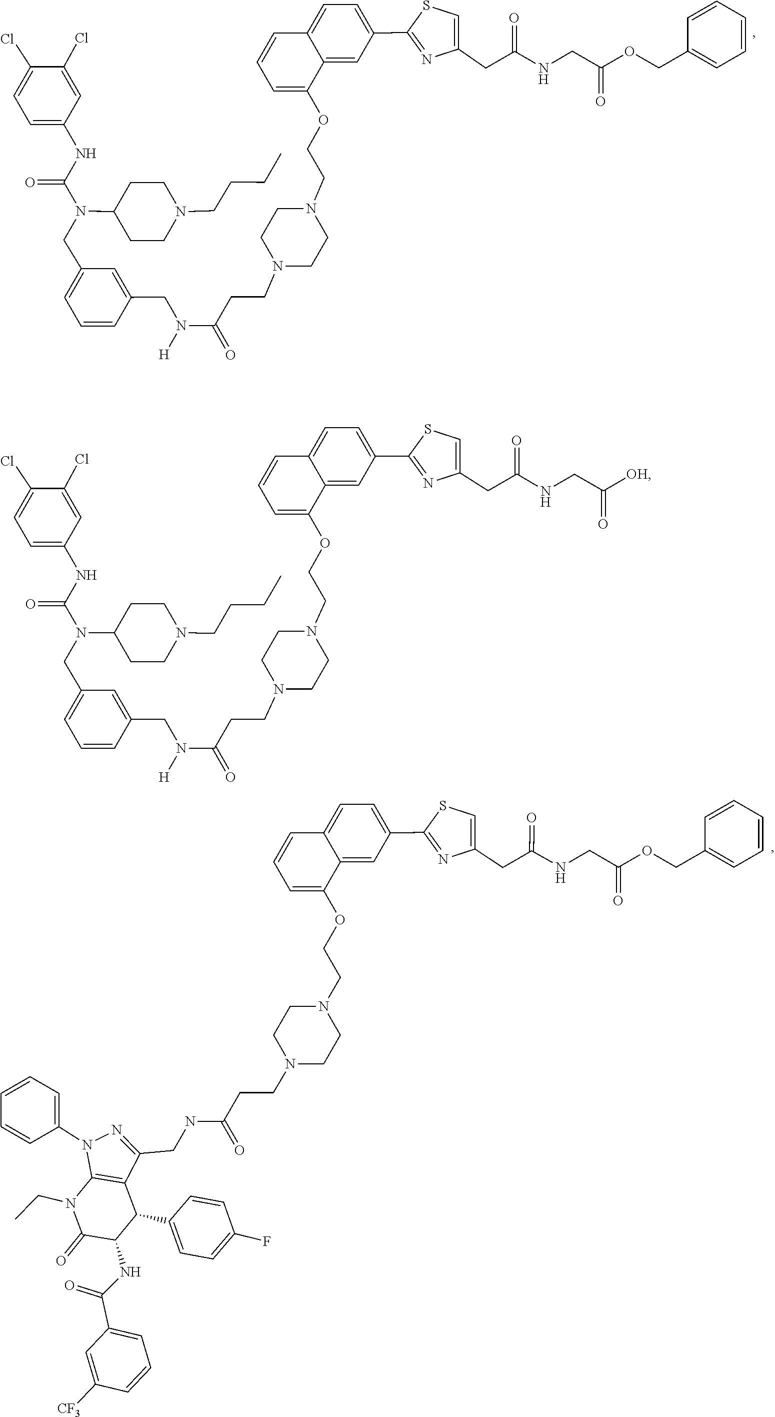

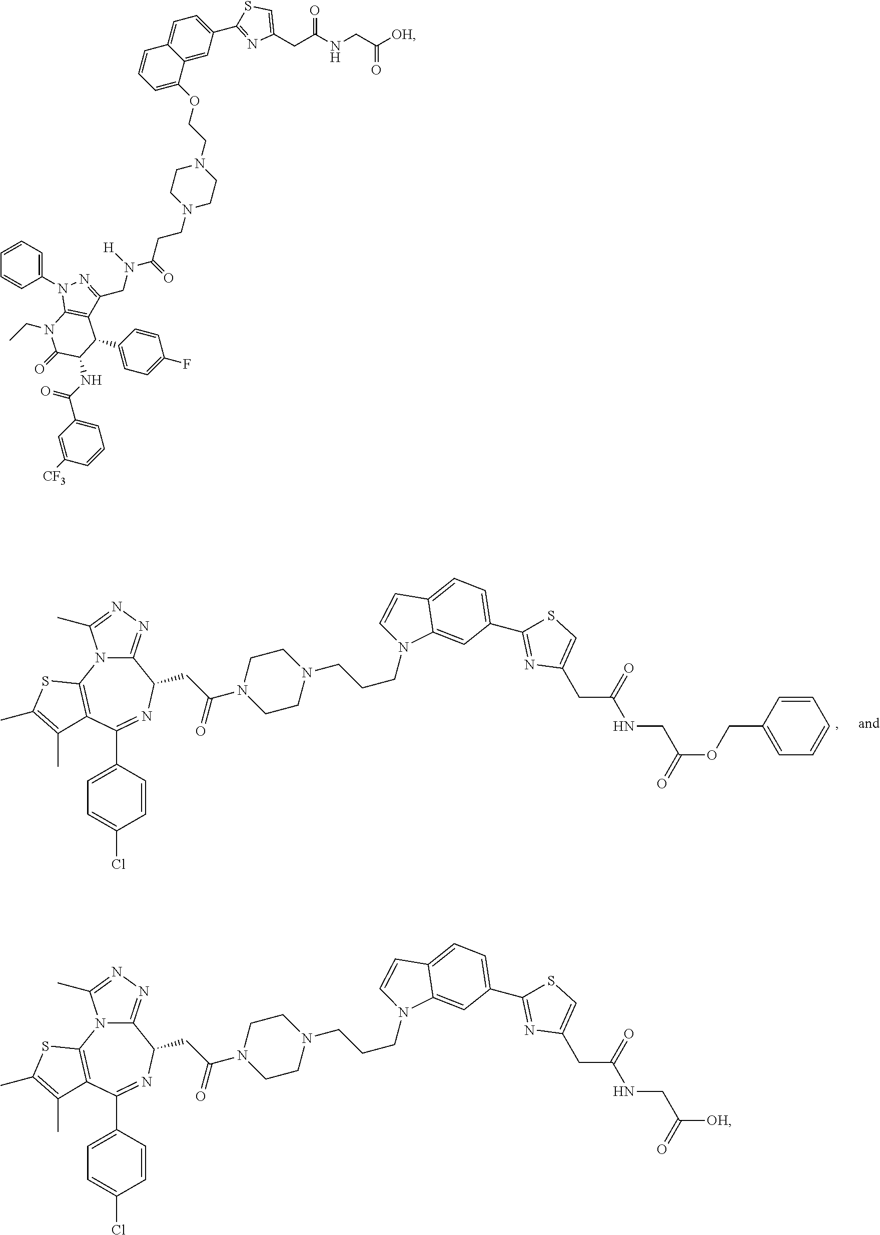 [SMILES: CCN1C(=O)[C@@H](NC(=O)c2cccc(C(F)(F)F)c2)[C@@H](c2ccc(F)cc2)c2c(CNC(=O)CCN3CCN(CCOc4cccc5ccc(-c6nc(CC(=O)NCC(=O)O)cs6)cc45)CC3)nn(-c3ccccc3)c21 ; Cc1sc2c(c1C)C(c1ccc(Cl)cc1)=N[C@@H](CC(=O)N1CCN(CCCn3ccc4ccc(-c5nc(CC(=O)NCC(=O)OCc6ccccc6)cs5)cc43)CC1)c1nnc(C)n1-2 ; Cc1sc2c(c1C)C(c1ccc(Cl)cc1)=N[C@@H](CC(=O)N1CCN(CCCn3ccc4ccc(-c5nc(CC(=O)NCC(=O)O)cs5)cc43)CC1)c1nnc(C)n1-2]

or a pharmaceutically acceptable salt thereof.

### D. Methods of Making a Compound

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-VIII, as described and exemplified below. The following examples are provided so that the invention might be more fully understood, are illustrative only, and should not be construed as limiting.

### 1. Route I

In one aspect, the disclosed compounds can be prepared as shown below.

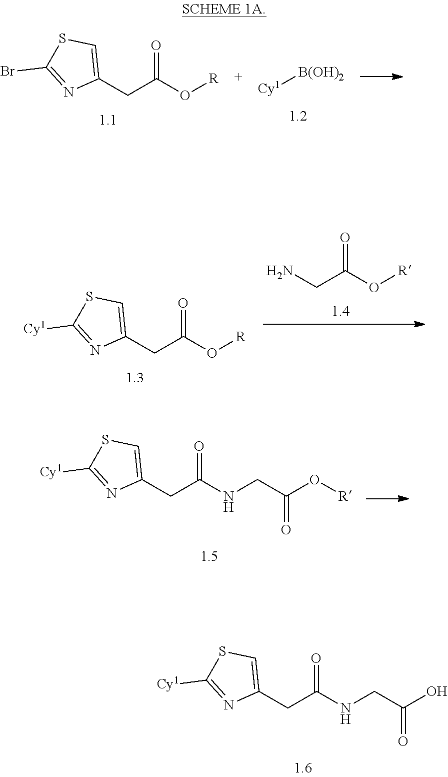

Compounds are represented in generic form, wherein R is a C1-C4 alkyl, R′ is selected from C1-C4 alkyl, C4-C6 cycloalkyl, —(C1-C4 alkyl)OC(O)(C1-C4 alkyl), and —CH₂C₆H₅, and with other substituents are as noted in compound descriptions elsewhere herein. A specific non-limiting example of the synthesis shown in Scheme 1A is provided below.

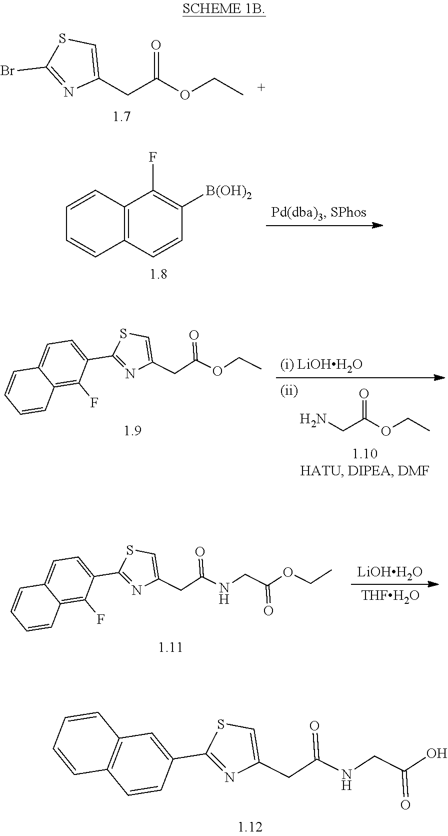

In one aspect, compounds of type 1.12, and similar compounds, can be prepared according to reaction Scheme 1B above. Thus, compounds of type 1.9 can be prepared by a coupling reaction between an appropriate 2-bromothiazole, e.g., 1.7 as shown above, and an appropriate aryl boronic acid, e.g., 1.8 as shown above. Appropriate 2-bromothiazoles and appropriate aryl boronic acids are commercially available or prepared by methods known to one skilled in the art. The coupling reaction is carried out in the presence of an appropriate catalyst, e.g., tris(dibenzylideneacetone)dipalladium(0), and an appropriate ligand, e.g., 2-dicyclohexylphosphino-2′,6′-dimethoxybiphenyl (SPhos). Compounds of type 1.11 can be prepared by reduction of an appropriate alkyl ester, e.g., 1.9 as shown above, followed by a coupling reaction with an appropriate glycinate, e.g., 1.10 as shown above. Appropriate glycinates are commercially available or prepared by methods known to one skilled in the art. The reduction is carried out in the presence of an appropriate base, e.g., aqueous lithium hydroxide. The coupling reaction is carried out in the presence of an appropriate coupling agent, e.g., hexafluorophosphate azabenzotriazole tetramethyl uranium (HATU), and an appropriate base, e.g., diisopropylethylamine (DIPEA), in an appropriate solvent, e.g., dimethylformamide (DMF). Compounds of type 1.12 can be prepared by reduction of an appropriate ester, e.g., 1.11 as shown above. The reduction is carried out in the presence of an appropriate base, e.g., aqueous lithium hydroxide, in an appropriate solvent system, e.g., tetrahydrofuran (THF) and water. As can be appreciated by one skilled in the art, the above reaction provides an example of a generalized approach wherein compounds similar in structure to the specific reactants above (compounds similar to compounds of type 1.1, 1.2, 1.3, 1.4, and 1.5), can be substituted in the reaction to provide substituted compounds similar to Formula 1.6.

### 2. Route II

In one aspect, the disclosed heterobifunctional compounds can be prepared as shown below.

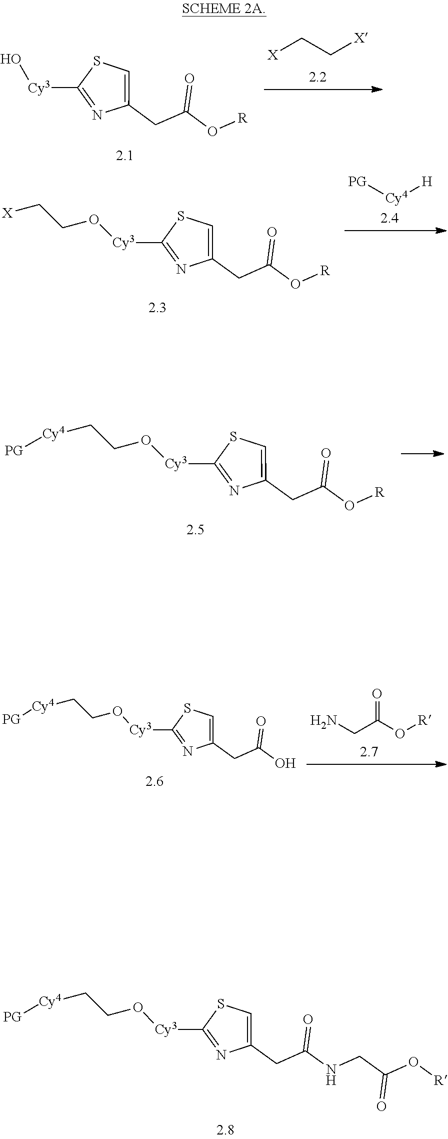

Compounds are represented in generic form, wherein each of X and X′ is independently halogen, R is C1-C4 alkyl, R′ is selected from C1-C4 alkyl, C4-C6 cycloalkyl, —(C1-C4 alkyl)OC(O)(C1-C4 alkyl), and —CH₂C₆H₅, PG is an amine protecting group, and with other substituents are as noted in compound descriptions elsewhere herein. A specific non-limiting example of the synthesis shown in Scheme 2A is provided below.

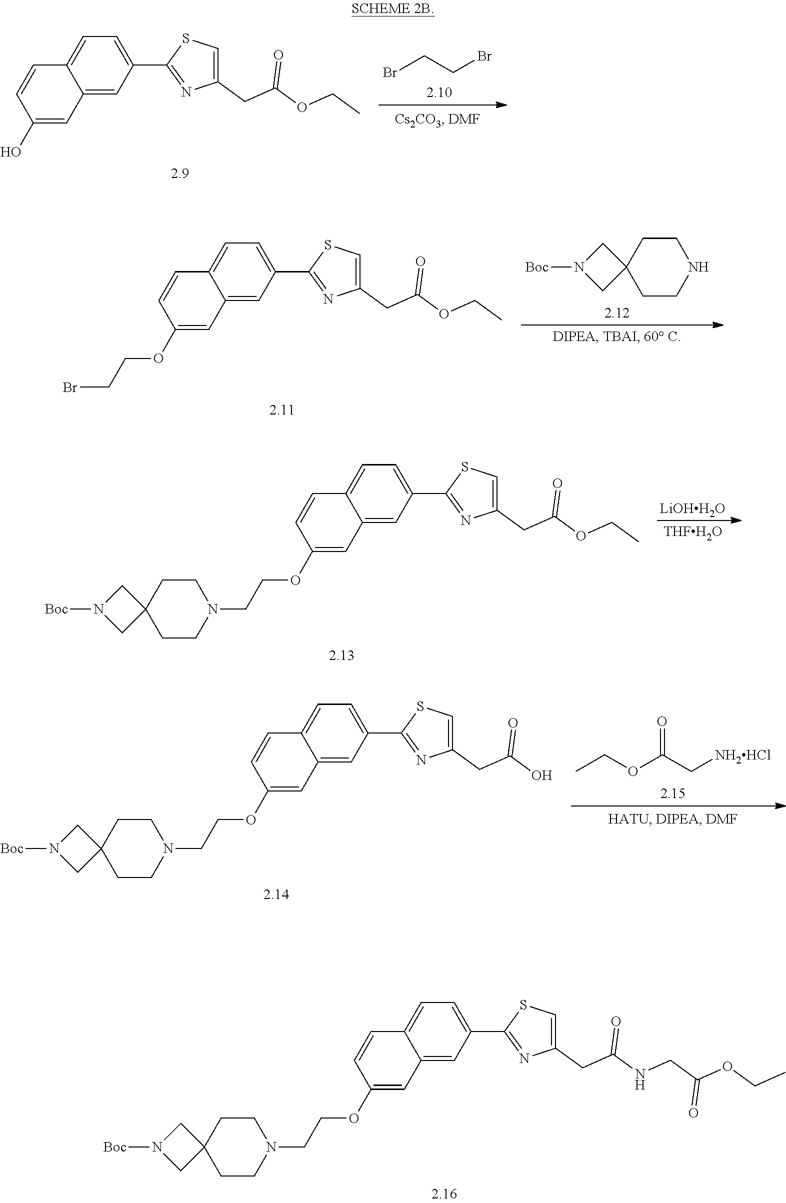

In one aspect, compounds of type 2.16, and similar compounds, can be prepared according to reaction Scheme 2B above. Thus, compounds of type 2.11 can be prepared by a coupling reaction between an appropriate aryl alcohol, e.g., 2.9 as shown above, and an appropriate dihalide, e.g., 2.10 as shown above. Appropriate aryl alcohols and appropriate dihalides are commercially available or prepared by methods known to one skilled in the art. The coupling reaction is carried out in the presence of an appropriate base, e.g., cesium carbonate, in an appropriate solvent, e.g., DMF. Compounds of type 2.13 can be prepared by a coupling reaction between an appropriate halide, e.g., 2.11 as shown above, and an appropriate cyclic amine, e.g., 2.12 as shown above. Appropriate cyclic amines are commercially available or prepared by methods known to one skilled in the art. The coupling reaction is carried out in the presence of an appropriate base, e.g., DIPEA, and an appropriate salt, e.g., tetra-n-butylammonium iodide, at an appropriate temperature, e.g., 60° C. Compounds of type 2.14 can be prepared by reduction of an appropriate ester, e.g., 2.13 as shown above. The reduction is carried out in the presence of an appropriate base, e.g., aqueous lithium hydroxide, in an appropriate solvent system, e.g., tetrahydrofuran (THF) and water. Compounds of type 2.16 can be prepared by a coupling reaction between an appropriate carboxylic acid, e.g., 2.14 as shown above, and an appropriate amine, e.g., 2.15 as shown above. Appropriate amines are commercially available or prepared by methods known to one of skill in the art. The coupling reaction is carried out in the presence of an appropriate coupling agent, e.g., HATU, and an appropriate base, e.g., DIPEA, in an appropriate solvent, e.g., DMF. As can be appreciated by one skilled in the art, the above reaction provides an example of a generalized approach wherein compounds similar in structure to the specific reactants above (compounds similar to compounds of type 2.1, 2.2, 2.3, 2.4, 2.5, 2.6, and 2.7), can be substituted in the reaction to provide substituted heterobifunctional compounds similar to Formula 2.8.

### 3. Route III

In one aspect, the disclosed heterobifunctional compounds can be prepared as shown below.

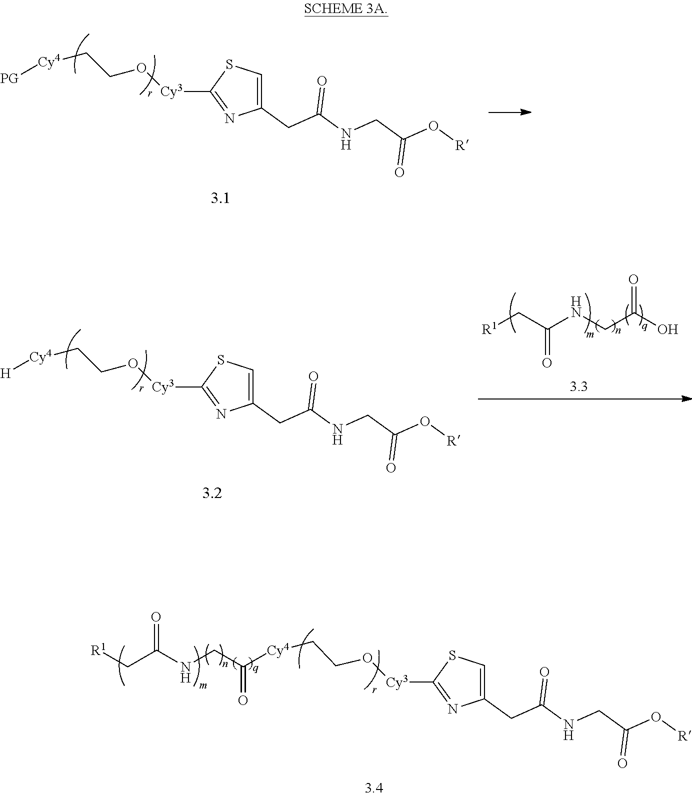

Compounds are represented in generic form, wherein R′ is selected from C1-C4 alkyl, C4-C6 cycloalkyl, —(C1-C4 alkyl)OC(O)(C1-C4 alkyl), and —CH₂C₆H₅, PG is an amine protecting group, and with other substituents are as noted in compound descriptions elsewhere herein. A specific non-limiting example of the synthesis shown in Scheme 3A is provided below.

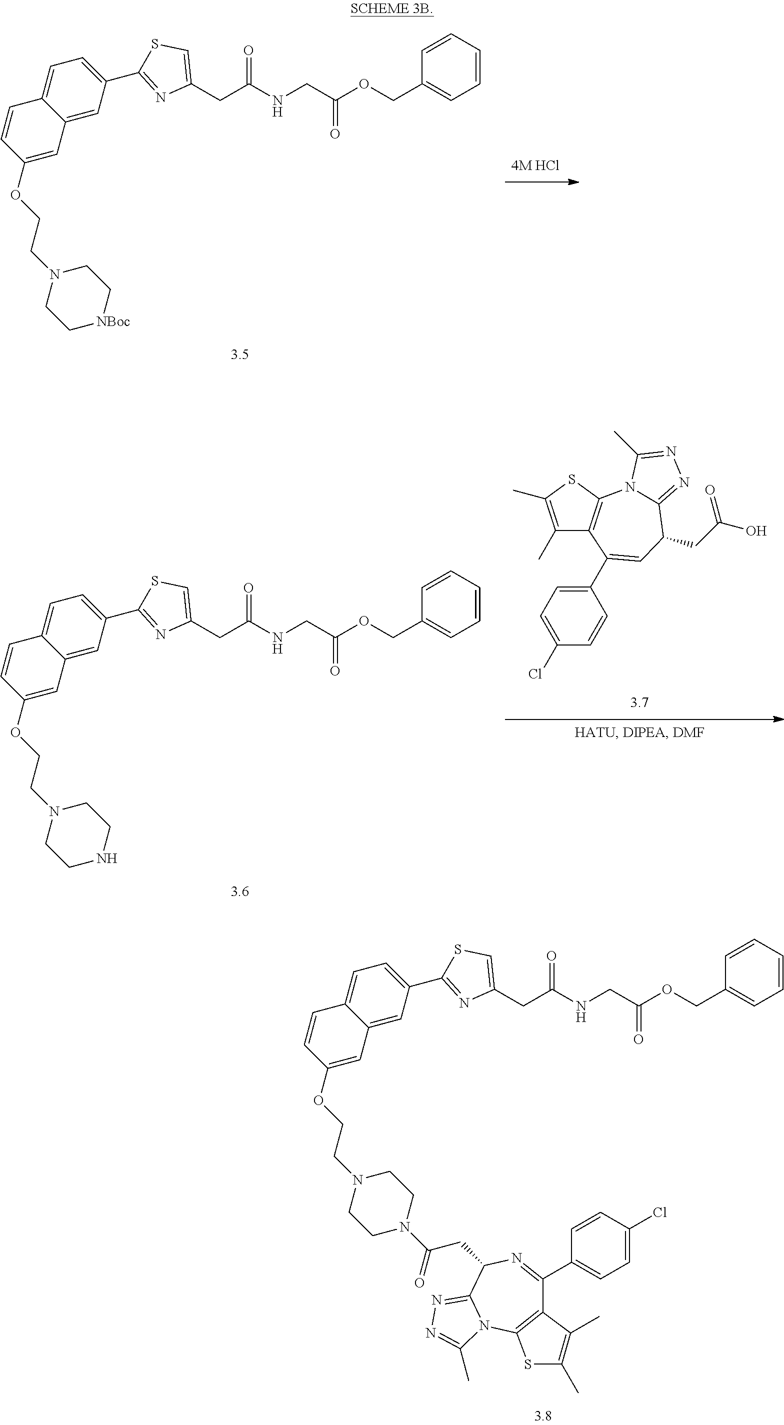 [SMILES: CC(C)(C)OC(=O)N1CCN(CCOc2ccc3ccc(-c4nc(CC(=O)NCC(=O)OCc5ccccc5)cs4)cc3c2)CC1 ; O=C(Cc1csc(-c2ccc3ccc(OCCN4CCNCC4)cc3c2)n1)NCC(=O)OCc1ccccc1 ; Cc1sc2c(c1C)C(c1ccc(Cl)cc1)=C[C@@H](CC(=O)O)c1nnc(C)n1-2 ; Cc1sc2c(c1C)C(c1ccc(Cl)cc1)=N[C@@H](CC(=O)N1CCN(CCOc3ccc4ccc(-c5nc(CC(=O)NCC(=O)OCc6ccccc6)cs5)cc4c3)CC1)c1nnc(C)n1-2]

In one aspect, compounds of type 3.8, and similar compounds, can be prepared according to reaction Scheme 3B above. Thus, compounds of type 3.6 can be prepared by deprotection of an appropriate cyclic amine, e.g., 3.5 as shown above. The deprotection is carried out in the presence of an appropriate acid, e.g., 4M hydrochloric acid. Compounds of type 3.8 can be prepared by a coupling reaction between an appropriate amine, e.g., 3.6 as shown above, and an appropriate carboxylic acid, e.g., 3.7 as shown above. Appropriate carboxylic acids are commercially available or prepared by methods known to one skilled in the art. The coupling reaction is carried out in the presence of an appropriate coupling agent, e.g., HATU, and an appropriate base, e.g., DIPEA, in an appropriate solvent, e.g., DMF. As can be appreciated by one skilled in the art, the above reaction provides an example of a generalized approach wherein compounds similar in structure to the specific reactants above (compounds similar to compounds of type 3.1, 3.2, and 3.3), can be substituted in the reaction to provide substituted heterobifunctional compounds similar to Formula 3.4.

### 4. Route IV

In one aspect, the disclosed heterobifunctional compounds can be prepared as shown below.

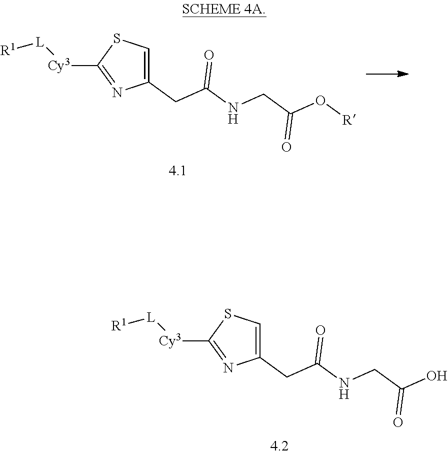

Compounds are represented in generic form, wherein R′ is selected from C1-C4 alkyl, C4-C6 cycloalkyl, —(C1-C4 alkyl)OC(O)(C1-C4 alkyl), and —CH₂C₆H₅, and with other substituents are as noted in compound descriptions elsewhere herein. A specific non-limiting example of the synthesis shown in Scheme 4A is provided below.

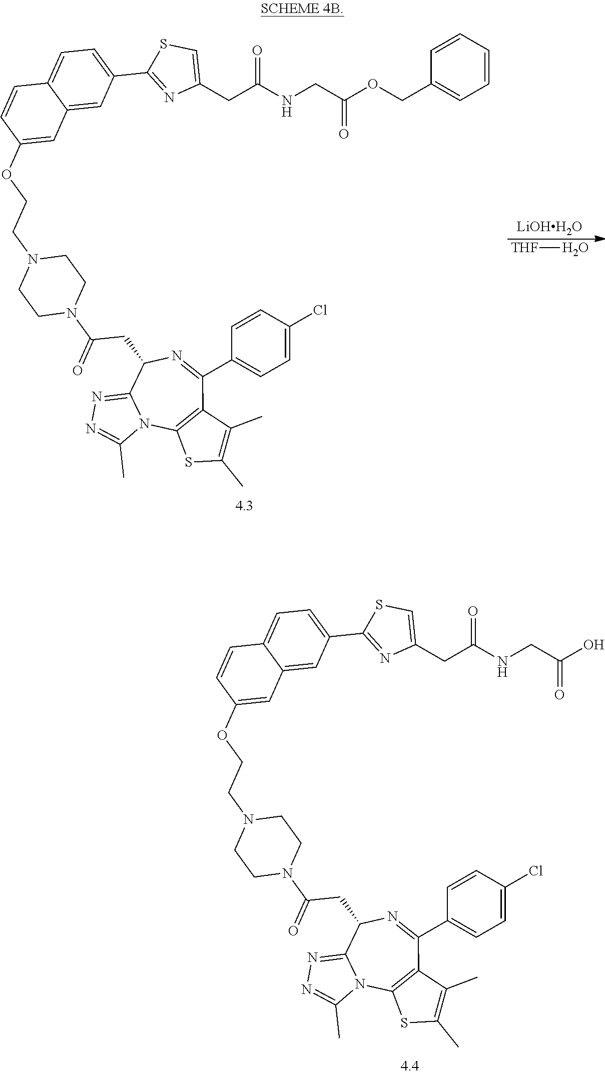

In one aspect, compounds of type 4.4, and similar compounds, can be prepared according to reaction Scheme 4B above. Thus, compounds of type 4.4 can be prepared by deprotection of an appropriate ester, e.g., 4.3 as shown above. The deprotection is carried out in the presence of an appropriate base, e.g., aqueous lithium hydroxide, in an appropriate solvent system, e.g., tetrahydrofuran and water. As can be appreciated by one skilled in the art, the above reaction provides an example of a generalized approach wherein compounds similar in structure to the specific reactants above (compounds similar to compounds of type 4.1), can be substituted in the reaction to provide substituted heterobifunctional compounds similar to Formula 4.2.

### 5. Route V

In one aspect, the disclosed heterobifunctional compounds can be prepared as shown

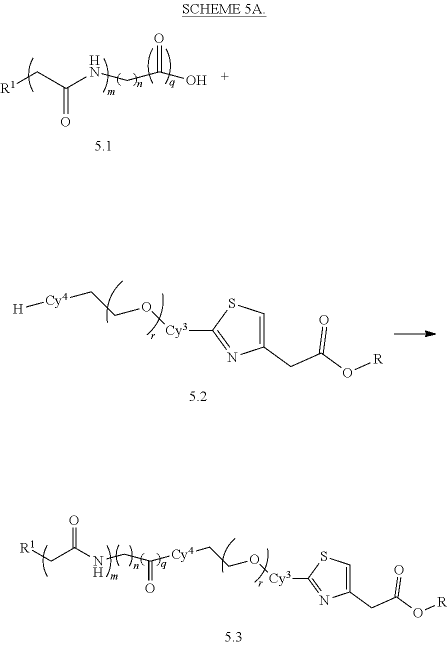

Compounds are represented in generic form, wherein R is C1-C4 alkyl, and with other substituents are as noted in compound descriptions elsewhere herein. A specific non-limiting example of the synthesis shown in Scheme 5A is provided below.

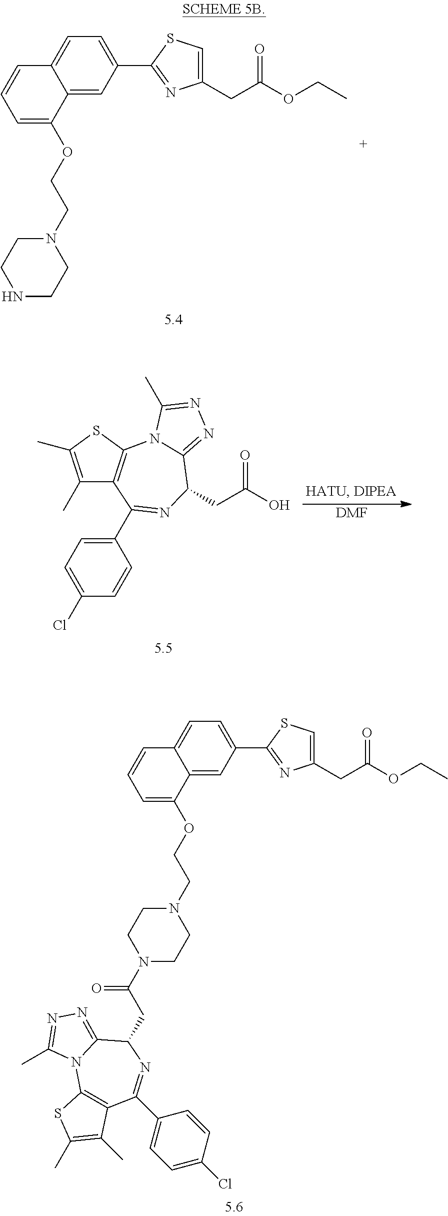

In one aspect, compounds of type 5.6, and similar compounds, can be prepared according to reaction Scheme 5B above. Thus, compounds of type 5.6 can be prepared by a coupling reaction between an appropriate amine, e.g., 5.4 as shown above, and an appropriate carboxylic acid, e.g., 5.5 as shown above. Appropriate amines and appropriate carboxylic acids are commercially available or prepared by methods known to one skilled in the art. The coupling reaction is carried out in the presence of an appropriate coupling agent, e.g., HATU, and an appropriate base, e.g., DIPEA, in an appropriate solvent, e.g., DMF. As can be appreciated by one skilled in the art, the above reaction provides an example of a generalized approach wherein compounds similar in structure to the specific reactants above (compounds similar to compounds of type 5.1 and 5.2), can be substituted in the reaction to provide substituted heterobifunctional compounds similar to Formula 5.3.

### 6. Route VI

In one aspect, the disclosed heterobifunctional compounds can be prepared as shown below.

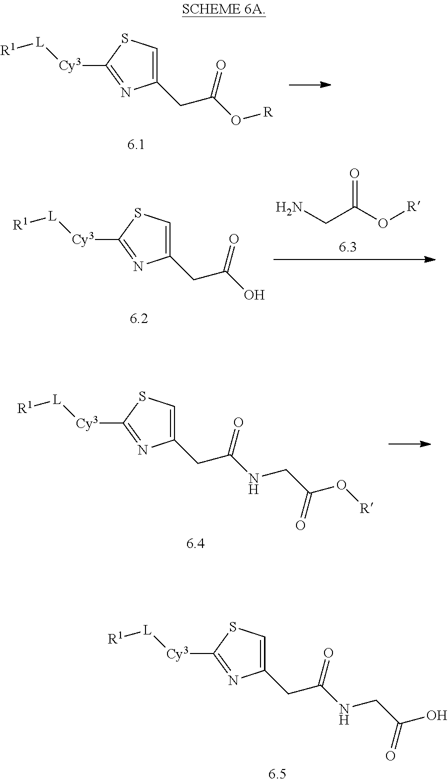

Compounds are represented in generic form, wherein R is C1-C4 alkyl, R′ is selected from C1-C4 alkyl, C4-C6 cycloalkyl, —(C1-C4 alkyl)OC(O)(C1-C4 alkyl), and —CH₂C₆H₅, and with other substituents are as noted in compound descriptions elsewhere herein. A specific non-limiting example of the synthesis shown in Scheme 6A is provided below.

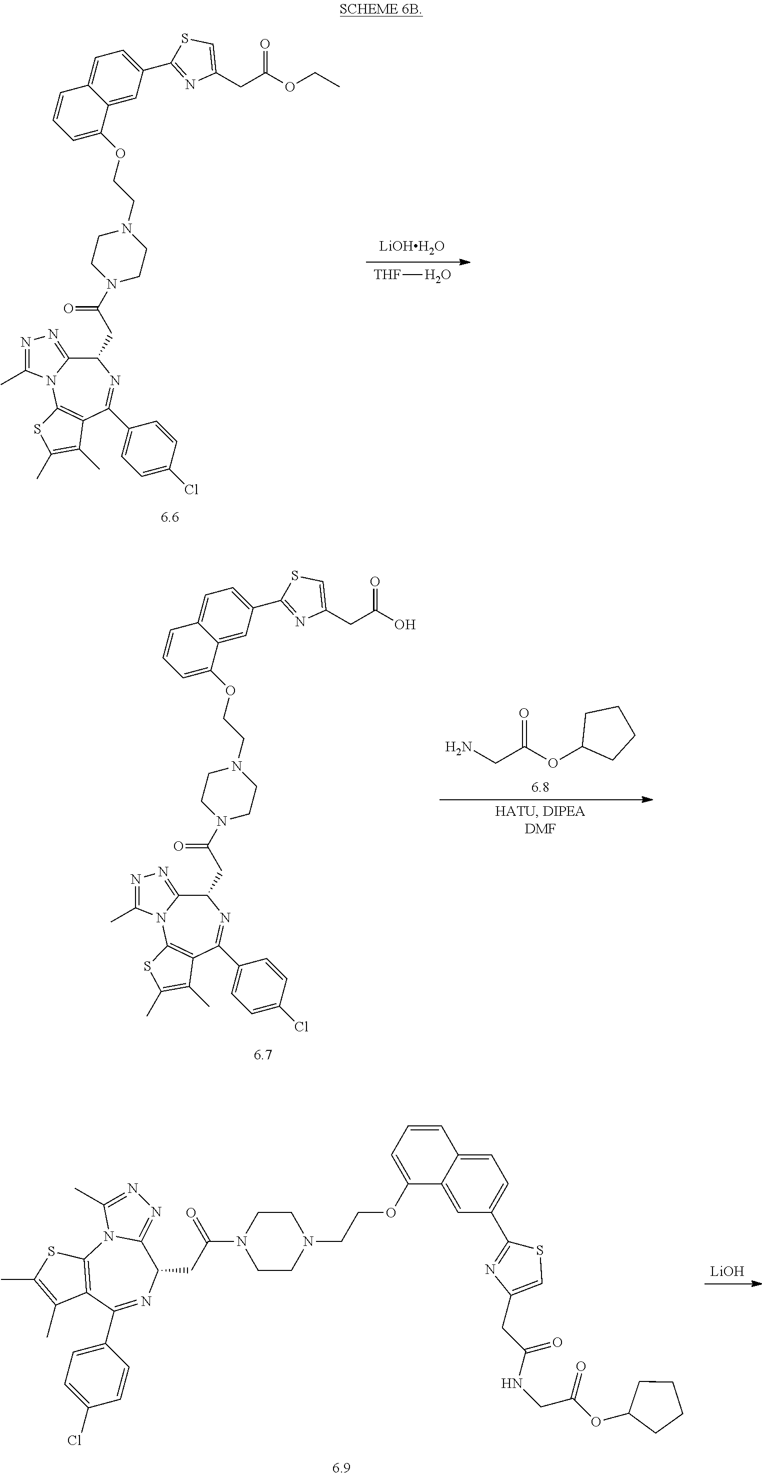

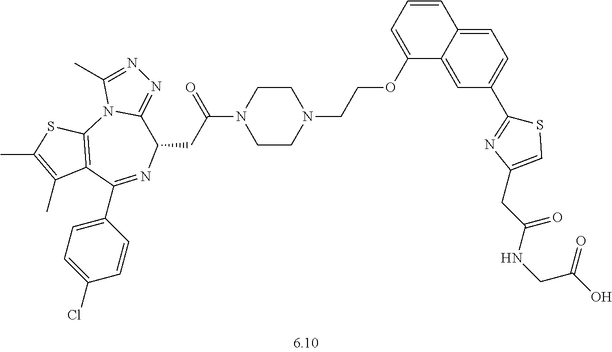 [SMILES: Cc1sc2c(c1C)C(c1ccc(Cl)cc1)=N[C@@H](CC(=O)N1CCN(CCOc3cccc4ccc(-c5nc(CC(=O)NCC(=O)O)cs5)cc34)CC1)c1nnc(C)n1-2]

In one aspect, compounds of type 6.10, and similar compounds, can be prepared according to reaction Scheme 6B above. Thus, compounds of type 6.7 can be prepared by reduction of an appropriate ester, e.g., 6.6 as shown above. The reduction is carried out in the presence of an appropriate base, e.g., aqueous lithium hydroxide, in an appropriate solvent system, e.g., tetrahydrofuran and water. Compounds of type 6.9 can be prepared by a coupling reaction between an appropriate carboxylic acid, e.g., 6.7 as shown above, and an appropriate amine, e.g., 6.8 as shown above. Appropriate amines are commercially available or prepared by methods known to one skilled in the art. The coupling reaction is carried out in the presence of an appropriate coupling agent, e.g., HATU, and an appropriate base, e.g., DIPEA, in an appropriate solvent, e.g., DMF. Compounds of type 6.10 can be prepared by reduction of an appropriate ester, e.g., 6.9 as shown above. The reduction is carried out in the presence of an appropriate base, e.g., lithium hydroxide. As can be appreciated by one skilled in the art, the above reaction provides an example of a generalized approach wherein compounds similar in structure to the specific reactants above (compounds similar to compounds of type 6.1, 6.2, 6.3, and 6.4), can be substituted in the reaction to provide substituted heterobifunctional compounds similar to Formula 6.5.

### 7. Route VII

In one aspect, the disclosed heterobifunctional compounds can be prepared as shown below.

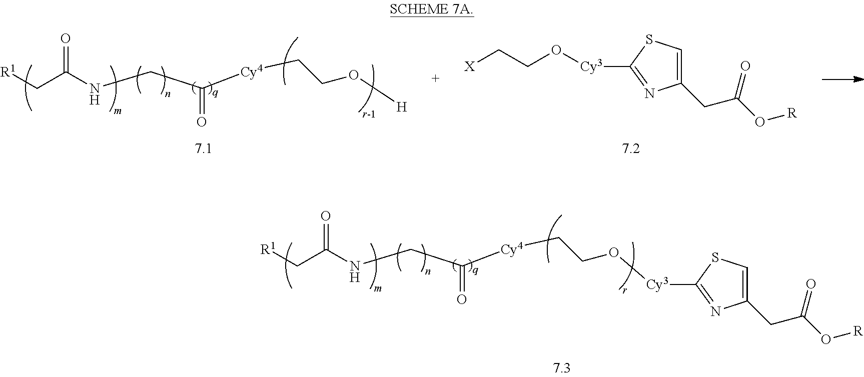

Compounds are represented in generic form, wherein R is C₁-C₄ alkyl, R′ is selected from C₁-C₄ alkyl, C₄-C₆ cycloalkyl, —(C₁-C₄ alkyl)OC(O)(C₁-C₄ alkyl), and —CH₂C₆H₅, and with other substituents are as noted in compound descriptions elsewhere herein. A specific non-limiting example of the synthesis shown in Scheme 7A is provided below.

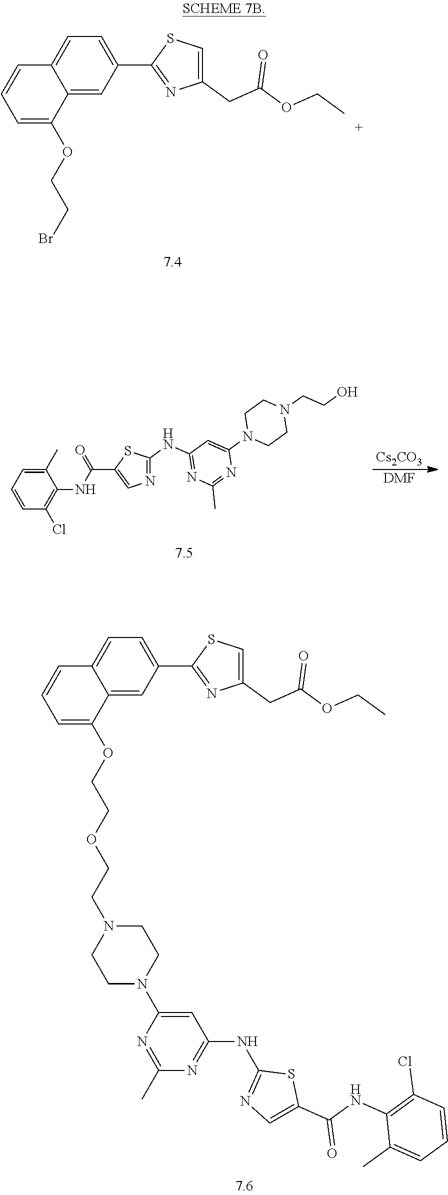

In one aspect, compounds of type 7.6, and similar compounds, can be prepared according to reaction Scheme 7B above. Thus, compounds of type 7.6 can be prepared by an alkylation reaction between an appropriate alkyl halide, e.g., 7.4 as shown above, and an appropriate alcohol, e.g., 7.5 as shown above. Appropriate alkyl halides and appropriate alcohols are commercially available or prepared by methods known to one skilled in the art. The alkylation reaction is carried out in the presence of an appropriate base, e.g., cesium carbonate, in an appropriate solvent, e.g., DMF. As can be appreciated by one skilled in the art, the above reaction provides an example of a generalized approach wherein compounds similar in structure to the specific reactants above (compounds similar to compounds of type 7.1 and 7.2), can be substituted in the reaction to provide substituted heterobifunctional compounds similar to Formula 7.3.

### 8. Route VIII

In one aspect, the disclosed heterobifunctional compounds can be prepared as shown below.

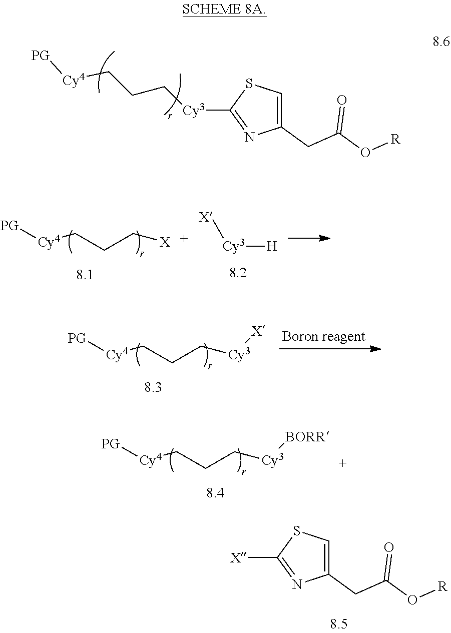

Compounds are represented in generic form, wherein each of X, X′, and X″ is independently halogen, R is C1-C4 alkyl, and with other substituents are as noted in compound descriptions elsewhere herein. A specific non-limiting example of the synthesis shown in Scheme 8A is provided below.

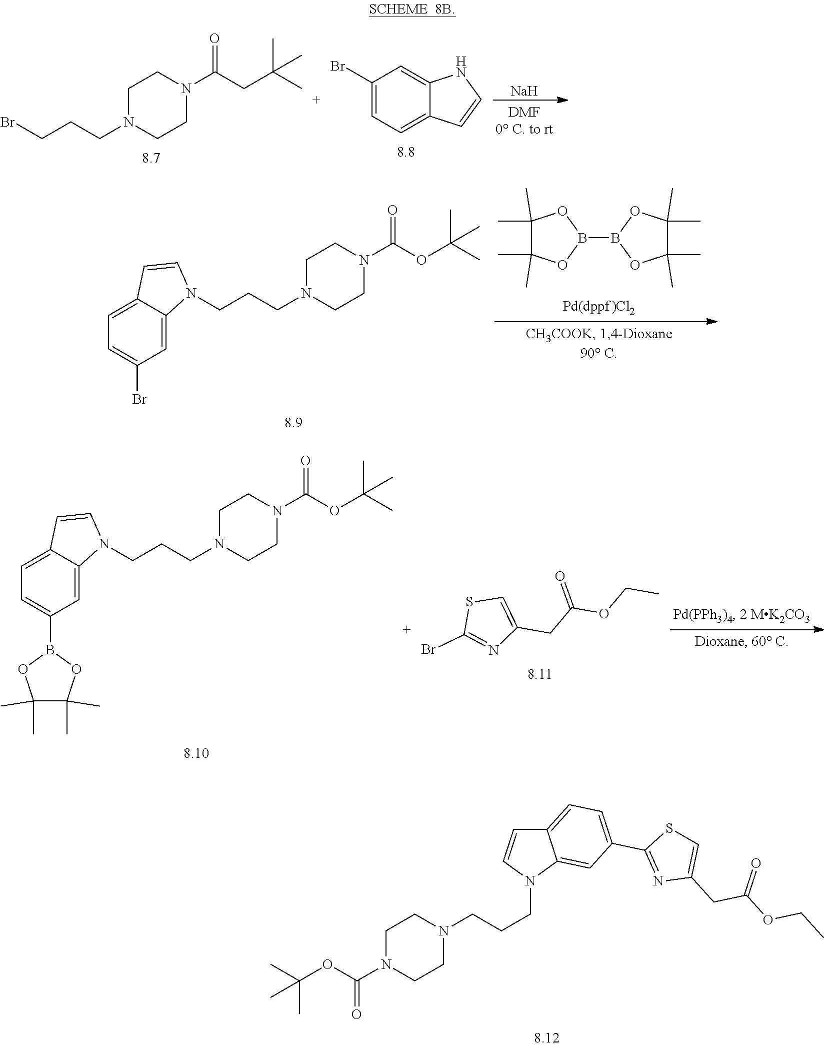

In one aspect, compounds of type 8.12, and similar compounds, can be prepared according to reaction Scheme 8B above. Thus, compounds of type 8.9 can be prepared by an alkylation reaction between an appropriate alkyl halide, e.g., 8.7 as shown above, and an appropriate amine, e.g., 8.8 as shown above. Appropriate alkyl halides and appropriate amines are commercially available or prepared by methods known to one skilled in the art. The alkylation reaction is carried out in the presence of an appropriate base, e.g., sodium hydride, in an appropriate solvent, e.g., DMF, at an appropriate temperature, e.g., 0° C. to room temperature. Compounds of type 8.10 can be prepared by boronation of an appropriate aryl halide, e.g., 8.9 as shown above. The boronation is carried out in the presence of an appropriate boronic acid, e.g., 4,4,4′,4′,5,5,5′,5′-octamethyl-2,2′-bi(1,3,2-dioxaborolane), an appropriate catalyst, e.g., [1,1′-bis(diphenylphosphino)ferrocene]dichloropalladium(II), and an appropriate salt, e.g., potassium acetate, in an appropriate solvent, e.g., 1,4-dioxane, at an appropriate temperature, e.g., 90° C. Compounds of type 8.12 can be prepared by a coupling reaction between an appropriate aryl boronic acid, e.g., 8.10 as shown above, and an appropriate 2-bromothiazole, e.g., 8.11 as shown above. Appropriate 2-bromothiazoles are commercially available or prepared by methods known to one skilled in the art. The coupling reaction is carried out in the presence of an appropriate catalyst, e.g., palladium-tetrakis(triphenylphosphine), and an appropriate base, e.g., 2M potassium carbonate, in an appropriate solvent, e.g., dioxane, at an appropriate temperature, e.g., 60° C. As can be appreciated by one skilled in the art, the above reaction provides an example of a generalized approach wherein compounds similar in structure to the specific reactants above (compounds similar to compounds of type 8.1, 8.2, 8.3, 8.4, and 8.5), can be substituted in the reaction to provide substituted heterobifunctional compounds similar to Formula 8.6.

### E. Pharmaceutical Compositions

In one aspect, disclosed are pharmaceutical compositions comprising a disclosed compound, or a pharmaceutically acceptable salt thereof and a pharmaceutically acceptable carrier.

Thus, in one aspect, disclosed are pharmaceutical composition comprising a therapeutically effective amount of a compound having a structure represented by a formula:

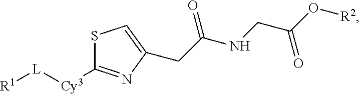

wherein L is a linker; wherein R¹ is a residue of a small molecule having a molecular weight of from about 150 g/mol to about 600 g/mol and a binding affinity (Kᵢ) to a target protein of at least about 20 μM; wherein R² is selected from hydrogen, C1-C4 alkyl, C4-C6 cycloalkyl, —(C1-C4 alkyl)OC(O)(C1-C4 alkyl), and —CH₂C₆H₅; and wherein Cy³ is a 9- or 10-membered heterobicycle, a 10-membered biaryl, or a 9- or 10-membered heterobiaryl, and is substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹; wherein each occurrence of R¹⁰, when present, is independently selected from hydrogen, C₁-C₄ alkyl, —(C₁-C₄ alkyl)O(C₁-C₄ alkyl), —(C₁-C₄ alkyl)O(C₁-C₄ alkyl)N₃, and —(C₁-C₄ alkyl)Cy²; wherein Cy², when present, is a C₂-C₅ heterocycloalkyl substituted with 0, 1, 2, or 3 groups independently selected from halogen, —N₃, —CN, —NH₂, —OH, —NO₂, C₁-C₄ alkyl, C₂-C₄ alkenyl, C₁-C₄ haloalkyl, C₁-C₄ cyanoalkyl, C₁-C₄ hydroxyalkyl, C₁-C₄ alkoxy, C₁-C₄ alkylamino, (C₁-C₄)(C₁-C₄) dialkylamino, C₁-C₄ aminoalkyl, —C(O)H, —C(O)(C₁-C₄ alkyl), —CO₂H, and —CO₂(C₁-C₄ alkyl); wherein each occurrence of R^{D}, when present, is independently selected from C₁-C₄ alkyl, C₁-C₄ hydroxyalkyl, and Ar¹; wherein Ar¹, when present, is a C₆ aryl or a C₂-C₅ heteroaryl, and is substituted with 0, 1, 2, or 3 groups independently selected from halogen, —CN, —NH₂, —NO₂, C₁-C₄ alkyl, C₂-C₄ alkenyl, C₁-C₄ haloalkyl, C₁-C₄ cyanoalkyl, C₁-C₄ hydroxyalkyl, C₁-C₄ alkylamino, (C₁-C₄)(C₁-C₄) dialkylamino, and C₁-C₄ aminoalkyl, or a pharmaceutically acceptable salt thereof.

In a further aspect, the linker has a molecular weight of from about 100 g/mol to about 200 g/mol. In a still further aspect, the linker has a length of from four to sixteen atoms comprising carbon, nitrogen, and oxygen. In yet a further aspect, the linker has a structure represented by a formula:

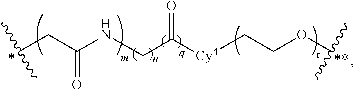

wherein * is connected to R¹ and ** is connected to Cy³; wherein m is 0 or 1; wherein n is 1, 2, or 3; wherein q is 0 or 1; wherein r is 1, 2, 3, or 4; and wherein Cy⁴ is a structure selected from:

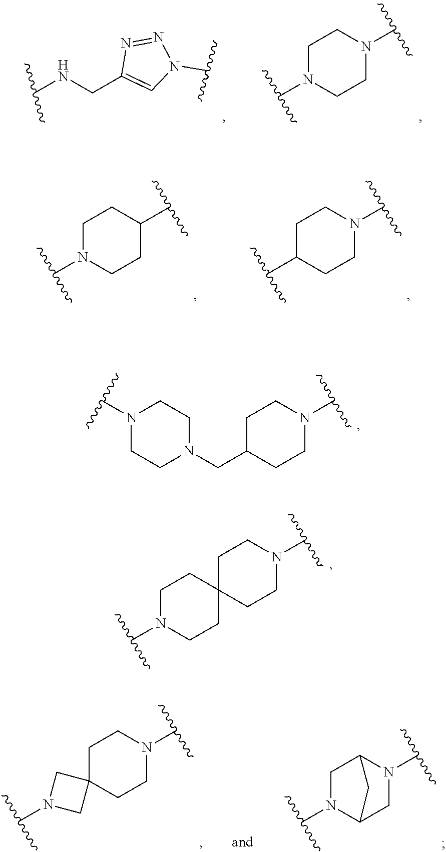

In a further aspect, the residue of a small molecule has a structure selected from:

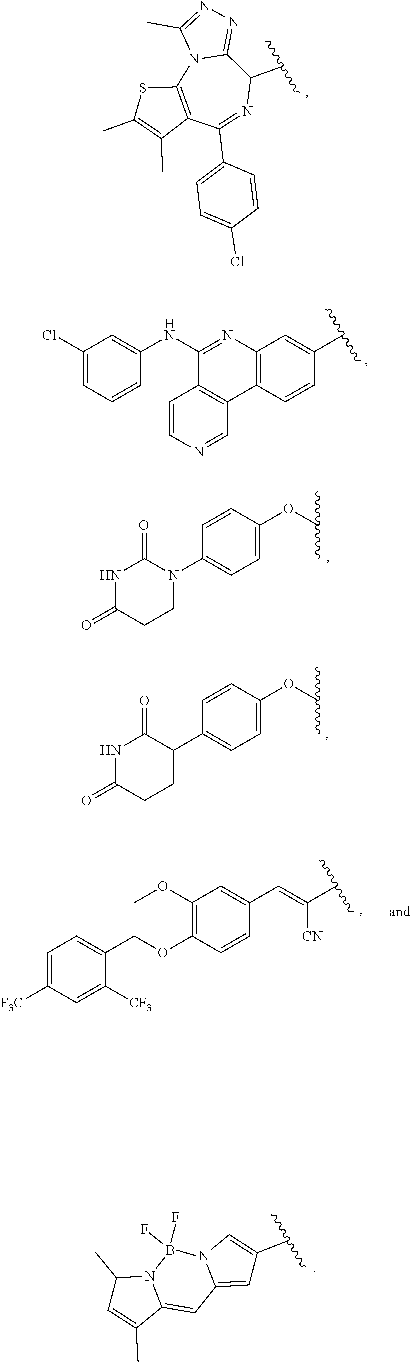

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, e.g., administration by drops or injection into the ear, insufflation (such as into the ear), intravenous, topical, or oral administration.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, PA 1990.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, an effective amount is a therapeutically effective amount. In a still further aspect, an effective amount is a prophylactically effective amount.

In a further aspect, the pharmaceutical composition is administered to a mammal. In a still further aspect, the mammal is a human. In an even further aspect, the human is a patient.

In a further aspect, the pharmaceutical composition is used to treat a disorder a disorder of uncontrolled cellular proliferation such as, for example, cancer.

It is understood that the disclosed compositions can be prepared from the disclosed compounds. It is also understood that the disclosed compositions can be employed in the disclosed methods of using.

### F. Methods of Treating a Disorder of Uncontrolled Cellular Proliferation in a Subject

In various aspects, the compounds and compositions disclosed herein are useful for treating, preventing, ameliorating, controlling, or reducing the risk of a variety of disorders related to the activity of a target protein, such that degradation of the target protein is desired. Exemplary disorders include, but are not limited to, disorders of uncontrolled cellular proliferation (e.g., cancer). Thus, in one aspect, disclosed are methods of treating a disorder of uncontrolled cellular proliferation in a subject in need thereof, the method comprising administering to the subject an effective amount of at least one disclosed heterobifunctional compound or a pharmaceutically acceptable salt thereof.

Thus, in one aspect, disclosed are methods of treating a disorder of uncontrolled cellular proliferation in a subject in need thereof, the method comprising administering to the subject an effective amount of a compound having a structure represented by a formula:

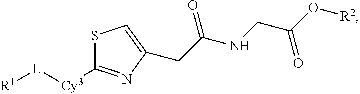

wherein L is a linker; wherein R¹ is a residue of a small molecule having a molecular weight of from about 150 g/mol to about 600 g/mol and a binding affinity (Kᵢ) to a target protein of at least about 20 μM; wherein R² is selected from hydrogen, C1-C4 alkyl, C4-C6 cycloalkyl, —(C1-C4 alkyl)OC(O)(C1-C4 alkyl), and —CH₂C₆H₅; and wherein Cy³ is a 9- or 10-membered heterobicycle, a 10-membered biaryl, or a 9- or 10-membered heterobiaryl, and is substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹; wherein each occurrence of R¹⁰, when present, is independently selected from hydrogen, C1-C4 alkyl, —(C1-C4 alkyl)O(C1-C4 alkyl), —(C1-C4 alkyl)O(C1-C4 alkyl)N₃, and —(C1-C4 alkyl)Cy²; wherein Cy², when present, is a C2-C5 heterocycloalkyl substituted with 0, 1, 2, or 3 groups independently selected from halogen, —N₃, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 alkoxy, C1-C4 alkylamino, (C1-C4)(C1-C4) dialkylamino, C1-C4 aminoalkyl, —C(O)H, —C(O)(C1-C4 alkyl), —CO₂H, and —CO₂(C1-C4 alkyl); wherein each occurrence of R¹¹, when present, is independently selected from C1-C4 alkyl, C1-C4 hydroxyalkyl, and Ar¹; wherein Ar¹, when present, is a C6 aryl or a C₂-C₅ heteroaryl, and is substituted with 0, 1, 2, or 3 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl, or a pharmaceutically acceptable salt thereof.

In various aspects, the disclosed compounds can be used in combination with one or more other drugs in the treatment, prevention, control, amelioration, or reduction of risk of a disorder of uncontrolled cellular proliferation for which the disclosed compounds or the other drugs can have utility, where the combination of the drugs together are safer or more effective than either drug alone. Such other drug(s) can be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and a disclosed compound is preferred. However, the combination therapy can also include therapies in which a disclosed compound and one or more other drugs are administered on different overlapping schedules. It is also contemplated that when used in combination with one or more other active ingredients, the disclosed compounds and the other active ingredients can be used in lower doses than when each is used singly. Accordingly, the pharmaceutical compositions include those that contain one or more other active ingredients, in addition to a compound of the present invention.

In a further aspect, the compound exhibits degradation of a target protein.

In a further aspect, the subject is a mammal. In a still further aspect, the mammal is human.

In a further aspect, the subject has been diagnosed with a need for treatment of the disorder prior to the administering step. In a still further aspect, the subject is at risk for developing the disorder prior to the administering step.

In a further aspect, the method further comprises identifying a subject at risk for developing the disorder prior to the administering step.

In a further aspect, the effective amount is a therapeutically effective amount. In a still further aspect, the effective amount is a prophylactically effective amount.

In a further aspect, the disorder is a cancer. Exemplary cancers include, but are not limited to, a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma).

In a still further aspect, the cancer is leukemia.

### G. Methods of Degrading a Target Protein in a Cell

In one aspect, disclosed are methods of degrading a target protein in a cell using the ubiquitin E3 ligase KLHDC2, the method comprising the step of contacting the cell with an effective amount of a disclosed heterobifunctional compound, or a pharmaceutically acceptable salt thereof.

Thus, in one aspect, disclosed are methods of degrading a target protein in a cell, the method comprising contacting the cell with an effective amount of a compound having a structure represented by a formula:

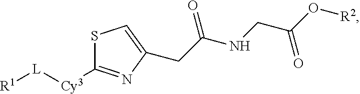

wherein L is a linker; wherein R¹ is a residue of a small molecule having a molecular weight of from about 150 g/mol to about 600 g/mol and a binding affinity (Kᵢ) to a target protein of at least about 20 μM; wherein R² is selected from hydrogen, C1-C4 alkyl, C4-C6 cycloalkyl, —(C1-C4 alkyl)OC(O)(C1-C4 alkyl), and —CH₂C₆H₅; and wherein Cy³ is a 9- or 10-membered heterobicycle, a 10-membered biaryl, or a 9- or 10-membered heterobiaryl, and is substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹; wherein each occurrence of R¹⁰, when present, is independently selected from hydrogen, C1-C4 alkyl, —(C1-C4 alkyl)O(C1-C4 alkyl), —(C1-C4 alkyl)O(C1-C4 alkyl)N₃, and —(C1-C4 alkyl)Cy²; wherein Cy², when present, is a C2-C5 heterocycloalkyl substituted with 0, 1, 2, or 3 groups independently selected from halogen, —N₃, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 alkoxy, C1-C4 alkylamino, (C1-C4)(C1-C4) dialkylamino, C1-C4 aminoalkyl, —C(O)H, —C(O)(C1-C4 alkyl), —CO₂H, and —CO₂(C1-C4 alkyl); wherein each occurrence of R¹¹, when present, is independently selected from C1-C4 alkyl, C1-C4 hydroxyalkyl, and Ar¹; wherein Ar¹, when present, is a C6 aryl or a C2-C5 heteroaryl, and is substituted with 0, 1, 2, or 3 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl, or a pharmaceutically acceptable salt thereof.

In a further aspect, the cell is mammalian. In a still further aspect, the cell is human. In yet a further aspect, the cell has been isolated from a mammal prior to the contacting step.

In a further aspect, contacting is ex vivo. In a still further aspect, contacting is in vitro. In yet a further aspect, contacting is via administration to a mammal.

In a further aspect, the mammal has been diagnosed with a need for degradation of a target protein prior to the administering step. In a still further aspect, the mammal has been diagnosed with a need for treatment of a disorder related to activity of the target protein prior to the administering step. In yet a further aspect, the disorder is a disorder of uncontrolled cellular proliferation (e.g., cancer).

In a further aspect, the mammal has been diagnosed with a need for modulating degrading a target protein prior to the administering step.

### H. Methods of Degrading a Target Protein in a Subject

In one aspect, disclosed are methods of degrading a target protein in a subject in need thereof, the method comprising the step of administering to the subject an effective amount of a heterobifunctional compound, or a pharmaceutically acceptable salt thereof.

Thus, in one aspect, disclosed are methods of degrading a target protein in a subject in need thereof, the method comprising administering to the subject an effective amount of a compound having a structure represented by a formula:

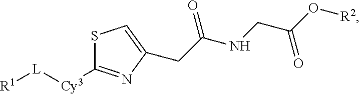

wherein L is a linker; wherein R¹ is a residue of a small molecule having a molecular weight of from about 150 g/mol to about 600 g/mol and a binding affinity (Kᵢ) to a target protein of at least about 20 μM; wherein R² is selected from hydrogen, C1-C4 alkyl, C4-C6 cycloalkyl, —(C1-C4 alkyl)OC(O)(C1-C4 alkyl), and —CH₂C₆H₅; and wherein Cy³ is a 9- or 10-membered heterobicycle, a 10-membered biaryl, or a 9- or 10-membered heterobiaryl, and is substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹; wherein each occurrence of R¹⁰, when present, is independently selected from hydrogen, C1-C4 alkyl, —(C1-C4 alkyl)O(C1-C4 alkyl), —(C1-C4 alkyl)O(C1-C4 alkyl)N₃, and —(C1-C4 alkyl)Cy²; wherein Cy², when present, is a C2-C5 heterocycloalkyl substituted with 0, 1, 2, or 3 groups independently selected from halogen, —N₃, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 alkoxy, C1-C4 alkylamino, (C1-C4)(C1-C4) dialkylamino, C1-C4 aminoalkyl, —C(O)H, —C(O)(C1-C4 alkyl), —CO₂H, and —CO₂(C1-C4 alkyl); wherein each occurrence of R¹¹, when present, is independently selected from C1-C4 alkyl, C1-C4 hydroxyalkyl, and Ar¹; wherein Ar¹, when present, is a C6 aryl or a C2-C5 heteroaryl, and is substituted with 0, 1, 2, or 3 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl, or a pharmaceutically acceptable salt thereof.

In various aspects, the subject is a mammal. In a further aspect, the subject is a human.

In various aspects, the subject has been diagnosed with a need for degrading the target protein prior to the administering step. In a further aspect, the method further comprises identifying a subject in need of degradation of the target protein.

### I. Methods of Using the Compositions

Provided are methods of using of a disclosed composition or medicament. In one aspect, the method of use is directed to the treatment of a disorder. In a further aspect, the disclosed compounds can be used as single agents or in combination with one or more other drugs in the treatment, prevention, control, amelioration, or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

The pharmaceutical compositions and methods of the present invention can further comprise other therapeutically active compounds as noted herein which are usually applied in the treatment of the above mentioned pathological conditions.

### 1. Manufacture of a Medicament

In one aspect, the invention relates to a method for the manufacture of a medicament for degrading a target protein in a mammal, the method comprising combining a therapeutically effective amount of a disclosed compound or product of a disclosed method with a pharmaceutically acceptable carrier or diluent. Exemplary disorders that can benefit from such degradation include, but are not limited to, cancer.

As regards these applications, the present method includes the administration to an animal, particularly a mammal, and more particularly a human, of a therapeutically effective amount of the compound effective in the degradation of a target protein. The dose administered to an animal, particularly a human, in the context of the present invention should be sufficient to affect a therapeutic response in the animal over a reasonable time frame. One skilled in the art will recognize that dosage will depend upon a variety of factors including the condition of the animal, the body weight of the animal, as well as the severity and stage of the disorder.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

### 2. Use of Compounds and Compositions

Also provided are the uses of the disclosed compounds and compositions. Thus, in one aspect, the invention relates to the uses of proteolysis-targeting chimeras (PROTACs) useful in, for example, the treatment of cancer.

In a further aspect, the invention relates to the use of a disclosed compound or product of a disclosed method in the manufacture of a medicament for targeted protein degradation such as, for example, disorders of uncontrolled cellular proliferation (e.g., cancer).

In a further aspect, the use relates to a process for preparing a pharmaceutical composition comprising a therapeutically effective amount of a disclosed compound or a product of a disclosed method, and a pharmaceutically acceptable carrier, for use as a medicament.

In a further aspect, the use relates to a process for preparing a pharmaceutical composition comprising a therapeutically effective amount of a disclosed compound or a product of a disclosed method, wherein a pharmaceutically acceptable carrier is intimately mixed with a therapeutically effective amount of the disclosed compound or the product of a disclosed method.

In various aspects, the use relates to the treatment of a disorder of uncontrolled cellular proliferation in a vertebrate animal. In a further aspect, the use relates to the treatment of a disorder of uncontrolled cellular proliferation in a human subject.

In a further aspect, the use is treatment of cancer.

In a further aspect, the use is degradation of a target protein.

It is understood that the disclosed uses can be employed in connection with the disclosed compounds, methods, compositions, and kits.

In a further aspect, the invention relates to the use of a disclosed compound or composition in the manufacture of a medicament for degrading a target protein.

### 3. Kits

In one aspect, disclosed are kits comprising a disclosed compound and one or more of: (a) an agent known to treat a disorder of uncontrolled cellular proliferation; (b) instructions for administering the compound in connection with treating a disorder of uncontrolled cellular proliferation; and (c) instructions for treating a disorder of uncontrolled cellular proliferation.

Thus, in one aspect, disclosed are kits comprising a compound having a structure represented by a formula:

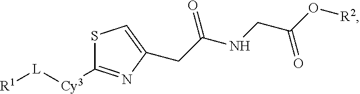

wherein L is a linker; wherein R¹ is a residue of a small molecule having a molecular weight of from about 150 g/mol to about 600 g/mol and a binding affinity (Kᵢ) to a target protein of at least about 20 μM; wherein R² is selected from hydrogen, C1-C4 alkyl, C4-C6 cycloalkyl, —(C1-C4 alkyl)OC(O)(C1-C4 alkyl), and —CH₂C₆H₅; and wherein Cy³ is a 9- or 10-membered heterobicycle, a 10-membered biaryl, or a 9- or 10-membered heterobiaryl, and is substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹; wherein each occurrence of R¹⁰, when present, is independently selected from hydrogen, C1-C4 alkyl, —(C1-C4 alkyl)O(C1-C4 alkyl), —(C1-C4 alkyl)O(C1-C4 alkyl)N₃, and —(C1-C4 alkyl)Cy²; wherein Cy², when present, is a C2-C5 heterocycloalkyl substituted with 0, 1, 2, or 3 groups independently selected from halogen, —N₃, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 alkoxy, C1-C4 alkylamino, (C1-C4)(C1-C4) dialkylamino, C1-C4 aminoalkyl, —C(O)H, —C(O)(C1-C4 alkyl), —CO₂H, and —CO₂(C1-C4 alkyl); wherein each occurrence of R¹¹, when present, is independently selected from C1-C4 alkyl, C1-C4 hydroxyalkyl, and Ar¹; wherein Ar¹, when present, is a C6 aryl or a C2-C5 heteroaryl, and is substituted with 0, 1, 2, or 3 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl, or a pharmaceutically acceptable salt thereof, and one or more of: (a) an agent known to treat a disorder of uncontrolled cellular proliferation; (b) instructions for administering the compound in connection with treating a disorder of uncontrolled cellular proliferation; and (c) instructions for treating a disorder of uncontrolled cellular proliferation.

In various aspects, the agents and pharmaceutical compositions described herein can be provided in a kit. The kit can also include combinations of the agents and pharmaceutical compositions described herein.

In various aspects, the informational material can be descriptive, instructional, marketing or other material that relates to the methods described herein and/or to the use of the agents for the methods described herein. For example, the informational material may relate to the use of the agents herein to treat a subject who has, or who is at risk for developing, a disorder as detailed elsewhere herein. The kits can also include paraphernalia for administering the agents of this invention to a cell (in culture or in vivo) and/or for administering a cell to a patient.

In various aspects, the informational material can include instructions for administering the pharmaceutical composition and/or cell(s) in a suitable manner to treat a human, e.g., in a suitable dose, dosage form, or mode of administration (e.g., a dose, dosage form, or mode of administration described herein). In a further aspect, the informational material can include instructions to administer the pharmaceutical composition to a suitable subject, e.g., a human having, or at risk for developing, a disorder of uncontrolled cellular proliferation.

In various aspects, the composition of the kit can include other ingredients, such as a solvent or buffer, a stabilizer, a preservative, a fragrance or other cosmetic ingredient. In such aspects, the kit can include instructions for admixing the agent and the other ingredients, or for using one or more compounds together with the other ingredients.

In a further aspect, the compound and the at least one agent are co-formulated.

In a still further aspect, the compound and the at least one agent are co-packaged.

In a further aspect, the agent is a chemotherapeutic agent. In a still further aspect, the chemotherapeutic agent is selected from an alkylating agent, an antimetabolite agent, an antineoplastic antibiotic agent, a mitotic inhibitor agent, and a mTor inhibitor agent.

In a further aspect, the chemotherapeutic agent is an antineoplastic agent. Examples of antineoplastic agents include, but are not limited to, doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

In a further aspect, the chemotherapeutic agent is an antimetabolite agent. Examples of antimetabolite agents include, but are not limited to, gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

In a further aspect, the chemotherapeutic agent is an alkylating agent. Examples of alkylating agents include, but are not limited to, carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof.

In a further aspect, the chemotherapeutic agent is a mitotic inhibitor agent. Examples of mitotic inhibitor agents include, but are not limited to, irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof.

In a further aspect, the chemotherapeutic agent is a mTor inhibitor agent. Examples of mTor inhibitor agents include, but are not limited to, everolimus, siroliumus, and temsirolimus, or a pharmaceutically acceptable salt, hydrate, solvate, or polymorph thereof.

In a further aspect, the kit further comprises a plurality of dosage forms, the plurality comprising one or more doses; wherein each dose comprises an effective amount of the compound and the agent. In a still further aspect, the effective amount is a therapeutically effective amount. In yet a further aspect, the effective amount is a prophylactically effective amount. In an even further aspect, each dose of the compound and the agent are co-packaged. In a still further aspect, each dose of the compound and the agent are co-formulated.

In a further aspect, the disorder is a cancer.

### 4. Subjects

In various aspects, the subject of the herein disclosed methods is a vertebrate, e.g., a mammal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

In some aspects of the disclosed methods, the subject has been diagnosed with a need for treatment prior to the administering step. In some aspects of the disclosed method, the subject has been diagnosed with a disorder of uncontrolled cellular proliferation prior to the administering step. In some aspects of the disclosed methods, the subject has been identified with a need for treatment prior to the administering step. In one aspect, a subject can be treated prophylactically with a compound or composition disclosed herein, as discussed herein elsewhere.

### A. Dosage

Toxicity and therapeutic efficacy of the agents and pharmaceutical compositions described herein can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and can be expressed as the ratio LD₅₀/ED₅₀. Polypeptides or other compounds that exhibit large therapeutic indices are preferred.

Data obtained from cell culture assays and further animal studies can be used in formulating a range of dosage for use in humans. The dosage of such compounds lies preferably within a range of circulating concentrations that include the ED₅₀ with little or no toxicity, and with little or no adverse effect on a human's ability to hear. The dosage may vary within this range depending upon the dosage form employed and the route of administration utilized. For any agents used in the methods described herein, the therapeutically effective dose can be estimated initially from cell culture assays. A dose can be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Exemplary dosage amounts of a differentiation agent are at least from about 0.01 to 3000 mg per day, e.g., at least about 0.00001, 0.0001, 0.001, 0.01, 0.1, 1, 2, 5, 10, 25, 50, 100, 200, 500, 1000, 2000, or 3000 mg per kg per day, or more.

The formulations and routes of administration can be tailored to the disease or disorder being treated, and for the specific human being treated. For example, a subject can receive a dose of the agent once or twice or more daily for one week, one month, six months, one year, or more. The treatment can continue indefinitely, such as throughout the lifetime of the human. Treatment can be administered at regular or irregular intervals (once every other day or twice per week), and the dosage and timing of the administration can be adjusted throughout the course of the treatment. The dosage can remain constant over the course of the treatment regimen, or it can be decreased or increased over the course of the treatment.

In various aspects, the dosage facilitates an intended purpose for both prophylaxis and treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., (1990) Chapter 27 in Remington's Pharmaceutical Sciences, 18th Ed., Gennaro, ed., Mack Publishing Co., Easton, PA). In general, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., (1996) Chapter 3, In: Goodman & Gilman's The Pharmacological Basis of Therapeutics, 9th Ed., Hardman et al., eds., McGraw-Hill, New York, NY).

b. Routes of Administration

Also provided are routes of administering the disclosed compounds and compositions. The compounds and compositions of the present invention can be administered by direct therapy using systemic administration and/or local administration. In various aspects, the route of administration can be determined by a patient's health care provider or clinician, for example following an evaluation of the patient. In various aspects, an individual patient's therapy may be customized, e.g., the type of agent used, the routes of administration, and the frequency of administration can be personalized. Alternatively, therapy may be performed using a standard course of treatment, e.g., using pre-selected agents and pre-selected routes of administration and frequency of administration.

Systemic routes of administration can include, but are not limited to, parenteral routes of administration, e.g., intravenous injection, intramuscular injection, and intraperitoneal injection; enteral routes of administration e.g., administration by the oral route, lozenges, compressed tablets, pills, tablets, capsules, drops (e.g., ear drops), syrups, suspensions and emulsions; rectal administration, e.g., a rectal suppository or enema; a vaginal suppository; a urethral suppository; transdermal routes of administration; and inhalation (e.g., nasal sprays).

In various aspects, the modes of administration described above may be combined in any order.

### J. Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Chemistry Experimentals

### a. General Procedure for the Synthesis of Naphthalene-, Quinoline-, and Isoquinoline-Based KLHDC2 Ligands

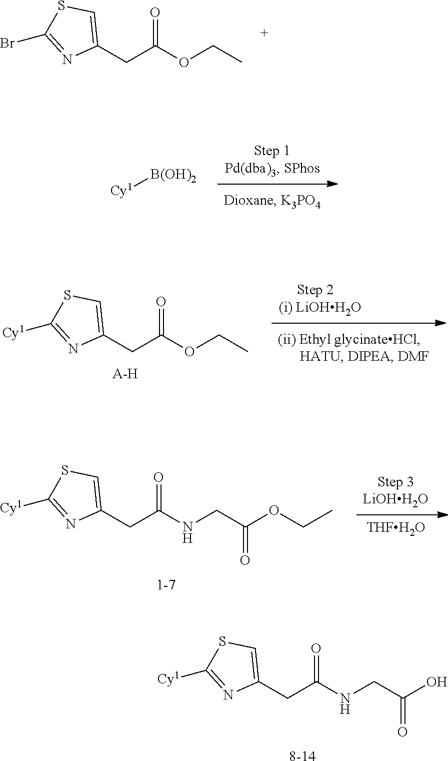

### i. Step 1

### (a) Synthesis of Ethyl 2-(2-(Naphthalen-2-yl)Thiazol-4-yl)Acetate (A)

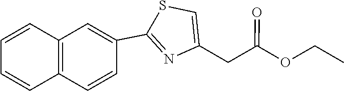 [SMILES: CCOC(=O)Cc1csc(-c2ccc3ccccc3c2)n1]

A mixture of ethyl 2-(2-bromothiazol-4-yl)acetate (200 mg, 0.8 mmol), naphthalen-2-ylboronic acid (275 mg, 1.59 mmol), potassium phosphate tribasic (1.2 ml, 2.39 mmol; 2M solution in water), and SPhos (33 mg, 0.0.08 mmol) in Dioxane (4 ml) was stirred for 5 min while purging with nitrogen. Next, tris(dibenzylideneacetone)dipalladium(0) (36 mg, 0.04 mmol) was added. The reaction mixture was purged with nitrogen for additional 2 min then stirred at 60° C. for 30 min. The reaction mixture was then cooled to room temperature and quenched with water. The compound was extracted with EtOAc (2×50 mL). The combined EtOAc was dried and evaporated under vacuum. The residue was subjected to flash column purification, product eluted at 60% EA in Hexanes to get product ethyl 2-(2-(naphthalen-2-yl)thiazol-4-yl)acetate (229 mg, 93%) as a brown solid. ¹H NMR (500 MHz, CDCl₃) δ 8.41-8.31 (m, 1H), 7.97 (dd, J=8.5, 1.8 Hz, 1H), 7.88-7.74 (m, 3H), 7.45 (dt, J=6.2, 3.4 Hz, 2H), 7.17 (d, J=0.9 Hz, 1H), 4.17 (q, J=7.1 Hz, 2H), 3.87 (d, J=0.8 Hz, 2H), 1.24 (t, J=7.1 Hz, 3H); ¹³C NMR (125 MHz, CDCl₃) δ 170.5, 150.0, 134.1, 133.3, 130.9, 128.7, 127.8, 127.0, 126.8, 126.0, 124.1, 118.0, 116.3, 109.5, 61.2, 37.2, 14.2; HRMS (ESI) m/z calcd for C₁₇H₁₆NO₂S [M+H]+, 298.0902; found, 298.0884.

### (b) Synthesis of Ethyl 2-(2-(Isoquinolin-8-yl)Thiazol-4-yl)Acetate (B)

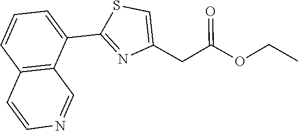 [SMILES: CCOC(=O)Cc1csc(-c2cccc3ccncc23)n1]

Compound B (80 mg, 33%) was synthesized using the same procedure employed for the synthesis of compound A, yielding a yellow solid. ¹H NMR (500 MHz, CDCl₃) δ 10.21 (s, 1H), 8.61 (d, J=5.7 Hz, 1H), 8.01-7.82 (m, 2H), 7.80-7.62 (m, 2H), 7.38 (s, 1H), 4.25 (q, J=7.1 Hz, 2H), 3.99 (s, 2H), 1.32 (t, J=7.2 Hz, 3H); ¹³C NMR (125 MHz, CDCl₃) δ 170.3, 165.3, 151.4, 150.3, 143.4, 136.4, 131.5, 129.5, 129.3, 128.8, 125.5, 120.4, 117.5, 61.3, 37.3, 14.3; HRMS (ESI) m/z calcd for C₁₆H₁₅N₂O₂S [M+H]⁺, 299.0854; found, 299.0851.

### (c) Synthesis of Ethyl 2-(2-(6-Methoxynaphthalen-2-yl)Thiazol-4-yl)Acetate (C)

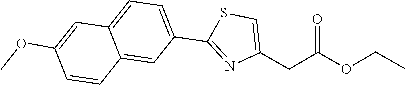 [SMILES: CCOC(=O)Cc1csc(-c2ccc3cc(OC)ccc3c2)n1]

Compound C (250 mg, 95%) was synthesized using the same procedure employed for the synthesis of compound A, yielding a yellow solid. ¹H NMR (500 MHz, CDCl₃) δ 8.25 (d, J=1.8 Hz, 1H), 7.91 (dd, J=8.6, 1.8 Hz, 1H), 7.69 (dd, J=14.7, 8.7 Hz, 2H), 7.15-7.04 (m, 2H), 6.99 (ddd, J=10.7, 5.1, 2.5 Hz, 1H), 4.14 (q, J=7.1 Hz, 2H), 3.85 (s, 5H), 1.22 (t, J=7.1 Hz, 3H); ¹³C NMR (125 MHz, CDCl₃) δ 170.6, 158.6, 149.8, 135.5, 130.2, 128.4, 127.8, 125.8, 124.7, 119.1, 118.3, 115.8, 109.7, 106.0, 105.8, 61.2, 55.4, 37.2, 14.2; HRMS (ESI) m/z calcd for C₁₈H₁₈NO₃S [M+H]⁺, 328.1007; found, 328.0994.

### (d) Synthesis of Ethyl 2-(2-(Quinolin-8-yl)Thiazol-4-yl)Acetate (D)

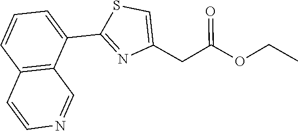 [SMILES: CCOC(=O)Cc1csc(-c2cccc3ccncc23)n1]

Compound D (100 mg, 84%) was synthesized using the same procedure employed for the synthesis of compound A, yielding a yellow oil. ¹H NMR (400 MHz, CD₃OD) δ 8.91 (dd, J=4.2, 1.8 Hz, 1H), 8.64 (dd, J=7.5, 1.4 Hz, 1H), 8.26 (dd, J=8.3, 1.8 Hz, 1H), 7.88 (dd, J=8.2, 1.4 Hz, 1H), 7.58 (dd, J=8.1, 7.4 Hz, 1H), 7.46 (dd, J=8.3, 4.2 Hz, 1H), 7.41 (t, J=0.8 Hz, 1H), 4.13 (q, J=7.1 Hz, 2H), 3.83 (s, 2H), 1.19 (t, J=7.2 Hz, 3H); ¹³C NMR (100 MHz, CD₃OD) δ 172.6, 164.0, 150.8, 149.3, 145.3, 138.0, 130.8, 129.9, 129.5, 127.6, 122.8, 121.8, 62.2, 37.7, 14.5; HRMS (ESI) m/z calcd for C₁₆H₁₅N₂O₂S[M+H]⁺, 299.0854; found, 299.0851.

### (e) Synthesis of Ethyl 2-(2-(Naphthalen-1-yl)Thiazol-4-yl)Acetate (E)

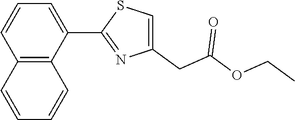 [SMILES: CCOC(=O)Cc1csc(-c2cccc3ccccc23)n1]

Compound E (200 mg, 84%) was synthesized using the same procedure employed for the synthesis of compound A, yielding a brown solid. ¹H NMR (500 MHz, CDCl₃) δ 8.76-8.69 (m, 1H), 7.96-7.86 (m, 2H), 7.80 (dd, J=7.2, 1.2 Hz, 1H), 7.60-7.47 (m, 3H), 7.33 (d, J=0.8 Hz, 1H), 4.26 (q, J=7.1 Hz, 2H), 3.99 (d, J=0.9 Hz, 2H), 1.33 (t, J=7.1 Hz, 3H); ¹³C NMR (125 MHz, CDCl₃) δ 170.5, 167.1, 149.6, 134.0, 130.8, 130.6, 130.4, 128.5, 128.3, 127.3, 126.4, 125.8, 125.0, 116.9, 61.1, 37.4, 14.3; HRMS (ESI) m/z calcd for C₁₇H₁₆NO₂S[M+H]⁺, 298.0902; found, 298.0919.

### (f) Synthesis of Ethyl 2-(2-(1-Fluoronaphthalen-2-yl)Thiazol-4-yl)Acetate (F)

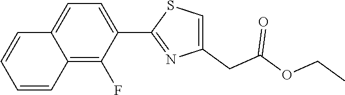 [SMILES: CCOC(=O)Cc1csc(-c2ccc3ccccc3c2F)n1]

Compound F (50 mg, 79%) was synthesized using the same procedure employed for the synthesis of compound A, yielding a white solid. ¹H NMR (400 MHz, CDCl₃) δ 8.35 (dd, J=8.7, 7.2 Hz, 1H), 8.24-8.15 (m, 1H), 7.91-7.83 (m, 1H), 7.69 (d, J=8.7 Hz, 1H), 7.61-7.57 (m, 2H), 7.37 (d, J=0.9 Hz, 1H), 4.24 (q, J=7.1 Hz, 2H), 3.95 (d, J=0.9 Hz, 2H), 1.31 (t, J=7.1 Hz, 3H); ¹³C NMR (100 MHz, CDCl₃) δ 170.5, 148.8, 129.0, 128.4, 127.8, 127.6, 127.6, 126.9, 125.4, 124.8, 124.7, 123.9, 123.9, 121.2, 121.1, 117.6, 117.5, 61.1, 37.2, 14.2; HRMS (ESI) m/z calcd for C₁₇H₁₅FNO₂S[M+H]*, 316.0808; found, 316.0808.

### (g) Synthesis of Ethyl 2-(2-(7-Methoxynaphthalen-2-yl)Thiazol-4-yl)Acetate (G)

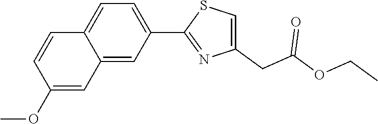 [SMILES: CCOC(=O)Cc1csc(-c2ccc3ccc(OC)cc3c2)n1]

Compound G (45 mg, 69%) was synthesized using the same procedure employed for the synthesis of compound A, yielding a yellow liquid. ¹H NMR (400 MHz, CDCl₃) δ 8.35-8.32 (m, 1H), 7.89 (dd, J=8.5, 1.8 Hz, 1H), 7.80 (d, J=8.5 Hz, 1H), 7.74 (d, J=8.8 Hz, 1H), 7.23 (t, J=0.9 Hz, 1H), 7.22-7.15 (m, 2H), 4.23 (q, J=7.2 Hz, 2H), 3.93 (s, 5H), 1.31 (t, J=7.1 Hz, 3H); ¹³C NMR (100 MHz, CDCl₃) δ 170.5, 168.1, 158.2, 150.0, 134.5, 131.4, 129.7, 129.3, 128.4, 124.8, 121.9, 119.9, 116.1, 106.4, 61.1, 55.4, 37.3, 14.2; HRMS (ESI) m/z calcd for C₁₈H₁₈NO₃S[M+H]⁺, 328.1007; found, 328.0994.

### (h) Synthesis of Ethyl 2-(2-(7-Hydroxynaphthalen-2-yl)Thiazol-4-yl)Acetate (H)

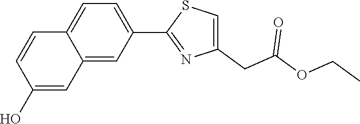 [SMILES: CCOC(=O)Cc1csc(-c2ccc3ccc(O)cc3c2)n1]

Compound H (180 mg, 72%) was synthesized using the same procedure employed for the synthesis of compound A, yielding a yellow solid. ¹H NMR (400 MHz, CDCl₃) δ 8.20-8.16 (m, 1H), 7.80 (dd, J=8.5, 1.8 Hz, 1H), 7.74 (d, J=8.6 Hz, 1H), 7.69 (d, J=8.8 Hz, 1H), 7.23 (d, J=0.9 Hz, 1H), 7.14 (d, J=2.4 Hz, 1H), 7.10 (dd, J=8.8, 2.5 Hz, 1H), 4.24 (q, J=7.1 Hz, 2H), 3.94 (d, J=0.9 Hz, 2H), 1.30 (t, J=7.1 Hz, 3H); ¹³C NMR (150 MHz, CDCl₃) δ 170.8, 154.4, 149.8, 134.5, 131.2, 129.6, 129.4, 128.5, 124.4, 121.7, 119.1, 116.4, 110.2, 61.3, 37.2, 14.2; HRMS (ESI) m/z calcd for C₁₇H₁₆NO₃S[M+H]⁺, 314.0851; found, 314.0838.

### ii. Step 2

### (a) Synthesis of Ethyl (2-(2-(Naphthalen-2-yl)Thiazol-4-yl)Acetyl)Glycinate (1)

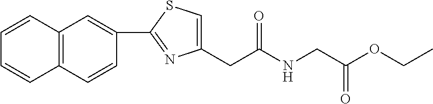 [SMILES: CCOC(=O)CNC(=O)Cc1csc(-c2ccc3ccccc3c2)n1]

A mixture containing 100 mg (0.34 mmoL) of ethyl 2-(2-(naphthalen-2-yl)thiazol-4-yl)acetate was dissolved in a 2:1 mixture of THF-water (3 mL). To this solution, 28 mg of LiOH·H₂O (0.67 mmol) was added, and the reaction was allowed to proceed for 1 h. The reaction was then neutralized to pH=7 by adding 1N HCl. The resulting mixture was concentrated under a high vacuum to give crude acid (90 mg). For the next step, the crude acid (90 mg, 0.33 mmoL) was dissolved in DMF along with HATU (152 mg, 0.40 mmoL), and ethyl glycinate hydrochloride (93 mg, 0.668 mmoL) at room temperature. Diisopropyl ethyl amine (0.18 mL, 1.00 mmoL) was then added to the mixture. After 10 mins, the reaction mixture was quenched by the addition of water. The resulting compound was extracted using EtOAC (2×10 mL), dried over Na₂SO₄ and the organic layer was concentrated under vacuum to obtain the crude product. The crude product underwent purification through silica-gel flash column chromatography, resulting in the isolation of pure 1 (80 mg, 67%) as a white solid. ¹H NMR (500 MHz, CDCl₃) δ 8.49 (d, J=1.7 Hz, 1H), 8.08 (dd, J=8.6, 1.8 Hz, 1H), 8.02-7.79 (m, 3H), 7.73 (d, J=5.2 Hz, 1H), 7.53 (dp, J=6.8, 3.4 Hz, 2H), 7.15 (d, J=0.7 Hz, 1H), 4.21 (q, J=7.1 Hz, 2H), 4.09 (d, J=5.0 Hz, 2H), 3.85 (s, 2H), 1.25 (t, J=7.1 Hz, 3H); ¹³C NMR (125 MHz, CDCl₃) δ 169.8, 169.3, 169.0, 150.6, 134.2, 133.3, 130.6, 128.8, 128.7, 127.9, 127.2, 126.9, 126.2, 123.9, 116.1, 61.5, 41.8, 38.9, 14.2; HRMS (ESI) m/z calcd for C₁₉H₁₉N₂O₃S [M+H]⁺, 355.1116; found, 355.1107.

### (b) Synthesis of Ethyl (2-(2-(Isoquinolin-8-yl)Thiazol-4-yl)Acetyl)Glycinate (2)

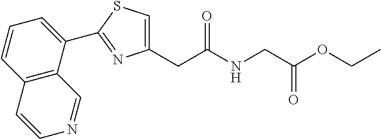 [SMILES: CCOC(=O)CNC(=O)Cc1csc(-c2cccc3ccncc23)n1]

Compound 2 (80 mg, 50%) was synthesized using the same procedure employed for the synthesis of compound 1, yielding a yellow oil. ¹H NMR (600 MHz, CDCl₃) δ 10.20 (s, 1H), 8.59 (d, J=5.8 Hz, 1H), 7.92 (dd, J=11.2, 7.7 Hz, 2H), 7.81-7.67 (m, 2H), 7.37 (s, 1H), 7.21 (q, J=5.8, 4.8 Hz, 1H), 4.19-4.12 (m, 2H), 4.05 (d, J=5.4 Hz, 2H), 3.92 (s, 2H), 1.19 (t, J=7.1 Hz, 3H); ¹³C NMR (150 MHz, CDCl₃) δ 168.7, 168.4, 165.0, 149.9, 149.9, 141.9, 135.5, 130.2, 128.9, 128.5, 127.9, 124.3, 119.8, 116.8, 60.4, 40.6, 38.1, 13.0. HRMS (ESI) m/z calcd for C₁₈H₁₈N₃O₃S [M+H]⁺, 356.1069; found, 356.1059.

### (c) Synthesis of Ethyl (2-(2-(6-Methoxynaphthalen-2-yl)Thiazol-4-yl)Acetyl)Glycinate (3)

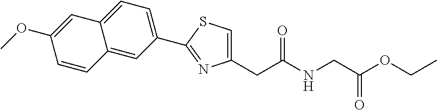 [SMILES: CCOC(=O)CNC(=O)Cc1csc(-c2ccc3cc(OC)ccc3c2)n1]

Compound 3 (100 mg, 78%) was synthesized using the same procedure employed for the synthesis of compound 1, yielding a white solid. ¹H NMR (500 MHz, CDCl₃) δ 8.44 (d, J=1.8 Hz, 1H), 8.05 (dd, J=8.6, 1.8 Hz, 1H), 7.82 (dd, J=20.0, 8.8 Hz, 2H), 7.73 (d, J=5.1 Hz, 1H), 7.20 (dd, J=8.9, 2.5 Hz, 1H), 7.16 (d, J=2.5 Hz, 1H), 7.12 (s, 1H), 4.21 (q, J=7.1 Hz, 2H), 4.09 (d, J=5.0 Hz, 2H), 3.95 (s, 3H), 3.84 (s, 2H), 1.26 (t, J=7.1 Hz, 3H); ¹³C NMR (125 MHz, CDCl₃) δ 169.8, 169.3, 169.3, 158.7, 150.4, 135.7, 130.2, 128.7, 128.6, 127.6, 126.0, 124.5, 119.8, 115.6, 105.9, 61.5, 55.4, 41.8, 38.9, 14.2; HRMS (ESI) m/z calcd for C₂₀H₂₁N₂O₄S [M+H]⁺, 385.1222; found, 385.1227.

### (d) Synthesis of Ethyl (2-(2-(Quinolin-8-yl)Thiazol-4-yl)Acetyl)Glycinate (4)

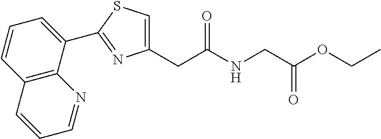 [SMILES: CCOC(=O)CNC(=O)Cc1csc(-c2cccc3cccnc23)n1]

Compound 4 (70 mg, 58%) was synthesized using the same procedure employed for the synthesis of compound 1, yielding a yellow solid. ¹H NMR (400 MHz, CDCl₃) δ 9.06 (dd, J=4.2, 1.8 Hz, 1H), 8.99 (dd, J=7.5, 1.4 Hz, 1H), 8.26 (dd, J=8.3, 1.8 Hz, 1H), 7.91 (dd, J=8.1, 1.4 Hz, 1H), 7.86 (s, 1H), 7.71 (dd, J=8.1, 7.5 Hz, 1H), 7.52 (dd, J=8.3, 4.2 Hz, 1H), 7.31 (d, J=0.9 Hz, 1H), 4.20 (q, J=7.2 Hz, 2H), 4.09 (d, J=5.0 Hz, 2H), 3.89 (s, 2H), 1.26 (t, 7.1 Hz, 3H); ¹³C NMR (100 MHz, CDCl₃) δ 169.6, 169.5, 163.1, 149.3, 148.3, 129.3, 128.7, 128.1, 126.5, 121.3, 120.1, 119.9, 61.2, 41.6, 38.7, 14.0; HRMS (ESI) m/z calcd for C₁₈H₁₈N₃O₃S[M+H]⁺, 356.1069; found, 356.1059.

### (e) Synthesis of Ethyl (2-(2-(Naphthalen-1-yl)Thiazol-4-yl)Acetyl)Glycinate (5)

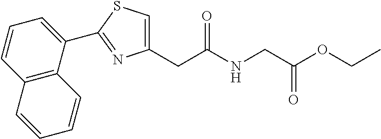 [SMILES: CCOC(=O)CNC(=O)Cc1csc(-c2cccc3ccccc23)n1]

Compound 5 (60 mg, 51%) was synthesized using the same procedure employed for the synthesis of compound 1, yielding a brown solid. ¹H NMR (500 MHz, CDCl₃) δ 8.74-8.69 (m, 1H), 7.96 (d, J=8.2 Hz, 1H), 7.92 (dd, J=7.6, 1.8 Hz, 1H), 7.83 (dd, J=7.1, 1.1 Hz, 1H), 7.62-7.51 (m, 3H), 7.31 (s, 1H), 7.29 (s, 1H), 4.16 (q, J=7.1 Hz, 2H), 4.06 (d, J=5.3 Hz, 2H), 3.93 (s, 2H), 1.20 (t, J=7.1 Hz, 3H); ¹³C NMR (125 MHz, CDCl₃) δ 169.6, 169.4, 168.2, 150.2, 134.0, 130.7, 130.6, 130.5, 128.7, 128.4, 127.5, 126.5, 125.7, 125.1, 117.1, 61.4, 41.6, 39.2, 14.1; HRMS (ESI) m/z calcd for C₁₉H₁₉N₂O₃S[M+H]⁺, 355.1116; found, 355.1107.

### (f) Synthesis of Ethyl (2-(2-(1-Fluoronaphthalen-2-yl)Thiazol-4-yl)Acetyl)Glycinate (6)

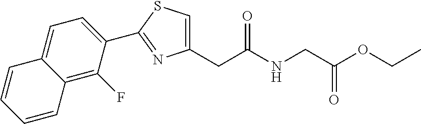 [SMILES: CCOC(=O)CNC(=O)Cc1csc(-c2ccc3ccccc3c2F)n1]

Compound 6 (35 mg, 59%) was synthesized using the same procedure employed for the synthesis of compound 1, yielding a white solid. ¹H NMR (400 MHz, CDCl₃) δ 8.43 (dd, J=8.7, 7.2 Hz, 1H), 8.24-8.18 (m, 1H), 7.87 (dt, J=6.4, 2.2 Hz, 1H), 7.73 (d, J=8.7 Hz, 1H), 7.65 (s, 1H), 7.62-7.58 (m, 2H), 7.29 (d, J=0.8 Hz, 1H), 4.21 (q, J=7.2 Hz, 2H), 4.09 (d, J=5.0 Hz, 2H), 3.88 (d, J=0.8 Hz, 2H), 1.25 (t, J=7.1 Hz, 3H); ¹³C NMR (100 MHz, CDCl₃) δ 169.8, 169.3, 149.3, 135.3, 135.2, 128.0, 127.6, 127.0, 124.5, 124.1, 121.2, 117.7, 117.6, 61.5, 41.8, 38.8, 14.1; HRMS (ESI) m/z calcd for C₁₉H₁₈FN₂O₃S[M+H]⁺, 373.1022; found, 373.1029.

### (g) Synthesis of Ethyl (2-(2-(7-Methoxynaphthalen-2-yl)Thiazol-4-yl)Acetyl)Glycinate (7)

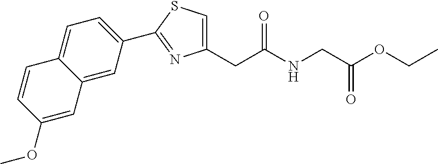 [SMILES: CCOC(=O)CNC(=O)Cc1csc(-c2ccc3ccc(OC)cc3c2)n1]

Compound 7 (30 mg, 56%) was synthesized using the same procedure employed for the synthesis of compound 1, yielding a white solid. ¹H NMR (500 MHz, CDCl₃) δ 8.43 (d, J=1.7 Hz, 1H), 7.94 (dd, J=8.5, 1.7 Hz, 1H), 7.84 (d, J=8.4 Hz, 1H), 7.74 (dd, J=19.4, 7.1 Hz, 2H), 7.25 (d, J=2.7 Hz, 1H), 7.20 (dd, J=8.9, 2.5 Hz, 1H), 7.15 (d, J=3.9 Hz, 1H), 4.21 (q, J=7.1 Hz, 2H), 4.09 (d, J=5.0 Hz, 2H), 3.95 (s, 3H), 3.85 (s, 2H), 1.26 (t, J=7.2 Hz, 3H); ¹³C NMR (125 MHz, CDCl₃) δ 169.8, 169.2, 169.2, 158.3, 150.5, 134.6, 131.0, 129.8, 129.3, 128.6, 125.0, 121.7, 120.1, 116.1, 106.4, 61.4, 55.4, 41.8, 38.9, 14.2; HRMS (ESI) m/z calcd for C₂₀H₂₁N₂O₄S[M+H]⁺, 385.1222; found, 385.1227.

### iii. Step 3

### (a) (2-(2-(Naphthalen-2-yl)Thiazol-4-yl)Acetyl)Glycine (8)

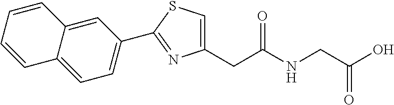 [SMILES: O=C(O)CNC(=O)Cc1csc(-c2ccc3ccccc3c2)n1]

In a stirred solution, ethyl (2-(2-(naphthalen-2-yl)thiazol-4-yl)acetyl)glycinate (100 mg, 0.28 mmol) was dissolved in a 2:1 mixture of THF and water (3 mL). At room temperature, LiOH H₂O (24 mg, 0.56 mmol) was introduced into the mixture. The reaction mixture was stirred for 1 h under the same conditions. After neutralization with 1N HCl, the resulting compound was extracted with ethyl acetate (2×EtOAc), dried using sodium sulfate, and concentrated, yielding the pure product 8 (50 mg, 54%) in the form of a white solid. ¹H NMR (500 MHz, CD₃OD) δ 8.46 (d, J=1.8 Hz, 1H), 8.05 (dd, J=8.5, 1.8 Hz, 1H), 7.99-7.91 (m, 2H), 7.88 (dt, J=7.2, 3.5 Hz, 1H), 7.54 (dt, J=6.2, 3.4 Hz, 2H), 7.42 (s, 1H), 3.98 (s, 2H), 3.86 (s, 2H); ¹³C NMR (126 MHz, CD₃OD) δ 173.1, 172.1, 169.7, 151.9, 135.3, 134.4, 131.7, 129.7, 129.5, 128.6, 128.0, 127.7, 126.9, 124.7, 117.6, 42.3, 39.1; HRMS (ESI) m/z calcd for C₁₇H₁₅N₂O₃S [M+H]⁺, 327.0803; found, 327.0813.

### (b) Synthesis of (2-(2-(Isoquinolin-8-yl)Thiazol-4-yl)Acetyl)Glycine (9)

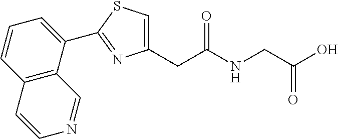 [SMILES: O=C(O)CNC(=O)Cc1csc(-c2cccc3ccncc23)n1]

Compound 9 (25 mg, 90%) was synthesized using the same procedure employed for the synthesis of compound 8, yielding a yellow solid. ¹H NMR (500 MHz, DMSO-d₆) δ 10.21 (s, 1H), 8.60 (d, J=5.6 Hz, 1H), 8.13 (dd, J=18.1, 6.8 Hz, 2H), 8.04 (dd, J=7.3, 1.1 Hz, 1H), 7.94 (dd, J=5.6, 0.9 Hz, 1H), 7.87 (dd, J=8.3, 7.2 Hz, 1H), 7.71 (s, 1H), 3.83 (s, 2H), 3.68 (d, J=5.4 Hz, 2H); ¹³C NMR (125 MHz, DMSO-d₆) δ 171.5, 169.2, 164.9, 152.7, 151.2, 143.8, 136.4, 130.9, 130.6, 129.9, 129.4, 124.9, 121.1, 118.5, 42.5, 38.7; HRMS (ESI) m/z calcd for C₁₆H₁₄N₃O₃S [M+H]⁺, 328.0756; found, 328.0772.

### (c) Synthesis of (2-(2-(6-Methoxynaphthalen-2-yl)Thiazol-4-yl)Acetyl)Glycine (10)

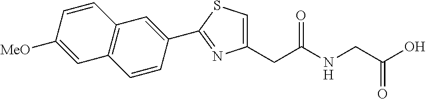 [SMILES: COc1ccc2cc(-c3nc(CC(=O)NCC(=O)O)cs3)ccc2c1]

Compound 10 (50 mg, 77%) was synthesized using the same procedure employed for the synthesis of compound 9, yielding a white solid. ¹H NMR (500 MHz, DMSO-d₆) δ 8.42 (d, J=6.0 Hz, 2H), 8.04-7.96 (m, 2H), 7.91 (d, J=8.6 Hz, 1H), 7.48 (s, 1H), 7.38 (d, J=2.5 Hz, 1H), 7.23 (dd, J=9.0, 2.5 Hz, 1H), 3.90 (s, 3H), 3.82 (d, J=5.8 Hz, 2H), 3.74 (s, 2H); ¹³C NMR (126 MHz, DMSO-d₆) δ 171.8, 169.6, 167.2, 158.7, 152.0, 135.6, 130.7, 128.8, 128.7, 128.1, 125.7, 124.6, 120.0, 116.8, 106.6, 55.8, 41.3, 38.5; HRMS (ESI) m/z calcd for C₁₈H₁₇N₂O₄S [M+H]⁺, 357.0909; found, 357.0909.

### (d) Synthesis of (2-(2-(Quinolin-8-yl)Thiazol-4-yl)Acetyl)Glycine (11)

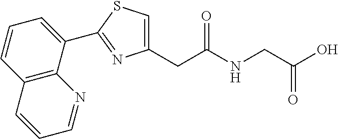 [SMILES: O=C(O)CNC(=O)Cc1csc(-c2cccc3cccnc23)n1]

Compound 11 (40 mg, 62%) was synthesized using the same procedure employed for the synthesis of compound 9, yielding a yellow solid. ¹H NMR (500 MHz, DMSO-d₆) δ 9.10 (dd, J=4.2, 1.8 Hz, 1H), 8.81 (dd, J=7.6, 1.3 Hz, 1H), 8.53 (dd, J=8.4, 1.8 Hz, 1H), 8.43 (t, J=5.8 Hz, 1H), 8.11 (dd, J=8.2, 1.4 Hz, 1H), 7.76 (t, J=7.8 Hz, 1H), 7.69 (dd, J=8.3, 4.1 Hz, 1H), 7.60 (s, 1H), 3.82 (d, J=5.8 Hz, 2H), 3.78 (s, 2H); ¹³C NMR (125 MHz, DMSO-d₆) δ 171.8, 169.9, 161.1, 150.6, 150.1, 143.7, 137.7, 130.4, 130.2, 128.6, 128.6, 127.1, 122.4, 120.7, 41.4, 38.7; HRMS (ESI) m/z calcd for C₁₆H₁₄N₃O₃S [M+H]⁺, 328.0756; found, 328.0772.

### (e) Synthesis of (2-(2-(Naphthalen-1-yl)Thiazol-4-yl)Acetyl)Glycine (12)

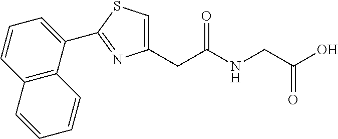 [SMILES: O=C(O)CNC(=O)Cc1csc(-c2cccc3ccccc23)n1]

Compound 12 (30 mg, 81%) was synthesized using the same procedure employed for the synthesis of compound 9, yielding a white solid. ¹H NMR (500 MHz, CD₃OD) δ 8.64-8.59 (m, 1H), 8.01 (d, J=8.3 Hz, 1H), 7.98-7.93 (m, 1H), 7.81 (d, J=7.1 Hz, 1H), 7.62-7.53 (m, 4H), 3.99 (s, 2H), 3.91 (s, 2H); ¹³C NMR (125 MHz, CD₃OD) δ 171.5, 171.4, 167.6, 150.5, 134.1, 130.5, 130.4, 130.3, 128.1, 128.0, 127.0, 126.2, 125.4, 124.8, 117.4, 40.7, 37.9; HRMS (ESI) m/z calcd for C₁₇H₁₅N₂O₃S [M+H]⁺, 327.0803; found, 327.0813.

### (f) Synthesis of (2-(2-(1-Fluoronaphthalen-2-yl)Thiazol-4-yl)Acetyl)Glycine (13)

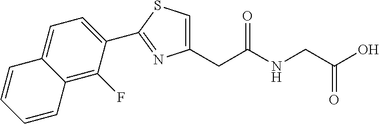 [SMILES: O=C(O)CNC(=O)Cc1csc(-c2ccc3ccccc3c2F)n1]

Compound 13 (10 mg, 83%) was synthesized using the same procedure employed for the synthesis of compound 9, yielding a white solid. ¹H NMR (500 MHz, DMSO-d₆) δ 8.45 (t, J=5.9 Hz, 1H), 8.31 (t, J=8.0 Hz, 1H), 8.24-8.17 (m, 1H), 8.07 (d, J=7.4 Hz, 1H), 7.91 (d, J=8.7 Hz, 1H), 7.74-7.67 (m, 3H), 3.83 (d, J=5.9 Hz, 2H), 3.80 (s, 2H); ¹³C NMR (125 MHz, DMSO-d₆) δ 171.8, 169.6, 156.8, 151.2, 135.1, 135.0, 128.8, 128.3, 128.3, 128.2, 125.0, 124.9, 124.7, 124.6, 123.3, 123.2, 121.1, 121.0, 119.0, 118.9, 115.9, 115.8, 41.3, 38.4; HRMS (ESI) m/z calcd for C₁₇H₁₄FN₂O₃S [M+H]⁺, 345.0709; found, 345.0718.

### (g) Synthesis of (2-(2-(7-Methoxynaphthalen-2-yl)Thiazol-4-yl)Acetyl)Glycine (14)

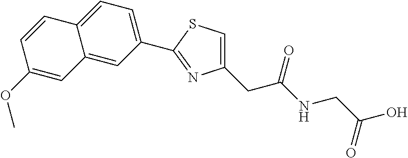 [SMILES: COc1ccc2ccc(-c3nc(CC(=O)NCC(=O)O)cs3)cc2c1]

Compound 14 (20 mg, 86%) was synthesized using the same procedure employed for the synthesis of compound 9, yielding a white solid. ¹H NMR (500 MHz, DMSO-d₆) δ 8.43 (s, 2H), 7.98-7.84 (m, 3H), 7.53 (d, J=2.1 Hz, 2H), 7.23 (dt, J=9.0, 2.3 Hz, 1H), 3.90 (d, J=1.7 Hz, 3H), 3.83 (d, J=5.8 Hz, 2H), 3.76 (s, 2H); ¹³C NMR (125 MHz, DMSO-d₆)) δ 171.8, 169.6, 167.1, 158.4, 152.2, 134.8, 131.4, 129.7, 129.6, 129.0, 124.7, 121.7, 121.7, 120.2, 117.3, 107.2, 55.8, 41.3, 38.5; HRMS (ESI) m/z calcd for C₁₈H₁₇N₂O₄S [M+H]⁺, 357.0909; found, 357.0909.

### b. Synthesis of Exemplary PROTAC Linkers (15 and 15a)

### i. Step 1: Synthesis of Ethyl 2-(2-(8-Hydroxynaphthalen-2-yl)Thiazol-4-yl)Acetate (I)

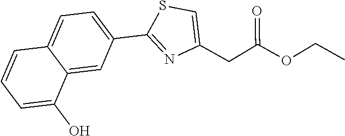 [SMILES: CCOC(=O)Cc1csc(-c2ccc3cccc(O)c3c2)n1]

A mixture containing of 2-(2-bromothiazol-4-yl)acetate (500 mg, 2.00 mmol), 7-(4,4,5,5-tetramethyl-1,3,2-dioxaborolan-2-yl)naphthalen-1-ol (540 mg, 2.00 mmol), K₂CO₃ (3 ml, 6.00 mmol; 2M soln. in water) in dioxane (4.0 ml) was stirred for 5 min while being purged with nitrogen gas. Afterward, tetrakis(triphenylphosphine)palladium(0) (116 mg, 0.1 mmol) was added to the mixture. The reaction mixture was then purged with nitrogen for an additional 2 min then stirred at 60° C. for overnight. The reaction mixture was then cooled to room temperature and quenched with water. The compound was extracted with EtOAc (2×50 mL). The combined EtOAc was dried and evaporated under vacuum. The resulting residue was subjected to flash column purification (30% EtOAc in Hexane) to get product ethyl 2-(2-(naphthalen-2-yl)thiazol-4-yl)acetate I (390 mg, 62%) as a brown solid. ¹H NMR (500 MHz, CDCl₃) δ 8.77 (s, 1H), 8.03 (dt, J=8.6, 1.6 Hz, 1H), 7.81 (d, J=8.6 Hz, 1H), 7.40 (d, J=8.2 Hz, 1H), 7.35-7.29 (m, 1H), 7.24 (s, 1H), 6.83 (d, J=7.4 Hz, 1H), 5.97 (s, 1H), 4.24 (q, J=7.2 Hz, 2H), 3.95 (s, 2H), 1.31 (t, J=7.1 Hz, 3H); ¹³C NMR (125 MHz, CDCl₃) δ 170.6, 168.4, 152.3, 149.8, 135.3, 130.2, 128.4, 127.2, 124.5, 124.3, 120.3, 116.2, 109.5, 61.2, 37.2, 14.2; HRMS (ESI) m/z calcd for C₁₇H₁₆NO₃S[M+H]⁺, 314.0851; found, 314.0838.

### ii. Step 2: Synthesis of Ethyl 2-(2-(8-(2-Methoxyethoxy)Naphthalen-2-yl)Thiazol-4-yl)Acetate (J)

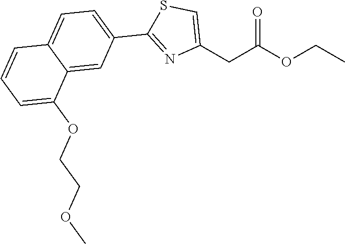 [SMILES: CCOC(=O)Cc1csc(-c2ccc3cccc(OCCOC)c3c2)n1]

To a solution of ethyl 2-(2-(8-hydroxynaphthalen-2-yl)thiazol-4-yl)acetate (100 mg, 0.31 mmoL) and K₂CO₃ (132 mg, 0.96 mmoL) in Acetone (5.0 mL) was added 1-bromo-2-methoxyethane (0.06 mL, 0.64 mmoL) and heated at 60° C. overnight. Evaporated the reaction mixtures and the residue was dissolved in water. The resulting compound was extracted with ethyl acetate (2×20 mL), dried over sodium sulfate, and concentrated. The crude product was subsequently subjected to purification using silica-gel flash column chromatography (Eluents: 25% EtOAc in hexane), resulting in the isolation of J (100 mg, 84%) as a brown liquid. ¹H NMR (500 MHz, CDCl₃) δ 8.80 (d, J=1.8 Hz, 1H), 8.10 (dd, J=8.6, 1.9 Hz, 1H), 7.83 (d, J=8.6 Hz, 1H), 7.49-7.33 (m, 2H), 7.22 (s, 1H), 6.85 (dd, J=7.2, 1.3 Hz, 1H), 4.33 (dd, J=5.6, 4.2 Hz, 2H), 4.23 (q, J=7.1 Hz, 2H), 3.99-3.89 (m, 4H), 3.54 (s, 3H), 1.31 (t, J=7.1 Hz, 3H); ¹³C NMR (125 MHz, CDCl₃) δ 170.4, 168.2, 154.9, 149.7, 135.0, 130.2, 128.1, 127.0, 125.4, 124.4, 120.6, 120.2, 115.9, 105.7, 70.9, 67.9, 60.9, 59.3, 37.1, 14.1; HRMS (ESI) m/z calcd for C₂₀H₂₂NO₄S[M+H]⁺, 372.1270; found, 372.1277.

### iii. Step 3: Synthesis of Ethyl (2-(2-(8-(2-Methoxyethoxy)Naphthalen-2-yl)Thiazol-4-yl)Acetyl)Glycinate (15)

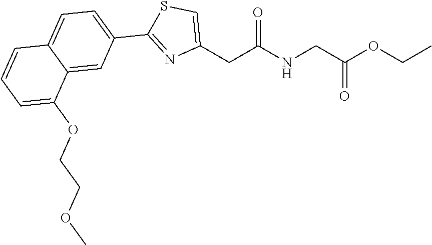 [SMILES: CCOC(=O)CNC(=O)Cc1csc(-c2ccc3cccc(OCCOC)c3c2)n1]

Compound 15 (78 mg, 62%) was synthesized using the same procedure employed for the synthesis of compound 1, yielding a brown solid. ¹H NMR (500 MHz, CDCl₃) δ 8.81 (d, J=1.8 Hz, 1H), 8.17 (dd, J=8.6, 1.9 Hz, 1H), 7.87 (d, J=8.6 Hz, 1H), 7.62 (t, J=5.2 Hz, 1H), 7.52-7.36 (m, 2H), 7.14 (d, J=0.9 Hz, 1H), 6.88 (dd, J=7.1, 1.6 Hz, 1H), 4.34 (dd, J=5.5, 4.1 Hz, 2H), 4.19 (q, J=7.1 Hz, 2H), 4.08 (d, J=5.1 Hz, 2H), 3.98-3.87 (m, 2H), 3.85 (s, 2H), 3.54 (s, 3H), 1.24 (t, J=7.2 Hz, 3H); ¹³C NMR (125 MHz, CDCl₃) δ 169.6, 169.3, 169.3, 155.0, 150.3, 135.2, 129.9, 128.3, 127.2, 125.4, 124.2, 120.8, 120.3, 115.9, 105.8, 70.9, 67.9, 61.3, 59.3, 41.6, 38.8, 14.0; HRMS (ESI) m/z calcd for C₂₂H₂₅N₂O₅S[M+H]⁺, 429.1484; found, 429.1498.

### iv. 2-(2-(8-(2-Methoxyethoxy)Naphthalen-2-yl)Thiazol-4-yl)Acetyl)Glycine (15A)

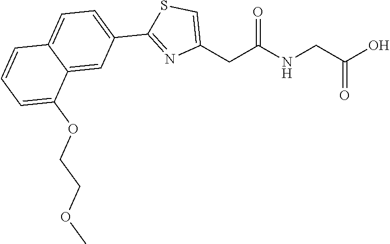 [SMILES: COCCOc1cccc2ccc(-c3nc(CC(=O)NCC(=O)O)cs3)cc12]

Compound 15A (25 mg, 64%) was synthesized using the same procedure employed for the synthesis of compound 8, yielding a yellow solid. ¹H NMR (500 MHz, DMSO-d6) δ 8.71 (d, J=1.8 Hz, 1H), 8.45 (t, J=5.9 Hz, 1H), 8.06 (dd, J=8.6, 1.9 Hz, 1H), 7.99 (d, J=8.6 Hz, 1H), 7.57-7.46 (m, 3H), 7.08 (d, J=7.5 Hz, 1H), 4.38-4.32 (m, 2H), 3.86 (dd, J=5.6, 3.5 Hz, 2H), 3.83 (d, J=5.8 Hz, 2H), 3.78 (s, 2H), 3.42 (s, 3H); 13C NMR (125 MHz, DMSO-d6) δ 171.7, 169.7, 167.0, 154.9, 152.3, 135.1, 130.3, 129.2, 128.2, 125.2, 124.6, 120.5, 119.5, 117.2, 107.0, 70.8, 68.2, 58.9, 41.3, 38.5; HRMS (ESI) m/z calcd for C₂₀H₂₁N₂O₅S[M+H]⁺, 401.1171; found, 401.1176.

### c. Synthesis of Additional PROTAC Linkers (16-18)

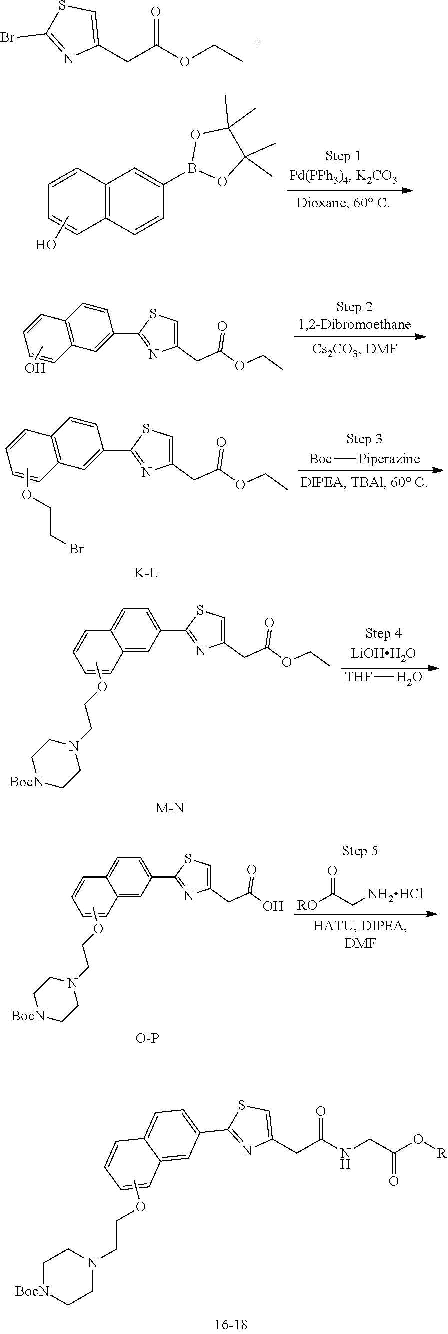

### i. STEP 2

### (a) Synthesis of Ethyl 2-(2-(8-(2-Bromoethoxy)Naphthalen-2-yl)Thiazol-4-yl)Acetate (K)

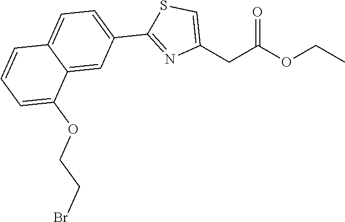 [SMILES: CCOC(=O)Cc1csc(-c2ccc3cccc(OCCBr)c3c2)n1]

To a stirred solution of ethyl 2-(2-(8-hydroxynaphthalen-2-yl)thiazol-4-yl)acetate (400 mg, 1.28 mmoL) and cesium carbonate (1.25 g, 2.83 mmoL) in DMF (6.0 mL) was added 1,2-dibromoethane (0.33 mL, 3.83 mmoL) at room temperature for 3 days. Water (15 mL) was added to the reaction mixture and the product was extracted with ethyl acetate (2×30 mL). The organic layer was dried over sodium sulfate and concentrated. The residue was purified on silica-gel flash column chromatography (Eluents: 20% EtOAc in hexane) to yield K (230 mg, 43%) as a yellow liquid. ¹H NMR (500 MHz, CDCl₃) δ 8.80 (d, J=1.8 Hz, 1H), 8.11 (dd, J=8.5, 1.8 Hz, 1H), 7.84 (d, J=8.6 Hz, 1H), 7.51-7.36 (m, 2H), 7.24 (s, 1H), 6.84 (d, J=7.5 Hz, 1H), 4.50 (t, J=6.4 Hz, 2H), 4.24 (q, J=7.1 Hz, 2H), 3.94 (s, 2H), 3.82 (t, J=6.3 Hz, 2H), 1.31 (t, J=7.1 Hz, 3H); ¹³C NMR (125 MHz, CDCl₃) δ 170.4, 168.1, 154.2, 149.8, 135.1, 130.5, 128.2, 126.8, 125.4, 124.6, 120.8, 120.4, 116.0, 105.8, 68.0, 61.0, 37.1, 28.9, 14.1; HRMS (ESI) m/z calcd for C₁₉H₁₉BrNO₃S[M+H]⁺, 420.0269; found, 420.0289.

### (b) Synthesis of Ethyl 2-(2-(7-(2-Bromoethoxy)Naphthalen-2-yl)Thiazol-4-yl)Acetate (L)

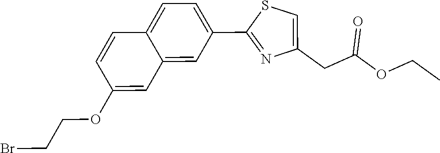 [SMILES: CCOC(=O)Cc1csc(-c2ccc3ccc(OCCBr)cc3c2)n1]

Compound L (80 mg, 40%) was synthesized using the same procedure employed for the synthesis of compound K, yielding a yellow oil. ¹H NMR (400 MHz, CDCl₃) δ 8.32 (d, J=1.7 Hz, 1H), 7.91 (dd, J=8.5, 1.8 Hz, 1H), 7.85-7.73 (m, 2H), 7.24 (d, J=0.9 Hz, 1H), 7.23-7.19 (m, 2H), 4.42 (t, J=6.3 Hz, 2H), 4.24 (q, J=7.2 Hz, 2H), 3.93 (d, J=0.9 Hz, 2H), 3.72 (t, J=6.3 Hz, 2H), 1.31 (t, J=7.1 Hz, 3H); ¹³C NMR (150 MHz, CDCl₃) δ 170.4, 168.0, 156.7, 150.0, 134.4, 131.5, 129.9, 129.6, 128.5, 124.8, 122.3, 119.9, 116.2, 107.6, 67.9, 61.1, 37.3, 28.9, 14.2; HRMS (ESI) m/z calcd for C₁₉H₁₉BrNO₃S[M+H]⁺, 420.0269; found, 420.0289.

### ii. Step 3

### (a) Synthesis of Tert-Butyl 4-(2-((7-(4-(2-Ethoxy-2-Oxoethyl)Thiazol-2-yl)Naphthalen-1-yl)Oxy)Ethyl)Piperazine-1-Carboxylate (M)

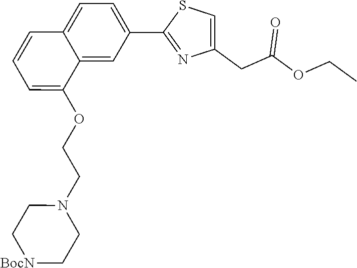 [SMILES: CCOC(=O)Cc1csc(-c2ccc3cccc(OCCN4CCN(C(=O)OC(C)(C)C)CC4)c3c2)n1]

In a stirred solution, ethyl 2-(2-(8-(2-bromoethoxy)naphthalen-2-yl)thiazol-4-yl)acetate (120 mg, 0.28 mmol), tert-butyl piperazine-1-carboxylate (64 mg, 0.34 mmol), and tetrabutylammonium iodide (5 mg, 0.014 mmol) were combined in 2.0 mL of DMF. Diisopropyl ethylamine (0.10 mL, 0.57 mmol) was added to the mixture at room temperature, followed by overnight heating at 60° C. The reaction mixture was quenched with water and the product was extracted with ethyl acetate (2×30 mL). The organic layer was dried over sodium sulfate and concentrated. The residue was purified on silica-gel flash column chromatography (Eluents: 80% EtOAc in hexane) to yield M (120 mg, 80%) as a colorless oil. ¹H NMR (500 MHz, CDCl₃) δ 8.74 (d, J=1.8 Hz, 1H), 8.11 (dd, J=8.6, 1.8 Hz, 1H), 7.84 (d, J=8.5 Hz, 1H), 7.47-7.36 (m, 2H), 7.23 (s, 1H), 6.85 (dd, J=7.1, 1.4 Hz, 1H), 4.33 (t, J=5.7 Hz, 2H), 4.23 (q, J=7.1 Hz, 2H), 3.93 (s, 2H), 3.48 (t, J=5.1 Hz, 4H), 3.02 (t, J=5.7 Hz, 2H), 2.65 (t, J=5.0 Hz, 4H), 1.46 (s, 9H), 1.31 (t, J=7.1 Hz, 3H); ¹³C NMR (125 MHz, CDCl₃) δ 170.4, 168.1, 154.8, 154.6, 149.8, 135.0, 130.3, 128.2, 127.0, 125.4, 124.4, 120.5, 120.2, 115.9, 105.5, 79.5, 66.5, 61.0, 57.1, 53.4, 37.2, 28.3, 14.1; HRMS (ESI) m/z calcd for C₂₈H₃₆N₃O₅S[M+H]⁺, 526.2376; found, 526.2402.

### (b) Synthesis of Tert-Butyl 4-(2-((7-(4-(2-Ethoxy-2-Oxoethyl)Thiazol-2-yl)Naphthalen-2-yl)Oxy)Ethyl)Piperazine-1-Carboxylate (N)

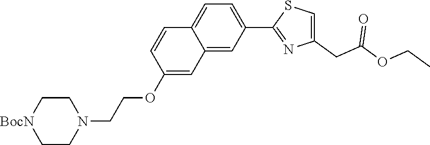 [SMILES: CCOC(=O)Cc1csc(-c2ccc3ccc(OCCN4CCN(C(=O)OC(C)(C)C)CC4)cc3c2)n1]

Compound N (70 mg, 70%) was synthesized using the same procedure employed for the synthesis of compound M, yielding a colorless oil. ¹H NMR (500 MHz, CD₃OD) δ 8.32 (s, 1H), 7.87-7.82 (m, 2H), 7.78 (s, 1H), 7.41 (s, 1H), 7.32 (d, J=2.5 Hz, 1H), 7.19 (dd, J=9.0, 2.4 Hz, 1H), 4.28-4.17 (m, 4H), 3.90 (s, 2H), 3.46 (t, J=4.9 Hz, 4H), 2.87 (t, J=5.4 Hz, 2H), 2.57 (t, J=5.1 Hz, 4H), 1.46 (s, 9H), 1.28 (t, J=7.1 Hz, 3H); 13C NMR (125 MHz, CD₃OD) δ 170.8, 168.5, 157.5, 155.0, 150.0, 134.6, 131.0, 129.8, 129.0, 128.2, 124.6, 121.3, 119.9, 116.7, 107.0, 79.9, 65.3, 60.8, 56.8, 53.0, 36.2, 27.3, 13.1; HRMS (ESI) m/z calcd for C₂₈H₃₆N₃O₅S[M+H]⁺, 526.2376; found, 526.2355.

### iii. Step 4

### (a) Synthesis of 2-(2-(8-(2-(4-(Tert-Butoxycarbonyl)Piperazin-1-yl)Ethoxy)Naphthalen-2-yl)Thiazol-4-yl)Acetic Acid (O)

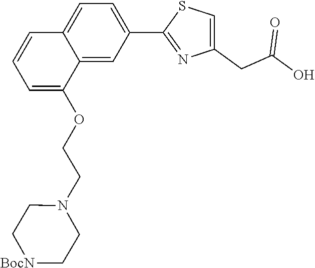 [SMILES: CC(C)(C)OC(=O)N1CCN(CCOc2cccc3ccc(-c4nc(CC(=O)O)cs4)cc23)CC1]

In a solution containing tert-butyl 4-(2-((7-(4-(2-ethoxy-2-oxoethyl)thiazol-2-yl)naphthalen-1-yl)oxy)ethyl)piperazine-1-carboxylate (50 mg, 0.09 mmol) in a 2:1 mixture of THF and water (1.5 mL), LiOH H₂O (4.8 mg, 0.11 mmol) was introduced at room temperature and stirred for 1 h. Following neutralization with 1N HCl, the compound was extracted with ethyl acetate (2×20 mL), dried over sodium sulfate, and concentrated to yield the pure compound O (40 mg, 85%) as a yellow solid. ¹H NMR (600 MHz, DMSO-d₆) δ 8.72 (d, J=1.8 Hz, 1H), 8.11 (dd, J=8.6, 1.9 Hz, 1H), 8.03 (d, J=8.6 Hz, 1H), 7.57-7.49 (m, 2H), 7.47 (s, 1H), 7.16-7.08 (m, 1H), 4.40 (t, J=5.8 Hz, 2H), 3.63 (s, 2H), 3.41-3.31 (m, 4H), 2.99 (t, J=5.8 Hz, 2H), 2.62 (t, J=5.1 Hz, 4H), 1.45 (s, 9H); ¹³C NMR (150 MHz, DMSO-d₆) δ 172.4, 166.0, 154.8, 154.3, 146.2, 135.0, 130.7, 129.0, 128.0, 125.3, 124.5, 120.4, 119.5, 107.0, 79.2, 66.7, 56.8, 53.3, 29.7, 28.5; HRMS (ESI) m/z calcd for C₂₆H₃₂N₃O₅S[M+H]⁺, 498.2063; found, 498.2066.

### (b) Synthesis of 2-(2-(7-(2-(4-(Tert-Butoxycarbonyl)Piperazin-1-yl)Ethoxy)Naphthalen-2-yl)Thiazol-4-yl)Acetic Acid (P)

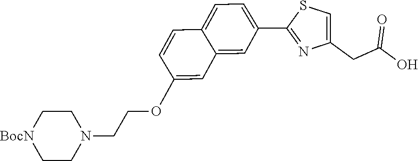 [SMILES: CC(C)(C)OC(=O)N1CCN(CCOc2ccc3ccc(-c4nc(CC(=O)O)cs4)cc3c2)CC1]

Compound P (58 mg, 88%) was synthesized using the same procedure employed for the synthesis of compound 0, yielding a white solid. ¹H NMR (600 MHz, DMSO-d₆) δ 10.58 (s, 1H), 8.50-8.37 (m, 1H), 8.03-7.86 (m, 3H), 7.59 (d, J=33.2 Hz, 2H), 7.36-7.21 (m, 1H), 4.53 (s, 2H), 4.03 (s, 2H), 3.82 (s, 1H), 3.71-3.42 (m, 4H), 2.29-2.35 (m, 1H), 3.20-2.92 (m, 2H), 2.50-2.45 (m, 2H), 1.42 (s, 9H). ¹³C NMR (150 MHz, DMSO-d₆) δ 172.0, 167.0, 153.8, 151.3, 134.6, 131.4, 129.9, 129.2, 124.7, 122.1, 120.2, 118.0, 108.6, 39.6, 37.3, 28.4; HRMS (ESI) m/z calcd for C₂₆H₃₂N₃O_{5S}[M+H]⁺, 498.2063; found, 498.2066.

### iv. STEP 5

### (a) Synthesis of Tert-Butyl 4-(2-((7-(4-(2-((2-Ethoxy-2-Oxoethyl)Amino)-2-Oxoethyl)Thiazol-2-yl)Naphthalen-1-yl)Oxy)Ethyl)Piperazine-1-Carboxylate (16)

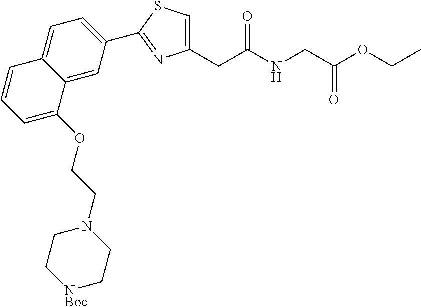 [SMILES: CCOC(=O)CNC(=O)Cc1csc(-c2ccc3cccc(OCCN4CCN(C(=O)OC(C)(C)C)CC4)c3c2)n1]

A solution comprising 2-(2-(8-(2-(4-(tert-butoxycarbonyl)piperazin-1-yl)ethoxy)naphthalen-2-yl)thiazol-4-yl)acetic acid (40 mg, 0.08 mmol), ethyl glycinate hydrochloride (13.5 mg, 0.096 mmol), and HATU (34 mg, 0.088 mmol) in DMF (1.0 mL) was stirred. N,N-Diisopropylethylamine (0.043 mL, 0.24 mmol) was then added at room temperature, and stirred for 15 minutes under the same conditions. The reaction mixture was subsequently quenched with water, followed by extraction with EtOAc (2×10 mL). The organic phase was then dried over Na₂SO₄ and concentrated under vacuum to obtain the crude product. This crude product was purified using silica-gel flash column chromatography (Eluents: 80% EtOAc in hexane) to yield 16 (35 mg, 75%) as colorless oil. ¹H NMR (500 MHz, CDCl₃) δ 8.77 (d, J=1.8 Hz, 1H), 8.18 (dd, J=8.6, 1.8 Hz, 1H), 7.87 (d, J=8.6 Hz, 1H), 7.62 (t, J=5.2 Hz, 1H), 7.52-7.36 (m, 2H), 7.17 (s, 1H), 6.86 (dd, J=6.8, 1.7 Hz, 1H), 4.35 (t, J=5.6 Hz, 2H), 4.19 (q, J=7.1 Hz, 2H), 4.07 (d, J=5.2 Hz, 2H), 3.86 (s, 2H), 3.50 (t, J=5.1 Hz, 4H), 3.05 (t, J=5.6 Hz, 2H), 2.69 (s, 4H), 1.46 (s, 9H), 1.24 (t, J=7.2 Hz, 3H); ¹³C NMR (125 MHz, CDCl₃) δ 169.6, 169.3, 169.1, 154.8, 154.6, 150.4, 135.2, 130.0, 128.3, 127.2, 125.4, 124.2, 120.6, 120.2, 115.9, 105.6, 79.7, 66.5, 61.3, 57.1, 53.4, 41.6, 38.8, 28.3, 14.0; HRMS (ESI) m/z calcd for C₃₀H₃₉N₄O₆S[M+H]⁺, 583.2590; found, 583.2598.

### (b) Synthesis of Tert-Butyl 4-(2-((7-(4-(2-((2-(Benzyloxy)-2-Oxoethyl)Amino)-2-Oxoethyl)Thiazol-2-yl)Naphthalen-1-yl)Oxy)Ethyl)Piperazine-1-Carboxylate (17)

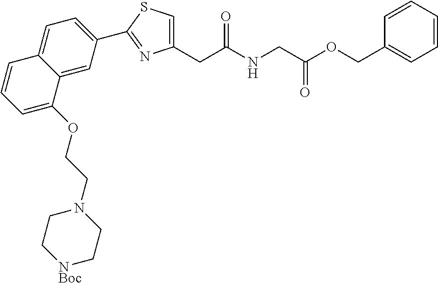 [SMILES: CC(C)(C)OC(=O)N1CCN(CCOc2cccc3ccc(-c4nc(CC(=O)NCC(=O)OCc5ccccc5)cs4)cc23)CC1]

Compound 17 (150 mg, 77%) was prepared following the identical procedure used for synthesizing compound 16, using benzyl glycinate hydrochloride as the amine. ¹H NMR (500 MHz, CDCl₃) δ 8.76 (d, J=1.9 Hz, 1H), 8.15 (dd, J=8.5, 1.8 Hz, 1H), 7.85 (d, J=8.6 Hz, 1H), 7.70 (t, J=5.2 Hz, 1H), 7.48-7.36 (m, 2H), 7.30 (s, 6H), 7.15 (s, 1H), 6.85 (dd, J=6.8, 1.8 Hz, 1H), 5.16 (s, 2H), 4.32 (t, J=5.6 Hz, 2H), 4.11 (d, J=4.1 Hz, 2H), 3.85 (s, 2H), 3.48 (t, J=5.1 Hz, 4H), 3.01 (t, J=5.6 Hz, 2H), 2.66 (s, 4H), 1.45 (s, 9H); ¹³C NMR (125 MHz, CDCl₃) δ 169.4, 169.3, 169.0, 154.7, 154.6, 150.3, 135.1, 135.0, 129.9, 128.4, 128.2, 128.1, 127.2, 125.3, 124.2, 120.6, 120.1, 115.9, 105.5, 79.5, 66.9, 66.5, 60.2, 57.0, 53.3, 41.5, 38.7, 28.2; HRMS (ESI) m/z calcd for C₃₅H₄₁N₄O_{6S}[M+H]⁺, 645.2747; found, 645.2730.

### (c) Synthesis of Tert-Butyl 4-(2-((7-(4-(2-((2-(Benzyloxy)-2-Oxoethyl)Amino)-2-Oxoethyl)Thiazol-2-yl)Naphthalen-2-yl)Oxy)Ethyl)Piperazine-1-Carboxylate (18)

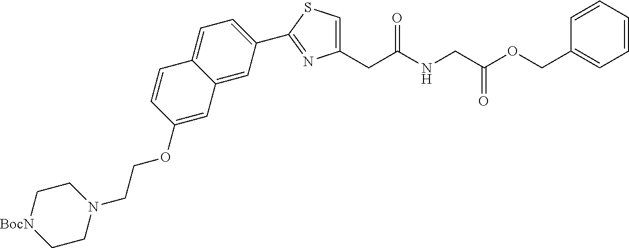 [SMILES: CC(C)(C)OC(=O)N1CCN(CCOc2ccc3ccc(-c4nc(CC(=O)NCC(=O)OCc5ccccc5)cs4)cc3c2)CC1]

Compound 18 (50 mg, 77%) was prepared following the identical procedure used for synthesizing compound 17, yielding a yellow solid. ¹H NMR (500 MHz, CDCl₃) δ 8.32 (s, 1H), 7.87 (d, J=8.6 Hz, 1H), 7.76 (d, J=8.5 Hz, 1H), 7.75-7.61 (m, 2H), 7.27 (d, J=14.7 Hz, 4H), 7.22-7.12 (m, 3H), 7.08 (s, 1H), 5.12 (s, 2H), 4.18 (t, J=5.7 Hz, 2H), 4.12-4.02 (m, 2H), 3.79 (s, 2H), 3.41 (t, J=5.0 Hz, 4H), 2.92-2.76 (m, 2H), 2.51 (t, J=5.0 Hz, 4H), 1.41 (d, J=1.5 Hz, 9H); ¹³C NMR (125 MHz, CDCl₃) δ 169.7, 169.4, 169.1, 157.4, 154.7, 150.5, 135.2, 134.5, 131.1, 129.9, 129.4, 128.7, 128.6, 128.6, 128.5, 128.3, 125.0, 121.8, 120.3, 116.2, 107.3, 79.7, 67.1, 65.9, 60.4, 57.2, 53.4, 41.8, 38.9, 28.4; HRMS (ESI) m/z calcd for C₃₅H₄₁N₄O₆S[M+H]⁺, 645.2747; found, 645.2730.

### d. Synthesis of Exemplary PROTAC Linker (19) (A) Ethyl 2-(2-(8-(2-(Piperazin-1-yl)Ethoxy)Naphthalen-2-yl)Thiazol-4-yl)Acetate (Q)

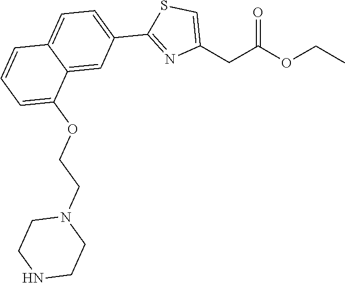 [SMILES: CCOC(=O)Cc1csc(-c2ccc3cccc(OCCN4CCNCC4)c3c2)n1]

In a stirred solution, tert-butyl 4-(2-((7-(4-(2-ethoxy-2-oxoethyl)thiazol-2-yl)naphthalen-1-yl)oxy)ethyl)piperazine-1-carboxylate (150 mg, 0.28 mmol) was dissolved in CH₂Cl₂ (2.0 mL), and then 4M HCl in dioxane (2.0 mL) was added at room temperature. The reaction mixture was stirred for 1 hour under the same temperature, after which it was evaporated to yield the pure product Q (140 mg, 98%) as a white solid. ¹H NMR (600 MHz, D₂O) δ 8.47 (d, J=4.0 Hz, 1H), 7.93-7.76 (m, 2H), 7.60-7.41 (m, 3H), 6.96 (dd, J=7.7, 3.9 Hz, 1H), 4.68-4.48 (m, 2H), 4.27 (q, J=7.1 Hz, 2H), 3.97 (d, J=4.2 Hz, 2H), 3.92 (dd, J=6.3, 3.1 Hz, 2H), 3.86 (s, 4H), 3.70 (t, J=5.5 Hz, 4H), 1.31 (t, J=7.1 Hz, 3H); ¹³C NMR (150 MHz, D₂O) δ 172.6, 169.8, 153.2, 135.0, 128.8, 128.1, 124.2, 124.1, 124.1, 121.1, 119.9, 118.7, 106.6, 62.5, 61.8, 56.1, 48.9, 40.6, 35.6, 13.3; HRMS (ESI) m/z calcd for C₂₃H₂₈N₃O₃S[M+H]⁺, 426.1851; found, 426.1845.

### (b) Synthesis of (2-(2-(8-(2-(Piperazin-1-yl)Ethoxy)Naphthalen-2-yl)Thiazol-4-yl)Acetyl)Glycine (19)

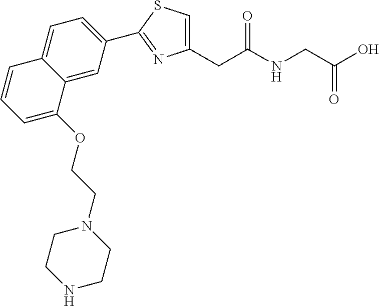 [SMILES: O=C(O)CNC(=O)Cc1csc(-c2ccc3cccc(OCCN4CCNCC4)c3c2)n1]

In a solution containing of tert-butyl 4-(2-((7-(4-(2-((2-ethoxy-2-oxoethyl)amino)-2-oxoethyl)thiazol-2-yl)naphthalen-1-yl)oxy)ethyl)piperazine-1-carboxylate (30 mg, 0.05 mmoL) in a mixture of THF and water (1.5 mL, 2:1 ratio), LiOH H₂O (2.5 mg, 0.051 mmoL) was added at room temperature. The reaction mixture was stirred at ambient temperature for 1 h. Following this, the mixture was neutralized using 1N HCl, and the resulting acid derivative was extracted with EtAOc (2×10 mL), dried over Na₂SO₄, and concentrated under vacuum to give acid derivative. This acid derivative was then dissolved in THF (1.0 mL), and 4M HCl in dioxane (1.0 mL) was introduced into the reaction mixture. After 1 h, the reaction mixture evaporated to yield 19 (16 mg, 68%) as a white solid. ¹H NMR (500 MHz, D₂O) δ 8.02 (s, 1H), 7.15-6.89 (m, 4H), 6.85 (d, J=8.2 Hz, 1H), 6.25 (d, J=7.7 Hz, 1H), 3.58 (d, J=11.5 Hz, 6H), 2.96 (d, J=6.0 Hz, 4H), 2.59 (d, J=13.0 Hz, 6H); ¹³C NMR (125 MHz, D₂O) δ 176.3, 171.5, 168.6, 153.9, 150.1, 134.4, 128.6, 128.1, 127.6, 124.4, 123.7, 120.2, 118.9, 117.6, 105.8, 64.8, 55.8, 49.1, 43.4, 42.4, 37.6; HRMS (ESI) m/z calcd for C₂₃H₂₇N₄O₄S [M+H]⁺, 455.1753; found, 455.1739.

### e. Synthesis of JQ1 PROTACS 5328, 5329, 5407, and 5408

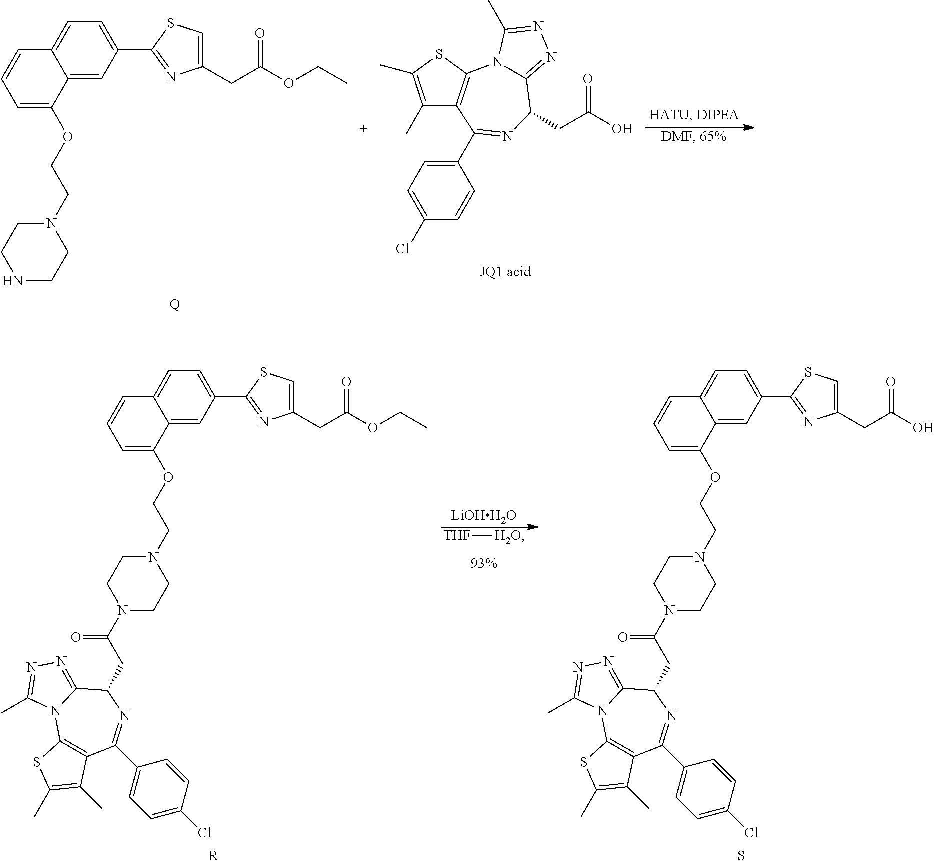

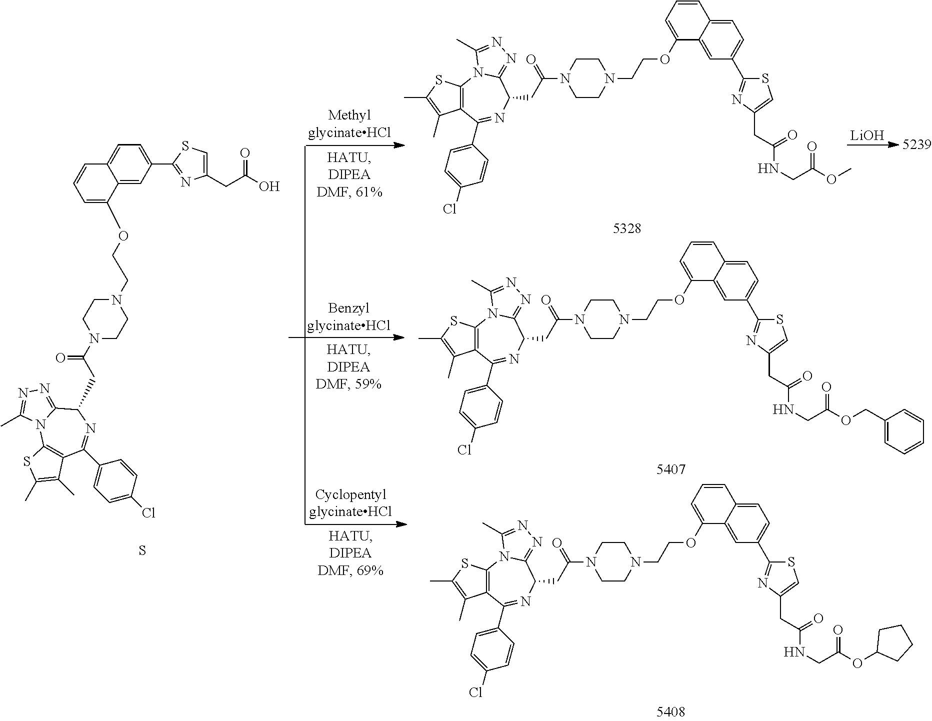

### i. Synthesis of Ethyl (S)-2-(2-(8-(2-(4-(2-(4-(4-Chlorophenyl)-2,3,9-Trimethyl-6H-Thieno [3,2-F][1,2,4]Triazolo [4,3-A][1,4]Diazepin-6-yl)Acetyl)Piperazin-1-yl)Ethoxy)Naphthalen-2-yl)Thiazol-4-yl)Acetate (R)

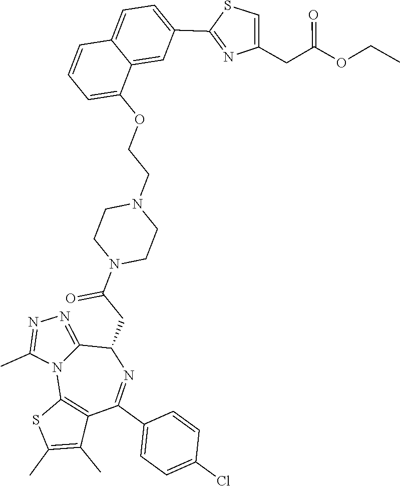 [SMILES: CCOC(=O)Cc1csc(-c2ccc3cccc(OCCN4CCN(C(=O)C[C@@H]5N=C(c6ccc(Cl)cc6)c6c(sc(C)c6C)-n6c(C)nnc65)CC4)c3c2)n1]

In a stirred solution, a mixture of ethyl 2-(2-(8-(2-(piperazin-1-yl)ethoxy)naphthalen-2-yl)thiazol-4-yl)acetate dihydrochloride Q (100 mg, 0.20 mmol), (S)-2-(4-(4-chlorophenyl)-2,3,9-trimethyl-6H-thieno[3,2-f][1,2,4]triazolo[4,3-a][1,4]diazepin-6-yl)acetic acid (80 mg, 0.20 mmol), and HATU (92 mg, 0.24 mmol) in DMF (2.0 mL), N,N-diisopropylethylamine (0.11 mL, 0.60 mmol) was added at room temperature. The reaction mixture was stirred for 15 min. before being quenched with water. The resulting compound was extracted with ethyl acetate (2×10 mL), dried over Na₂SO₄, and concentrated to yield a crude residue. This residue was subsequently purified through silica-gel flash column chromatography (Eluents: 20% MeOH in EtOAc), resulting in the isolation of R (105 mg, 65%) as a colorless oil. ¹H NMR (600 MHz, CD₃OD) δ 8.79 (d, J=1.8 Hz, 1H), 8.01 (dd, J=8.5, 1.9 Hz, 1H), 7.88 (d, J=8.6 Hz, 1H), 7.49-7.44 (m, 2H), 7.41 (dt, J=7.1, 2.2 Hz, 2H), 7.36 (d, J=8.9 Hz, 3H), 7.00 (dd, J=6.3, 2.3 Hz, 1H), 4.67 (dd, J=7.8, 6.0 Hz, 1H), 4.41 (t, J=5.2 Hz, 2H), 4.17 (q, J=7.1 Hz, 2H), 3.90-3.82 (m, 4H), 3.74 (t, J=5.3 Hz, 2H), 3.65 (dd, J=16.3, 7.8 Hz, 1H), 3.58-3.53 (m, 1H), 3.14 (d, J=5.4 Hz, 2H), 2.99-2.94 (m, 2H), 2.84 (p, J=5.7 Hz, 2H), 2.68 (s, 3H), 2.45 (s, 3H), 1.67 (s, 3H), 1.25 (t, J=6.7 Hz, 3H); ¹³C NMR (150 MHz, CD₃OD) δ 170.7, 168.5, 164.7, 155.8, 154.7, 150.7, 150.1, 136.8, 136.5, 135.3, 132.1, 131.8, 130.7, 130.6, 129.9, 129.9, 128.4, 128.3, 127.3, 125.3, 123.8, 120.1, 120.0, 116.7, 105.9, 65.9, 60.8, 60.2, 56.6, 54.0, 53.2, 52.9, 45.1, 41.3, 36.2, 13.1, 13.0, 11.5, 10.2; HRMS (ESI) m/z calcd for C₄₂H₄₃ClN₇O₄S₂[M+H]⁺, 808.2506; found, 808.2537.

### ii. Synthesis of (S)-2-(2-(8-(2-(4-(2-(4-(4-Chlorophenyl)-2,3,9-Trimethyl-6H-Thieno[3,2-F][1,2,4]Triazolo[4,3-A][1,4]Diazepin-6-yl)Acetyl)Piperazin-1-yl)Ethoxy)Naphthalen-2-yl)Thiazol-4-yl)Acetic Acid (S)

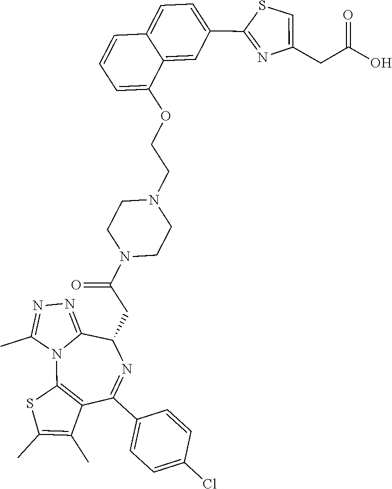 [SMILES: Cc1sc2c(c1C)C(c1ccc(Cl)cc1)=N[C@@H](CC(=O)N1CCN(CCOc3cccc4ccc(-c5nc(CC(=O)O)cs5)cc34)CC1)c1nnc(C)n1-2]

In a solution containing ethyl (S)-2-(2-(8-(2-(4-(2-(4-(4-chlorophenyl)-2,3,9-trimethyl-6H-thieno[3,2-f][1,2,4]triazolo[4,3-a][1,4]diazepin-6-yl)acetyl)piperazin-1-yl)ethoxy)naphthalen-2-yl)thiazol-4-yl)acetate (100 mg, 0.12 mmol) in a 2:1 mixture of THF-water (3.0 mL), LiOH H₂O (5.7 mg, 0.16 mmol) was introduced at room temperature and stirred for 1 h. After neutralization with TN HCl, the resulting compound precipitated and was washed with water, yielding S (90 mg, 93%) as a white solid. ¹H NMR (600 MHz, DMSO-d₆) δ 8.74 (d, J=1.9 Hz, 1H), 8.12 (dd, J=8.6, 1.9 Hz, 1H), 8.03 (d, J=8.6 Hz, 1H), 7.61-7.48 (m, 6H), 7.40 (s, 1H), 7.16 (d, J=7.5 Hz, 1H), 4.64 (t, J=6.7 Hz, 1H), 4.43 (t, J=5.8 Hz, 2H), 3.77 (t, J=5.3 Hz, 2H), 3.68 (dd, J=16.3, 7.2 Hz, 1H), 3.65-3.54 (m, 2H), 3.51 (s, 2H), 3.50-3.45 (m, 1H), 3.04 (t, J=5.8 Hz, 2H), 2.80-2.75 (m, 2H), 2.68-2.67 (m, 2H), 2.66 (s, 3H), 2.47 (s, 3H), 1.69 (s, 3H); ¹³C NMR (150 MHz, DMSO-d₆) δ 168.5, 165.6, 163.3, 156.8, 155.7, 154.8, 150.2, 137.2, 135.6, 134.9, 132.7, 130.6, 129.0, 127.9, 125.3, 124.5, 120.4, 119.4, 115.2, 106.9, 66.7, 56.8, 54.6, 53.8, 53.4, 45.6, 42.4, 41.8, 35.2, 14.5, 13.2, 11.8; HRMS (ESI) m/z calcd for C₄₀H₃₉ClN₇O₄S₂[M+H]⁺, 780.2193; found, 780.2209.

### iii. Synthesis of Methyl (S)-(2-(2-(8-(2-(4-(2-(4-(4-Chlorophenyl)-2,3,9-Trimethyl-6H-Thieno[3,2-F][1,2,4]Triazolo[4,3-A][1,4]Diazepin-6-yl)Acetyl)Piperazin-1-yl)Ethoxy)Naphthalen-2-yl)Thiazol-4-yl)Acetyl)Glycinate (5328)

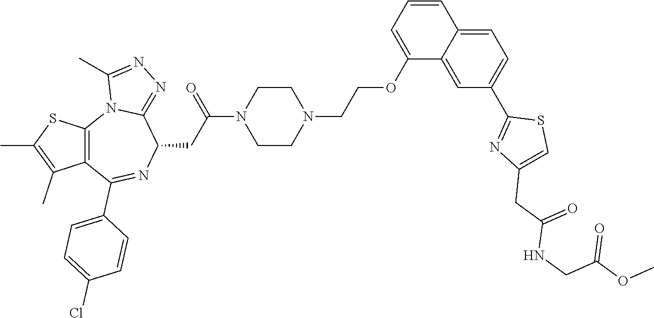

Compound 5328 (8 mg, 61%) was prepared following the same procedure as employed for the synthesis of compound 5407, resulting in the formation of a white solid. ¹H NMR (500 MHz, DMSO-d₆) δ 8.70 (s, 1H), 8.54 (t, J=5.9 Hz, 1H), 8.06 (dd, J=8.6, 1.9 Hz, 1H), 7.99 (d, J=8.6 Hz, 1H), 7.58-7.39 (m, 7H), 7.11 (d, J=7.4 Hz, 1H), 4.57 (t, J=6.7 Hz, 1H), 4.38 (t, J=5.7 Hz, 2H), 3.90 (d, J=5.9 Hz, 2H), 3.76 (s, 2H), 3.70 (t, J=5.2 Hz, 2H), 3.63 (s, 3H), 3.61-3.46 (m, 3H), 3.41 (dd, J=16.4, 6.4 Hz, 1H), 2.98 (t, J=5.6 Hz, 2H), 2.71 (q, J=5.1 Hz, 2H), 2.60 (d, J=5.9 Hz, 5H), 2.41 (s, 3H), 1.62 (s, 3H); ¹³C NMR (125 MHz, DMSO-d₆) δ 170.8, 169.8, 168.6, 167.1, 163.3, 155.7, 154.9, 152.1, 150.2, 137.2, 135.6, 135.1, 132.7, 131.1, 130.6, 130.4, 130.1, 129.1, 129.0, 128.9, 128.9, 128.2, 125.3, 124.5, 120.5, 119.7, 117.3, 107.1, 66.8, 60.2, 56.8, 54.6, 53.8, 53.4, 52.2, 45.6, 41.8, 41.3, 38.5, 35.2, 14.5, 14.5, 13.2, 11.8; HRMS (ESI) m/z calcd for C₄₃H₄₄ClN₈O₅S₂ [M+H], 851.2565; found, 851.2574.

### iv. Synthesis of (S)-(2-(2-(8-(2-(4-(2-(4-(4-Chlorophenyl)-2,3,9-Trimethyl-6H-Thieno[3,2-F][1,2,4]Triazolo[4,3-A][1,4]Diazepin-6-yl)Acetyl)Piperazin-1-yl)Ethoxy)Naphthalen-2-yl)Thiazol-4-yl)Acetyl)Glycine (5329)

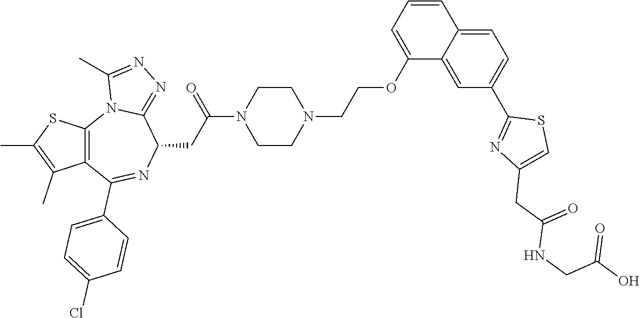 [SMILES: Cc1sc2c(c1C)C(c1ccc(Cl)cc1)=N[C@@H](CC(=O)N1CCN(CCOc3cccc4ccc(-c5nc(CC(=O)NCC(=O)O)cs5)cc34)CC1)c1nnc(C)n1-2]

In a solution containing methyl (S)-(2-(2-(8-(2-(4-(2-(4-(4-chlorophenyl)-2,3,9-trimethyl-6H-thieno[3,2-f][1,2,4]triazolo[4,3-a][1,4]diazepin-6-yl)acetyl)piperazin-1-yl)ethoxy)naphthalen-2-yl)thiazol-4-yl)acetyl)glycinate (25 mg, 0.029 mmol) dissolved in a 2:1 mixture of THF-water (1.5 mL), LiOH H₂O (1.8 mg, 0.044 mmol) was added at room temperature and stirred for 1 h. After neutralization with 1N HCl, the resulting compound precipitated and was washed with water, yielding 5329 (20 mg, 81%) as a white solid. ¹H NMR (500 MHz, DMSO-d₆) δ 8.69 (s, 1H), 8.42 (t, J=5.8 Hz, 1H), 8.11 (dd, J=8.6, 1.9 Hz, 1H), 8.02 (d, J=8.6 Hz, 1H), 7.65-7.41 (m, 7H), 7.16 (d, J=7.7 Hz, 1H), 4.68 (d, J=6.0 Hz, 2H), 4.58 (t, J=6.7 Hz, 1H), 4.47 (t, J=13.5 Hz, 2H), 3.93-3.65 (m, 10H), 3.44 (s, 2H), 3.20 (d, J=35.6 Hz, 2H), 2.60 (s, 3H), 2.42 (s, 3H), 1.63 (s, 3H); ¹³C NMR (125 MHz, DMSO-d₆) δ 171.8, 169.6, 167.0, 163.5, 155.6, 154.0, 152.2, 150.4, 139.7, 137.2, 135.7, 135.1, 132.7, 131.3, 130.7, 130.6, 130.4, 130.1, 129.2, 129.0, 128.0, 125.0, 124.7, 121.3, 119.8, 117.5, 107.3, 63.4, 55.0, 54.3, 52.3, 52.0, 49.0, 42.2, 41.5, 38.3, 35.1, 34.9, 31.2, 14.5, 13.2, 11.8; HRMS (ESI) m/z calcd for C₄₂H₄₂ClN₈O₅S₂ [M+H]⁺, 837.2408; found, 837.2429.

### v. Synthesis of Benzyl (S)-(2-(2-(8-(2-(4-(2-(4-(4-Chlorophenyl)-2,3,9-Trimethyl-6H-Thieno[3,2-F][1,2,4]Triazolo[4,3-A][1,4]Diazepin-6-yl)Acetyl)Piperazin-1-yl)Ethoxy)Naphthalen-2-yl)Thiazol-4-yl)Acetyl)Glycinate (5407)

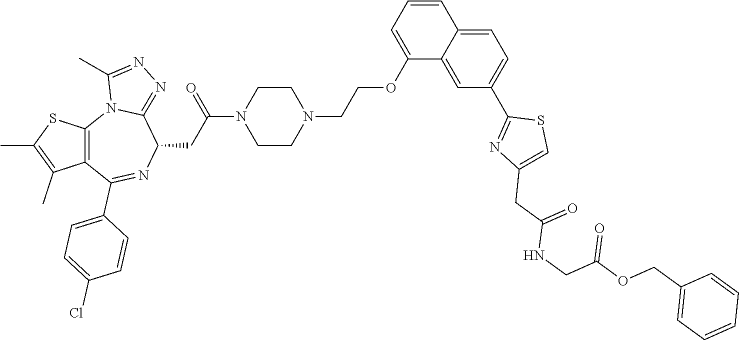 [SMILES: Cc1sc2c(c1C)C(c1ccc(Cl)cc1)=N[C@@H](CC(=O)N1CCN(CCOc3cccc4ccc(-c5nc(CC(=O)NCC(=O)OCc6ccccc6)cs5)cc34)CC1)c1nnc(C)n1-2]

A mixture comprising (S)-2-(2-(8-(2-(4-(2-(4-(4-chlorophenyl)-2,3,9-trimethyl-6H-thieno[3,2-f][1,2,4]triazolo[4,3-a][1,4]diazepin-6-yl)acetyl)piperazin-1-yl)ethoxy)naphthalen-2-yl)thiazol-4-yl)acetic acid (10 mg, 0.013 mmoL), benzyl glycinate hydrochloride (3 mg, 0.014 mmoL), HATU (5.5 mg, 0.014 mmoL), and DMF (0.5 mL) was treated with N,N-diisopropylethylamine (0.007 mL, 0.038 mmoL) at room temperature. The reaction mixture was stirred for 15 minutes before being quenched with water. The resulting compound was extracted with ethyl acetate (2×5 mL), dried over Na₂SO₄, and concentrated to obtain a crude residue. This crude product was subsequently purified through silica-gel flash column chromatography (30% methanol in ethyl acetate), resulting in the isolation of 5407 (7 mg, 59%) as a white solid. ¹H NMR (600 MHz, CDCl₃) δ 8.78 (d, J=1.9 Hz, 1H), 8.16 (dd, J=8.5, 1.8 Hz, 1H), 7.87 (d, J=8.5 Hz, 1H), 7.76 (t, J=5.3 Hz, 1H), 7.48-7.37 (m, 4H), 7.35-7.28 (m, 6H), 7.16 (d, J=0.8 Hz, 1H), 6.88 (dd, J=7.1, 1.5 Hz, 1H), 5.16 (s, 2H), 4.84-4.78 (m, 1H), 4.37 (t, J=5.4 Hz, 2H), 4.14-4.10 (m, 2H), 3.93-3.87 (m, 1H), 3.85 (d, J=0.8 Hz, 2H), 3.85-3.81 (m, 1H), 3.76 (ddt, J=11.4, 9.7, 5.7 Hz, 1H), 3.71-3.58 (m, 3H), 3.07 (t, J=5.5 Hz, 2H), 2.93-2.76 (m, 3H), 2.77-2.67 (m, 1H), 2.66 (s, 3H), 2.39 (s, 3H), 1.66 (s, 3H); ¹³C NMR (150 MHz, CDCl₃) δ 169.6, 169.5, 169.1, 168.9, 163.7, 155.9, 155.0, 150.5, 149.9, 136.8, 136.7, 135.3, 132.2, 130.9, 130.7, 130.6, 130.2, 129.8, 128.7, 128.6, 128.4, 128.4, 128.3, 127.3, 125.5, 124.4, 120.8, 120.4, 116.2, 105.8; HRMS (ESI) m/z calcd for C₄₉H₄₈ClN₈O₅S₂[M+H]⁺, 927.2878; found, 927.2874.

### vi. Synthesis of Cyclopentyl (S)-(2-(2-(8-(2-(4-(2-(4-(4-Chlorophenyl)-2,3,9-Trimethyl-6H-Thieno[3,2-F][1,2,4]Triazolo[4,3-A][1,4]Diazepin-6-yl)Acetyl)Piperazin-1-yl)Ethoxy)Naphthalen-2-yl)Thiazol-4-yl)Acetyl)Glycinate (5408)

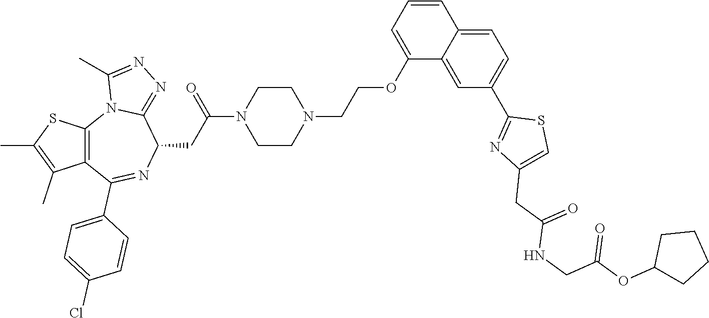 [SMILES: Cc1sc2c(c1C)C(c1ccc(Cl)cc1)=N[C@@H](CC(=O)N1CCN(CCOc3cccc4ccc(-c5nc(CC(=O)NCC(=O)OC6CCCC6)cs5)cc34)CC1)c1nnc(C)n1-2]

Compound 5408 (25 mg, 69%) was prepared following the same procedure as employed for the synthesis of compound 5407, resulting in the formation of a white solid. ¹H NMR (600 MHz, Acetone-d₆) δ 8.93 (d, J=1.8 Hz, 1H), 8.13 (dd, J=8.5, 1.9 Hz, 1H), 7.97 (d, J=8.5 Hz, 1H), 7.86-7.79 (m, 1H), 7.55-7.47 (m, 5H), 7.43-7.39 (m, 2H), 7.08 (dd, J=7.5, 1.2 Hz, 1H), 5.12 (td, J=6.0, 3.2 Hz, 1H), 4.73 (t, J=6.7 Hz, 1H), 4.45 (t, J=5.3 Hz, 2H), 3.91 (d, J=5.8 Hz, 2H), 3.87-3.81 (m, 4H), 3.67 (dd, J=10.7, 5.3 Hz, 3H), 3.48 (dd, J=16.0, 6.8 Hz, 1H), 3.07 (t, J=5.3 Hz, 2H), 2.92-2.87 (m, 2H), 2.75 (t, J=5.2 Hz, 2H), 2.61 (s, 3H), 2.46 (s, 3H), 1.85-1.76 (m, 2H), 1.71 (s, 3H), 1.69-1.59 (m, 4H), 1.56-1.48 (m, 2H); ¹³C NMR (150 MHz, Acetone-d₆) δ 169.4, 169.0, 168.3, 167.5, 163.0, 155.8, 155.1, 151.9, 149.6, 137.4, 135.7, 135.2, 132.7, 130.7, 130.4, 130.3, 130.2, 130.2, 128.4, 128.4, 127.6, 125.6, 124.2, 120.1, 120.1, 116.2, 106.3, 77.3, 67.1, 56.8, 54.6, 53.9, 53.4, 45.7, 41.3, 38.5, 32.3, 23.4, 13.6, 12.1, 10.9; HRMS (ESI) m/z calcd for C₄₇H₅₀ClN₈O₅S₂[M+H], 905.3034; found, 905.3066.

### f. Synthesis of JQ1 PROTACS 5341 and 5342

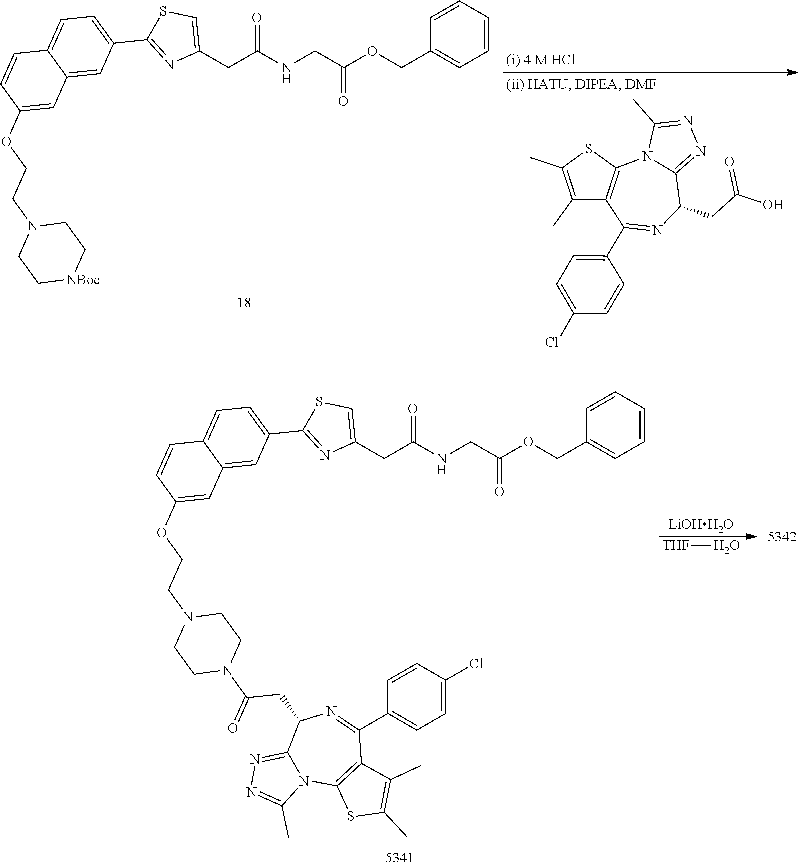

### i. Synthesis of Benzyl (S)-(2-(2-(7-(2-(4-(2-(4-(4-Chlorophenyl)-2,3,9-Trimethyl-6H-Thieno[3,2-F][1,2,4]Triazolo[4,3-A][1,4]Diazepin-6-yl)Acetyl)Piperazin-1-yl)Ethoxy)Naphthalen-2-yl)Thiazol-4-yl)Acetyl)Glycinate (5341)

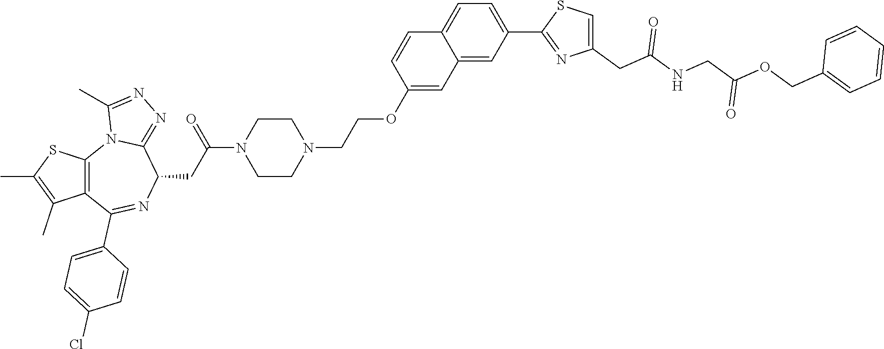 [SMILES: Cc1sc2c(c1C)C(c1ccc(Cl)cc1)=N[C@@H](CC(=O)N1CCN(CCOc3ccc4ccc(-c5nc(CC(=O)NCC(=O)OCc6ccccc6)cs5)cc4c3)CC1)c1nnc(C)n1-2]

A mixture containing 50 mg (0.078 mmol) of tert-butyl 4-(2-((7-(4-(2-((2-(benzyloxy)-2-oxoethyl)amino)-2-oxoethyl)thiazol-2-yl)naphthalen-2-yl)oxy)ethyl)piperazine-1-carboxylate in 1.0 mL of THF was subjected to treatment with 1.0 mL of 4M HCl in dioxane at room temperature. The resulting mixture was stirred for 1 hour, leading to the evaporation of the solvent and the formation of pure amine. Subsequently, the obtained amine (45 mg, 0.078 mmol) was mixed with (S)-2-(4-(4-chlorophenyl)-2,3,9-trimethyl-6H-thieno[3,2-f][1,2,4]triazolo[4,3-a][1,4]diazepin-6-yl)acetic acid (32 mg, 0.081 mmol) and HATU (34 mg, 0.089 mmol) in 1.0 mL of DMF. To this mixture, N,N-diisopropylethylamine (0.043 mL, 0.24 mmoL) was added, and the reaction was stirred for 15 mins. Quenched with water, and the resulting compound was extracted with ethyl acetate (2×20 mL). The organic phase was dried over Na₂SO₄ and concentrated. This crude product was subsequently purified through silica-gel flash column chromatography (Eluents: 25% MeOH in EtOAc), resulting in the isolation of compound 5341 (50 mg, 67%) as a white solid. ¹H NMR (500 MHz, CD₂Cl₂) δ 8.41 (d, J=1.7 Hz, 1H), 7.95 (dd, J=8.4, 1.8 Hz, 1H), 7.85 (d, J=8.5 Hz, 1H), 7.79 (d, J=8.9 Hz, 1H), 7.55 (t, J=5.4 Hz, 1H), 7.43 (d, J=8.3 Hz, 2H), 7.37-7.27 (m, 8H), 7.23-7.17 (m, 2H), 5.17 (s, 2H), 4.75 (t, J=6.7 Hz, 1H), 4.28 (t, J=5.5 Hz, 2H), 4.18-4.00 (m, 3H), 3.82 (s, 3H), 3.78-3.58 (m, 3H), 3.50 (dd, J=16.0, 6.8 Hz, 1H), 2.96 (t, J=5.6 Hz, 2H), 2.77 (s, 2H), 2.72-2.65 (m, 2H), 2.62 (s, 3H), 2.39 (s, 3H), 1.68 (s, 3H); ¹³C NMR (125 MHz, CD₂Cl₂) δ 170.8, 169.7, 169.1, 168.8, 168.6, 163.5, 157.4, 156.0, 150.8, 150.0, 137.0, 136.4, 135.5, 134.5, 132.3, 131.2, 130.9, 130.7, 130.4, 129.9, 129.8, 129.4, 128.5, 128.5, 128.4, 128.2, 124.9, 121.7, 120.1, 116.2, 107.3, 66.9, 57.1, 54.5, 45.5, 41.7, 38.9, 35.3, 14.1, 12.8, 11.6; HRMS (ESI) m/z calcd for C₄₉H₄₈ClN₈O₅S₂ [M+H]⁺, 927.2878; found, 927.2874.

### ii. Synthesis of (S)-(2-(2-(7-(2-(4-(2-(4-(4-Chlorophenyl)-2,3,9-Trimethyl-6H-Thieno[3,2-F][1,2,4]Triazolo[4,3-A][1,4]Diazepin-6-yl)Acetyl)Piperazin-1-yl)Ethoxy)Naphthalen-2-yl)Thiazol-4-yl)Acetyl)Glycine (5342)

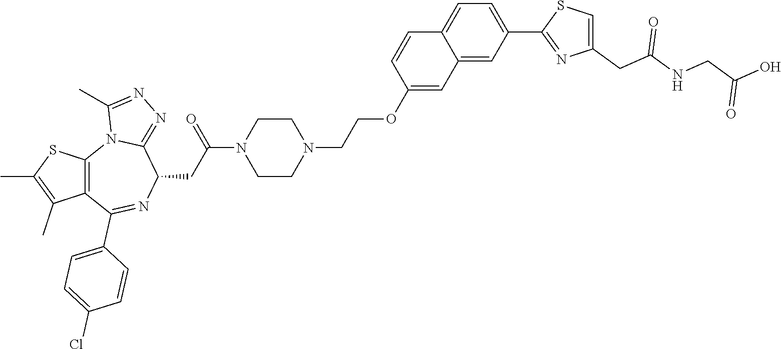 [SMILES: Cc1sc2c(c1C)C(c1ccc(Cl)cc1)=N[C@@H](CC(=O)N1CCN(CCOc3ccc4ccc(-c5nc(CC(=O)NCC(=O)O)cs5)cc4c3)CC1)c1nnc(C)n1-2]

In a solution containing benzyl (S)-(2-(2-(7-(2-(4-(2-(4-(4-chlorophenyl)-2,3,9-trimethyl-6H-thieno[3,2-f][1,2,4]triazolo[4,3-a][1,4]diazepin-6-yl)acetyl)piperazin-1-yl)ethoxy)naphthalen-2-yl)thiazol-4-yl)acetyl)glycinate (25 mg, 0.027 mmol) dissolved in a 2:1 mixture of THF-water (1.5 mL), LiOH H₂O (1.7 mg, 0.040 mmol) was added at room temperature and stirred for 1 h. After neutralization with 1N HCl, the resulting compound extracted with a mixture of 2-MeTHF and EtOAc (1:1 ratio; 2×20 mL), resulting in the isolation of 5342 (16 mg, 71%) as a yellow solid. ¹H NMR (500 MHz, DMSO-d₆) δ 8.45 (s, 2H), 8.03-7.89 (m, 2H), 7.65 (d, J=2.5 Hz, 1H), 7.55 (s, 1H), 7.51 (d, J=8.3 Hz, 1H), 7.45 (d, J=8.3 Hz, 1H), 7.32 (d, J=4.1 Hz, 3H), 4.60 (d, J=5.8 Hz, 2H), 4.55-4.33 (m, 3H), 3.83 (d, J=5.8 Hz, 2H), 3.77 (s, 3H), 3.71 (t, J=8.2 Hz, 5H), 3.52-3.48 (m, 1H), 3.25-3.10 (m, 3H), 2.62 (s, 3H), 2.43 (s, 3H), 1.64 (s, 3H); ¹³C NMR (126 MHz, DMSO-d₆) δ 171.8, 169.6, 166.9, 163.6, 156.6, 152.2, 150.5, 143.0, 139.7, 137.1, 135.8, 134.6, 131.5, 131.4, 130.7, 130.4, 130.1, 129.9, 129.0, 128.5, 127.1, 126.9, 125.4, 124.7, 122.1, 117.5, 108.6, 63.3, 54.9, 54.3, 52.1, 51.9, 42.4, 41.3, 38.5, 34.8, 34.1, 14.5, 13.2, 11.8; HRMS (ESI) m/z calcd for C₄₂H₄₂ClN₈O₅S₂ [M+H]⁺, 837.2408; found, 837.2429.

### g. Synthesis of JQ1 PROTACS 5372 and 5373

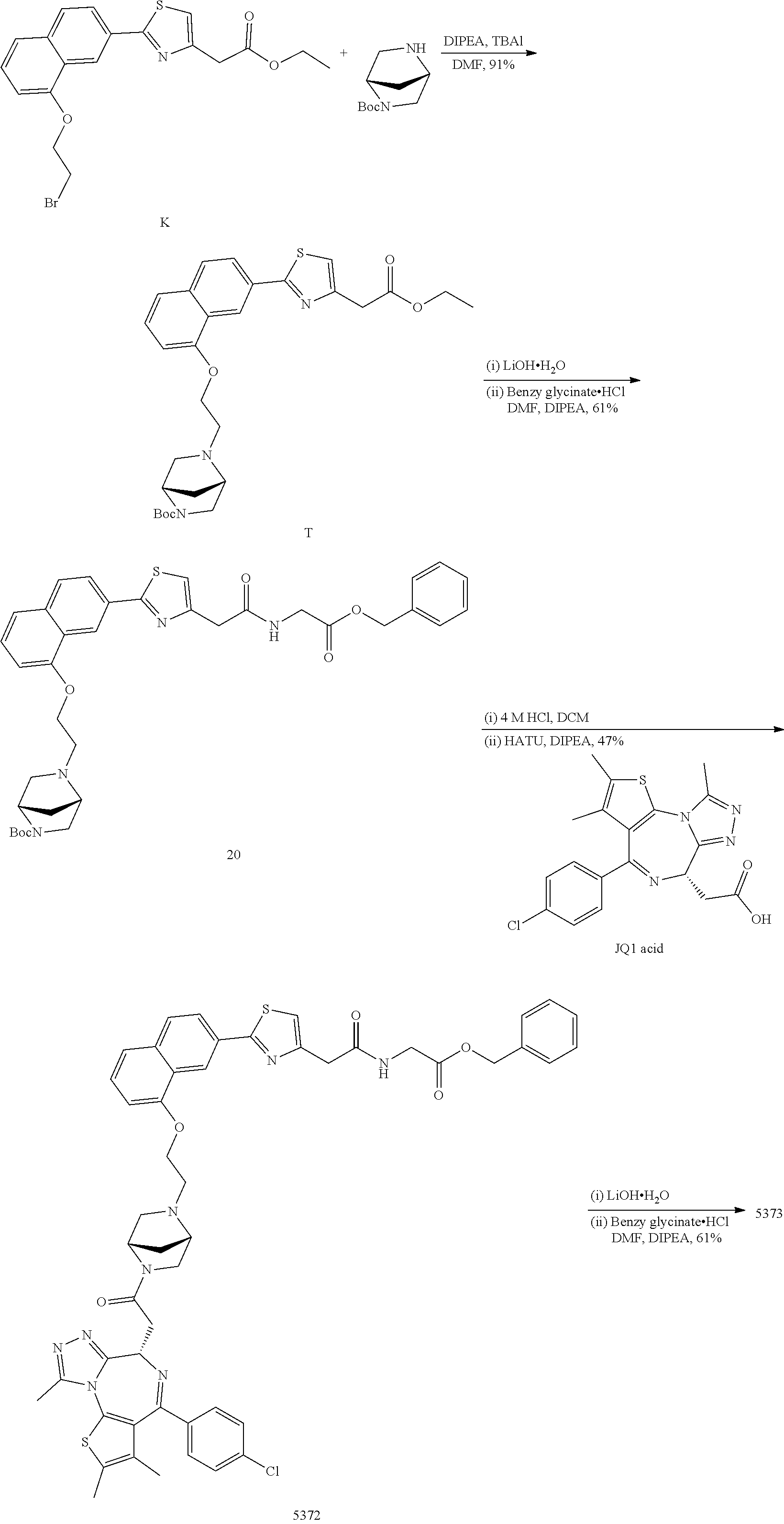

### i. Synthesis of Tert-Butyl (1S,4S)-5-(2-((7-(4-(2-Ethoxy-2-Oxoethyl)Thiazol-2-yl)Naphthalen-1-yl)Oxy)Ethyl)-2,5-Diazabicyclo[2.2.1]Heptane-2-Carboxylate (T)

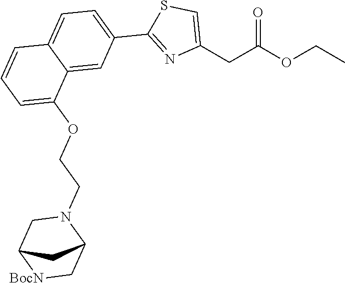 [SMILES: CCOC(=O)Cc1csc(-c2ccc3cccc(OCCN4C[C@@H]5C[C@H]4CN5C(=O)OC(C)(C)C)c3c2)n1]

Compound T (280 mg, 91%) was synthesized using the identical procedure employed for the production of compound M starting from K, with tert-butyl (1S,4S)-2,5-diazabicyclo[2.2.1]heptane-2-carboxylate as the amine. ¹H NMR (500 MHz, CDCl₃) δ 8.66 (s, 1H), 8.04 (d, J=8.5 Hz, 1H), 7.76 (d, J=8.6 Hz, 1H), 7.34 (d, J=8.4 Hz, 2H), 7.15 (d, J=2.4 Hz, 1H), 6.77 (d, J=7.0 Hz, 1H), 4.23-4.14 (m, 4H), 3.86 (s, 2H), 3.71-3.50 (m, 2H), 3.28-3.01 (m, 4H), 2.72 (dd, J=52.0, 9.5 Hz, 1H), 1.83 (d, J=9.8 Hz, 1H), 1.76-1.59 (m, 2H), 1.40 (s, 9H), 1.24 (t, J=7.2 Hz, 3H); ¹³C NMR (125 MHz, CDCl₃) δ 170.5, 168.2, 150.0, 135.2, 130.4, 127.2, 120.2, 105.5, 79.4, 79.3, 68.6, 68.5, 62.3, 62.1, 61.5, 61.2, 61.1, 60.4, 57.9, 56.8, 53.0, 52.7, 49.7, 48.9, 37.3, 28.6, 14.2; HRMS (ESI) m/z calcd for C₂₉H₃₆N₃O₅S[M+H]⁺, 538.2376; found, 538.2359.

### ii. Synthesis of Tert-Butyl (1S,4S)-5-(2-((7-(4-(2-((2-(Benzyloxy)-2-Oxoethyl)Amino)-2-Oxoethyl)Thiazol-2-yl)Naphthalen-1-yl)Oxy)Ethyl)-2,5-Diazabicyclo[2.2.1]Heptane-2-Carboxylate (20)

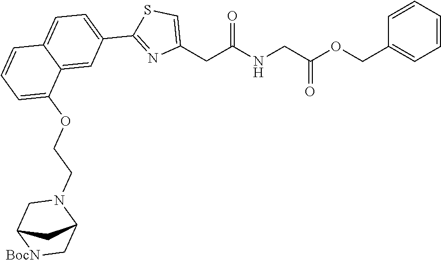 [SMILES: CC(C)(C)OC(=O)N1C[C@@H]2C[C@H]1CN2CCOc1cccc2ccc(-c3nc(CC(=O)NCC(=O)OCc4ccccc4)cs3)cc12]

In a solution containing tert-butyl (1S,4S)-5-(2-((7-(4-(2-ethoxy-2-oxoethyl)thiazol-2-yl)naphthalen-1-yl)oxy)ethyl)-2,5-diazabicyclo[2.2.1]heptane-2-carboxylate (280 mg, 0.52 mmol) in a 2:1 mixture of THF and water (3.0 mL), LiOH H₂O (44 mg, 1.04 mmol) was introduced at room temperature and stirred for 1 h. Following neutralization with 1N HCl, the compound was extracted with ethyl acetate (2×20 mL), dried over Na₂SO₄, and concentrated to yield pure acid. To the obtained acid (165 mg, 0.32 mmoL), benzyl glycinate·HCl (98 mg, 0.49 mmoL), and HATU (185 mg, 0.49 mmoL) were added in DMF (3.0 mL). N,N-Diisopropylethylamine (0.0.17 mL, 0.97 mmol) was then added at room temperature, and stirred for 15 minutes under the same conditions. The reaction mixture was subsequently quenched with water, followed by extraction with EtOAc (2×30 mL). The organic phase was then dried over Na₂SO₄ and concentrated under vacuum to obtain the crude product. This crude product was purified using silica-gel flash column chromatography (Eluents: 80% EtOAc in hexane) to yield 20 (130 mg, 42%) as colorless oil. ¹H NMR (400 MHz, CDCl₃) δ 8.81 (d, J=12.8 Hz, 1H), 8.00 (t, J=10.6 Hz, 1H), 7.83 (d, J=8.6 Hz, 1H), 7.63 (d, J=19.2 Hz, 1H), 7.48-7.38 (m, 2H), 7.37-7.27 (m, 5H), 7.18 (d, J=0.7 Hz, 1H), 6.85 (dd, J=7.3, 1.3 Hz, 1H), 5.15 (s, 2H), 4.40 (s, 2H), 4.22-4.07 (m, 2H), 3.86 (d, J=2.4 Hz, 2H), 3.72-3.66 (m, 1H), 3.49 (d, J=13.4 Hz, 4H), 3.04 (s, 1H), 2.96-2.81 (m, 2H), 2.08 (d, J=12.4 Hz, 1H), 1.91 (d, J=12.9 Hz, 1H), 1.45 (s, 9H); ¹³C NMR (150 MHz, CDCl₃) δ 171.2, 168.8, 155.5, 135.2, 130.0, 128.9, 128.7, 128.7, 128.6, 128.6, 128.6, 128.4, 128.1, 127.4, 125.2, 124.5, 116.4, 68.1, 67.1, 62.1, 55.8, 43.7, 41.7, 38.7, 28.5, 14.2, 12.5; HRMS (ESI) m/z calcd for C₃₆H₄₁N₄O₆S[M+H]⁺, 657.2747; found, 657.2740.

### iii. Synthesis of Benzyl (2-(2-(8-(2-((1S,4S)-5-(2-((S)-4-(4-Chlorophenyl)-2,3,9-Trimethyl-6H-Thieno[3,2-F][1,2,4]Triazolo[4,3-A][1,4]Diazepin-6-yl)Acetyl)-2,5-Diazabicyclo[2.2.1]Heptan-2-yl)Ethoxy)Naphthalen-2-yl)Thiazol-4-yl)Acetyl)Glycinate (5372)

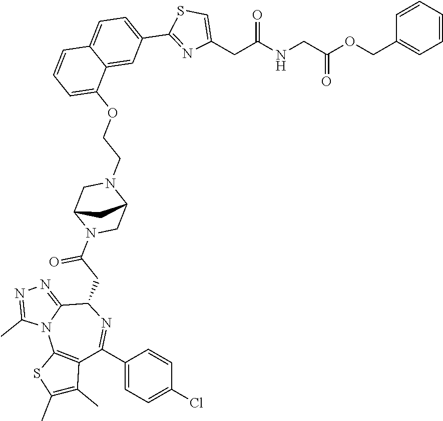 [SMILES: Cc1sc2c(c1C)C(c1ccc(Cl)cc1)=N[C@@H](CC(=O)N1C[C@@H]3C[C@H]1CN3CCOc1cccc3ccc(-c4nc(CC(=O)NCC(=O)OCc5ccccc5)cs4)cc13)c1nnc(C)n1-2]

Compound 5372 (60 mg, 47%) was prepared following the same procedure as employed for the synthesis of compound 5341 starting from 20, and it yielded a white solid. ¹H NMR (500 MHz, Acetone-d₆; 1:1.2 ratio of rotamers) δ 8.94 (d, J=1.8 Hz, 1H, major rotamer), 8.85 (d, J=1.8 Hz, 1H, minor rotamer), 8.09-8.03 (m, 3H, major rotamer), 7.95-7.93 (m, 3H minor rotamer), 7.54-7.21 (m, 11H, both rotamer), 7.03 (ddd, J=11.7, 7.0, 1.6 Hz, 1H, both rotamer), 5.12 (d, J=4.8 Hz, 2H, both rotamer), 4.90 (s, 1H, minor rotamer), 4.80-4.71 (m, 3H, 2H major rotamer, 1H minor rotamer), 4.48-4.32 (m, 3H, 2H major rotamer, 1H minor rotamer), 3.99-3.95 (m, 5H, both rotamer), 3.79 (d, J=13.2 Hz, 4H, both rotamer), 3.64 (dd, J=15.3, 7.5 Hz, 1H, minor rotamer), 3.57 (d, J=6.9 Hz, 1H, major rotamer), 3.51-3.45 (m, 2H, both rotamer), 3.39-3.27 (m, 2H, minor rotamer), 2.89 (d, J=9.4 Hz, 1H, major rotamer), 2.64-2.57 (m, 1H, major rotamer), 2.51 (s, 3H, minor rotamer), 2.43 (d, J=3.8 Hz, 6H, major rotamer), 1.99-1.96 (m, 1H, minor rotamer), 1.96 (s, 3H, minor rotamer), 1.95-1.93 (m, 1H, major rotamer), 1.85-1.83 (m, 1H, minor rotamer), 1.70 (s, 3H, major rotamer). 1.63 (s, 3H, minor rotamer); ¹³C NMR (125 MHz, Acetone-d₆) δ 169.6, 169.6, 169.1, 167.7, 167.6, 167.3, 167.2, 163.4, 163.3, 155.7, 155.6, 155.3, 155.2, 151.8, 151.7, 149.8, 149.7, 137.5, 137.4, 137.3, 136.2, 136.2, 135.8, 135.6, 135.2, 135.2, 132.8, 132.7, 130.8, 130.7, 130.3, 130.3, 130.3, 130.3, 130.2, 130.1, 130.0, 128.4, 128.4, 128.3, 128.3, 128.0, 128.0, 127.6, 127.5, 125.5, 125.4, 124.1, 120.1, 120.0, 120.0, 119.9, 116.2, 116.0, 105.7, 69.4, 66.1, 66.0, 62.2, 60.6, 59.6, 59.0, 54.8, 54.6, 53.9, 52.5, 51.7, 41.0, 38.3, 13.6, 13.6, 12.1, 10.9, 10.8; HRMS (ESI) m/z calcd for C₅₀H₄₈ClN₈O₅S₂[M+H]⁺, 939.2878; found, 939.2917.

### iv. Synthesis of (2-(2-(8-(2-((1S,4S)-5-(2-((S)-4-(4-Chlorophenyl)-2,3,9-Trimethyl-6H-Thieno[3,2-F][1,2,4]Triazolo[4,3-A][1,4]Diazepin-6-yl)Acetyl)-2,5-Diazabicyclo[2.2.1]Heptan-2-yl)Ethoxy)Naphthalen-2-yl)Thiazol-4-yl)Acetyl)Glycine (5373)

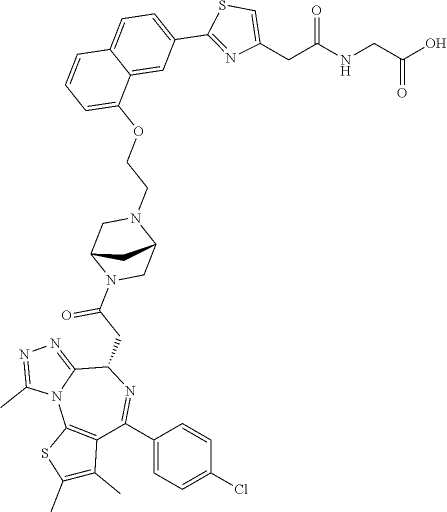 [SMILES: Cc1sc2c(c1C)C(c1ccc(Cl)cc1)=N[C@@H](CC(=O)N1C[C@@H]3C[C@H]1CN3CCOc1cccc3ccc(-c4nc(CC(=O)NCC(=O)O)cs4)cc13)c1nnc(C)n1-2]

Compound 5373 (7 mg, 31%) was prepared following the same procedure as employed for the synthesis of compound 5329 starting from 5372, and it yielded a yellow solid. ¹H NMR (500 MHz, Acetonitrile-d₃, 2:1 ratio of rotamers) δ 8.66 (d, J=1.9 Hz, 1H, minor rotamer), 8.41 (d, J=1.9 Hz, 1H, major rotamer), 8.05 (dd, J=8.6, 2.0 Hz, 1H, minor), 7.92 (td, J=8.9, 3.1 Hz, 1H, both rotamer), 7.84 (d, J=8.5 Hz, 1H, minor rotamer), 7.46 (q, J=4.5, 3.8 Hz, 3H, major rotamer), 7.34 (d, J=7.2 Hz, 4H, 1H major rotamer, 3H minor rotamer), 7.23 (d, J=7.7 Hz, 2H, both rotamer), 7.04-6.89 (m, 3H, both rotamer), 4.84 (s, 1H, major rotamer), 4.69 (s, 1H, minor rotamer), 4.61 (ddd, J=16.4, 9.1, 5.1 Hz, 1H, both rotamer), 4.37 (dt, J=9.3, 4.5 Hz, 1H, major rotamer), 4.28 (td, J=9.5, 8.5, 4.3 Hz, 2H, major rotamer), 4.14 (d, J=10.1 Hz, 1H, minor rotamer), 3.90 (s, 1H, minor rotamer), 3.87-3.83 (m, 1H, minor rotamer), 3.84-3.80 (m, 2H, major rotamer), 3.76-3.71 (m, 7H, 3H major rotamer, 4H minor rotamer), 3.69-3.60 (m, 5H, 4H major rotamer, 3H minor rotamer), 3.43-3.30 (m, 2H, major rotamer), 3.23 (ddd, J=21.6, 10.3, 6.8 Hz, 5H, 3H minor rotamer, 2H major rotamer), 3.16-3.13 (m, 1H, minor rotamer), 2.76 (d, J=9.8 Hz, 1H, minor rotamer), 2.61 (s, 3H, major rotamer), 2.52 (s, 3H, minor rotamer), 2.32 (s, 3H, major rotamer), 2.30 (s, 3H, minor rotamer), 2.03-2.01 (m, 1H, major rotamer), 1.83-1.82 (m, 1H, minor rotamer), 1.55 (s, 3H, minor rotamer), 1.49 (s, 3H, major rotamer). ¹³C NMR (125 MHz, CD₃CN) δ 175.7, 171.3, 169.5, 169.3, 169.3, 169.2, 165.6, 165.4, 156.3, 155.9, 155.3, 151.9, 151.7, 151.4, 151.1, 137.4, 137.4, 136.7, 136.1, 135.7, 135.7, 132.4, 132.2, 132.1, 131.1, 131.0, 130.8, 130.8, 130.4, 130.4, 130.2, 129.3, 129.1, 129.0, 128.7, 128.5, 128.3, 125.5, 124.6, 124.2, 121.1, 120.8, 120.7, 117.7, 106.7, 106.3, 69.1, 62.7, 62.5, 62.4, 60.6, 60.1, 56.7, 54.6, 54.6, 53.4, 52.6, 50.6, 49.3, 44.1, 38.6, 38.5, 37.0, 36.5, 36.2, 35.6, 14.0, 13.9, 12.7, 11.5; HRMS (ESI) m/z calcd for C₄₃H₄₂ClN₈O₅S₂[M+H]⁺, 849.2408; found, 849.2414.

### h. Synthesis of Dasatanib PROTACS 5343 and 5350

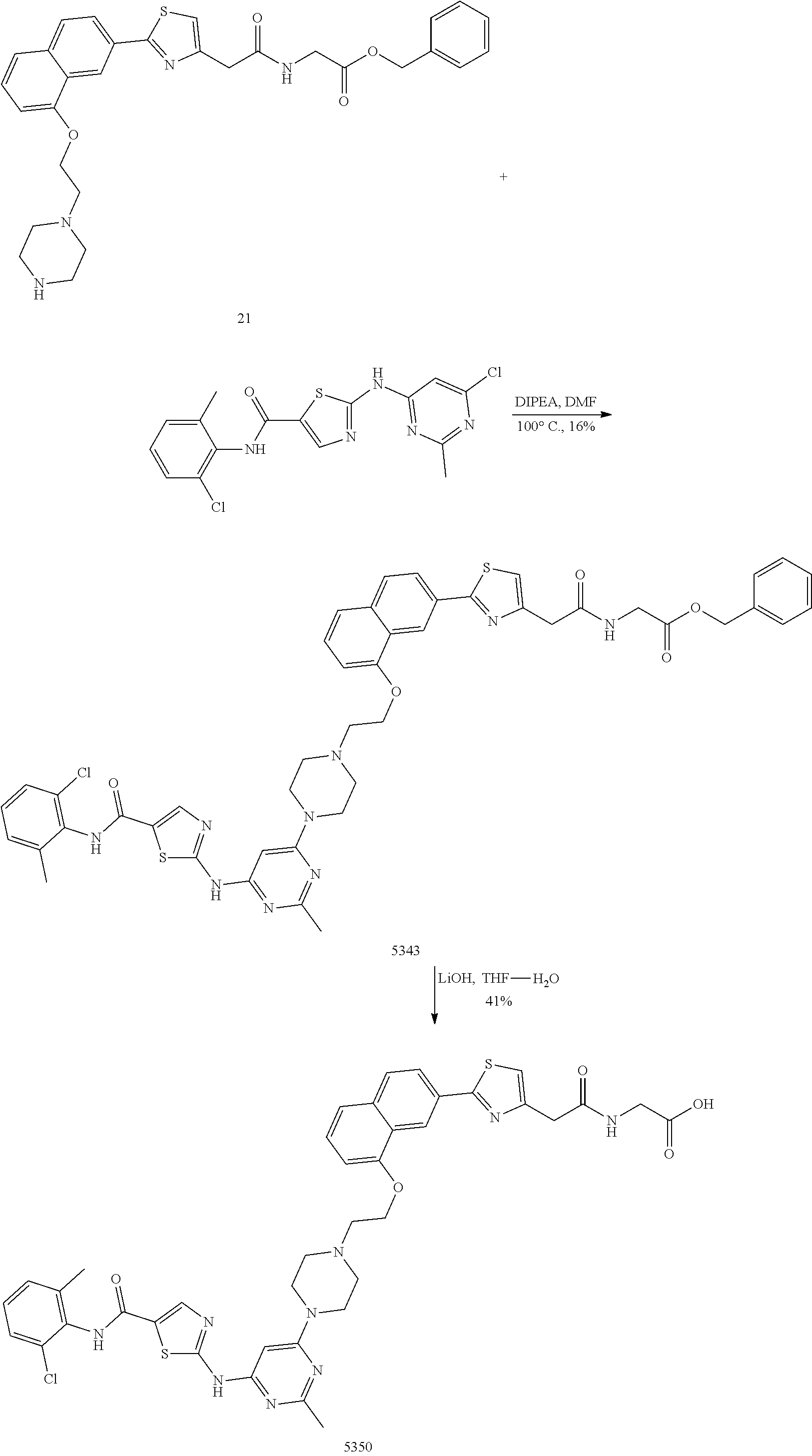

### i. Synthesis of Benzyl (2-(2-(8-(2-(4-(6-((5-((2-Chloro-6-Methylphenyl)Carbamoyl)Thiazol-2-yl)Amino)-2-Methylpyrimidin-4-yl)Piperazin-1-yl)Ethoxy)Naphthalen-2-yl)Thiazol-4-yl)Acetyl)Glycinate (5343)

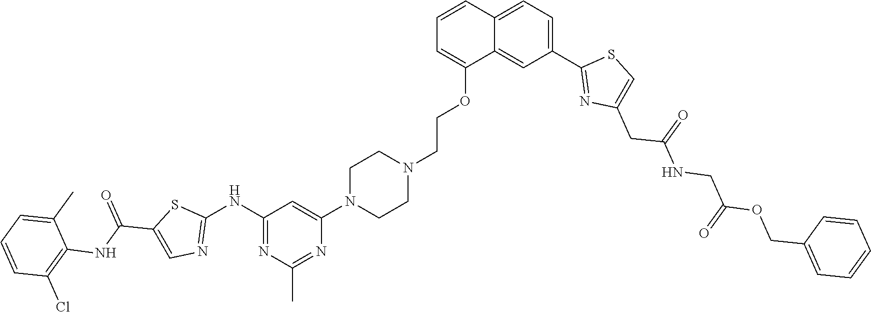 [SMILES: Cc1nc(Nc2ncc(C(=O)Nc3c(C)cccc3Cl)s2)cc(N2CCN(CCOc3cccc4ccc(-c5nc(CC(=O)NCC(=O)OCc6ccccc6)cs5)cc34)CC2)n1]

A solution of benzyl (2-(2-(8-(2-(piperazin-1-yl)ethoxy)naphthalen-2-yl)thiazol-4-yl)acetyl)glycinate dihydrochloride (25 mg, 0.04 mmol) and 2-((6-chloro-2-methylpyrimidin-4-yl)amino)-N-(2-chloro-6-methylphenyl)thiazole-5-carboxamide (16 mg, 0.04 mmol) in dimethylformamide (1.0 mL) was stirred. At room temperature, N,N-diisopropylethylamine (0.022 mL, 0.12 mmol) was introduced to the mixture. The reaction mixture was then heated to 100° C. and allowed to react overnight. Afterward, the reaction was quenched with water, and the resulting compound was extracted with EtOAc (2×10 mL), dried over Na₂SO₄, and concentrated. The crude product was subjected to purification via silica-gel flash column chromatography, yielding 5343 (12 mg, 33%) as a yellow solid. ¹H NMR (500 MHz, CDCl₃) δ 8.83 (d, J=1.8 Hz, 1H), 8.10-7.92 (m, 2H), 7.85 (d, J=8.6 Hz, 1H), 7.51 (t, J=5.3 Hz, 1H), 7.43 (d, J=6.4 Hz, 3H), 7.33-7.23 (m, 4H), 7.16 (d, J=8.4 Hz, 3H), 6.87 (dd, J=6.4, 2.2 Hz, 1H), 5.83 (s, 1H), 5.12 (s, 2H), 4.37 (t, J=5.1 Hz, 2H), 4.20-4.08 (m, 3H), 3.89 (s, 2H), 3.68 (t, J=4.8 Hz, 4H), 3.03 (t, J=5.2 Hz, 2H), 2.95-2.83 (m, 3H), 2.49 (s, 3H), 2.34 (s, 3H); ¹³C NMR (125 MHz, CDCl₃) δ 170.0, 169.6, 169.5, 162.9, 162.6, 156.6, 155.1, 150.4, 135.3, 135.1, 130.0, 129.4, 128.6, 128.5, 128.3, 127.4, 125.5, 124.5, 120.8, 120.4, 116.5, 105.8, 67.3, 67.2, 65.9, 60.4, 57.2, 53.6, 44.1, 41.7, 39.1, 39.1, 25.7, 19.1, 15.3; HRMS (ESI) m/z calcd for C₄₆H₄₅ClN₉O₅S₂ [M+H]⁺, 902.2674; found, 902.2687.

### ii. Synthesis of (2-(2-(8-(2-(4-(6-((5-((2-Chloro-6-Methylphenyl)Carbamoyl)Thiazol-2-yl)Amino)-2-Methylpyrimidin-4-yl)Piperazin-1-yl)Ethoxy)Naphthalen-2-yl)Thiazol-4-yl)Acetyl)Glycine (5350)

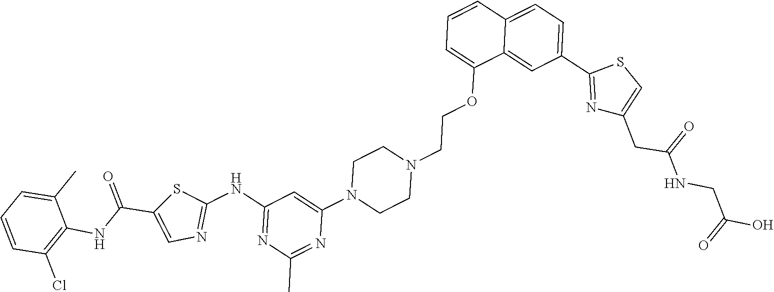 [SMILES: Cc1nc(Nc2ncc(C(=O)Nc3c(C)cccc3Cl)s2)cc(N2CCN(CCOc3cccc4ccc(-c5nc(CC(=O)NCC(=O)O)cs5)cc34)CC2)n1]

In a solution containing benzyl (2-(2-(8-(2-(4-(6-((5-((2-chloro-6-methylphenyl)carbamoyl)thiazol-2-yl)amino)-2-methylpyrimidin-4-yl)piperazin-1-yl)ethoxy)naphthalen-2-yl)thiazol-4-yl)acetyl)glycinate (30 mg, 0.033 mmol) dissolved in a 2:1 mixture of THF-water (1.5 mL), LiOH H₂O (2.8 mg, 0.066 mmol) was added at room temperature and stirred for 1 h. After neutralization with 1N HCl, the resulting compound precipitated and was washed with water, yielding 5350 (11 mg, 41%) as a white solid. ¹H NMR (500 MHz, Acetone-d₆) δ 8.82 (d, J=3.7 Hz, 1H), 8.22-8.09 (m, 2H), 7.95 (dd, J=8.0, 4.6 Hz, 1H), 7.59-7.39 (m, 3H), 7.35-7.17 (m, 4H), 7.03 (d, J=7.1 Hz, 1H), 6.21 (d, J=4.1 Hz, 1H), 4.43 (t, J=5.2 Hz, 2H), 3.85 (s, 2H), 3.71 (d, J=3.5 Hz, 2H), 3.68 (t, J=4.7 Hz, 4H), 3.06 (t, J=5.2 Hz, 2H), 2.79 (t, J=4.9 Hz, 4H), 2.40 (d, J=3.9 Hz, 3H), 2.27 (s, 3H); ¹³C NMR (125 MHz, Acetone-d₆) δ 174.1, 169.8, 168.1, 165.8, 163.0, 159.4, 157.3, 154.9, 151.8, 140.5, 139.2, 135.2, 133.5, 132.7, 130.2, 129.0, 128.7, 127.7, 127.0, 125.4, 124.3, 120.1, 120.1, 117.0, 106.2, 93.5, 83.1, 66.4, 56.7, 52.9, 43.9, 43.8, 38.4, 24.9, 18.0; HRMS (ESI) m/z calcd for C₃₉H₃₉ClN₉O₅S₂ [M+H]⁺, 812.2204; found, 812.2188.

### i. Synthesis of Dasatanib PROTACS 5374 and 5375

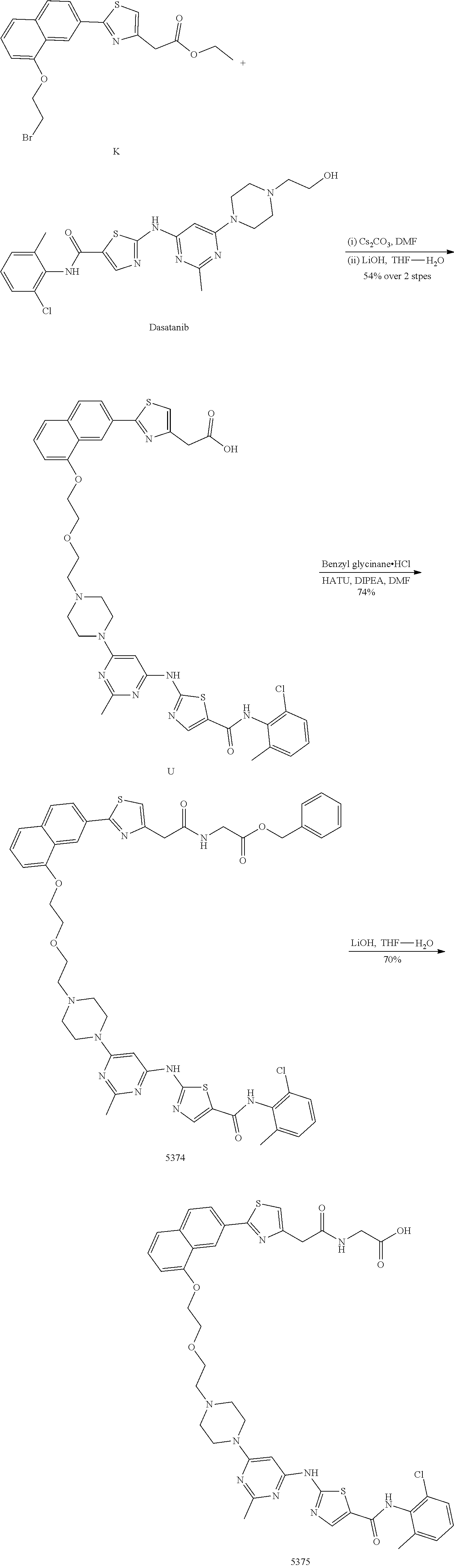

### i. 2-(2-(8-(2-(2-(4-(6-((5-((2-Chloro-6-Methylphenyl)Carbamoyl)Thiazol-2-yl)Amino)-2-Methylpyrimidin-4-yl)Piperazin-1-yl)Ethoxy)Ethoxy)Naphthalen-2-yl)Thiazol-4-yl)Acetic Acid (U)

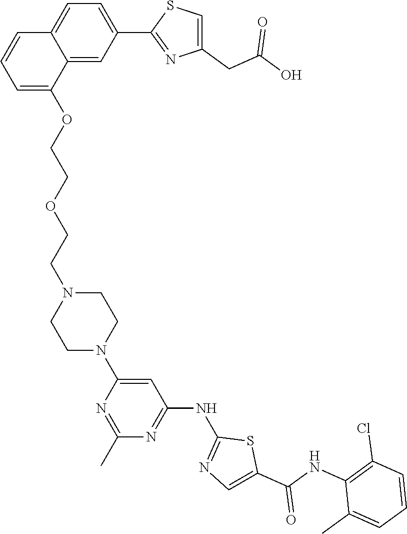 [SMILES: Cc1nc(Nc2ncc(C(=O)Nc3c(C)cccc3Cl)s2)cc(N2CCN(CCOCCOc3cccc4ccc(-c5nc(CC(=O)O)cs5)cc34)CC2)n1]

A solution of ethyl 2-(2-(8-(2-bromoethoxy)naphthalen-2-yl)thiazol-4-yl)acetate (50 mg, 0.12 mmol) and N-(2-chloro-6-methylphenyl)-2-((6-(4-(2-hydroxyethyl)piperazin-1-yl)-2-methylpyrimidin-4-yl)amino)thiazole-5-carboxamide (58 mg, 0.12 mmol) in DMF (1.0 mL) was treated with Cs₂CO₃ (16 mg, 0.12 mmoL). The reaction mixture was then heated at 60° C. for 3 h. the reaction was quenched with water, and the resulting mixture was extracted with EtOAc (2×20 mL), dried over Na₂SO₄, and concentrated. The crude product was dissolved in a 2:1 mixture of THF-H₂O (1.5 mL). LiOH·H₂O (3.5 mg, 0.085 mmoL) was added to the reaction mixture and stirred for 1 h. The reaction mixture was evaporated, and the resulting residue was subjected to purification via reverse silica-gel flash column chromatography (Eluents: 10% CH₃CN in H₂O), yielding U (60 mg, 73%) as a colorless oil. ¹H NMR (500 MHz, DMSO-d₆) δ 10.91 (s, 1H), 8.57 (d, J=8.7 Hz, 2H), 8.03-7.86 (m, 2H), 7.56-7.40 (m, 2H), 7.36 (dd, J=7.7, 1.8 Hz, 1H), 7.30-7.19 (m, 3H), 7.07 (d, J=7.6 Hz, 1H), 6.41 (s, 1H), 5.01 (t, J=5.0 Hz, 2H), 4.60 (t, J=5.0 Hz, 2H), 3.53 (t, J=5.5 Hz, 5H), 3.50-3.46 (m, 3H), 2.42 (s, 3H), 2.36-2.34 (m, 6H), 2.23 (s, 3H); 13C NMR (125 MHz, DMSO-d₆) δ 172.7, 165.7, 164.7, 163.5, 163.2, 160.8, 158.2, 156.7, 154.9, 140.7, 139.3, 134.9, 134.6, 133.1, 130.8, 129.3, 128.9, 128.3, 128.1, 127.7, 127.4, 125.2, 124.9, 120.6, 119.4, 115.2, 106.5, 82.9, 66.2, 60.6, 58.9, 53.2, 45.9, 44.1, 42.2, 25.5, 18.9; HRMS (ESI) m/z calcd for C₃₉H₄₀ClN₈O₅S₂[M+H]⁺, 799.2252; found, 799.2245.

### ii. Synthesis of Benzyl (2-(2-(8-(2-(2-(4-(6-((5-((2-Chloro-6-Methylphenyl)Carbamoyl)Thiazol-2-yl)Amino)-2-Methylpyrimidin-4-yl)Piperazin-1-yl)Ethoxy)Ethoxy)Naphthalen-2-yl)Thiazol-4-yl)Acetyl)Glycinate (5374)

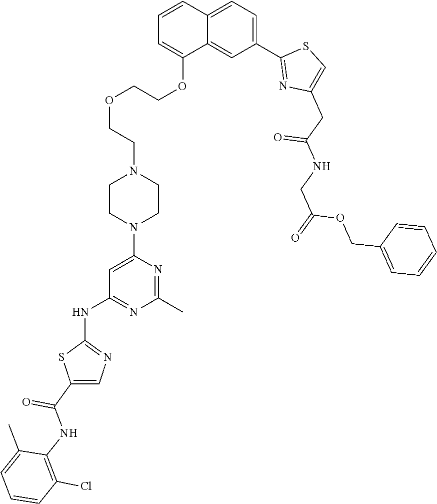 [SMILES: Cc1nc(Nc2ncc(C(=O)Nc3c(C)cccc3Cl)s2)cc(N2CCN(CCOCCOc3cccc4ccc(-c5nc(CC(=O)NCC(=O)OCc6ccccc6)cs5)cc34)CC2)n1]

Compound 5374 (35 mg, 74%) was prepared following the same procedure as employed for the synthesis of compound 5407, resulting in the formation of a white solid. ¹H NMR (500 MHz, CDCl₃) δ 8.61 (s, 1H), 8.09 (dd, J=8.8, 1.9 Hz, 1H), 7.85 (d, J=8.6 Hz, 1H), 7.60 (t, J=5.1 Hz, 1H), 7.54-7.35 (m, 5H), 7.29 (q, J=6.1, 4.7 Hz, 4H), 7.23-7.13 (m, 3H), 6.93 (d, J=7.3 Hz, 1H), 6.18 (s, 1H), 5.17 (d, J=10.2 Hz, 1H), 5.11 (s, 2H), 4.84 (d, J=5.4 Hz, 2H), 4.73 (t, J=5.4 Hz, 2H), 4.11 (t, J=6.6 Hz, 2H), 3.86 (s, 2H), 3.56 (t, J=5.3 Hz, 2H), 3.50-3.35 (m, 4H), 3.03-2.99 (m, 2H), 2.53 (s, 3H), 2.44 (t, J=5.3 Hz, 2H), 2.34 (s, 3H), 2.31 (t, J=4.9 Hz, 2H); ¹³C NMR (125 MHz, CDCl₃) δ 169.6, 169.5, 169.0, 163.4, 158.7, 154.5, 150.7, 135.1, 130.3, 129.4, 128.9, 128.8, 128.6, 128.6, 128.5, 128.3, 127.3, 124.7, 120.7, 120.6, 116.2, 105.7, 82.4, 68.2, 67.1, 65.6, 60.4, 59.2, 57.7, 52.2, 46.9, 45.6, 43.8, 41.7, 38.9, 25.3, 19.2; HRMS (ESI) m/z calcd for C₄₈H₄₉ClN₉O₆S₂[M+H], 946.2936; found, 946.2947.

### iii. Synthesis of (2-(2-(8-(2-(2-(4-(6-((5-((2-Chloro-6-Methylphenyl)Carbamoyl)Thiazol-2-yl)Amino)-2-Methylpyrimidin-4-yl)Piperazin-1-yl)Ethoxy)Ethoxy)Naphthalen-2-yl)Thiazol-4-yl)Acetyl)Glycine (5375)

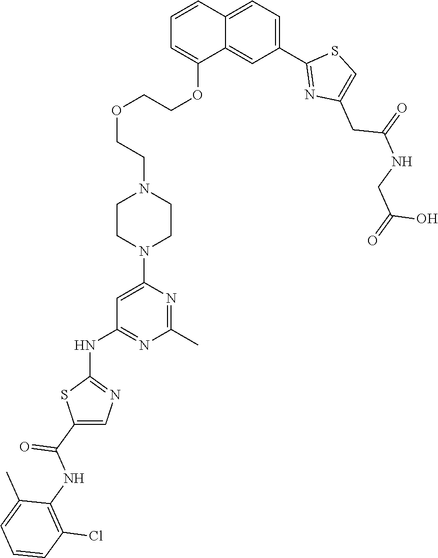 [SMILES: Cc1nc(Nc2ncc(C(=O)Nc3c(C)cccc3Cl)s2)cc(N2CCN(CCOCCOc3cccc4ccc(-c5nc(CC(=O)NCC(=O)O)cs5)cc34)CC2)n1]

Compound 5375 (19 mg, 70%) was prepared following the same procedure as employed for the synthesis of compound 5329, resulting in the formation of a yellow solid. ¹H NMR (600 MHz, CD₃CN) δ 8.24 (d, J=1.8 Hz, 1H), 8.18 (s, 1H), 8.02-7.97 (m, 1H), 7.88 (d, J=8.6 Hz, 1H), 7.49-7.41 (m, 3H), 7.38 (s, 1H), 7.33 (dd, J=7.2, 2.3 Hz, 1H), 7.28-7.17 (m, 3H), 7.02-6.97 (m, 1H), 6.40 (s, 1H), 4.82 (t, J=5.0 Hz, 2H), 4.73 (t, J=5.0 Hz, 2H), 3.87-3.82 (m, 2H), 3.80 (s, 2H), 3.77-3.67 (m, 2H), 3.30-3.17 (m, 1H), 3.12-3.01 (m, 6H), 3.02-2.88 (m, 1H), 2.46 (s, 3H), 2.26-2.24 (m, 2H), 2.23 (s, 3H); ¹³C NMR (150 MHz, CD₃CN) δ 173.2, 171.6, 169.1, 166.0, 164.0, 163.3, 161.9, 159.6, 154.9, 151.7, 140.7, 139.8, 135.7, 133.3, 130.6, 129.7, 129.3, 129.2, 128.4, 127.7, 127.4, 125.5, 124.7, 120.9, 120.9, 106.7, 84.2, 65.9, 58.9, 55.6, 55.5, 51.9, 47.0, 42.2, 41.7, 41.3, 38.5, 24.8, 18.3; HRMS (ESI) m/z calcd for C₄₁H₄₃ClN₉O₆S₂[M+H]⁺, 856.2466; found, 856.2480.

### j. Synthesis of DCN1 PROTACS 5376 and 5377

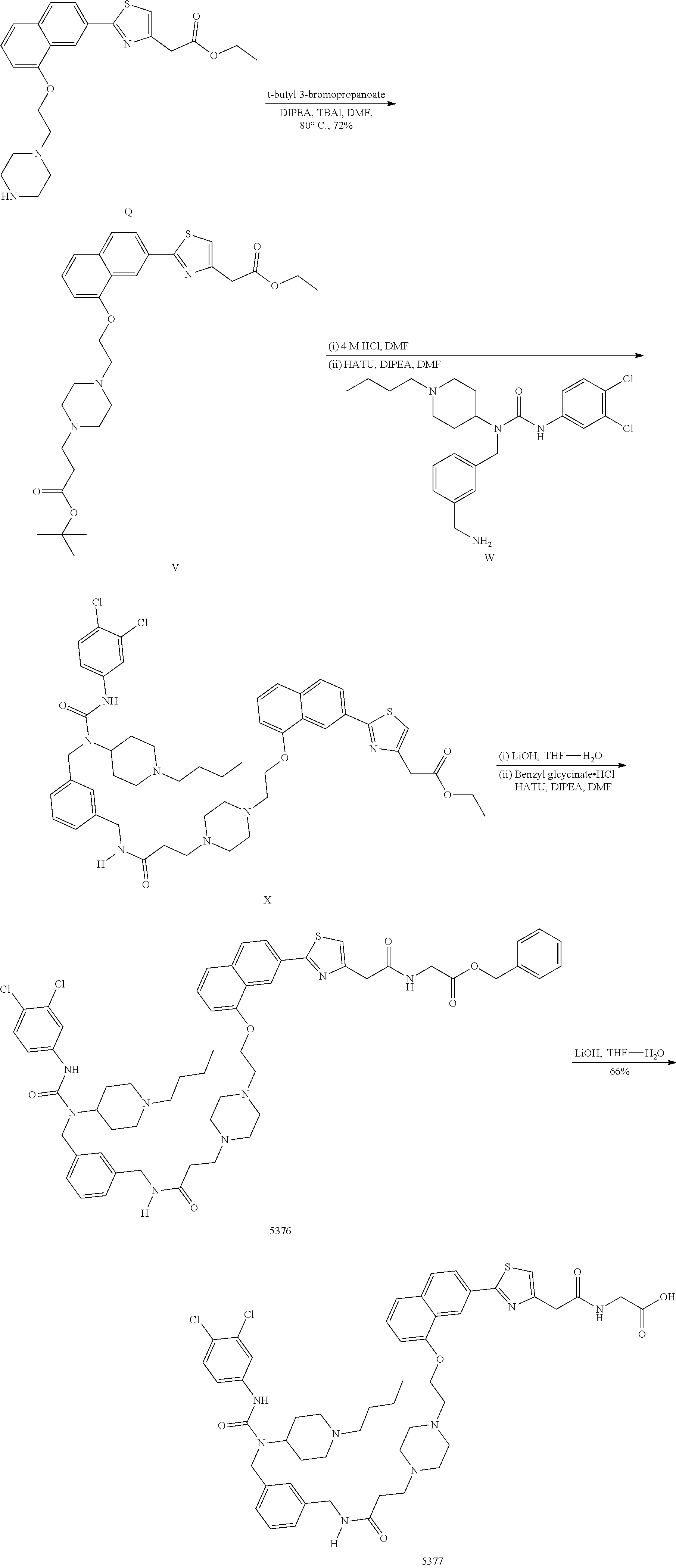

### i. Synthesis of Tert-Butyl 3-(4-(2-((7-(4-(2-Ethoxy-2-Oxoethyl)Thiazol-2-yl)Naphthalen-1-yl)Oxy)Ethyl)Piperazin-1-yl)Propanoate (V)

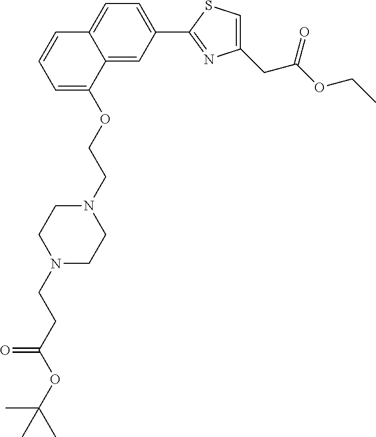 [SMILES: CCOC(=O)Cc1csc(-c2ccc3cccc(OCCN4CCN(CCC(=O)OC(C)(C)C)CC4)c3c2)n1]

In a stirred solution, ethyl 2-(2-(8-(2-(piperazin-1-yl)ethoxy)naphthalen-2-yl)thiazol-4-yl)acetate dihydrochloride (50 mg, 0.10 mmol), tert-butyl 3-bromopropanoate (23 mg, 0.11 mmol), and tetrabutylammonium iodide (3.7 mg, 0.01 mmol) were combined in DMF (1.0 mL). Diisopropylethylamine (0.05 mL, 0.30 mmol) was added to the mixture at room temperature, followed by overnight heating at 80° C. The reaction mixture was quenched with water (10 mL) and the product was extracted with ethyl acetate (2×10 mL). The organic layer was dried over Na₂SO₄, and concentrated. The residue was purified on silica-gel flash column chromatography (Eluents: 10% MeOH in EtOAc) to yield V (40 mg, 72%) as a colorless oil. ¹H NMR (500 MHz, CDCl₃) δ 8.74 (d, J=1.9 Hz, 1H), 8.10 (dd, J=8.6, 1.9 Hz, 1H), 7.83 (d, J=8.6 Hz, 1H), 7.46-7.33 (m, 2H), 7.22 (s, 1H), 6.83 (dd, J=6.9, 1.7 Hz, 1H), 4.32 (t, J=5.8 Hz, 2H), 4.26 (s, 2H), 3.92 (s, 2H), 3.02 (t, J=5.8 Hz, 2H), 2.82-2.61 (m, 6H), 2.56 (s, 4H), 2.42 (t, J=7.5 Hz, 2H), 1.43 (s, 9H), 1.31 (t, J=7.1 Hz, 3H); ¹³C NMR (125 MHz, CDCl₃) δ 171.7, 170.4, 168.1, 154.8, 149.8, 135.0, 130.3, 128.1, 127.0, 125.4, 124.3, 120.5, 120.1, 115.9, 105.4, 80.3, 66.6, 60.9, 57.0, 53.6, 53.6, 52.8, 37.1, 33.4, 28.0, 14.1; HRMS (ESI) m/z calcd for C₃₀H₄₀N₃O₅S[M+H]⁺, 554.2689; found, 554.2703.

### ii. Synthesis of Ethyl 2-(2-(8-(2-(4-(3-((3-((1-(1-Butylpiperidin-4-yl)-3-(3,4-Dichlorophenyl)Ureido)Methyl)Benzyl)Amino)-3-Oxopropyl)Piperazin-1-yl)Ethoxy)Naphthalen-2-yl)Thiazol-4-yl)Acetate (X)

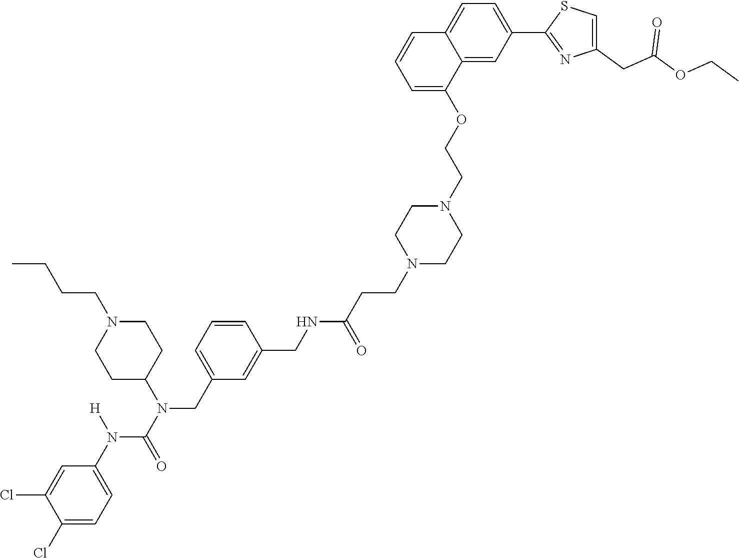 [SMILES: CCCCN1CCC(N(Cc2cccc(CNC(=O)CCN3CCN(CCOc4cccc5ccc(-c6nc(CC(=O)OCC)cs6)cc45)CC3)c2)C(=O)Nc2ccc(Cl)c(Cl)c2)CC1]

A mixture containing 40 mg (0.072 mmol) of tert-butyl 3-(4-(2-((7-(4-(2-ethoxy-2-oxoethyl)thiazol-2-yl)naphthalen-1-yl)oxy)ethyl)piperazin-1-yl)propanoate in 1.0 mL of CH₂Cl₂ was subjected to treatment with 1.0 mL of 4M HCl in dioxane at room temperature. The resulting mixture was stirred for 1 hour, leading to the evaporation of the solvent and the formation of pure amine. Subsequently, the obtained amine (35 mg, 0.070 mmol) was mixed with 1-(3-(aminomethyl)benzyl)-1-(1-butylpiperidin-4-yl)-3-(3,4-dichlorophenyl)urea (33 mg, 0.070 mmol) and HATU (32 mg, 0.084 mmol) in 1.0 mL of DMF. To this mixture, N,N-diisopropylethylamine (0.038 mL, 0.21 mmoL) was added, and the reaction was stirred for 15 mins. Quenched with water, and the resulting compound was extracted with ethyl acetate (2×10 mL). The organic phase was dried over Na₂SO₄ and concentrated. This crude product was subsequently purified through silica-gel flash column chromatography (Eluents: 30% MeOH in EtOAc), resulting in the isolation of compound X (30 mg, 45%) as a colorless oil. ¹H NMR (500 MHz, Acetone-d₆) δ 8.79 (d, J=2.0 Hz, 1H), 8.06 (dt, J=9.0, 2.6 Hz, 1H), 7.91 (dd, J=8.6, 2.5 Hz, 1H), 7.83 (d, J=2.5 Hz, 1H), 7.51-7.42 (m, 4H), 7.39 (dd, J=8.8, 2.5 Hz, 1H), 7.32 (d, J=8.9 Hz, 1H), 7.26 (s, 1H), 7.22-7.08 (m, 3H), 7.01 (dd, J=6.9, 1.8 Hz, 1H), 4.63 (s, 2H), 4.36-4.28 (m, 4H), 4.21-4.07 (m, 3H), 3.89 (d, J=4.1 Hz, 3H), 3.33-3.20 (m, 2H), 2.96 (t, J=5.6 Hz, 3H), 2.79-2.56 (m, 13H), 2.44 (t, J=7.1 Hz, 2H), 2.06-2.00 (m, 3H), 1.81-1.68 (m, 2H), 1.55 (ddd, J=12.1, 9.9, 6.4 Hz, 2H), 1.27-1.19 (m, 5H), 0.83 (t, J=7.3 Hz, 3H); ¹³C NMR (125 MHz, Acetone-d₆) δ 172.4, 170.9, 170.4, 170.3, 167.7, 155.4, 154.9, 150.5, 140.5, 139.6, 139.4, 135.2, 131.2, 130.2, 130.0, 128.6, 128.5, 127.8, 126.0, 125.6, 125.3, 125.0, 124.2, 124.1, 121.4, 120.1, 120.1, 120.0, 120.0, 119.9, 116.9, 106.2, 66.4, 60.7, 60.7, 60.0, 56.9, 56.5, 53.8, 53.0, 52.9, 52.3, 52.2, 45.4, 42.5, 36.7, 32.7, 27.1, 20.0, 13.7, 13.2; HRMS (ESI) m/z calcd for C₅₀H₆₂Cl₂N₇O₅S[M+H]f, 942.3910; found, 942.3911.

### iii. Synthesis of Benzyl (2-(2-(8-(2-(4-(3-((3-((1-(1-Butylpiperidin-4-yl)-3-(3,4-Dichlorophenyl)Ureido)Methyl)Benzyl)Amino)-3-Oxopropyl)Piperazin-1-yl)Ethoxy)Naphthalen-2-yl)Thiazol-4-yl)Acetyl)Glycinate (5376)

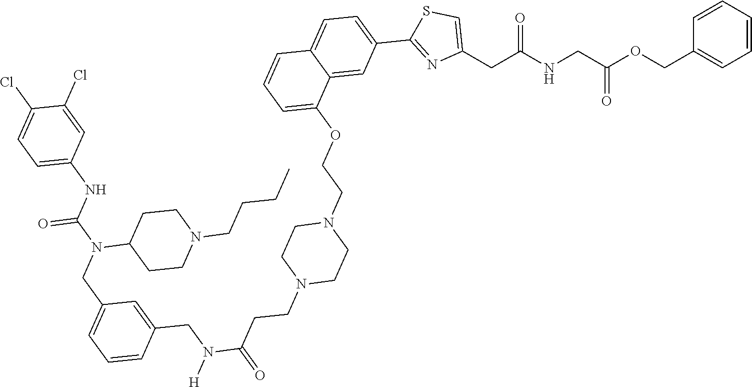 [SMILES: CCCCN1CCC(N(Cc2cccc(CNC(=O)CCN3CCN(CCOc4cccc5ccc(-c6nc(CC(=O)NCC(=O)OCc7ccccc7)cs6)cc45)CC3)c2)C(=O)Nc2ccc(Cl)c(Cl)c2)CC1]

Compound 5376 (18 mg, 52%) was prepared following the same procedure as employed for the synthesis of compound 5341 starting from X, and it yielded a white solid ¹H NMR (500 MHz, CDCl₃) δ 8.75 (s, 1H), 8.63 (d, J=6.1 Hz, 1H), 8.13 (d, J=8.5 Hz, 1H), 7.86 (d, J=8.7 Hz, 1H), 7.60 (d, J=5.5 Hz, 1H), 7.49-7.39 (m, 3H), 7.30 (t, J=6.0 Hz, 5H), 7.24-7.16 (m, 2H), 7.17-7.10 (m, 2H), 6.97 (dd, J=8.8, 2.4 Hz, 1H), 6.84 (d, J=7.0 Hz, 1H), 6.42 (s, 1H), 5.15 (d, J=2.8 Hz, 2H), 4.45 (s, 2H), 4.41 (d, J=5.6 Hz, 2H), 4.36-4.27 (m, 3H), 4.10 (dd, J=19.1, 6.1 Hz, 2H), 3.80 (s, 2H), 3.05-2.90 (m, 4H), 2.79-2.49 (m, 10H), 2.41 (t, J=6.2 Hz, 2H), 2.29 (t, J=7.9 Hz, 2H), 2.05 (d, J=4.7 Hz, 2H), 1.79-1.64 (m, 4H), 1.44-1.39 (m, 2H), 1.29-1.24 (m, 2H), 0.88 (t, J=7.3, 3H); ¹³C NMR (125 MHz, CDCl₃) δ 172.4, 169.6, 169.5, 169.2, 155.3, 154.9, 150.5, 140.0, 138.7, 138.2, 135.3, 135.2, 132.4, 130.1, 130.1, 129.5, 128.6, 128.5, 128.3, 127.4, 126.8, 125.5, 125.2, 124.9, 124.4, 121.3, 120.7, 120.3, 119.0, 116.1, 105.7, 67.1, 66.7, 58.2, 57.0, 53.6, 53.0, 52.4, 46.0, 42.9, 41.7, 38.9, 32.1, 29.9, 29.0, 20.8, 14.0; HRMS (ESI) m/z calcd for C₅₇H₆₇Cl₂N₈O₆S[M+H]⁺, 1061.4281; found, 1061.4274.

### iv. Synthesis of (2-(2-(8-(2-(4-(3-((3-((1-(1-Butylpiperidin-4-yl)-3-(3,4-Dichlorophenyl)Ureido)Methyl)Benzyl)Amino)-3-Oxopropyl)Piperazin-1-yl)Ethoxy)Naphthalen-2-yl)Thiazol-4-yl)Acetyl)Glycine (5377)

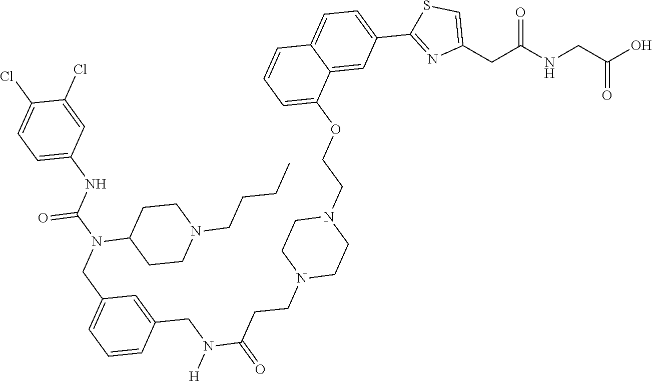 [SMILES: CCCCN1CCC(N(Cc2cccc(CNC(=O)CCN3CCN(CCOc4cccc5ccc(-c6nc(CC(=O)NCC(=O)O)cs6)cc45)CC3)c2)C(=O)Nc2ccc(Cl)c(Cl)c2)CC1]

Compound 5377 (6 mg, 66%) was prepared following the same procedure as employed for the synthesis of compound 5329 starting from 5376, and it yielded a white solid. ¹H NMR (500 MHz, CD₃OD) δ 8.77 (d, J=1.8 Hz, 1H), 7.89 (dd, J=8.6, 1.9 Hz, 1H), 7.79 (d, J=8.6 Hz, 1H), 7.57-7.53 (m, 1H), 7.40-7.32 (m, 2H), 7.29 (s, 1H), 7.21 (d, J=8.9 Hz, 1H), 7.19-7.05 (m, 4H), 6.96 (d, J=7.7 Hz, 1H), 6.87 (dd, J=6.8, 1.8 Hz, 1H), 4.45 (s, 2H), 4.34-4.19 (m, 5H), 3.77-3.64 (m, 4H), 3.36-3.29 (m, 2H), 3.04 (t, J=4.7 Hz, 2H), 2.91-2.56 (m, 14H), 2.39 (q, J=6.9, 6.4 Hz, 2H), 1.97-1.86 (m, 2H), 1.76-1.63 (m, 2H), 1.53-1.47 (m, 2H), 1.29-1.13 (m, 2H), 0.83 (t, J=7.4 Hz, 3H); ¹³C NMR (125 MHz, CD₃OD) δ 172.3, 170.5, 168.7, 156.0, 154.7, 151.2, 139.5, 139.1, 138.9, 135.3, 135.3, 131.5, 130.0, 129.9, 129.8, 129.8, 128.6, 128.4, 127.3, 126.0, 125.4, 125.3, 125.1, 124.8, 124.2, 122.2, 122.2, 120.3, 120.1, 120.0, 119.7, 116.7, 116.6, 105.8, 66.2, 56.1, 53.2, 52.1, 51.7, 51.6, 45.7, 43.5, 42.5, 38.1, 27.2, 26.1, 19.6, 12.5; HRMS (ESI) m/z calcd for C₅₀H₆₁Cl₂N₈O₆S[M+H]⁺, 971.3812; found, 971.3831.

### k. Synthesis of DCN1 PROTACS 5392 and 5393

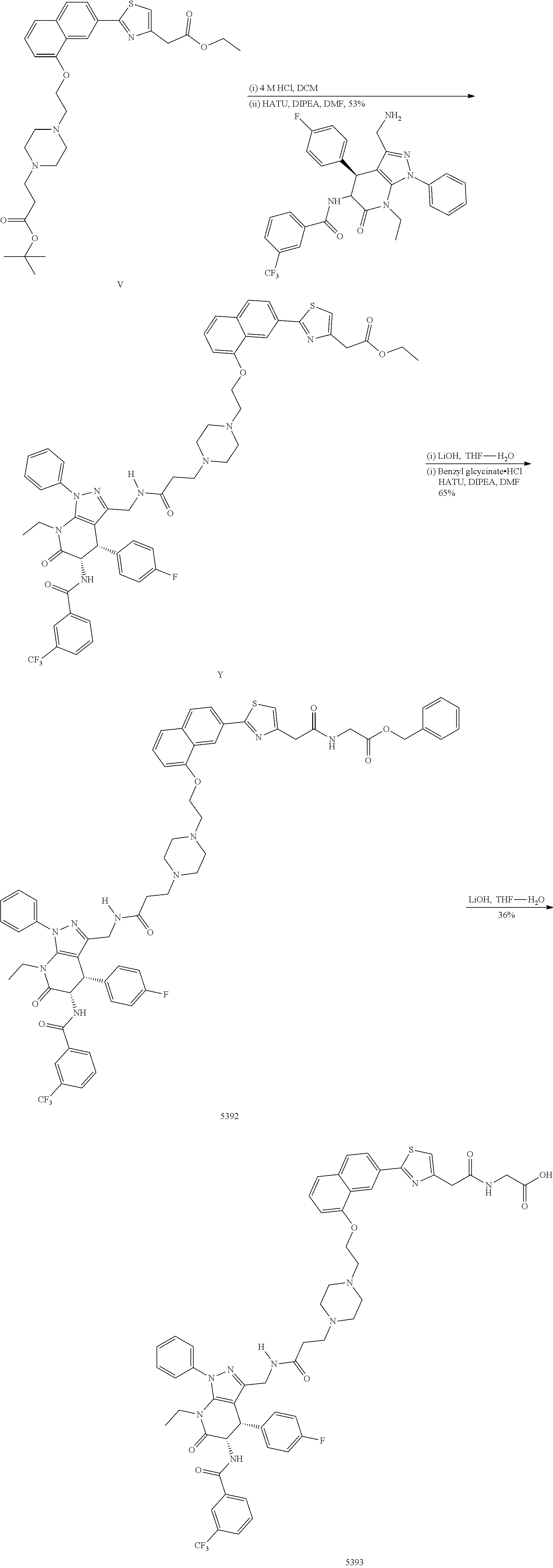

### i. Synthesis of Ethyl 2-(2-(8-(2-(4-(3-((((4S,5S)-7-Ethyl-4-(4-Fluorophenyl)-6-Oxo-1-Phenyl-5-(3-(Trifluoromethyl)Benzamido)-4,5,6,7-Tetrahydro-1H-Pyrazolo[3,4-B]Pyridin-3-yl)Methyl)Amino)-3-Oxopropyl)Piperazin-1-yl)Ethoxy)Naphthalen-2-yl)Thiazol-4-yl)Acetate (Y)

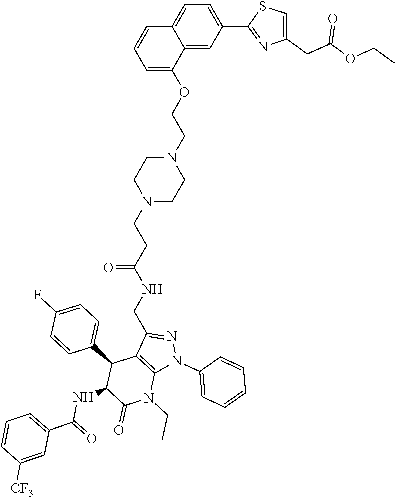 [SMILES: CCOC(=O)Cc1csc(-c2ccc3cccc(OCCN4CCN(CCC(=O)NCc5nn(-c6ccccc6)c6c5[C@H](c5ccc(F)cc5)[C@H](NC(=O)c5cccc(C(F)(F)F)c5)C(=O)N6CC)CC4)c3c2)n1]

Compound Y (55 mg, 53%) was synthesized using the identical procedure employed for the synthesis of compound X starting from V, with N-((4S,5S)-3-(aminomethyl)-7-ethyl-4-(4-fluorophenyl)-6-oxo-1-phenyl-4,5,6,7-tetrahydro-1H-pyrazolo[3,4-b]pyridin-5-yl)-3-(trifluoromethyl)benzamide as the amine, yielding a colorless oil. ¹H NMR (500 MHz, CDCl₃) δ 8.85 (t, J=4.7 Hz, 1H), 8.77 (d, J=1.8 Hz, 1H), 8.11 (dd, J=8.6, 1.8 Hz, 1H), 8.02 (s, 1H), 7.86 (t, J=8.7 Hz, 2H), 7.78 (d, J=7.8 Hz, 1H), 7.60-7.38 (m, 8H), 7.23 (s, 1H), 6.93 (dd, J=6.6, 5.0 Hz, 5H), 6.82 (d, J=7.2 Hz, 1H), 5.25 (t, J=6.6 Hz, 1H), 4.78 (d, J=7.3 Hz, 1H), 4.38 (dd, J=4.8, 3.2 Hz, 2H), 4.30-4.18 (m, 4H), 3.92 (s, 3H), 3.13 (dq, J=13.9, 6.9 Hz, 1H), 2.68-2.23 (m, 14H), 1.27 (dt, J=14.3, 7.1 Hz, 3H), 0.98 (t, J=7.1 Hz, 3H); ¹³C NMR (125 MHz, CDCl₃) δ 172.3, 170.4, 168.2, 167.8, 165.7, 154.9, 150.1, 147.0, 139.6, 139.1, 135.2, 134.6, 130.4, 130.2, 130.0, 129.9, 129.8, 129.5, 129.4, 128.4, 127.2, 125.7, 125.5, 124.6, 124.4, 124.3, 120.5, 120.3, 116.1, 115.7, 115.5, 105.6, 104.8, 66.4, 61.1, 56.7, 55.6, 53.5, 52.3, 39.2, 37.2, 36.6, 36.1, 31.8, 14.2, 12.8; HRMS (ESI) m/z calcd for C₅₅H₅₅F₄N₈O₆S[M+H]⁺, 1031.3901; found, 1031.3875.

### ii. Synthesis of Benzyl (2-(2-(8-(2-(4-(3-((((4S,5S)-7-Ethyl-4-(4-Fluorophenyl)-6-Oxo-1-Phenyl-5-(3-(Trifluoromethyl)Benzamido)-4,5,6,7-Tetrahydro-1H-Pyrazolo[3,4-B]Pyridin-3-yl)Methyl)Amino)-3-Oxopropyl)Piperazin-1-yl)Ethoxy)Naphthalen-2-yl)Thiazol-4-yl)Acetyl)Glycinate (5392)

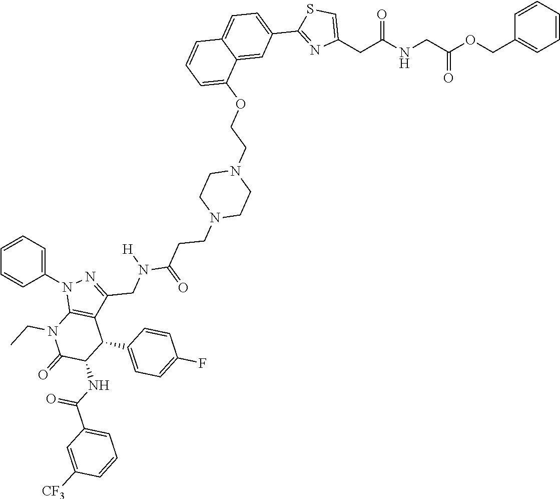

Compound 5392 (30 mg, 64%) was prepared following the same procedure as employed for the synthesis of compound 5341 starting from Y, and it yielded a white solid. ¹H NMR (500 MHz, CDCl₃) δ 8.71 (d, J=2.0 Hz, 1H), 8.67 (s, 1H), 8.13 (ddd, J=13.7, 8.6, 1.9 Hz, 1H), 7.98 (s, 1H), 7.83 (dd, J=8.6, 3.8 Hz, 2H), 7.74 (d, J=7.8 Hz, 1H), 7.55 (dt, J=24.1, 6.6 Hz, 2H), 7.28-7.25 (m, 3H), 7.48-7.37 (m, 7H), 7.22 (s, 1H), 7.12 (d, J=5.6 Hz, 1H), 6.93-6.84 (m, 5H), 6.79 (d, J=7.2 Hz, 1H), 5.20 (t, J=6.6 Hz, 1H), 5.10 (d, J=3.2 Hz, 2H), 4.75 (d, J=7.3 Hz, 1H), 4.32 (t, J=5.4 Hz, 2H), 4.17 (t, J=6.2 Hz, 2H), 4.07 (q, J=7.4 Hz, 3H), 3.89 (dt, J=15.8, 6.7 Hz, 1H), 3.79 (d, J=5.6 Hz, 2H), 3.18-3.04 (m, 1H), 3.03-2.93 (m, 3H), 2.78-2.63 (m, 2H), 2.59-2.36 (m, 6H), 2.31-2.17 (m, 2H), 1.21 (t, J=7.2 Hz, 3H); ¹³C NMR (125 MHz, CDCl₃) δ 172.3, 169.7, 169.5, 169.2, 167.7, 165.7, 154.9, 150.6, 147.0, 139.6, 139.1, 135.3, 135.2, 134.6, 130.2, 130.1, 130.0, 129.9, 129.7, 129.4, 128.6, 128.6, 128.5, 128.5, 128.3, 127.4, 125.7, 125.5, 124.4, 124.4, 120.7, 120.3, 116.1, 115.7, 115.5, 105.7, 104.9, 67.1, 66.5, 60.4, 56.7, 55.6, 53.5, 53.4, 52.3, 41.7, 39.3, 38.9, 36.6, 36.1, 31.8, 21.1, 14.2, 12.8; HRMS (ESI) m/z calcd for C₆₂H₆₀F₄N₉O₇S[M+H]⁺, 1150.4273; found, 1150.4268.

### iii. Synthesis of (2-(2-(8-(2-(4-(3-((((4S,5S)-7-Ethyl-4-(4-Fluorophenyl)-6-Oxo-1-Phenyl-5-(3-(Trifluoromethyl)Benzamido)-4,5,6,7-Tetrahydro-1H-Pyrazolo[3,4-B]Pyridin-3-yl)Methyl)Amino)-3-Oxopropyl)Piperazin-1-yl)Ethoxy)Naphthalen-2-yl)Thiazol-4-yl)Acetyl)Glycine (5293)

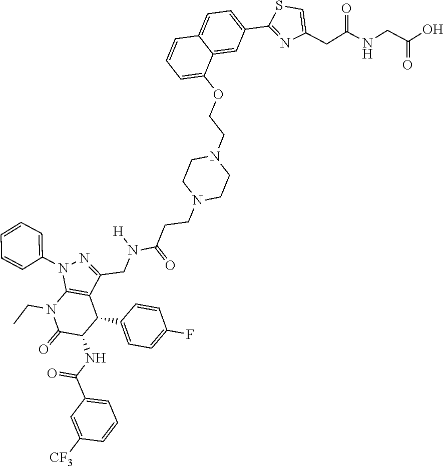

Compound 5393 (5 mg, 36%) was prepared following the same procedure as employed for the synthesis of compound 5329 starting from 5392, and it yielded a yellow solid. ¹H NMR (500 MHz, DMSO-d₆) δ 8.75 (d, J=5.7 Hz, 1H), 8.61 (d, J=7.5 Hz, 1H), 8.51-8.32 (m, 2H), 8.28-8.15 (m, 2H), 8.15-8.01 (m, 3H), 7.97 (d, J=7.5 Hz, 1H), 7.74 (dt, J=23.5, 8.2 Hz, 3H), 7.65-7.55 (m, 6H), 7.45 (q, J=13.1, 10.1 Hz, 1H), 7.16 (ddd, J=20.4, 12.5, 7.6 Hz, 2H), 6.98 (t, J=6.9 Hz, 1H), 5.54 (t, J=7.4 Hz, 1H), 4.59 (d, J=7.5 Hz, 1H), 4.45-4.23 (m, 3H), 4.16 (dd, J=15.4, 5.1 Hz, 1H), 3.97 (dq, J=14.9, 7.1 Hz, 1H), 3.93-3.75 (m, 5H), 3.27-3.16 (m, 2H), 3.15-2.85 (m, 2H), 2.80-2.4 (m, 4H), 2.45-2.026 (m, 4H), 2.10-1.79 (m, 2H), 0.96 (t, J=7.2 Hz, 3H); ¹³C NMR (125 MHz, DMSO) δ 171.9, 169.5, 167.8, 167.1, 165.8, 161.1, 154.8, 152.3, 147.6, 140.3, 139.5, 135.1, 135.1, 132.3, 130.7, 130.6, 130.4, 130.1, 130.0, 129.9, 129.4, 129.2, 128.2, 126.2, 126.1, 125.2, 124.6, 120.5, 119.6, 117.3, 115.6, 115.5, 106.9, 105.2, 56.3, 55.8, 52.2, 41.6, 38.5, 37.2, 36.3, 35.5, 31.2, 13.1; HRMS (ESI) m/z calcd for C₅₅H₅₄F₄N₉O₇S[M+H]⁺, 1060.3803; found, 1060.3813.

### l. Synthesis of Indole-Based KLHDC2 Ligands

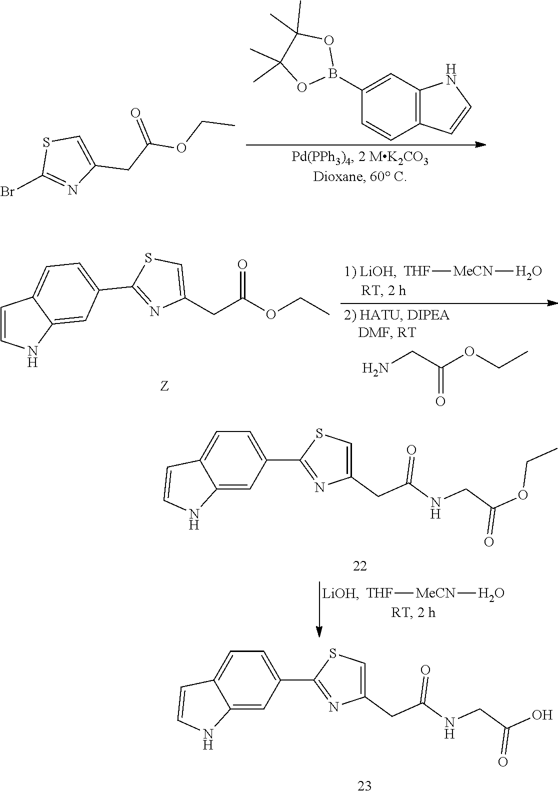

### i. Synthesis of Ethyl 2-(2-(1H-Indol-6-yl)Thiazol-4-yl)Acetate (Z)

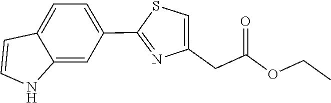 [SMILES: CCOC(=O)Cc1csc(-c2ccc3cc[nH]c3c2)n1]

¹H NMR (500 MHz, Chloroform-d) δ 8.69-8.63 (m, 1H), 8.19 (s, 1H), 7.65-7.50 (m, 2H), 7.25 (t, J=2.7 Hz, 1H), 7.13 (s, 1H), 6.49 (t, J=2.5 Hz, 1H), 4.15 (q, J=6.8 Hz, 2H), 3.91 (s, 2H), 1.23 (t, J=7.2 Hz, 3H). ESI-MS (M+1): 287.93.

### ii. Synthesis of Ethyl (2-(2-(1H-Indol-6-yl)Thiazol-4-yl)Acetyl)Glycinate (22)

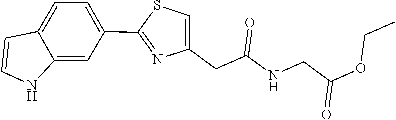 [SMILES: CCOC(=O)CNC(=O)Cc1csc(-c2ccc3cc[nH]c3c2)n1]

1H NMR (400 MHz, Chloroform-d) δ 8.93 (s, 1H), 8.07 (dt, J=1.7, 0.8 Hz, 1H), 7.82 (t, J=5.3 Hz, 1H), 7.68-7.49 (m, 2H), 7.31 (dd, J=3.2, 2.4 Hz, 1H), 7.04 (d, J=0.8 Hz, 1H), 6.55 (ddd, J=3.1, 2.0, 0.9 Hz, 1H), 4.17 (q, J=7.2 Hz, 2H), 4.08 (d, J=5.3 Hz, 2H), 3.88-3.82 (m, 2H), 1.23 (t, J=7.1 Hz, 3H). ESI-MS (M+1): 345.22

### iii. Synthesis of (2-(2-(1H-Indol-6-yl)Thiazol-4-yl)Acetyl)Glycine (23)

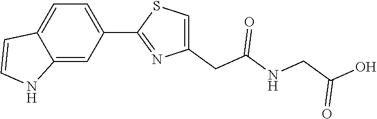 [SMILES: O=C(O)CNC(=O)Cc1csc(-c2ccc3cc[nH]c3c2)n1]

1H NMR (400 MHz, DMSO-d6) δ 11.32 (s, 1H), 8.39 (t, J=5.9 Hz, 1H), 7.99 (dt, J=1.6, 0.8 Hz, 1H), 7.64-7.54 (m, 2H), 7.51-7.47 (m, 1H), 7.38 (s, 1H), 6.50 (ddd, J=3.0, 2.0, 0.9 Hz, 1H), 3.82 (d, J=5.8 Hz, 2H), 3.72 (d, J=0.9 Hz, 2H). ESI-MS (M+1): 316.34.

### m. Synthesis of (2-(2-(1-(3-(Piperazin-1-yl)Propyl)-1H-Indol-6-yl)Thiazol-4-yl)Acetyl)Glycine (24)

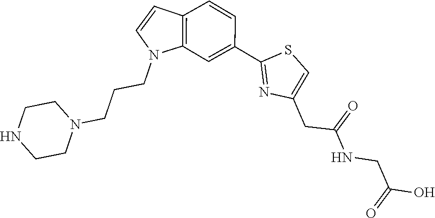 [SMILES: O=C(O)CNC(=O)Cc1csc(-c2ccc3ccn(CCCN4CCNCC4)c3c2)n1]

A mixture of tert-butyl 4-(3-(6-(4-(2-((2-(benzyloxy)-2-oxoethyl)amino)-2-oxoethyl)thiazol-2-yl)-1H-indol-1-yl)propyl)piperazine-1-carboxylate (1 eq) in DCM treated with 4N. HCl in 1,4-dioxane for 2 hours. After completion of the reaction mixture was evaporated and the solid residue was purified by flash C-18 column chromatography, product eluted at 1:1 water-acetonitrile to get benzyl (2-(2-(1-(3-(piperazin-1-yl)propyl)-1H-indol-6-yl)thiazol-4-yl)acetyl)glycinate (ESI-MS (M+1): 532.59) in quantitative yields. A small amount of this intermediate (200 mg, 0.376 mmol) was taken in THF-CH₃CN—H₂O (2:2:1) (2 mL) and treated with LiOH (36.0 mg, 1.505 mmol). The mixture was stirred at room temperature for 2 hours, followed by acidification with dilute aq. HCl and evaporation to give the crude product. The crude product was purified by a C18 flash column to give the desired product (2-(2-(1-(3-(piperazin-1-yl)propyl)-1H-indol-6-yl)thiazol-4-yl)acetyl)glycine. ¹H NMR (400 MHz, Deuterium Oxide) δ 7.96 (dd, J=1.7, 0.9 Hz, 1H), 7.66 (d, J=8.3 Hz, 1H), 7.51 (dd, J=8.3, 1.5 Hz, 1H), 7.37 (d, J=3.1 Hz, 1H), 7.32 (s, 1H), 6.70-6.48 (m, 1H), 4.27 (t, J=6.7 Hz, 2H), 3.84 (s, 4H), 3.43 (t, J=5.3 Hz, 4H), 3.24 (s, 4H), 3.04-2.82 (m, 2H), 2.22 (p, J=6.7 Hz, 2H). ESI-MS (M+1): 316.34.

### n. Synthesis of Indole PROTACS 5397 and 5399

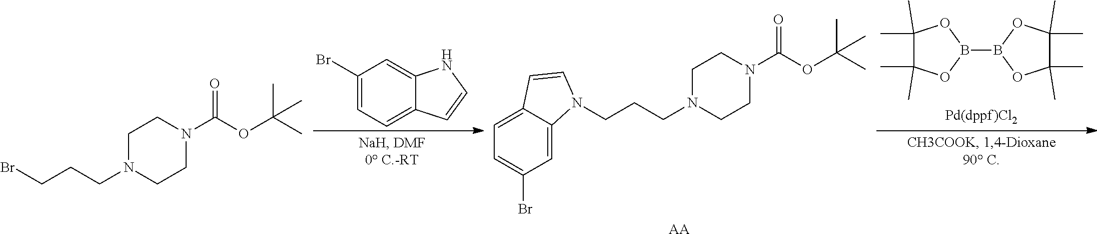

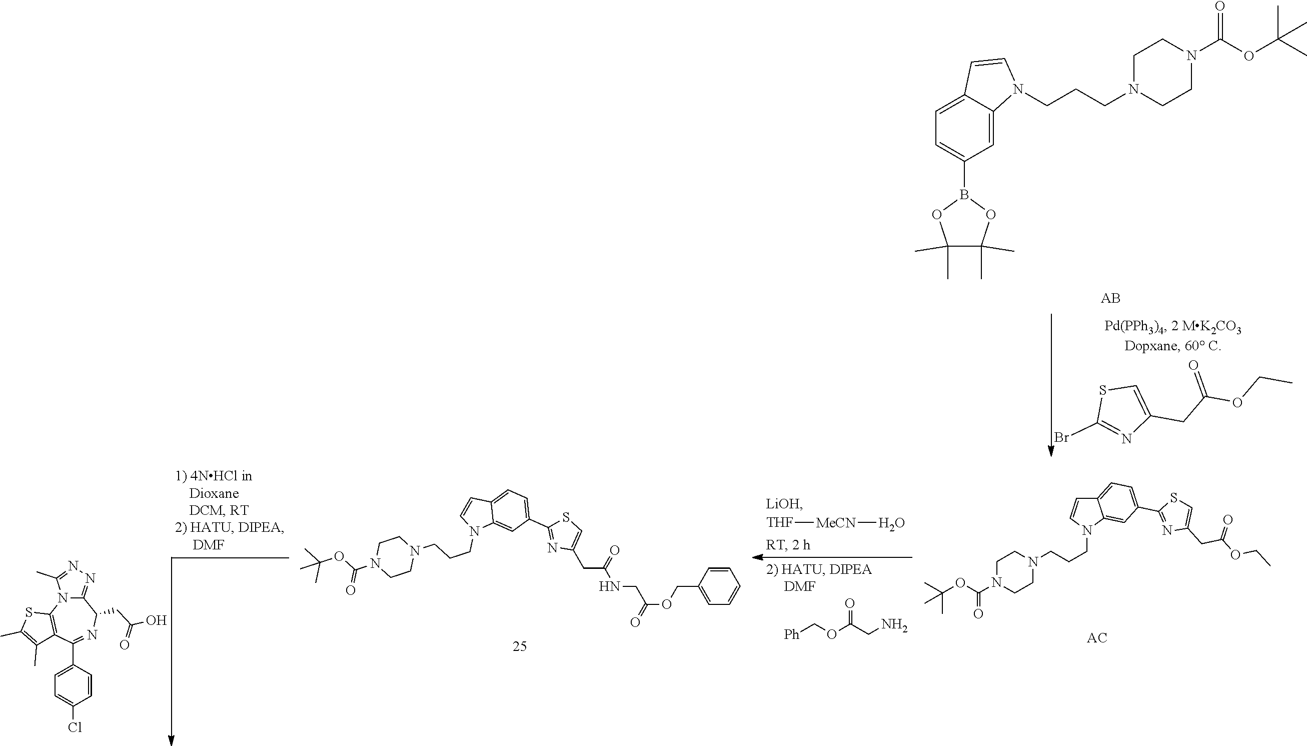

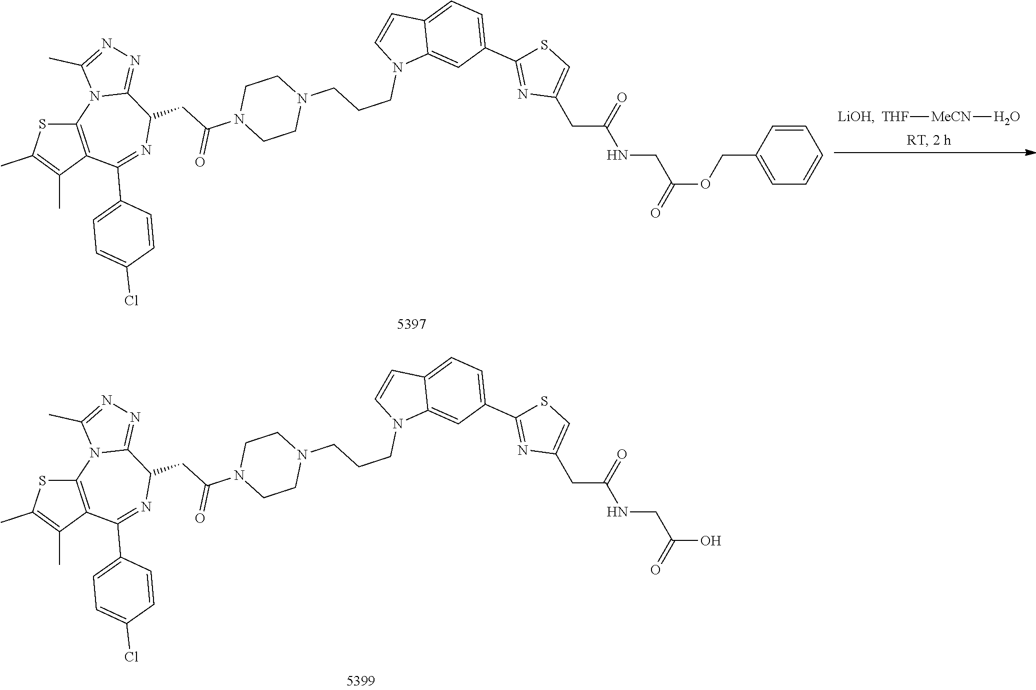

### i. Synthesis of Tert-Butyl 4-(3-(6-Bromo-1H-Indol-1-yl)Propyl)Piperazine-1-Carboxylate (AA)

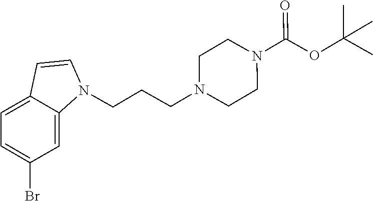 [SMILES: CC(C)(C)OC(=O)N1CCN(CCCn2ccc3ccc(Br)cc32)CC1]

To a mixture of 6-Bromoindole (0.702 g, 3.58 mmol) in DMF (5 ml) at 0° C. added NaH (0.087 g, 3.25 mmol) and the mixture was allowed to stir for 10 min, then 4-(2-Bromopropyl)-1-piperazinecarboxylic acid, 1,1-dimethylethyl ester (1 g, 3.25 mmol) was added. The reaction mixture stirred at RT for 2 h. After completion of reaction diluted with water, extracted with ethyl acetate, dried, evaporated, and the residue purified by flash column. The tert-butyl 4-(3-(6-bromo-1H-indol-1-yl)propyl)piperazine-1-carboxylate (1.05 g, 2.486 mmol, 76% yield) product eluted at 70% ethyl acetate in hexanes. ¹H NMR (500 MHz, Chloroform-d) δ 7.50 (d, J=1.6 Hz, 1H), 7.39 (d, J=8.4 Hz, 1H), 7.11 (dt, J=8.4, 1.2 Hz, 1H), 7.00 (d, J=3.2 Hz, 1H), 6.39 (d, J=3.1 Hz, 1H), 4.12 (t, J=6.5 Hz, 2H), 3.43 (s, 4H), 2.24 (d, J=53.7 Hz, 6H), 2.04-1.85 (m, 2H), 1.39 (d, J=1.0 Hz, 9H). ESI-MS (M+1): 424.10.

### ii. Synthesis of Tert-Butyl 4-(3-(6-(4,4,5,5-Tetramethyl-1,3,2-Dioxaborolan-2-yl)-1H-Indol-1-yl)Propyl)Piperazine-1-Carboxylate (AB)

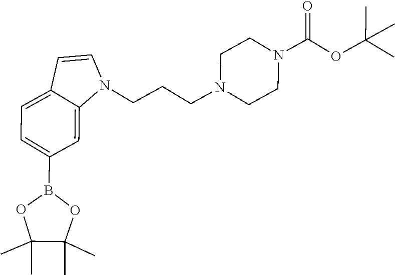 [SMILES: CC(C)(C)OC(=O)N1CCN(CCCn2ccc3ccc(B4OC(C)(C)C(C)(C)O4)cc32)CC1]

To a nitrogen gas purged mixture of tert-butyl 4-(3-(6-bromo-TH-indol-1-yl)propyl)piperazine-1-carboxylate (1.05 g, 2.486 mmol), Bis(pinacolato)diboron (0.758 g, 2.98 mmol) and potassium acetate (0.488 g, 4.97 mmol) in anhydrous 1,4-dioxane (10 ml) was added [1,1′-Bis(diphenylphosphino)ferrocene]dichloropalladium(II) (0.182 g, 0.249 mmol) and allowed to purge with nitrogen for additional 5 minutes. The reaction mix was then stirred at 90° C. for 6 hours. The reaction mixture was allowed to cool to room temperature, filtered and the filtrate was evaporated to dryness. The residue was subjected to flash column purification, product eluted at 60% ethyl acetate in hexanes to afford tert-butyl 4-(3-(6-(4,4,5,5-tetramethyl-1,3,2-dioxaborolan-2-yl)-1H-indol-1-yl)propyl)piperazine-1-carboxylate (750 mg, 1.598 mmol, 64.3% yield). ¹H NMR (500 MHz, Chloroform-d) δ 7.79 (s, 1H), 7.55 (d, J=7.9 Hz, 1H), 7.47 (dt, J=7.9, 1.1 Hz, 1H), 7.10 (d, J=3.0 Hz, 1H), 6.42 (t, J=2.6 Hz, 1H), 4.38-4.15 (m, 2H), 3.45 (s, 4H), 2.23 (d, J=76.4 Hz, 6H), 1.87 (d, J=7.2 Hz, 2H), 1.38 (s, 9H), 1.30 (s, 12H). ESI-MS (M+1): 469.26.

### iii. Synthesis of Tert-Butyl 4-(3-(6-(4-(2-Ethoxy-2-Oxoethyl)Thiazol-2-yl)-1H-Indol-1-yl)Propyl)Piperazine-1-Carboxylate (AC)

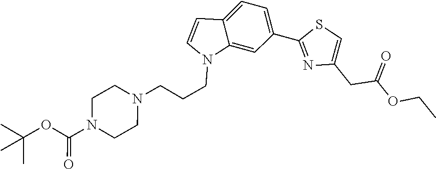 [SMILES: CCOC(=O)Cc1csc(-c2ccc3ccn(CCCN4CCN(C(=O)OC(C)(C)C)CC4)c3c2)n1]

Mixture of ethyl 2-(2-bromothiazol-4-yl)acetate (0.65 g, 2.60 mmol), tert-butyl 4-(3-(6-(4,4,5,5-tetramethyl-1,3,2-dioxaborolan-2-yl)-1H-indol-1-yl)propyl)piperazine-1-carboxylate (1.220 g, 2.60 mmol) and 2 M. potassium carbonate (2.60 ml, 5.20 mmol) in dioxane purged with nitrogen for 5 min then Tetrakis(triphenylphosphine)palladium(0) (0.300 g, 0.260 mmol) was added followed by additional 3 min purging with nitrogen. The reaction mixture was stirred at 60° C. for 5 h. After completion of reaction, the mixture was filtered evaporated, and subjected to flash column purification, product eluted at 100% EA to give tert-butyl 4-(3-(6-(4-(2-ethoxy-2-oxoethyl)thiazol-2-yl)-1H-indol-1-yl)propyl)piperazine-1-carboxylate (1.01 g, 1.970 mmol, 76% yield)¹H NMR (400 MHz, Chloroform-d) δ 7.94 (d, J=1.2 Hz, 1H), 7.65-7.57 (m, 1H), 7.49-7.37 (m, 1H), 7.15 (d, J=3.1 Hz, 1H), 6.45 (dd, J=3.0, 0.8 Hz, 1H), 4.26 (d, J=6.7 Hz, 2H), 4.16 (q, J=7.1 Hz, 2H), 3.83 (d, J=0.8 Hz, 2H), 3.52 (s, 4H), 2.38 (t, J=87.8 Hz, 6H), 1.78-1.46 (m, 2H), 1.37 (s, 9H), 1.24 (t, J=7.1 Hz, 3H). ESI-MS (M+1): 513.57.

### iv. Synthesis of Tert-Butyl 4-(3-(6-(4-(2-((2-(Benzyloxy)-2-Oxoethyl)Amino)-2-Oxoethyl)Thiazol-2-yl)-1H-Indol-1-yl)Propyl)Piperazine-1-Carboxylate (25)

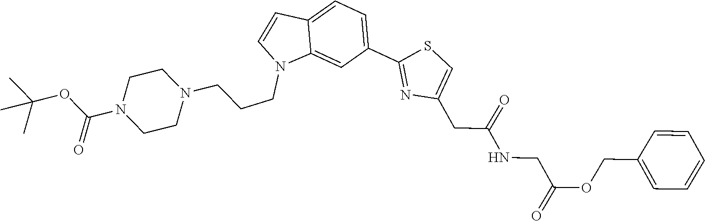 [SMILES: CC(C)(C)OC(=O)N1CCN(CCCn2ccc3ccc(-c4nc(CC(=O)NCC(=O)OCc5ccccc5)cs4)cc32)CC1]

A mixture of tert-butyl 4-(3-(6-(4-(2-ethoxy-2-oxoethyl)thiazol-2-yl)-1H-indol-1-yl)propyl)piperazine-1-carboxylate (1 g, 1.951 mmol) and LiOH (0.093 g, 3.90 mmol) in THF-CH₃CN—H₂O (2:2:1) (10 mL) stirred at room temperature for 2 hours. The reaction mixture was neutralized with 1 N. aq. HCl and evaporated to dryness. The residue was diluted with THF, filtered and the filtrate was evaporated to get crude 2-(2-(1-(3-(4-(tert-butoxycarbonyl)piperazin-1-yl)propyl)-1H-indol-6-yl)thiazol-4-yl)acetic acid (0.94 g, 1.940 mmol, 99% yield), which was subjected to the next reaction without further purification. The above obtained carboxylic acid was taken in DMF (5 mL) and HATU (1.106 g, 2.91 mmol), DIPEA (1.016 ml, 5.82 mmol) were added, before the mixture was allowed to stir for 10 minutes. Next, glycine benzyl ester hydrochloride (0.587 g, 2.91 mmol) was added. The reaction mixture was allowed to stir at room temperature overnight. The reaction mixture was diluted with water and extracted with ethyl acetate. The ethyl acetate was dried over Na₂SO₄, filtered, and evaporated to give the crude product. The crude product was then subjected to flash column chromatography to elute pure tert-butyl 4-(3-(6-(4-(2-((2-(benzyloxy)-2-oxoethyl)amino)-2-oxoethyl)thiazol-2-yl)-1H-indol-1-yl)propyl)piperazine-1-carboxylate at 10% methanol in DCM. ¹H NMR (400 MHz, Chloroform-d) δ 8.16 (q, J=1.0 Hz, 1H), 7.91 (t, J=5.0 Hz, 1H), 7.64 (t, J=1.0 Hz, 2H), 7.43-7.28 (m, 5H), 7.22 (d, J=3.1 Hz, 1H), 7.06 (d, J=0.8 Hz, 1H), 6.52 (dd, J=3.0, 0.8 Hz, 1H), 5.18 (s, 2H), 4.35 (t, J=6.5 Hz, 2H), 4.14 (d, J=5.0 Hz, 2H), 3.82 (d, J=0.8 Hz, 2H), 3.50 (s, 4H), 2.42 (s, 6H), 2.11 (s, 2H), 1.44 (s, 9H). ESI-MS (M+1): 632.27.

### v. Synthesis of Benzyl (S)-(2-(2-(1-(3-(4-(2-(4-(4-Chlorophenyl)-2,3,9-Trimethyl-6H-Thieno[3,2-F][1,2,4]Triazolo[4,3-A][1,4]Diazepin-6-yl)Acetyl)Piperazin-1-yl)Propyl)-1H-Indol-6-yl)Thiazol-4-yl)Acetyl)Glycinate (5397)

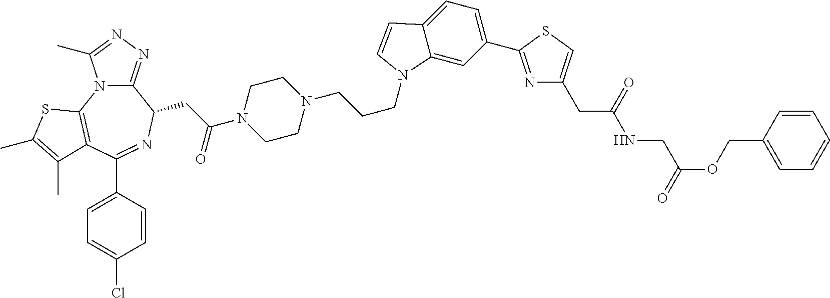 [SMILES: Cc1sc2c(c1C)C(c1ccc(Cl)cc1)=N[C@@H](CC(=O)N1CCN(CCCn3ccc4ccc(-c5nc(CC(=O)NCC(=O)OCc6ccccc6)cs5)cc43)CC1)c1nnc(C)n1-2]

To a pre-stirred mixture of benzyl (2-(2-(1-(3-(piperazin-1-yl)propyl)-1H-indol-6-yl)thiazol-4-yl)acetyl)glycinate, 2HCl (100 mg, 0.165 mmol), HATU (94 mg, 0.248 mmol), DIPEA (0.144 ml, 0.827 mmol) in DMF (2 ml) at RT was added (S)-2-(4-(4-chlorophenyl)-2,3,9-trimethyl-6H-thieno[3,2-f][1,2,4]triazolo[4,3-a][1,4]diazepin-6-yl)acetic acid (80 mg, 0.198 mmol). The reaction mixture continued to stir over night. The reaction mixture was diluted with water and the product that were separated by filtration were collected as solids. The crude product was subjected to flash column purification. The product eluted at 10% MeOH in DCM to afford benzyl (S)-(2-(2-(1-(3-(4-(2-(4-(4-chlorophenyl)-2,3,9-trimethyl-6H-thieno[3,2-f][1,2,4]triazolo[4,3-a][1,4]diazepin-6-yl)acetyl)piperazin-1-yl)propyl)-1H-indol-6-yl)thiazol-4-yl)acetyl)glycinate (50 mg, 33.1% yields). ¹H NMR (400 MHz, DMSO-d₆) δ 8.54 (t, J=5.8 Hz, 1H), 8.14-8.00 (m, 1H), 7.66-7.55 (m, 2H), 7.53 (d, J=3.1 Hz, 1H), 7.51-7.39 (m, 4H), 7.39-7.27 (m, 6H), 6.51 (dd, J=3.1, 0.8 Hz, 1H), 5.13 (s, 2H), 4.56 (t, J=6.7 Hz, 1H), 4.32 (t, J=6.4 Hz, 2H), 3.96 (d, J=5.8 Hz, 2H), 3.73 (s, 2H), 3.67 (s, 1H), 3.63-3.33 (m, 5H), 2.58 (s, 3H), 2.45-2.35 (m, 4H), 2.34-2.14 (m, 5H), 1.97 (t, J=6.6 Hz, 2H), 1.70-1.55 (m, 3H). ESI-MS (M+1): 914.71.

### vi. Synthesis of (S)-(2-(2-(1-(3-(4-(2-(4-(4-Chlorophenyl)-2,3,9-Trimethyl-6H-Thieno[3,2-F][1,2,4]Triazolo[4,3-A][1,4]Diazepin-6-yl)Acetyl)Piperazin-1-yl)Propyl)-1H-Indol-6-yl)Thiazol-4-yl)Acetyl)Glycine (5399)

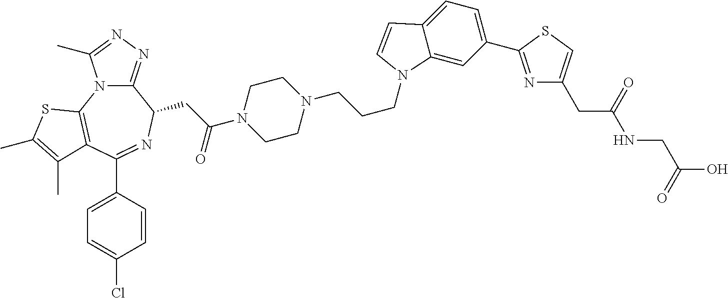 [SMILES: Cc1sc2c(c1C)C(c1ccc(Cl)cc1)=N[C@@H](CC(=O)N1CCN(CCCn3ccc4ccc(-c5nc(CC(=O)NCC(=O)O)cs5)cc43)CC1)c1nnc(C)n1-2]

The benzyl (S)-(2-(2-(1-(3-(4-(2-(4-(4-chlorophenyl)-2,3,9-trimethyl-6H-thieno[3,2-f][1,2,4]triazolo[4,3-a][1,4]diazepin-6-yl)acetyl)piperazin-1-yl)propyl)-1H-indol-6-yl)thiazol-4-yl)acetyl)glycinate (5397) was subjected to hydrolysis using LiOH as explained in previous method. The crude product was taken in water, treated with dilute aq. HCl to acidify and evaporated to dryness. The residue was treated with THF to crash out product as solids, which were collected by filtration, washed with additional THF and dried to get pure (S)-(2-(2-(1-(3-(4-(2-(4-(4-chlorophenyl)-2,3,9-trimethyl-6H-thieno[3,2-f][1,2,4]triazolo[4,3-a][1,4]diazepin-6-yl)acetyl)piperazin-1-yl)propyl)-1H-indol-6-yl)thiazol-4-yl)acetyl)glycine. 1H NMR (400 MHz, DMSO-d6) δ 8.41 (t, J=5.6 Hz, 1H), 8.13 (d, J=1.5 Hz, 1H), 7.63 (d, J=8.3 Hz, 1H), 7.57 (dd, J=8.2, 1.5 Hz, 1H), 7.53 (d, J=3.1 Hz, 1H), 7.52-7.38 (m, 4H), 7.31 (d, J=4.8 Hz, 1H), 6.51 (dd, J=3.0, 0.8 Hz, 1H), 4.57 (t, J=6.7 Hz, 1H), 4.33 (t, J=6.7 Hz, 2H), 3.78 (d, J=5.6 Hz, 2H), 3.75-3.63 (m, 10H), 3.57-3.44 (m, 1H), 2.59 (s, 3H), 2.41 (d, J=0.9 Hz, 3H), 2.38-2.23 (m, 3H), 2.01 (p, J=6.9 Hz, 2H), 1.67-1.59 (m, 3H). ESI-MS (M+1): 824.71.

### o. Synthesis of Morpholino-Phenyl-Based KLHDC2 Ligands

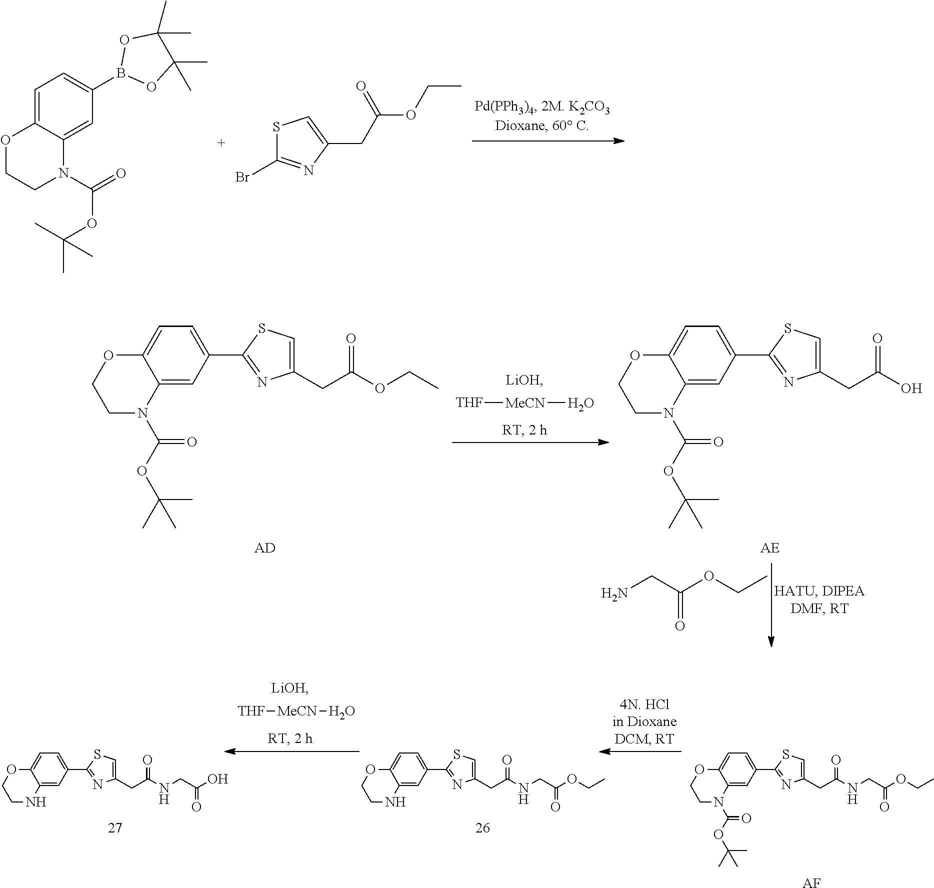

### i. Synthesis of Tert-Butyl 6-(4-(2-Ethoxy-2-Oxoethyl)Thiazol-2-yl)-2,3-Dihydro-4H-Benzo [B][1,4]Oxazine-4-Carboxylate (AD)

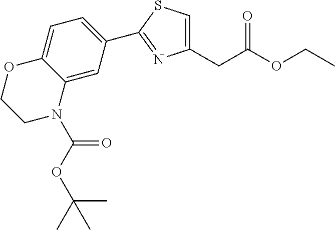 [SMILES: CCOC(=O)Cc1csc(-c2ccc3c(c2)N(C(=O)OC(C)(C)C)CCO3)n1]

A mix of ethyl 2-(2-bromothiazol-4-yl)acetate (1 g, 4.00 mmol), tert-butyl 6-(4,4,5,5-tetramethyl-1,3,2-dioxaborolan-2-yl)-2,3-dihydro-4H-benzo[b][1,4]oxazine-4-carboxylate (1.444 g, 4.00 mmol) and 2 M. potassium carbonate (6.00 ml, 11.99 mmol) in Dioxane (10 ml) was degassed with nitrogen gas for 5 min, then tetrakis(triphenylphosphine)palladium(0) (0.462 g, 0.400 mmol) was added followed by an additional 3 min of degassing. The mixture was then stirred at 60° C. for 5 h, the RM was filtered, evaporated, diluted with ethyl acetate, and washed with water. The ethyl acetate layer was dried, evaporated, and subjected to flash column purification. The pure product tert-butyl 6-(4-(2-ethoxy-2-oxoethyl)thiazol-2-yl)-2,3-dihydro-4H-benzo[b][1,4]oxazine-4-carboxylate (1.027 g, 2.54 mmol, 63.5% yield) eluted at 25% EA in Hexanes. ¹H NMR (500 MHz, Chloroform-d) δ 8.31 (s, 1H), 7.65 (d, J=8.5 Hz, 1H), 7.10 (s, 1H), 6.86 (d, J=8.5 Hz, 1H), 4.22 (t, J=4.6 Hz, 2H), 4.14 (q, J=7.1 Hz, 2H), 3.86 (s, 2H), 3.82 (t, J=4.6 Hz, 2H), 1.51 (s, 9H), 1.22 (t, J=7.1 Hz, 3H). ESI-MS (M+1): 406.02.

### ii. Synthesis of Tert-Butyl 6-(4-(2-((2-Ethoxy-2-Oxoethyl)Amino)-2-Oxoethyl)Thiazol-2-yl)-2,3-Dihydro-4H-Benzo [B][1,4]Oxazine-4-Carboxylate (AF)

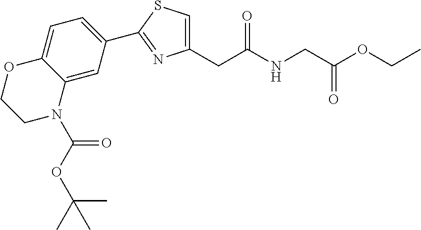 [SMILES: CCOC(=O)CNC(=O)Cc1csc(-c2ccc3c(c2)N(C(=O)OC(C)(C)C)CCO3)n1]

The above obtained intermediate was subjected to ester hydrolysis using LiOH in a THF-CH₃CN—H₂O (2:2:1) solvent system as explained in earlier protocols and the resulting carboxylate product (ESI-MS (M−1): 375.15) was used in the next reaction without further purification. A mixture of 2-(2-(4-(tert-butoxycarbonyl)-3,4-dihydro-2H-benzo[b][1,4]oxazin-6-yl)thiazol-4-yl)acetic acid (238 mg, 0.632 mmol), HATU (361 mg, 0.948 mmol) and DIPEA (0.331 ml, 1.897 mmol) in DMF (5 ml) was stirred for 10 min at room temperature, then glycine ethyl ester hydrochloride (132 mg, 0.948 mmol) was added and the reaction mixture was allowed to stir for 3 hours at room temperature. After completion of the reaction, the mixture was diluted with water, extracted with ethyl acetate, dried, and the resulting residue was purified by flash column chromatography to get tert-butyl 6-(4-(2-((2-ethoxy-2-oxoethyl)amino)-2-oxoethyl)thiazol-2-yl)-2,3-dihydro-4H-benzo[b][1,4]oxazine-4-carboxylate (172 mg, 0.373 mmol, 58.9% yield). ¹H NMR (500 MHz, Chloroform-d) δ 8.33 (s, 1H), 7.60 (dd, J=8.7, 2.2 Hz, 1H), 7.44 (d, J=5.5 Hz, 1H), 7.07 (s, 1H), 6.90 (d, J=8.5 Hz, 1H), 4.29-4.18 (m, 2H), 4.12 (q, J=7.1 Hz, 2H), 3.98 (d, J=5.3 Hz, 2H), 3.84 (t, J=4.6 Hz, 2H), 3.77 (s, 2H), 1.51 (s, 9H), 1.18 (t, J=7.1 Hz, 3H). ESI-MS (M+1): 462.17.

### iii. Synthesis of Ethyl (2-(2-(3,4-Dihydro-2H-Benzo[B][1,4]Oxazin-6-yl)Thiazol-4-yl)Acetyl)Glycinate (26)

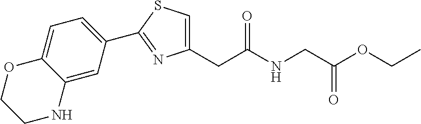 [SMILES: CCOC(=O)CNC(=O)Cc1csc(-c2ccc3c(c2)NCCO3)n1]

The boc protected amine (1 eq.) was taken in DCM and treated with 4 N. HCl in 1,4-dioxane (5 eq.) and stirred for 2 hours. The reaction mixture was dried and evaporated to get the product, which was used in the next reaction without further purification. ¹H NMR (500 MHz, DMSO-d₆) δ 8.50 (t, J=5.9 Hz, 1H), 7.35 (d, J=2.8 Hz, 1H), 7.26 (d, J=2.2 Hz, 1H), 7.12 (dd, J=8.2, 2.2 Hz, 1H), 6.77 (d, J=8.3 Hz, 1H), 4.20 (t, J=4.4 Hz, 2H), 4.10 (q, J=7.1 Hz, 2H), 3.87 (d, J=5.8 Hz, 2H), 3.68 (s, 2H), 3.57 (s, 2H), 1.19 (t, J=7.1 Hz, 3H). ESI-MS (M+1): 361.4.

### iv. Synthesis of (2-(2-(3,4-Dihydro-2H-Benzo[B][1,4]Oxazin-6-yl)Thiazol-4-yl)Acetyl)Glycine (27)

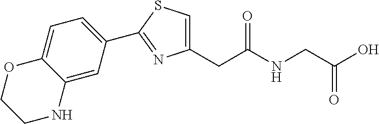 [SMILES: O=C(O)CNC(=O)Cc1csc(-c2ccc3c(c2)NCCO3)n1]

A mixture of ethyl (2-(2-(3,4-dihydro-2H-benzo[b][1,4]oxazin-6-yl)thiazol-4-yl)acetyl)glycinate, HCl (50 mg, 0.126 mmol), and LiOH (15.05 mg, 0.628 mmol) in 2.5 mL of THF-CH₃CN—H₂O (2:2:1) solvent was stirred for 2 hours. After completion of the reaction, the mixture was neutralized, evaporated, and the resulting residue purified by flash column chromatography to get (2-(2-(3,4-dihydro-2H-benzo[b][1,4]oxazin-6-yl)thiazol-4-yl)acetyl)glycine (31.5 g, 94 mmol, 7.52E+04% yield). ¹H NMR (500 MHz, DMSO-d₆) δ 8.31 (t, J=5.8 Hz, 1H), 7.33 (s, 1H), 7.19 (t, J=1.8 Hz, 1H), 7.03 (dt, J=8.1, 1.6 Hz, 1H), 6.72 (d, J=8.3 Hz, 1H), 6.07 (s, 1H), 4.17 (t, J=4.3 Hz, 2H), 3.77 (d, J=5.7 Hz, 2H), 3.66 (s, 2H), 3.32 (d, J=4.6 Hz, 2H). ¹³C NMR (126 MHz, DMSO) δ 171.73, 169.57, 167.57, 151.53, 145.35, 135.78, 126.81, 116.82, 115.49, 115.43, 112.34, 65.34, 41.53, 38.57. ESI-MS (M+1): 334.89.

### 2. Biological Experimentals

### A. Thermal Shift Method

To identify small molecules that bind to KLHDC2 the Sypro orange thermal stability assay was utilized. 100 nl 10 mM experimental compound was transferred to a 384-well PCR plate using the Labcyte ECHO 655T. Assay solution containing 0.5 μM KLHDC2, 25 mM HEPES, 200 mM NaCl and 5% Sypro Orange pH 7.5 was added to a final volume of 20 ul and a final experimental ligand concentration of 50 uM. DMSO alone was included as a negative control and peptide sub2 was included as a positive control at 100 uM. The fluorescence of Sypro orange in each sample was measured on a Quantstudio 5 or a Quantstudio 6 at temperatures ranging from 23 to 75° C. at 0.05° C./second. Data reduction resulting in thermal stability (calculated using the Boltzmann and first derivative minima equations) was completed in the Thermo Scientific protein thermal shift software v1.4.

b. Time-Resolved Fluorescence Energy Transfer (Tr-Fret) Assay

Test compounds dissolved in DMSO were transferred by Echo 655T (Labcyte, San Jose, CA) to 384-well low-volume solid black plates. A mixture of biotin-KLHDC2, terbium-streptavidin (Thermo Fisher) and a Bodipy-labeled ligand probe (Lee-4893) was dispensed into the assay plates at a final volume of 20 μL using a Tempest (Formulatrix, Bedford, MA), followed by incubation at room temperature for 1 h prior to measurement of the TR-FRET signal using a PHERAStar plate reader (BMG Labtech, Durham, NC), which was equipped with modules for excitation at 337 nm and emission at 490 and 520 nm. The integration start was set to 100 μs, and the integration time was set to 200 μs. The number of flashes was fixed at 100. The 520/490 ratio was used as the TR-FRET signal in calculations.

c. Ternary Complex Assay

To monitor the strength of ternary complex formation, PROTAC-substrate complexes were treated as pseudo-substrate inhibitors. The indicated stoichiometric concentrations of PROTAC—DCN1 (or DCN2) were pre-equilibrated with 25 nM NEDD8˜CUL2^{KLHDC2} at room temperature for 15 minutes. Ubiquitylation reactions were then initiated by the simultaneous addition of a model KLHDC2 substrate to 0.8 mM, and fluorescently labeled ubiquitin charged UBE2R2 to 0.3 mM. Reactions were quenched at 10 seconds by the addition of 2× Sample buffer and separated on 4-12% gradient gels. Gels were scanned on a typhoon imager and quantified for inhibition of model substrate ubiquitylation.

d. Cell Dosing Studies

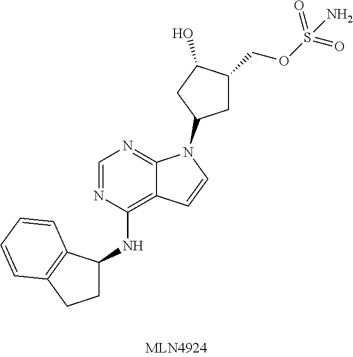

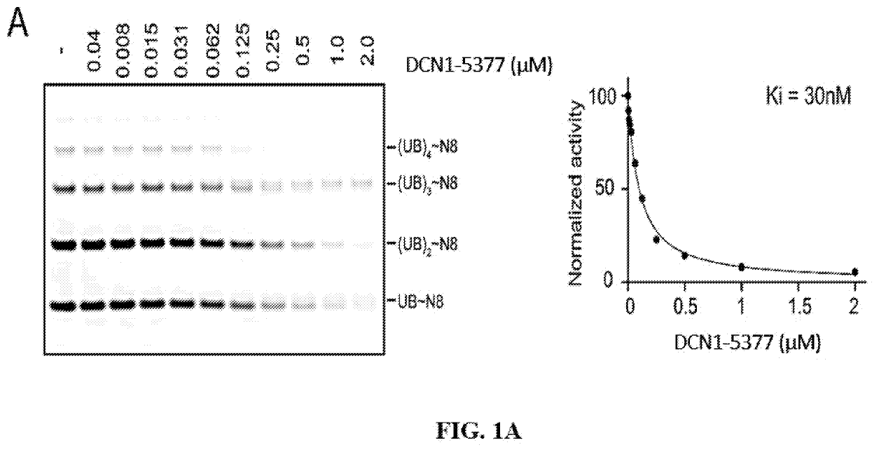

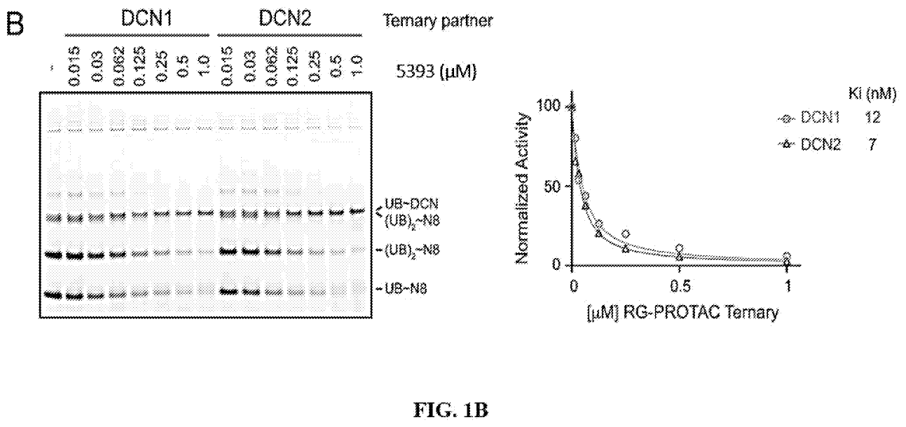

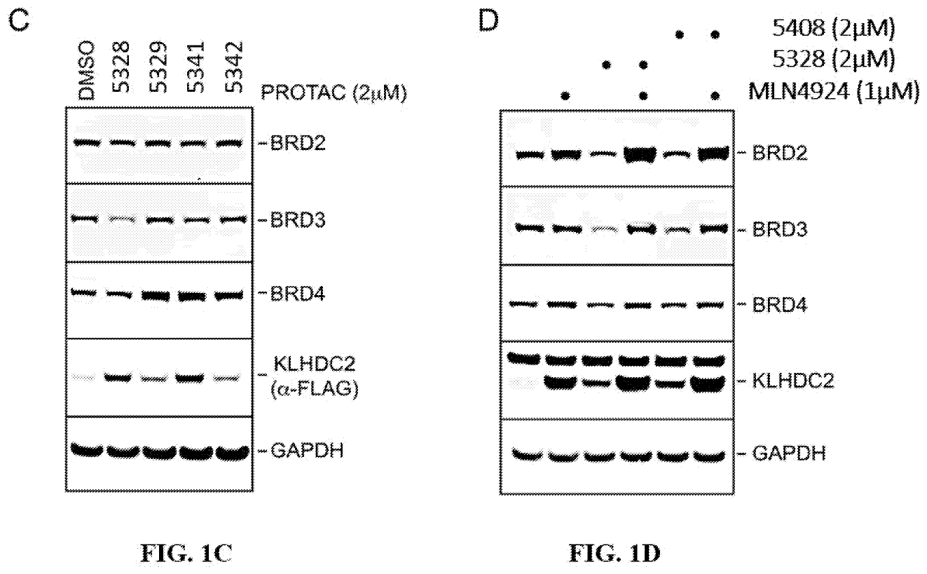

U2OS cells were dosed with 2 mM final concentration of the indicated PROTACs. After 24 hour incubation, cells were harvested and lysed. Where indicated, cells were co-dosed by the addition of MLN4924 (structure shown above) to 1 mM. Protein concentrations in the lysate were determined by BCA assay, and equivalent amounts of total protein were added to 2× sample buffer and separated on 4-12% gradient gels. After transfer to nitrocellulose membranes, westerns were performed with the indicated antibodies and developed with SuperSignal West chemiluminiscent substrate. See FIG. 1.

### 3. Exemplary KLHDC2 Ligands

A summary of the exemplary KLHDC2 ligands prepared along with their melting temperatures and half-maximal inhibitory concentrations is shown in Table 1 below.

TABLE 1

| | | ΔTₘ | IC₅₀ |
|---|---|---|---|
| | | (° C.) 50 | (μM) |
| | | μM | TR- |
| No. | Structure | ligand | FRET |
| i | 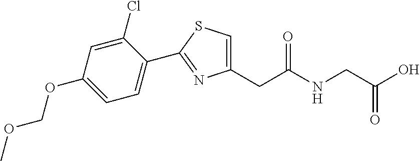 [SMILES: COCOc1ccc(-c2nc(CC(=O)NCC(=O)O)cs2)c(Cl)c1] | 3.52 ± 0.424 | 24.10 |
| 1 | 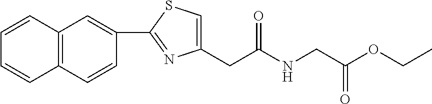 [SMILES: CCOC(=O)CNC(=O)Cc1csc(-c2ccc3ccccc3c2)n1] | — | — |
| 2 | 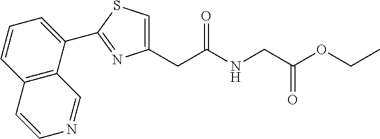 [SMILES: CCOC(=O)CNC(=O)Cc1csc(-c2cccc3ccncc23)n1] | — | — |
| 3 | 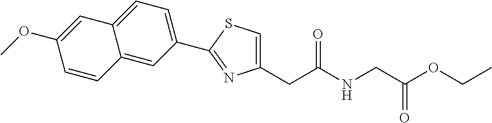 [SMILES: CCOC(=O)CNC(=O)Cc1csc(-c2ccc3cc(OC)ccc3c2)n1] | — | — |
| 4 | 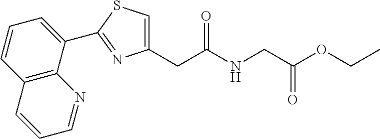 [SMILES: CCOC(=O)CNC(=O)Cc1csc(-c2cccc3cccnc23)n1] | — | — |
| 5 | 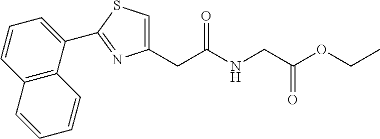 [SMILES: CCOC(=O)CNC(=O)Cc1csc(-c2cccc3ccccc23)n1] | — | — |
| 6 | 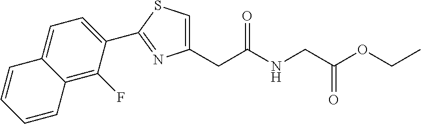 [SMILES: CCOC(=O)CNC(=O)Cc1csc(-c2ccc3ccccc3c2F)n1] | — | — |
| 7 | 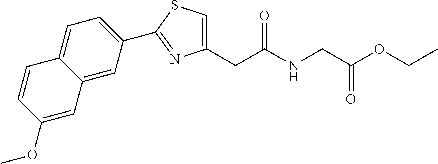 [SMILES: CCOC(=O)CNC(=O)Cc1csc(-c2ccc3ccc(OC)cc3c2)n1] | — | — |
| 8 | 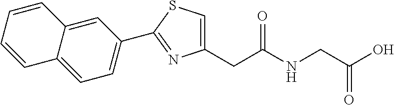 [SMILES: O=C(O)CNC(=O)Cc1csc(-c2ccc3ccccc3c2)n1] | 6.0 ± 0.0 | 3.9 |
| 9 | 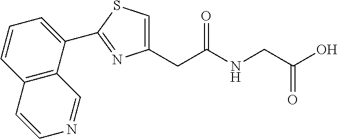 [SMILES: O=C(O)CNC(=O)Cc1csc(-c2cccc3ccncc23)n1] | 1.8 ± 0.0 | 20.3 |
| 10 | 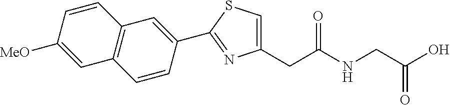 [SMILES: COc1ccc2cc(-c3nc(CC(=O)NCC(=O)O)cs3)ccc2c1] | 3.9 ± 0.1 | 9.6 |
| 11 | 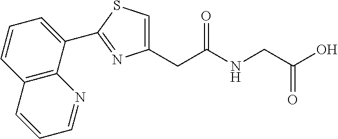 [SMILES: O=C(O)CNC(=O)Cc1csc(-c2cccc3cccnc23)n1] | 2.7 ± 0.4 | 7.1 |
| 12 | 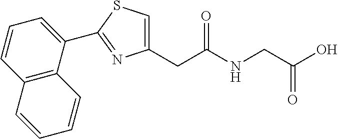 [SMILES: O=C(O)CNC(=O)Cc1csc(-c2cccc3ccccc23)n1] | 3.7 ± 0.0 | 8.8 |
| 13 | 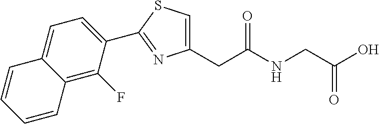 [SMILES: O=C(O)CNC(=O)Cc1csc(-c2ccc3ccccc3c2F)n1] | 6.0 ± 0.2 | 1.8 |
| 14 | 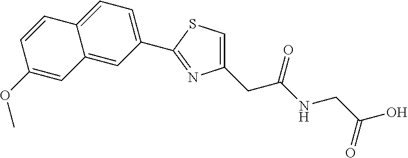 [SMILES: COc1ccc2ccc(-c3nc(CC(=O)NCC(=O)O)cs3)cc2c1] | 4.3 ± 0.2 | 1.7 |
| 15 | 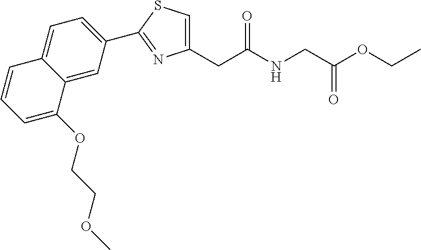 [SMILES: CCOC(=O)CNC(=O)Cc1csc(-c2ccc3cccc(OCCOC)c3c2)n1] | — | — |
| 22 | 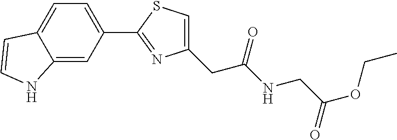 [SMILES: CCOC(=O)CNC(=O)Cc1csc(-c2ccc3cc[nH]c3c2)n1] | — | — |
| 23 | 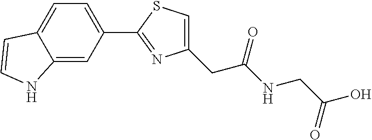 [SMILES: O=C(O)CNC(=O)Cc1csc(-c2ccc3cc[nH]c3c2)n1] | — | 1.1 |
| 26 | 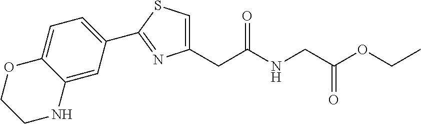 [SMILES: CCOC(=O)CNC(=O)Cc1csc(-c2ccc3c(c2)NCCO3)n1] | — | — |
| 27 | 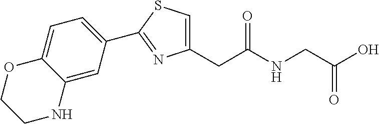 [SMILES: O=C(O)CNC(=O)Cc1csc(-c2ccc3c(c2)NCCO3)n1] | — | 3.0 |

### 4. Exemplary PROTAC Linkers

A summary of the exemplary PROTAC linkers prepared as detailed above is shown in Table 2 below.

TABLE 2

| | | ΔTₘ | |
|---|---|---|---|
| | | (° C.) | IC₅₀ |
| | | 50 | (μM) |
| | | μM | TR- |
| No. | Structure | ligand | FRET |
| 15A | 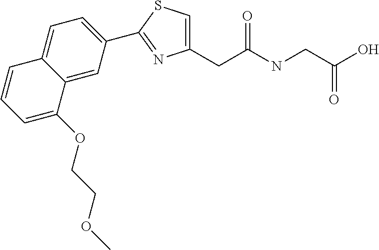 [SMILES: COCCOc1cccc2ccc(-c3nc(CC(=O)NCC(=O)O)cs3)cc12] | 4.3 ± 0.2 | 1.1 |
| 16 | 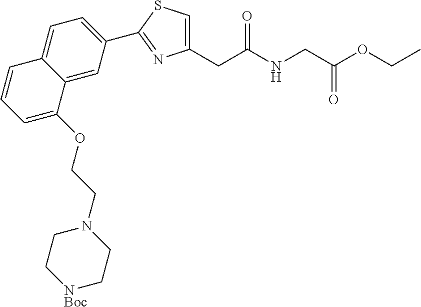 [SMILES: CCOC(=O)CNC(=O)Cc1csc(-c2ccc3cccc(OCCN4CCN(C(=O)OC(C)(C)C)CC4)c3c2)n1] | | |
| 17 | 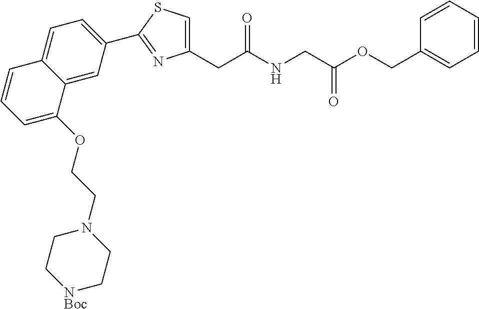 [SMILES: CC(C)(C)OC(=O)N1CCN(CCOc2cccc3ccc(-c4nc(CC(=O)NCC(=O)OCc5ccccc5)cs4)cc23)CC1] | | |
| 18 | 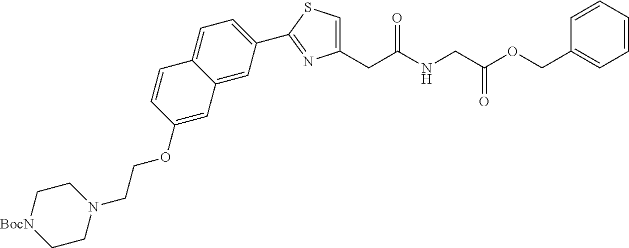 [SMILES: CC(C)(C)OC(=O)N1CCN(CCOc2ccc3ccc(-c4nc(CC(=O)NCC(=O)OCc5ccccc5)cs4)cc3c2)CC1] | | |
| 19 | 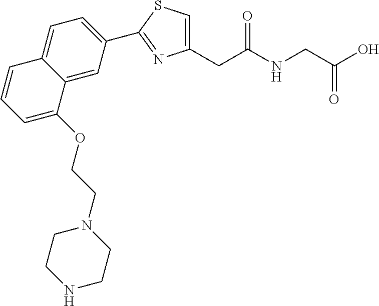 [SMILES: O=C(O)CNC(=O)Cc1csc(-c2ccc3cccc(OCCN4CCNCC4)c3c2)n1] | 4.8 ± 0.0 | 1.6 |
| 20 | 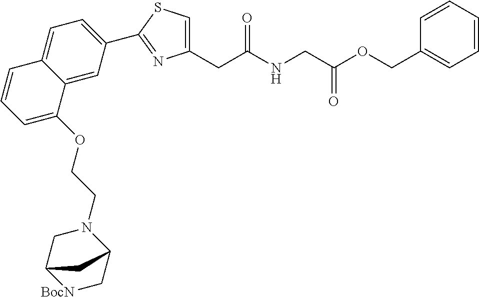 [SMILES: CC(C)(C)OC(=O)N1C[C@@H]2C[C@H]1CN2CCOc1cccc2ccc(-c3nc(CC(=O)NCC(=O)OCc4ccccc4)cs3)cc12] | | |
| 21 | 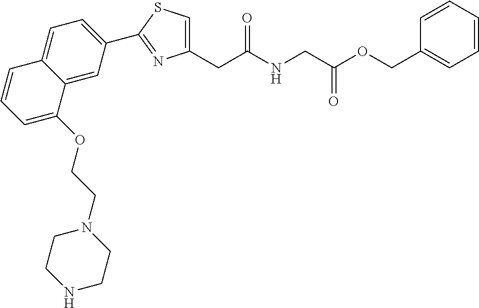 [SMILES: O=C(Cc1csc(-c2ccc3cccc(OCCN4CCNCC4)c3c2)n1)NCC(=O)OCc1ccccc1] | | |
| 24 | 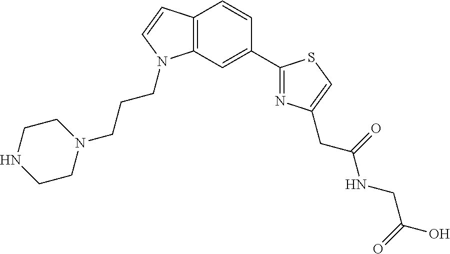 [SMILES: O=C(O)CNC(=O)Cc1csc(-c2ccc3ccn(CCCN4CCNCC4)c3c2)n1] | | 1.2 |
| 25 | 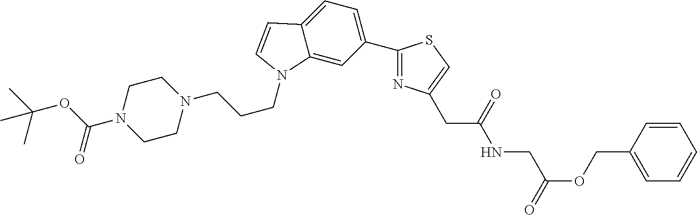 [SMILES: CC(C)(C)OC(=O)N1CCN(CCCn2ccc3ccc(-c4nc(CC(=O)NCC(=O)OCc5ccccc5)cs4)cc32)CC1] | | |

### 5. Exemplary PROTACS

A summary of exemplary PROTACS prepared as detailed above is shown in Table 3 below.

TABLE 3

| No. | Structure |
|---|---|
| 5328 | 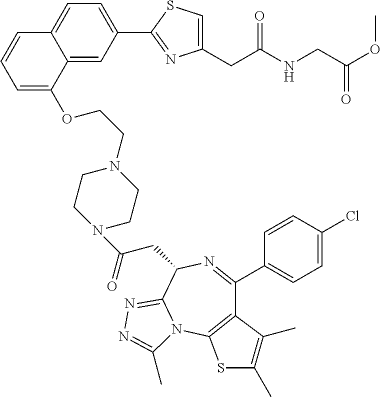 [SMILES: COC(=O)CNC(=O)Cc1csc(-c2ccc3cccc(OCCN4CCN(C(=O)C[C@@H]5N=C(c6ccc(Cl)cc6)c6c(sc(C)c6C)-n6c(C)nnc65)CC4)c3c2)n1] |
| 5329 | 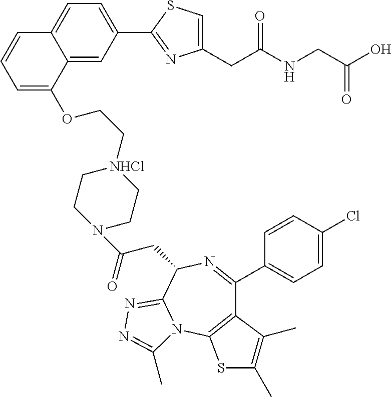 |
| 5407 | 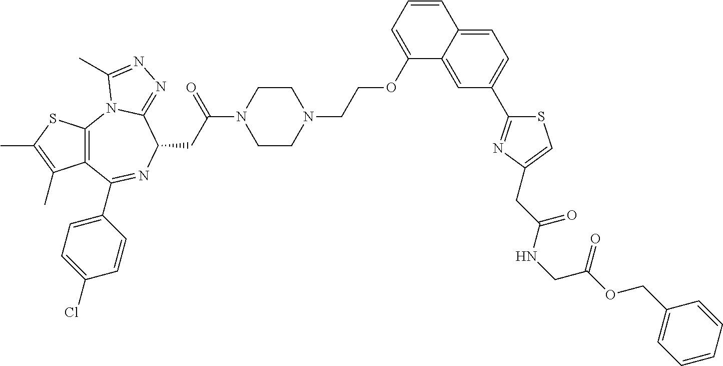 [SMILES: Cc1sc2c(c1C)C(c1ccc(Cl)cc1)=N[C@@H](CC(=O)N1CCN(CCOc3cccc4ccc(-c5nc(CC(=O)NCC(=O)OCc6ccccc6)cs5)cc34)CC1)c1nnc(C)n1-2] |
| 5408 | 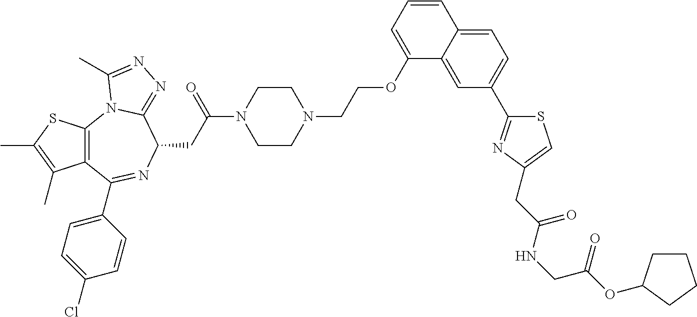 [SMILES: Cc1sc2c(c1C)C(c1ccc(Cl)cc1)=N[C@@H](CC(=O)N1CCN(CCOc3cccc4ccc(-c5nc(CC(=O)NCC(=O)OC6CCCC6)cs5)cc34)CC1)c1nnc(C)n1-2] |
| 5341 | 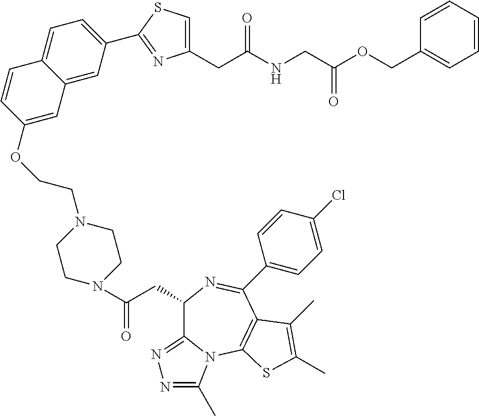 [SMILES: Cc1sc2c(c1C)C(c1ccc(Cl)cc1)=N[C@@H](CC(=O)N1CCN(CCOc3ccc4ccc(-c5nc(CC(=O)NCC(=O)OCc6ccccc6)cs5)cc4c3)CC1)c1nnc(C)n1-2] |
| 5342 | 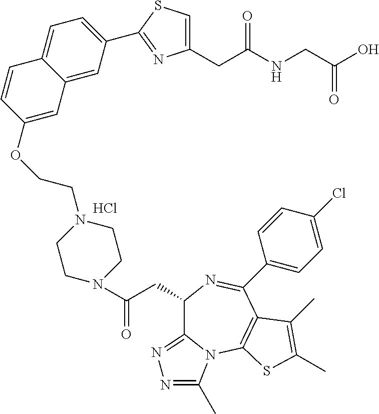 [SMILES: Cc1sc2c(c1C)C(c1ccc(Cl)cc1)=N[C@@H](CC(=O)N1CCN(CCOc3ccc4ccc(-c5nc(CC(=O)NCC(=O)O)cs5)cc4c3)CC1)c1nnc(C)n1-2 ; Cl] |
| 5372 | 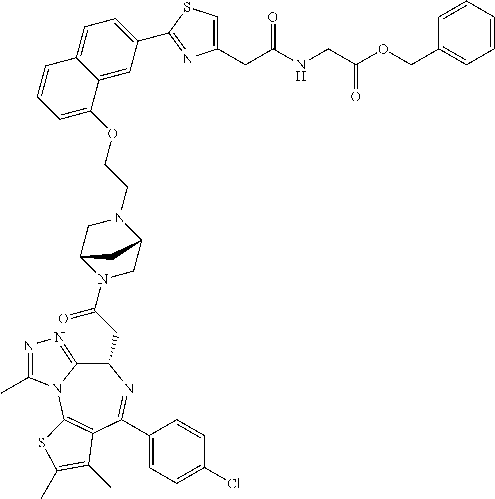 [SMILES: Cc1sc2c(c1C)C(c1ccc(Cl)cc1)=N[C@@H](CC(=O)N1C[C@@H]3C[C@H]1CN3CCOc1cccc3ccc(-c4nc(CC(=O)NCC(=O)OCc5ccccc5)cs4)cc13)c1nnc(C)n1-2] |
| 5373 | 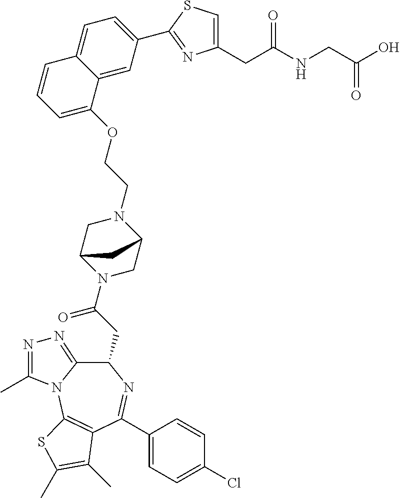 [SMILES: Cc1sc2c(c1C)C(c1ccc(Cl)cc1)=N[C@@H](CC(=O)N1C[C@@H]3C[C@H]1CN3CCOc1cccc3ccc(-c4nc(CC(=O)NCC(=O)O)cs4)cc13)c1nnc(C)n1-2] |
| 5343 | 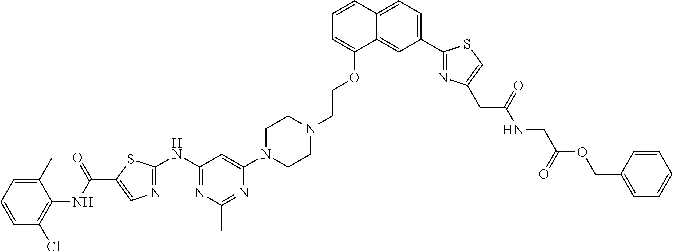 [SMILES: Cc1nc(Nc2ncc(C(=O)Nc3c(C)cccc3Cl)s2)cc(N2CCN(CCOc3cccc4ccc(-c5nc(CC(=O)NCC(=O)OCc6ccccc6)cs5)cc34)CC2)n1] |
| 5350 | 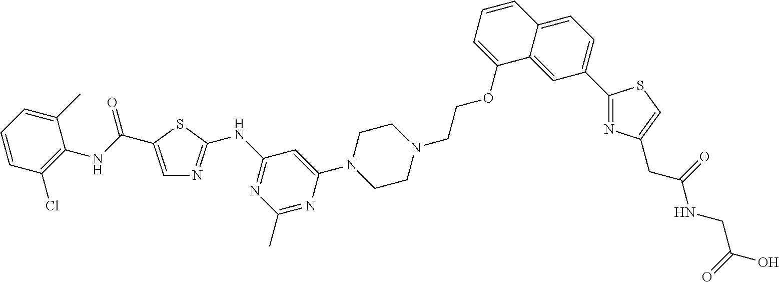 [SMILES: Cc1nc(Nc2ncc(C(=O)Nc3c(C)cccc3Cl)s2)cc(N2CCN(CCOc3cccc4ccc(-c5nc(CC(=O)NCC(=O)O)cs5)cc34)CC2)n1] |
| 5343 | 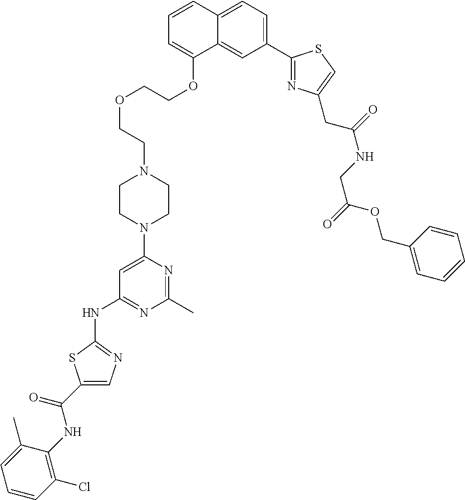 [SMILES: Cc1nc(Nc2ncc(C(=O)Nc3c(C)cccc3Cl)s2)cc(N2CCN(CCOCCOc3cccc4ccc(-c5nc(CC(=O)NCC(=O)OCc6ccccc6)cs5)cc34)CC2)n1] |
| 5375 | 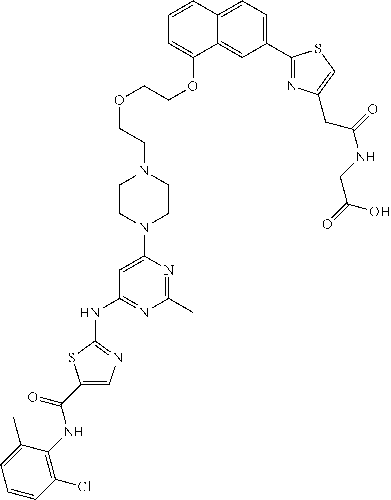 [SMILES: Cc1nc(Nc2ncc(C(=O)Nc3c(C)cccc3Cl)s2)cc(N2CCN(CCOCCOc3cccc4ccc(-c5nc(CC(=O)NCC(=O)O)cs5)cc34)CC2)n1] |
| 5376 | 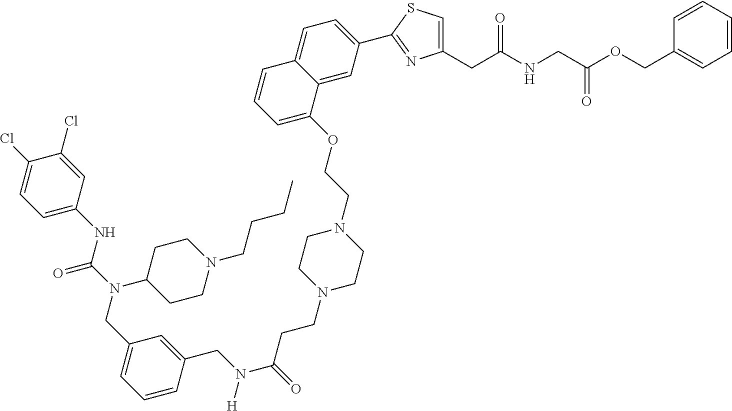 [SMILES: CCCCN1CCC(N(Cc2cccc(CNC(=O)CCN3CCN(CCOc4cccc5ccc(-c6nc(CC(=O)NCC(=O)OCc7ccccc7)cs6)cc45)CC3)c2)C(=O)Nc2ccc(Cl)c(Cl)c2)CC1] |
| 5377 | 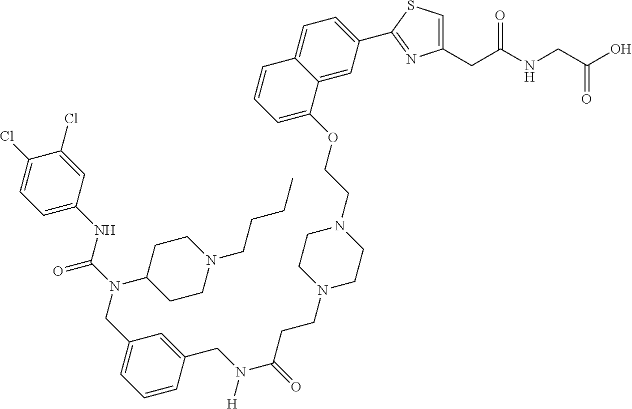 [SMILES: CCCCN1CCC(N(Cc2cccc(CNC(=O)CCN3CCN(CCOc4cccc5ccc(-c6nc(CC(=O)NCC(=O)O)cs6)cc45)CC3)c2)C(=O)Nc2ccc(Cl)c(Cl)c2)CC1] |
| 5392 | 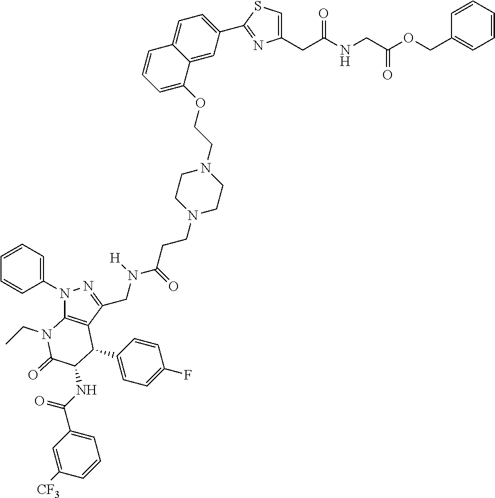 [SMILES: CCN1C(=O)[C@@H](NC(=O)c2cccc(C(F)(F)F)c2)[C@@H](c2ccc(F)cc2)c2c(CNC(=O)CCN3CCN(CCOc4cccc5ccc(-c6nc(CC(=O)NCC(=O)OCc7ccccc7)cs6)cc45)CC3)nn(-c3ccccc3)c21] |
| 5393 | 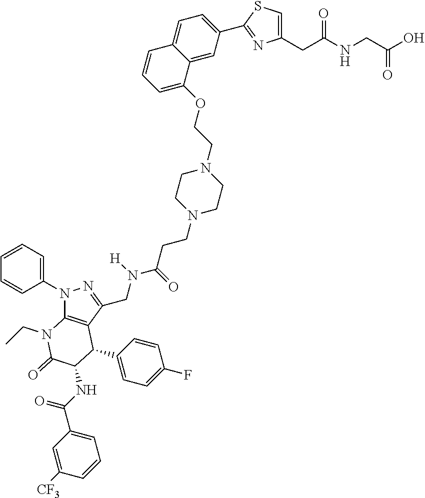 [SMILES: CCN1C(=O)[C@@H](NC(=O)c2cccc(C(F)(F)F)c2)[C@@H](c2ccc(F)cc2)c2c(CNC(=O)CCN3CCN(CCOc4cccc5ccc(-c6nc(CC(=O)NCC(=O)O)cs6)cc45)CC3)nn(-c3ccccc3)c21] |
| 5397 | 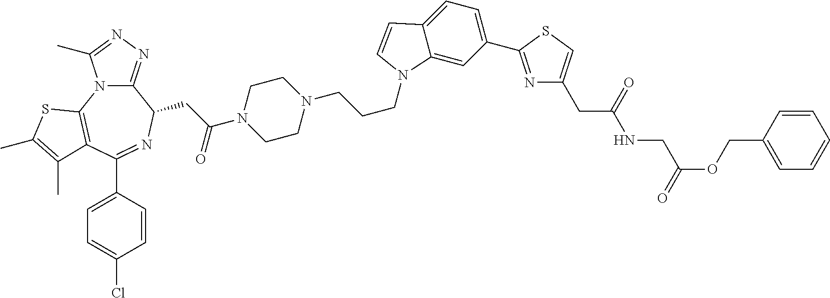 [SMILES: Cc1sc2c(c1C)C(c1ccc(Cl)cc1)=N[C@@H](CC(=O)N1CCN(CCCn3ccc4ccc(-c5nc(CC(=O)NCC(=O)OCc6ccccc6)cs5)cc43)CC1)c1nnc(C)n1-2] |
| 5399 | 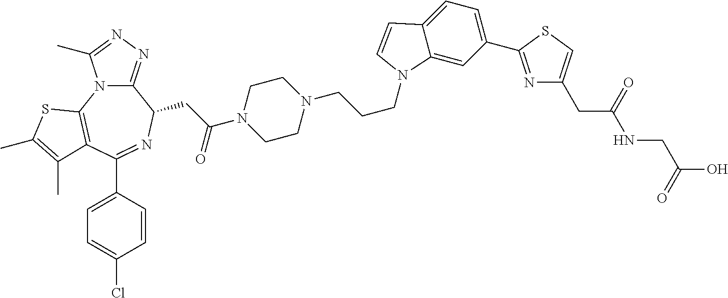 [SMILES: Cc1sc2c(c1C)C(c1ccc(Cl)cc1)=N[C@@H](CC(=O)N1CCN(CCCn3ccc4ccc(-c5nc(CC(=O)NCC(=O)O)cs5)cc43)CC1)c1nnc(C)n1-2] |

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having a structure represented by a formula: 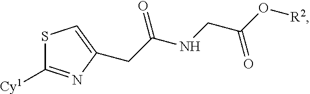
wherein R² is selected from hydrogen, C1-C4 alkyl, C4-C6 cycloalkyl, —(C1-C4 alkyl)OC(O)(C1-C4 alkyl), and —CH₂C₆H₅;
wherein Cy¹ is a 9- or 10-membered heterobicycle, a 10-membered biaryl, or a 9- or 10-membered heterobiaryl, and is substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹;
wherein each occurrence of R¹⁰, when present, is independently selected from hydrogen, C1-C4 alkyl, —(C1-C4 alkyl)O(C1-C4 alkyl), —(C1-C4 alkyl)O(C1-C4 alkyl)N₃, and —(C1-C4 alkyl)Cy²;
wherein Cy², when present, is a C2-C5 heterocycloalkyl substituted with 0, 1, 2, or 3 groups independently selected from halogen, —N₃, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 alkoxy, C1-C4 alkylamino, (C1-C4)(C1-C4) dialkylamino, C1-C4 aminoalkyl, —C(O)H, —C(O)(C1-C4 alkyl), —CO₂H, and —CO₂(C1-C4 alkyl);
wherein each occurrence of R^{D}, when present, is independently selected from C1-C4 alkyl, C1-C4 hydroxyalkyl, and Ar¹; and
wherein Ar¹, when present, is a C6 aryl or a C2-C5 heteroaryl, and is substituted with 0, 1, 2, or 3 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl,
or a pharmaceutically acceptable salt thereof.

2. The compound of claim 1, wherein R² is hydrogen.

3. The compound of claim 1, wherein Cy¹ is a 10-membered heterobicycle, a 10-membered biaryl, or a 9- or 10-membered heterobiaryl, and is substituted with 0, 1, 2, 3, or 4 groups independently selected from halogen, C1-C4 alkyl, —OR¹⁰, and —NHC(O)R¹¹.

4. The compound of claim 1, wherein the compound has a structure represented by a formula selected from: 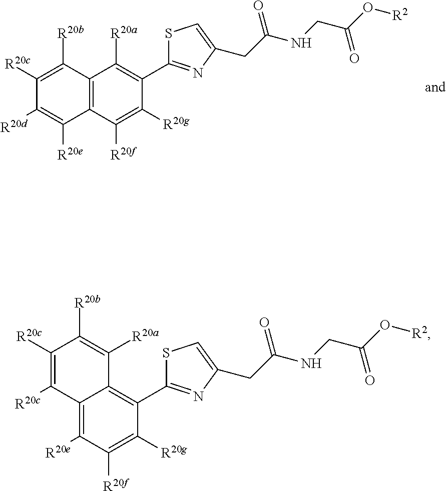
wherein each of R^{20a}, R^{20b}, R^{20c}, R^{20d}, R^{20e}, R^{20f}, and R^{20g} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹, provided that at least three of R^{20a}, R^{20b}, R^{20c}, R^{20d}, R^{20e}, R^{20f}, and R^{20g} are hydrogen,
or a pharmaceutically acceptable salt thereof.

5. The compound of claim 1, wherein the compound has a structure represented by a formula selected from: 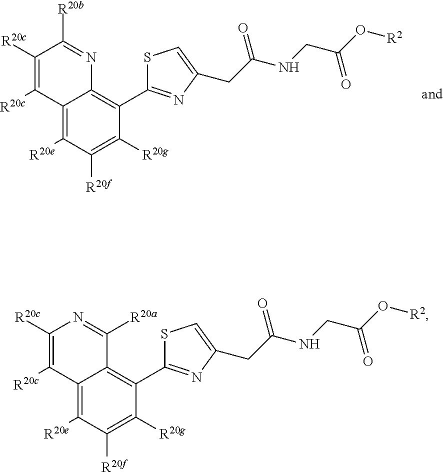
wherein each of R^{20a}, R^{20b}, R^{20c}, R^{20d}, R^{20e}, R^{20f}, and R^{20g} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹, provided that at least two of R^{20a}, R^{20b}, R^{20c}, R^{20d}, R^{20e}, R^{20f}, and R^{20g} are hydrogen,
or a pharmaceutically acceptable salt thereof.

6. The compound of claim 1, wherein the compound has a structure represented by a formula: 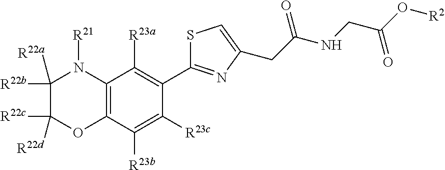
wherein R²¹ is selected from hydrogen and C1-C4 alkyl; and
wherein each of R^{22a}, R^{22b}, R^{22c}, R^{22d}, R^{23a}, R^{23b}, and R^{23c} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹,
provided that at least three of R²¹, R^{22a}, R^{22b}, R^{22c}, R^{22d}, R^{23a}, R^{23b}, and R^{23c} are hydrogen,
or a pharmaceutically acceptable salt thereof.

7. The compound of claim 1, wherein the compound has a structure represented by a formula: 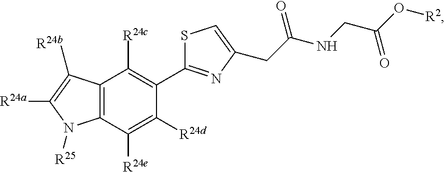
wherein each of R^{24a}, R^{24b}, R^{24c}, R^{24d}, and R^{24e} is independently selected from hydrogen, halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹; and
wherein R²⁵ is selected from hydrogen and C1-C4 alkyl,
provided that at least two of R^{24a}, R^{24b}, R^{24c}, R^{24d}, R^{24e}, and R²⁵ are hydrogen,
or a pharmaceutically acceptable salt thereof.

8. The compound of claim 1, wherein the compound is selected from: 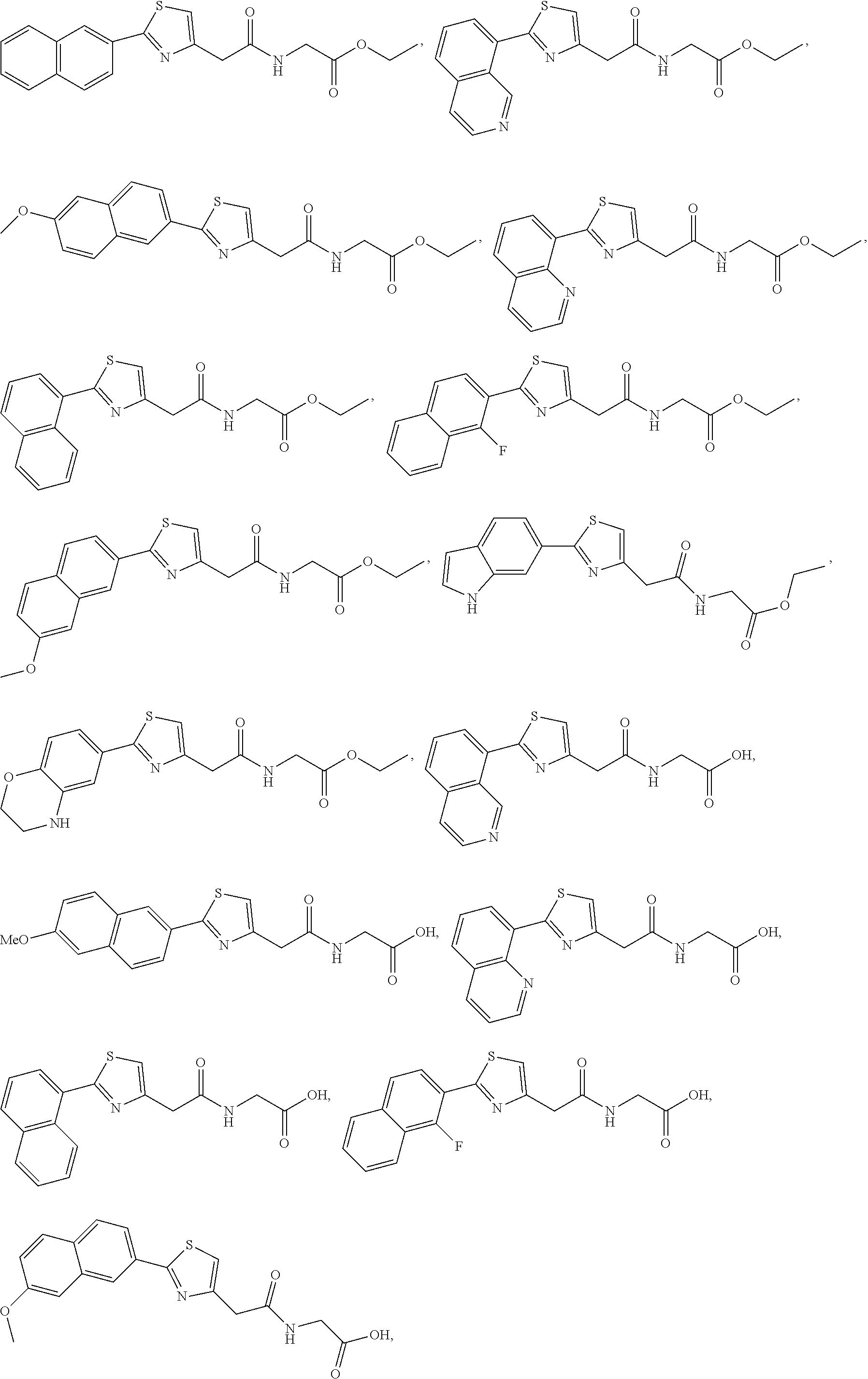 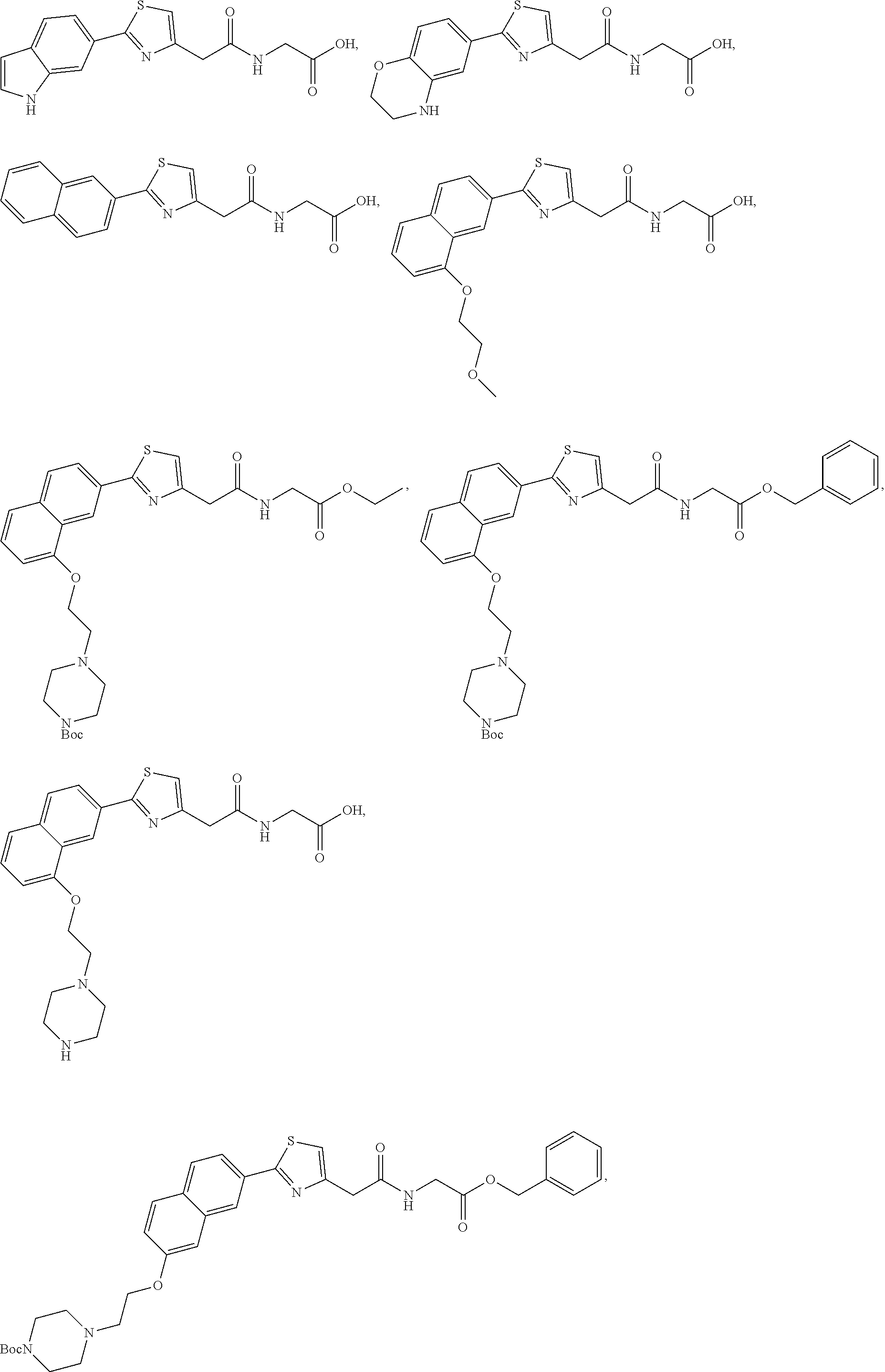 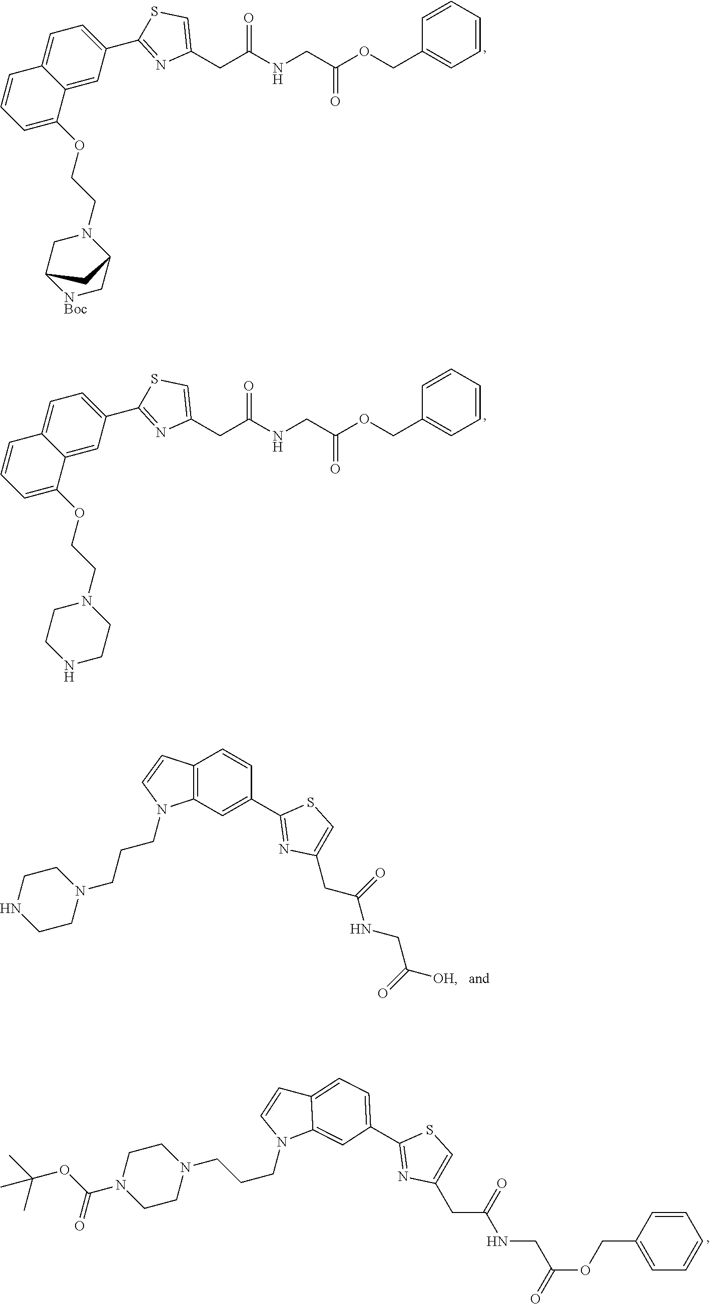
or a pharmaceutically acceptable salt thereof.

9. A compound having a structure represented by a formula: 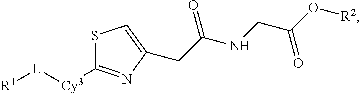
wherein L is a linker;
wherein R¹ is a residue of a small molecule having a molecular weight of from about 150 g/mol to about 600 g/mol and a binding affinity (Kᵢ) to a target protein of at least about 20 μM;
wherein R² is selected from hydrogen, C1-C4 alkyl, C4-C6 cycloalkyl, —(C1-C4 alkyl)OC(O)(C1-C4 alkyl), and —CH₂C₆H₅;
wherein Cy³ is a 9- or 10-membered heterobicycle, a 10-membered biaryl, or a 9- or 10-membered heterobiaryl, and is substituted with 0, 1, 2, 3, or 4 groups independently selected from halogen, —N₃, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, C1-C4 aminoalkyl, —OR¹⁰, and —NHC(O)R¹¹;
wherein each occurrence of R¹⁰, when present, is independently selected from hydrogen, C1-C4 alkyl, —(C1-C4 alkyl)O(C1-C4 alkyl), —(C1-C4 alkyl)O(C1-C4 alkyl)N₃, and —(C1-C4 alkyl)Cy²;
wherein Cy², when present, is a C2-C5 heterocycloalkyl substituted with 0, 1, 2, or 3 groups independently selected from halogen, —N₃, —CN, —NH₂, —OH, —NO₂, C1-C4 alkyl, C2-C4 alkenyl, C1-C4 haloalkyl, C1-C4 cyanoalkyl, C1—C4 hydroxyalkyl, C1-C4 alkoxy, C1-C4 alkylamino, (C1-C4)(C1-C4) dialkylamino, C1-C4 aminoalkyl, —C(O)H, —C(O)(C1-C4 alkyl), —CO₂H, and —CO₂(C1-C4 alkyl);
wherein each occurrence of R¹¹, when present, is independently selected from C1-C4 alkyl, C1-C4 hydroxyalkyl, and Ar¹;
wherein Ar¹, when present, is a C6 aryl or a C2-C5 heteroaryl, and is substituted with 0, 1, 2, or 3 groups independently selected from halogen, —CN, —NH₂, —NO₂, C1-C4 alkyl, C2-C4 alkenyl, C1-C4 haloalkyl, C1-C4 cyanoalkyl, C1-C4 hydroxyalkyl, C1-C4 alkylamino, (C1-C4)(C1-C4) dialkylamino, and C1-C4 aminoalkyl,
or a pharmaceutically acceptable salt thereof.

10. The compound of claim 9, wherein L is a structure represented by a formula: 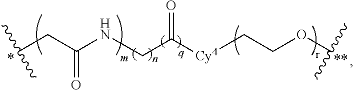
wherein * is connected to R¹, and ** is connected to Cy³;
wherein m is 0 or 1;
wherein n is 1, 2, or 3;
wherein q is 0 or 1;
wherein r is 1, 2, 3, or 4; and
wherein Cy⁴ is a structure selected from:

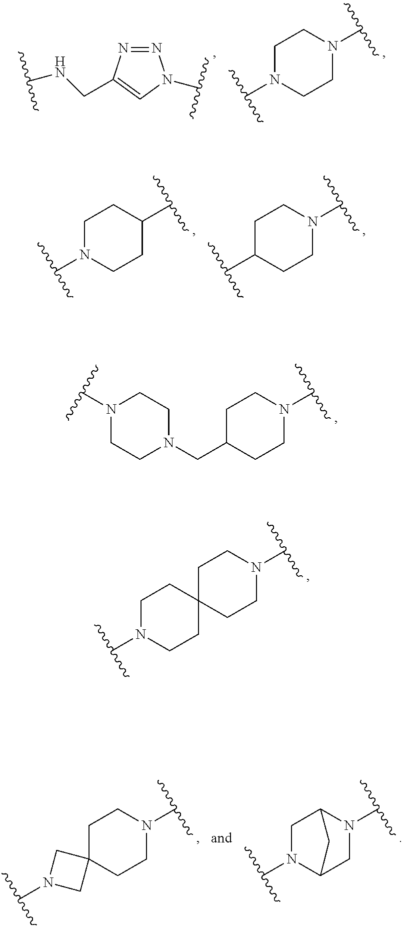

11. The compound of claim 9, wherein R¹ is a structure selected from:

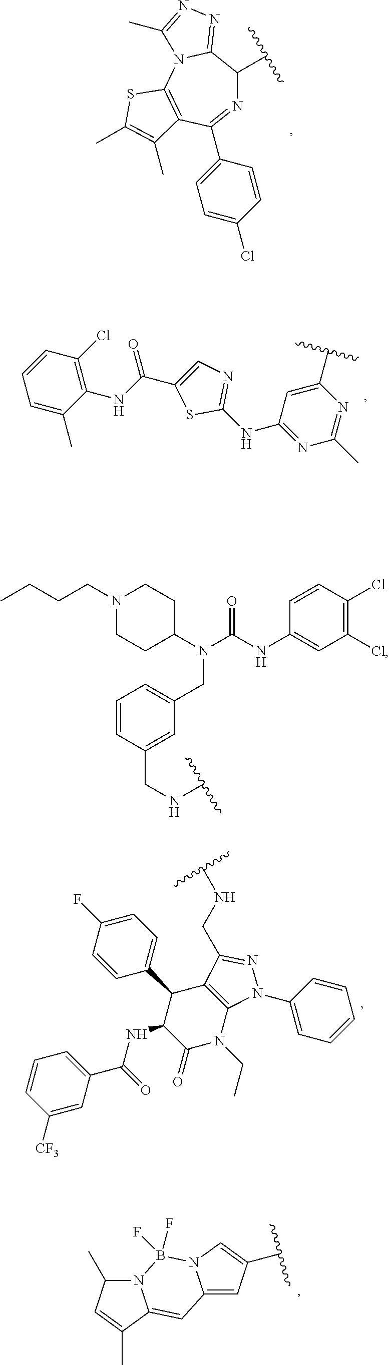

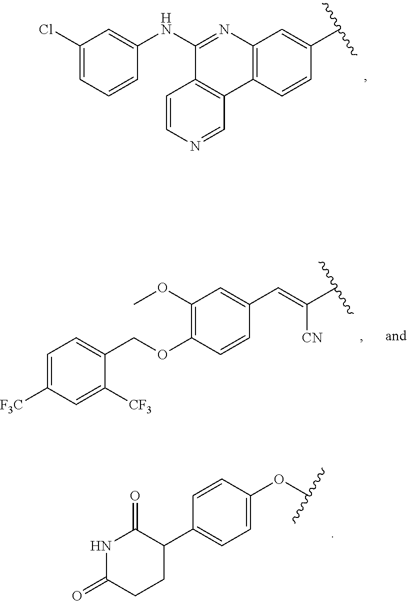 [SMILES: *c1ccc2c(c1)nc(Nc1cccc(Cl)c1)c1ccncc12 ; */C(C#N)=C/c1ccc(OCc2ccc(C(F)(F)F)cc2C(F)(F)F)c(OC)c1 ; *Oc1ccc(C2CCC(=O)NC2=O)cc1]

12. The compound of claim 9, wherein R² is selected from methyl, cyclopentyl, and —CH₂C₆H₅.

13. The compound of claim 9, wherein Cy³ is a 10-membered heterobicycle, a 10-membered biaryl, or a 10-membered heterobiaryl, and is substituted with 0, 1, or 2 groups independently selected from halogen, C1-C4 alkyl, —OR¹⁰, and —NHC(O)R¹¹.

14. The compound of claim 9, wherein the compound has a structure represented by a formula: 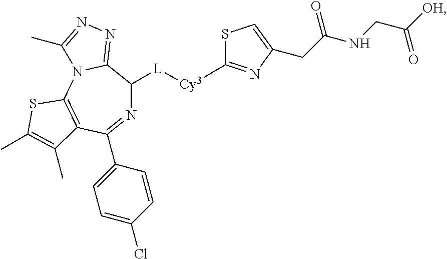
or a pharmaceutically acceptable salt thereof.

15. The compound of claim 9, wherein the compound is selected from: 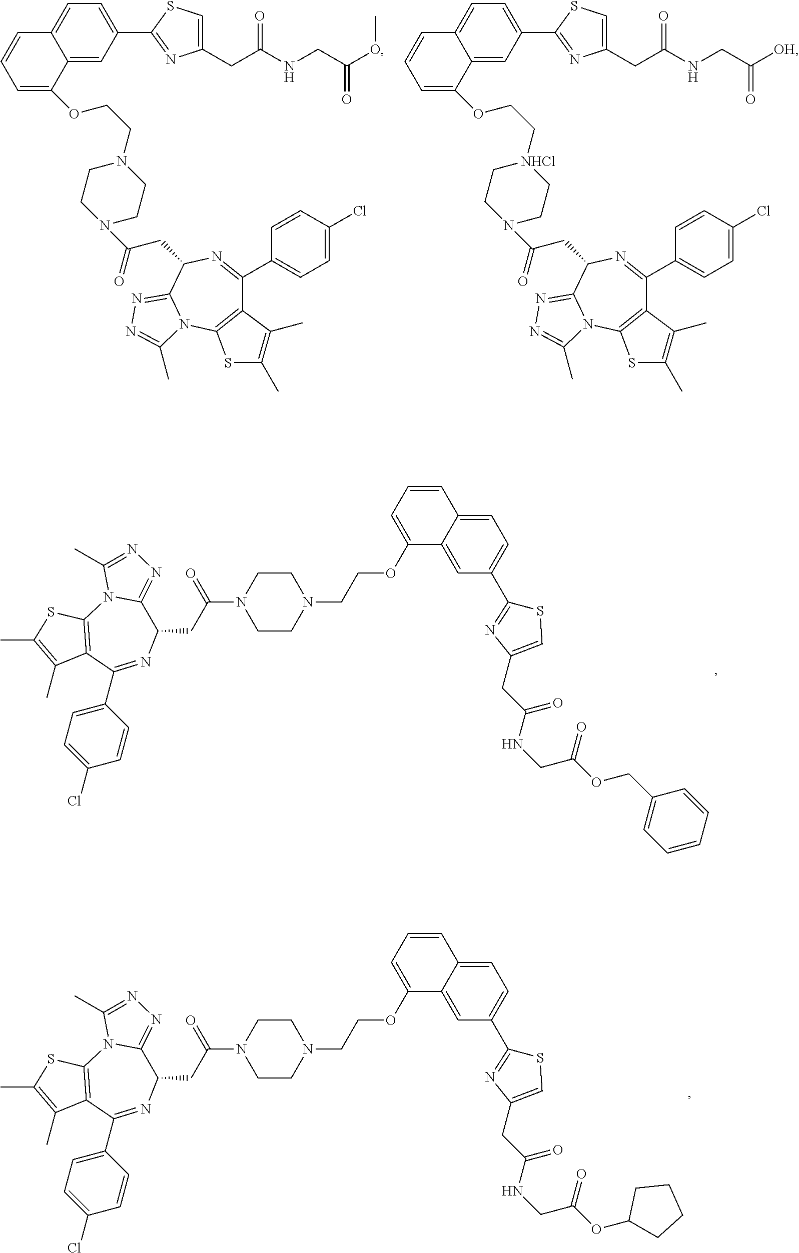 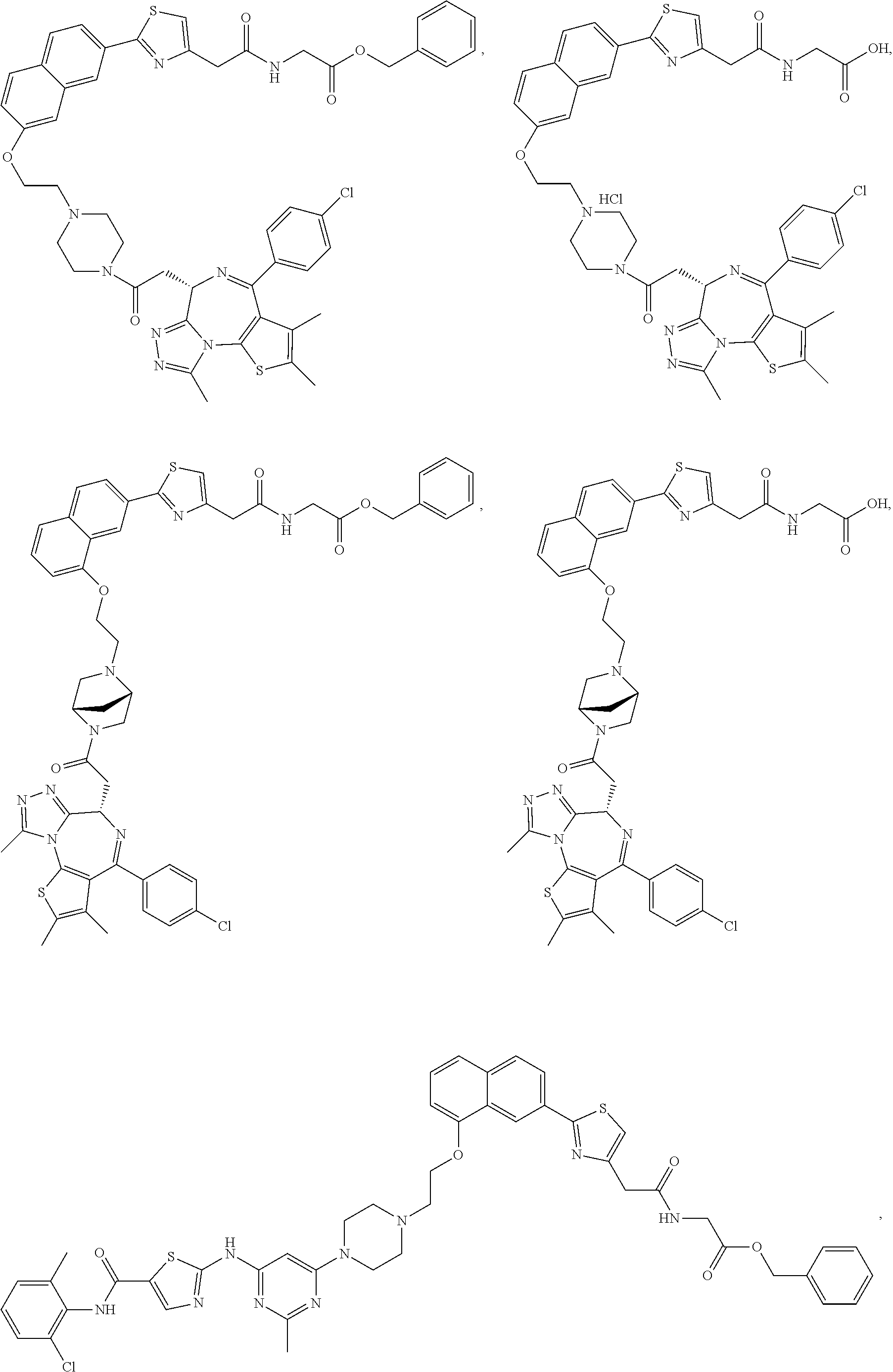 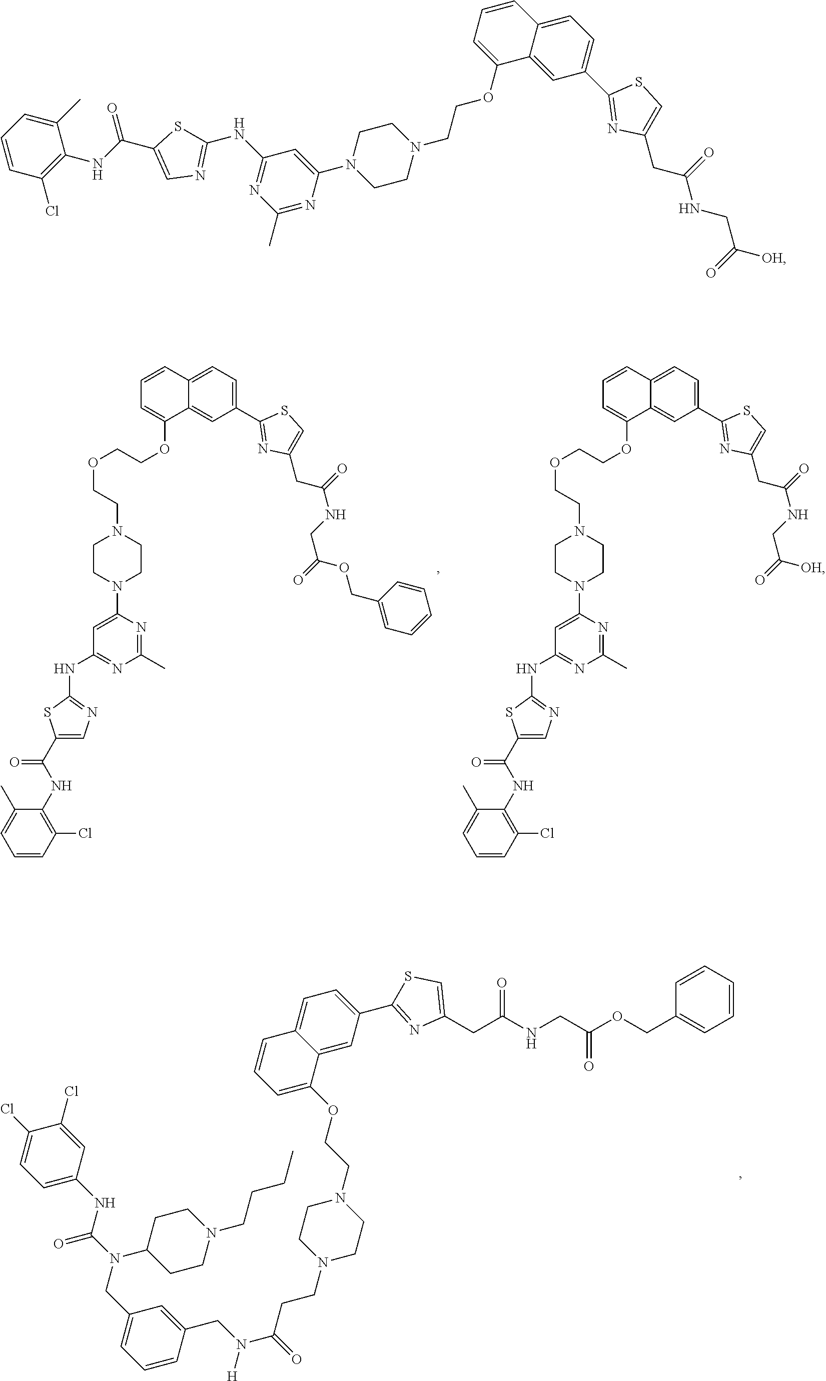 [SMILES: Cc1nc(Nc2ncc(C(=O)Nc3c(C)cccc3Cl)s2)cc(N2CCN(CCOc3cccc4ccc(-c5nc(CC(=O)NCC(=O)O)cs5)cc34)CC2)n1 ; Cc1nc(Nc2ncc(C(=O)Nc3c(C)cccc3Cl)s2)cc(N2CCN(CCOCCOc3cccc4ccc(-c5nc(CC(=O)NCC(=O)OCc6ccccc6)cs5)cc34)CC2)n1 ; Cc1nc(Nc2ncc(C(=O)Nc3c(C)cccc3Cl)s2)cc(N2CCN(CCOCCOc3cccc4ccc(-c5nc(CC(=O)NCC(=O)O)cs5)cc34)CC2)n1 ; CCCCN1CCC(N(Cc2cccc(CNC(=O)CCN3CCN(CCOc4cccc5ccc(-c6nc(CC(=O)NCC(=O)OCc7ccccc7)cs6)cc45)CC3)c2)C(=O)Nc2ccc(Cl)c(Cl)c2)CC1] 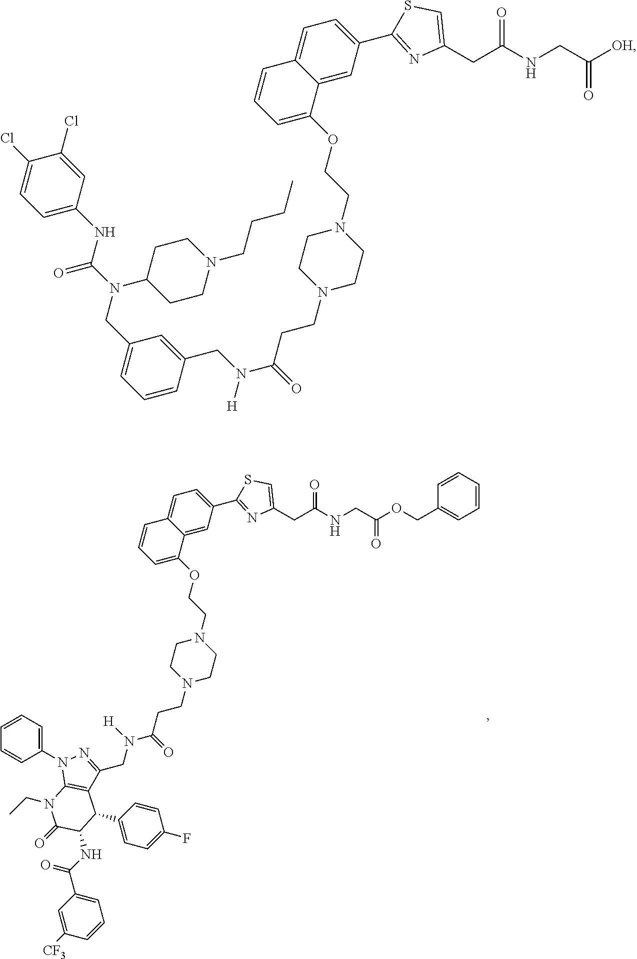 [SMILES: CCCCN1CCC(N(Cc2cccc(CNC(=O)CCN3CCN(CCOc4cccc5ccc(-c6nc(CC(=O)NCC(=O)O)cs6)cc45)CC3)c2)C(=O)Nc2ccc(Cl)c(Cl)c2)CC1 ; CCN1C(=O)[C@@H](NC(=O)c2cccc(C(F)(F)F)c2)[C@@H](c2ccc(F)cc2)c2c(CNC(=O)CCN3CCN(CCOc4cccc5ccc(-c6nc(CC(=O)NCC(=O)OCc7ccccc7)cs6)cc45)CC3)nn(-c3ccccc3)c21] 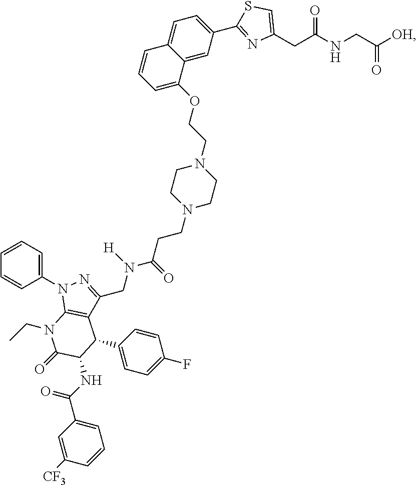 [SMILES: CCN1C(=O)[C@@H](NC(=O)c2cccc(C(F)(F)F)c2)[C@@H](c2ccc(F)cc2)c2c(CNC(=O)CCN3CCN(CCOc4cccc5ccc(-c6nc(CC(=O)NCC(=O)O)cs6)cc45)CC3)nn(-c3ccccc3)c21] 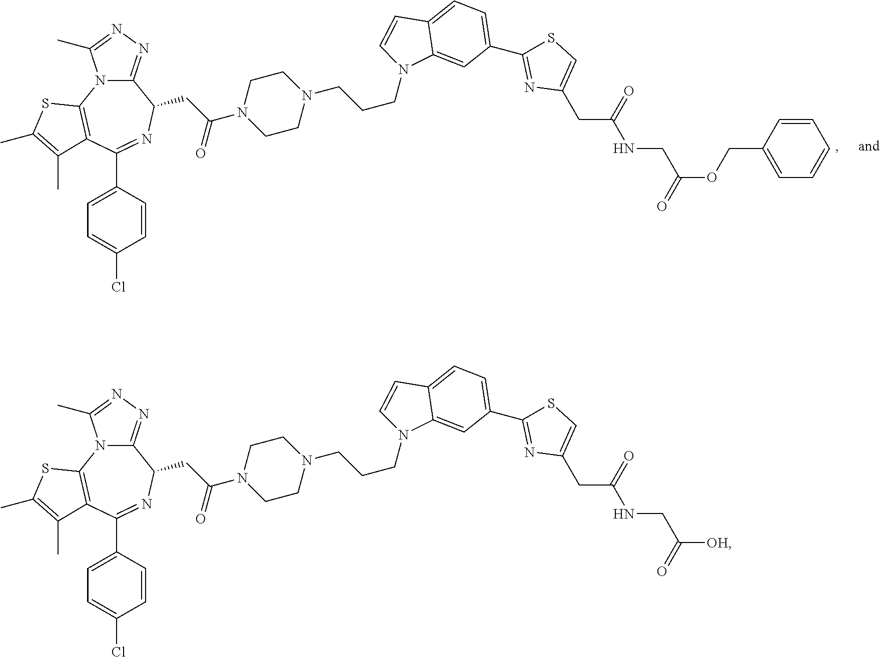 [SMILES: Cc1sc2c(c1C)C(c1ccc(Cl)cc1)=N[C@@H](CC(=O)N1CCN(CCCn3ccc4ccc(-c5nc(CC(=O)NCC(=O)OCc6ccccc6)cs5)cc43)CC1)c1nnc(C)n1-2 ; Cc1sc2c(c1C)C(c1ccc(Cl)cc1)=N[C@@H](CC(=O)N1CCN(CCCn3ccc4ccc(-c5nc(CC(=O)NCC(=O)O)cs5)cc43)CC1)c1nnc(C)n1-2]
or a pharmaceutically acceptable salt thereof.

16. A pharmaceutical composition comprising a therapeutically effective amount of the compound of claim 9 or a pharmaceutically acceptable salt thereof, and a pharmaceutically acceptable carrier.

17. A method of treating a disorder of uncontrolled cellular proliferation in a subject in need thereof, the method comprising administering to the subject an effective amount of the compound of claim 9 or a pharmaceutically acceptable salt thereof.

18. The method of claim 17, wherein the disorder is a cancer.

19. The method of claim 18, wherein the cancer is leukemia.

20. A method of degrading a target protein in a subject in need thereof, the method comprising administering to the subject an effective amount of the compound of claim 9.
